(19) United States
(12) Patent Application Publication
(10) Pub. No.: US 2022/0153746 A1
(43) Pub. Date: May 19, 2022

(54) NITROGEN OXIDE-DONATING PDE-5 AND/OR PDE-6 INHIBITOR COMPOUNDS, AND USES THEREOF

(71) Applicant: ILDONG PHARMACEUTICAL CO., LTD., Seoul (KR)

(72) Inventors: Yoon-Suk LEE, Hwaseong-si (KR); Kyung-Sun KIM, Hwaseong-si (KR); Jeong-Ah KIM, Hwaseong-si (KR); An-Na MOON, Hwaseong-si (KR); Dong-Keun SONG, Hwaseong-si (KR); Ju-Young JUNG, Hwaseong-si (KR)

(21) Appl. No.: 17/521,577

(22) Filed: Nov. 8, 2021

Related U.S. Application Data
(60) Provisional application No. 63/111,374, filed on Nov. 9, 2020.

(51) Int. Cl. C07D 487/04; A61P 27/06

(52) U.S. Cl. CPC C07D 487/04; A61P 27/06

(57) ABSTRACT

The present disclosure provides uses of phosphodiesterase 5 (PDE-5) and/or phosphodiesterase 6 (PDE-6) inhibitor compounds and uses of compositions including said compounds. In some embodiments, said compounds are nitrogen oxide (NO) donating PDE-5 and/or -6 inhibitor compounds that include a nitrogen oxide-containing donor substituent attached to a benzenesulfonamide group. The compounds can provide dual functionality for increasing protein kinase G (PKG) activity by inhibiting PDE-5 and PDE-6, and/or stimulating guanylate cyclase via donation of NO from the donor substituent of the compound. The present disclosure also provides methods of using said compounds and compositions for inhibiting PDE-5 and/or -6 and increasing activity of PKG. The compounds and compositions find use in the treatment of a variety of eye diseases. For example, the subject compounds may be used as a therapeutic agent for glaucoma, age-related macular degeneration, diabetic retinopathy, xerophthalmia, dry eye syndrome, cataracts or uveitis.

### 1. CROSS REFERENCE TO RELATED APPLICATIONS

This application claims the benefit of U.S. Provisional Application No. 63/111,374, filed Nov. 9, 2020, which is hereby incorporated in its entirety by reference.

### 2. BACKGROUND OF THE INVENTION

Vision refers to the sense of cognition through the eyes, and the ocular structure and processes for transmitting visual information are highly important. The front surface of the eye is comprised of the conjunctiva and the cornea, and within the sclera which surrounds the eyeball are the iris, ciliary bodies, the lens, vitreous body and the retina. Light which enters through the cornea is refracted by the lens, then passes through the vitreous body and creates an image on the retina which is delivered to the brain through the optic nerve. Humans cognize objects through the physiological process of visual information being transmitted from the eyes to the brain. Aging causes various degenerative changes in the eyeball. For example, 90% of macular degeneration cases are reported to be dry age-related macular degeneration which causes atrophy of photoreceptors in the retina. Exemplary degenerative diseases of the eye include macular degeneration, glaucoma and cataracts. Further, with the increase in time spent in front of computers and use of smart phones, prevalence of eye conditions such as xerophthalmia is continually rising.

Many diseases require invasive eye surgery or highly difficult surgery such as laser surgery for treatment. Eye diseases can be difficult to recover from once the eye has been damaged, and with the exception of eye drops for xerophthalmia, most therapeutic agents for eye disease are administered in injection form. Such injections may cause pain or hypersensitive reaction around the injection site, and due to the tedious method of administration, patient compliance is low. Therefore, for treatment of eye diseases, it is desirable to reduce the burden of drug administration on patients and improve compliance. Further, for treatment and alleviation of symptoms of eye diseases from which recovery is difficult, it is desirable to identify new therapeutic targets.

### 3. SUMMARY OF THE INVENTION

The present disclosure provides uses of phosphodiesterase 5 (PDE-5) and/or phosphodiesterase 6 (PDE-6) inhibitor compounds and uses of compositions including said compounds. In some embodiments, said compounds are nitrogen oxide (NO) donating PDE-5 and/or -6 inhibitor compounds that include a nitrogen oxide-containing donor substituent attached to a benzenesulfonamide group. The compounds can provide dual functionality for increasing protein kinase G (PKG) activity by inhibiting PDE-5 and PDE-6, and/or stimulating guanylate cyclase (sGC) via donation of nitrogen oxide (NO) from the donor substituent of the compound. The present disclosure also provides methods of using said compounds and compositions for inhibiting PDE-5 and/or -6 and increasing activity of protein kinase G (PKG). The compounds and compositions find use in therapeutic applications including in the treatment of a variety of eye diseases. For example, the subject compounds may be used as a therapeutic agent for glaucoma, age-related macular degeneration (AMD), diabetic retinopathy (DR), xerophthalmia, dry eye syndrome (DES), cataracts or uveitis. Also provided are methods of preparing said compounds and compositions, and synthetic precursors of said compounds.

In a first aspect, the present disclosure provides a method of treating an eye disease, the method comprising administering to an eye of a subject a therapeutically effective amount of a compound an ophthalmic composition comprising the compound, wherein the compound is of formula (I):

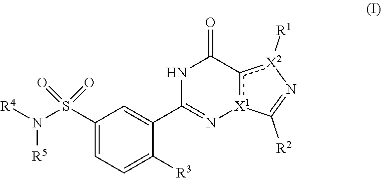

or a pharmaceutically acceptable salt, a solvate, a hydrate, a prodrug, or a stereoisomer thereof, wherein:

X¹ and X² are independently selected from N and C and at least one of X¹ and X² is N;

R¹ is H, or optionally substituted (C₁-C₅)alkyl;

R² is optionally substituted (C₁-C₅)alkyl;

R³ is optionally substituted (C₁-C₅)alkoxy;

R⁴ is —H or optionally substituted (C₁-C₅)alkyl, and R⁵ is a 4-membered carbocycle or heterocycle ring that is substituted with one or more R⁶,

or R⁴ and R⁵ together with the nitrogen atom to which they are attached are cyclically linked to form a 4-membered heterocycle that is substituted with one or more R⁶; and

and each R⁶ is independently selected from —O—NO₂, —OH, optionally substituted (C₁-C₅)alkyl, optionally substituted (C₁-C₁₀)alkylene, optionally substituted (C₂-C₁₀)alkenyl, optionally substituted (C₂-C₁₀)alkynyl, optionally substituted (C₁-C₅)alkoxy, optionally substituted (C₃-C₅)heterocycle, optionally substituted (C₁-C₅)alkyl-(C₃-C₅)heterocycle-, optionally substituted (C₃-C₅)heterocycle-(C₁-C₅)alkyl-, optionally substituted (C₁-C₅)alkyl-Z¹—(C₁-C₅)alkyl-, optionally substituted (C₁-C₅)alkyl-Z¹—(C₁-C₅)alkoxy-, optionally substituted (C₁-C₁₀)alkyl-NR¹—, optionally substituted (C₁-C₁₀)alkyl-Z¹—(C₁-C₅)alkyl-NR¹—, optionally substituted (C₁-C₁₀)alkoxy-Z¹—(C₁-C₅)alkyl-NR¹—, substituted (C₁-C₅)alkyl-(C₃-C₅)heterocycle-(C₁-C₅)alkyl-, substituted linear linker, and substituted branched linker, wherein Z¹ is —CO₂—, —O—, —OCO—, —CONH—, —NHCO—, or —NH—, and the substituents of each R⁶ are independently selected from —O—NO₂, —ONO, —OH, —NH₂, —COOH, halogen, (C₁-C₃)alkoxy and (C₁-C₃)alkyl.

In a second aspect there is provided a method of treating a PDE-5 and/or -6-related indication, the method comprising administering to a subject in need thereof a therapeutically effective amount of a compound of formula (I) or a pharmaceutically acceptable salt, a solvate, a hydrate, a prodrug, or a stereoisomer thereof.

It is understood that all variations of salts, solvates, hydrates, prodrugs and/or stereoisomers of the compounds of formula (I)-(IIIb) are meant to be encompassed by the present disclosure. The present disclosure is also meant to encompass compounds of formula (I)-(IIIb), or a salt (e.g., pharmaceutically acceptable salt) thereof, including a single stereoisomer, a mixture of stereoisomers and/or an isotopically labelled form of compounds of formula (I)-(IIIb), e.g., as described in any one of the embodiments herein.

In some embodiments of the compound of formula (I), wherein at least one R⁶ is substituted with —O—NO₂, —ONO, —OH or —NH₂.

In some embodiments, the PDE-5 and/or -6 inhibitor compound is a NO-donating PDE-5 and/or -6 inhibitor compound, and at least one R⁶ is substituted with —O—NO₂.

In some embodiments of the compound of formula (I), at least one R⁶ is substituted with —OH or —NH₂.

In some embodiments, R⁴ is —H and R⁵ is a substituted azetidine.

In some embodiments, R⁴ and R⁵ together with the nitrogen atom to which they are attached are cyclically linked to form a substituted azetidine.

In some embodiments, the compound of formula (I) is a compound of formula (II):

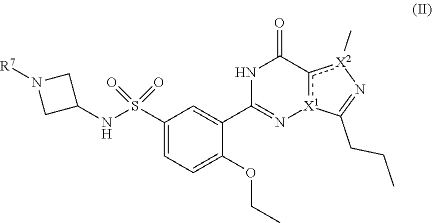

or a pharmaceutically acceptable salt, a solvate, a hydrate, a prodrug, or a stereoisomer thereof, wherein:

R⁷ is selected from —H, R⁷⁰, and R⁷¹—Z²—R⁷²;

R⁷⁰, R⁷¹ and R⁷² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, and optionally substituted (C₁-C₅)alkoxy, wherein the optional substituent is selected from —OH, —NH₂, and —O—NO₂; and

Z² is —CO₂—, —O—, —OCO—, —CONH—, —NHCO—, or —NH—.

In some embodiments of formula (II), Z² is —CO₂—, —OCO—, —O—, —CONH—, or —NH—.

In some embodiments, the compound of formula (I) is a compound of formula (III):

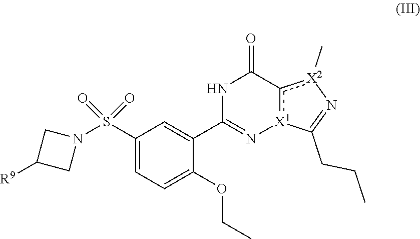

or a pharmaceutically acceptable salt a solvate, a hydrate, a prodrug, or a stereoisomer thereof wherein:

R⁹ is selected from —O—NO₂, —NR¹⁰R¹¹, —OR¹², R⁹⁰, and R⁹¹—Z³—R⁹²;

R⁹⁰, R⁹¹ and R⁹² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, optionally substituted (C₁-C₅)alkoxy, and optionally substituted (C₃-C₅)heterocycle-(C₁-C₅)alkyl-, and optionally substituted (C₁-C₅)alkyl-(C₃-C₅)heterocycle-(C₁-C₅)alkyl-, wherein the optional substituent is selected from —OH, —NH₂, and —O—NO₂;

Z³ is —CO₂—, —O—, —OCO—, —CONH—, —NHCO—, or —NH—; and

R¹⁰, R¹¹, and R¹² are independently —H, optionally substituted (C₁-C₅)alkyl, or optionally substituted (C₁-C₅)alkyl-Z¹—(C₁-C₅)alkyl-, wherein the optional substituent is selected from —OH, —NH₂, and —O—NO₂;

or R¹⁰ and R¹¹ together with the nitrogen atom to which they are attached are cyclically linked to form an optionally substituted heterocycle, wherein the optional substituent is selected from —OH, —O—NO₂. —CH₂OH, —CH₂CH₂OH, and —CH₂O—NO₂.

In some embodiments of formula (I)-(III), X¹ is N and X² is C.

In some embodiments of formula (I)-(III), X¹ is C and X² is N.

In a third aspect, the present disclosure provides a compound selected from compounds 26 to 73 of Table 1, or a pharmaceutically acceptable salt, a solvate, a hydrate, a prodrug, or a stereoisomer thereof.

In a four aspect, the present disclosure provides a pharmaceutical composition comprising a compound selected from compounds 26 to 73 of Table 1, or a pharmaceutically acceptable salt, a solvate, a hydrate, a prodrug, or a stereoisomer thereof, and a pharmaceutically acceptable excipient.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows the results of an intraocular pressure (IOP) lowering effect study with latanoprostene bunod (0.024%) in ocular normotensive rabbits at various time points after instillation of ophthalmic solutions. The left eyes of the tested animals in each group were dosed with vehicle solution at 50 μL per eye (control), while the right eyes received the same volume of a solution of the test compound (treatment).

FIG. 2 shows the results of the IOP lowering effect study with latanoprost (0.005%) in ocular normotensive rabbits at various time points after instillation of ophthalmic solutions (control solutions into left eyes and treatment solution into right eyes).

FIG. 3 shows the results of the IOP lowering effect study with exemplary compound 18 (10 mg/mL) in rabbits at various time points after instillation of ophthalmic solutions (control solutions into left eyes and treatment solution into right eyes).

FIG. 4 shows the results from test group 4 of the IOP lowering effect study with exemplary compound 18 (20 mg/mL) in rabbits at various time points after instillation of ophthalmic solutions (control solutions into left eyes and treatment solution into right eyes).

FIGS. 5A-5D show the change in IOP before and after treatment in each of groups 1-4 (normotensive rabbits) of an in vivo rabbit model (Mean±SEM), where * represents T test for pairs in each group. Comparison between right eye and left eye is P≤0.05; ** represents T test for pairs in each group, and P≤0.01 for comparison between right eye and left eye; and *** represents T test for pairs in each group, and P≤0.001 for comparison between right and left eyes. FIG. 5A shows the change in IOP before and after treatment with 0.024% latanoprostene bunod on day 1 (group 1). FIG. 5B shows the change in IOP before and after treatment with 0.005% latanoprostene bunod on day 1 (group 2). FIG. 5C shows the change in IOP before and after treatment with 10 mg/mL of compound 18 on day 1 (group 3). FIG. 5D shows the change in IOP before and after treatment with 20 mg/mL of compound 18 on day 1 (group 3).

FIGS. 6A-6D show the change in IOP before and after treatment in each of groups 5-8 (hypertensive rabbits) of an in vivo rabbit model (Mean±SEM), where * represents T test for pairs in each group. Comparison between right eye and left eye is P≤0.05. FIG. 6A shows the change in IOP before and after treatment with 0.024% latanoprostene bunod (group 5). FIG. 6B shows the change in IOP before and after treatment with 0.005% latanoprostene bunod (group 6). FIG. 6C shows the change in IOP before and after treatment with 10 mg/mL of compound 18 (group 7). FIG. 6D shows the change in IOP before and after treatment with 20 mg/mL of compound 18 (group 8).

FIG. 7 illustrates pharmacokinetics (PK) of exemplary NO-donating compound 18 and its metabolite compound 4. The graph shows mean plasma concentrations (Compound 18 or Compound 4) versus time on day 1 or day 7 for Group 2 animals in a study of Compound 18 following once daily treatment in rabbits for 7 consecutive days.

FIGS. 8-17 show the results of a tissue distribution (TD) phase of from a study of exemplary NO-donating compound 18 and its metabolite compound 4 following once daily topical application of compound 18 in Group 1 and 3 rabbits for 7 consecutive days. FIG. 8 illustrates a graph of mean plasma concentrations (Compound 18 and Compound 4) versus time for Group 1 and 3 rabbits for 7 consecutive days. Similarly, FIG. 9 (mean aqueous humor concentrations), FIG. 10 (mean vitreous humor concentrations), FIG. 11 (mean cornea concentrations), FIG. 12 (mean iris/ciliary concentrations), FIG. 13 (mean lens concentrations), FIG. 14 (mean optic nerve concentrations), FIG. 15 (mean retina concentrations), FIG. 16 (mean sclera/choroid concentrations), and FIG. 17 (mean trabecular meshwork concentrations) show graphs of Compound 18 and Compound 4 concentrations in various tissue over time for Groups 1 and 3 in the TD phase of the study.

FIG. 18 illustrates the distribution of Compound 18 and Compound 4 in plasma, aqueous humor, and vitreous humor versus time in the TD phase of the rabbit study.

FIG. 19 illustrates the distribution of Compound 18 in sclera/choroid, cornea, iris/ciliary body, lens, optic nerve, retina, and trabecular meshwork tissue over time in the TD phase of the rabbit study.

FIG. 20 illustrates the distribution of Compound 4 in sclera/choroid, cornea, iris/ciliary body, lens, optic nerve, retina, and trabecular meshwork tissue over time in the TD phase of the rabbit study.

### 5. DETAILED DESCRIPTION

### 5.1. PDE-5 and/or -6 Inhibitor Compounds

As summarized above, the present disclosure provides benzenesulfonamide containing compounds and compositions for use in inhibiting PDE-5 and/or -6 and increasing PKG activity. The compounds can include a benzenesulfonamide group linked to a fused heteroaryl, such as a bicyclic core structure of 1,6-dihydro-7H-pyrazolo[4,3-d]pyrimidin-7-one

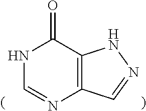

or a fused bicyclic core structure of imidazo[5,1-f][1,2,4]triazin-4(3H)-one

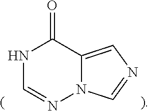

In the PDE-5 and/or -6 inhibitor compounds of the present disclosure, compounds containing the 1,6-dihydro-7H-pyrazolo[4,3-d]pyrimidine-7-one core can be substituted at the 5 position of the core structure with a substituted benzenesulfonamide group, and compounds containing the imidazo[5,1-f][1,2,4]triazin-4(3H)-one core can be substituted at the 2-position of the core structure with a substituted benzenesulfonamide group. In various embodiments as described herein, the benzenesulfonamide group may optionally be further substituted at the nitrogen. Compounds having such substituted benzenesulfonamide groups attached to the fused bicyclic cores of 1,6-dihydro-7H-pyrazolo[4,3-d]pyrimidin-7-one and imidazo[5,1-f][1,2,4]triazin-4(3H)-one described herein can have desirable biological activities (e.g., as described herein) and find use in a variety of therapeutic applications. The benzenesulfonamide group can be further substituted (e.g., at the nitrogen) with a substituent group comprising, e.g., one or more of an azetidine heterocycle ring and/or a short linear chain (e.g., an alkyl or alkoxy-alkyl chain).

The PDE-5 and/or -6 inhibitor compounds can further include a —O—NO₂ substituent to provide for a NO-donating PDE-5 and/or -6 inhibitor compound. Aspects of the present disclosure include dual action NO-donating and PDE-5 and/or -6 inhibiting compounds that are capable of stimulating guanylate cyclase (sGC) (e.g., via donation of nitrogen oxide (NO)) and inhibiting PDE-5 and/or -6. In some embodiments, the dual action compound provides a desirable synergic effect in activation of the PKG signaling pathway. Compounds containing a —O—NO₂ substituent can donate nitrogen oxide (NO, also known as nitric oxide) and leave behind a —OH group. The resulting —OH substituted compounds can also provide PDE-5 and/or -6 inhibition activity.

In a first aspect, the present disclosure provides a PDE-5 and/or -6 inhibitor compound of formula (I):

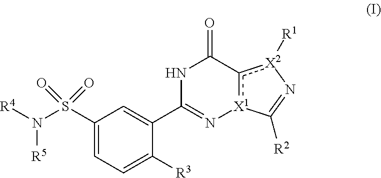

or a pharmaceutically acceptable salt, a solvate, a hydrate, a prodrug, or a stereoisomer thereof wherein:

X¹ and X² are independently selected from N and C and at least one of X¹ and X² is N;

R¹ is —H, or optionally substituted (C₁-C₅)alkyl;

R² is optionally substituted (C₁-C₅)alkyl;

R³ is optionally substituted (C₁-C₅)alkoxy;

R⁴ is —H or optionally substituted (C₁-C₅)alkyl, and R⁵ is a 4-membered carbocycle or heterocycle ring that is substituted with one or more R⁶,

or R⁴ and R⁵ together with the nitrogen atom to which they are attached are cyclically linked to form a 4-membered heterocycle that is substituted with one or more R⁶; and
and each R⁶ is independently selected from —OH, —O—NO₂, optionally substituted (C₁-C₅)alkyl, optionally substituted (C₁-C₁₀)alkylene, optionally substituted (C₂-C₁₀) alkenyl, optionally substituted (C₂-C₁₀)alkynyl, optionally substituted (C₁-C₅)alkoxy, optionally substituted (C₃-C₅)heterocycle, optionally substituted (C₁-C₅)alkyl-(C₃-C₅)heterocycle-, optionally substituted (C₃-C₅)heterocycle-(C₁-C₅)alkyl-, optionally substituted (C₁-C₅)alkyl-Z¹—(C₁-C₅)alkyl-, optionally substituted (C₁-C₅)alkyl-Z¹—(C₁-C₅)alkoxy-, optionally substituted (C₁-C₁₀)alkyl-NR¹—, optionally substituted (C₁-C₁₀)alkyl-Z¹—(C₁-C₅)alkyl-NR¹—, optionally substituted (C₁-C₁₀)alkoxy-Z¹—(C₁-C₅)alkyl-NR¹—, substituted (C₁-C₅)alkyl-(C₃-C₅)heterocycle-(C₁-C₅)alkyl-, substituted linear linker, and substituted branched linker, wherein Z¹ is —CO₂—, —O—, —OCO—, —CONH—, —NHCO—, or —NH—, and the substituents of each R⁶ are independently selected from —O—NO₂, —ONO, —OH, —NH₂, —COOH, halogen, (C₁-C₃)alkoxy and (C₁-C₃)alkyl.

In some embodiments, the PDE-5 and/or -6 inhibitor compound is a NO-donating PDE-5 and/or -6 inhibitor compound. In some embodiments of the compound of formula (I), at least one R⁶ is substituted with —O—NO₂.

In some embodiments of the compound of formula (I), wherein at least one R⁶ is substituted with —O—NO₂, —O—NO, —OH or —NH₂. In some embodiments of the compound of formula (I), at least one R⁶ is substituted with —OH or —NH₂.

In some embodiments of formula (I), R¹ is (C₁-C₅)alkyl. In another embodiment, R¹ is methyl.

In some embodiments of formula (I), R² is n-propyl.

In some embodiments of formula (I), R³ is ethoxy.

In some embodiments, the compound of formula (I) is a compound of formula (Ia):

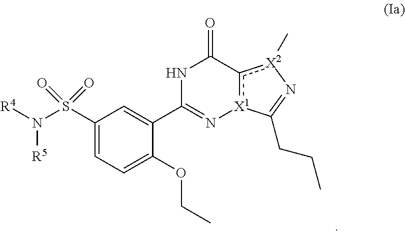

In some embodiments of formula (I)-(Ia), R⁵ is a substituted azetidine. In some embodiments, R⁵ is substituted azetidin-3-yl. In some embodiments, R⁵ is N-substituted azetidin-3-yl. In some embodiments, R⁵ is azetidine substituted with optionally substituted (C₁-C₅)alkyl, optionally substituted (C₁-C₁₀)alkylene, optionally substituted (C₂-C₁₀)alkenyl, optionally substituted (C₂-C₁₀)alkynyl, or optionally substituted (C₁-C₅)alkoxy. In some embodiments of formula (I)-(Ia), R⁴ is —H. In some embodiments of formula (I)-(Ia), R⁴ is (C₁-C₃)alkyl.

In some embodiments, X¹ is N and X² is C.

In some embodiments, X¹ is C and X² is N.

In some embodiments, the compound of formula (I) is a compound of formula (II):

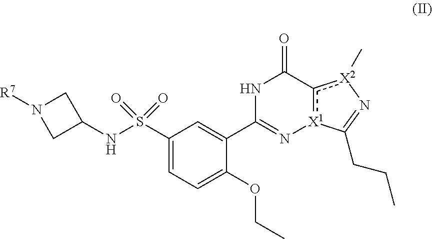

wherein:

R⁷ is selected from —H, R⁷⁰, and R⁷¹—Z²—R⁷²—;

R⁷⁰, R⁷¹ and R⁷² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, and optionally substituted (C₁-C₅)alkoxy, wherein the optional substituent is selected from —OH, —NH₂, and —O—NO₂; and

Z² is —CO₂—, —O—, —OCO—, —CONH—, —NHCO—, or —NH—.

In some embodiments of formula (I)-(II), X¹ is N and X² is C.

In some embodiments of formula (I)-(II), X¹ is C and X² is N.

In some embodiments of formula (II), the compound is of formula (IIa):

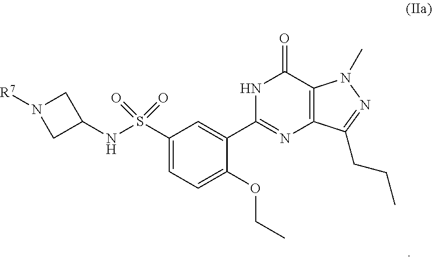

In some embodiments of formula (IIa), R⁷ is R⁷⁰. In some embodiments, R⁷⁰ is substituted (C₁-C₅)alkyl (e.g., substituted (C₂-C₅)alkyl).

In some embodiments of formula (IIa), R⁷ is

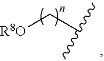

wherein R⁸ is —H or —NO₂, and n is 1, 2, 3, 4, or 5. In some embodiments, R⁸ is —H. In some embodiments, R⁸ is —NO₂. In some embodiments, n is 2. In some embodiments, n is 3. In some embodiments, n is 4.

In some embodiments of formula (IIa), the compound is selected from:

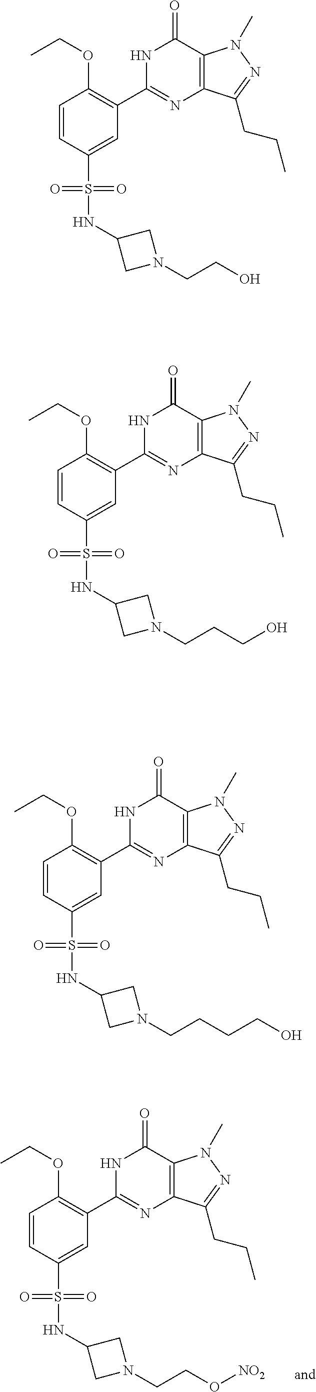

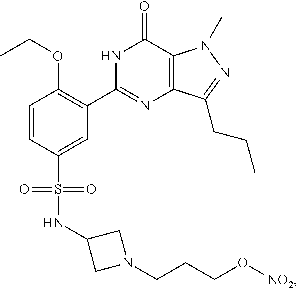 [SMILES: CCCc1nn(C)c2c(=O)[nH]c(-c3cc(S(=O)(=O)NC4CN(CCCO[N+](=O)[O-])C4)ccc3OCC)nc12]

or a pharmaceutically acceptable salt, a solvate, a hydrate, a prodrug, or a stereoisomer thereof.

In some embodiments of formula (II), the compound is of formula (IIb):

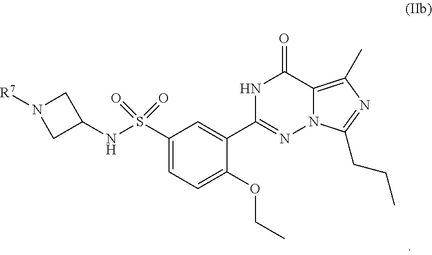

In some embodiments of formula (IIb), R⁷ is R⁷⁰. In some embodiments, R⁷⁰ is substituted (C₁-C₅)alkyl (e.g., substituted (C₂-C₅)alkyl).

In some embodiments of the compound of formula (IIb), R⁷ is

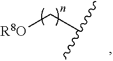

R⁸ is —H or —NO₂, and n is 1, 2, 3, 4, or 5. In some embodiments, R⁸ is —H. In some embodiments, R⁸ is —NO₂. In some embodiments, n is 2. In some embodiments, n is 3. In some embodiments, n is 4.

In some embodiments of formula (IIb), the compound is selected from:

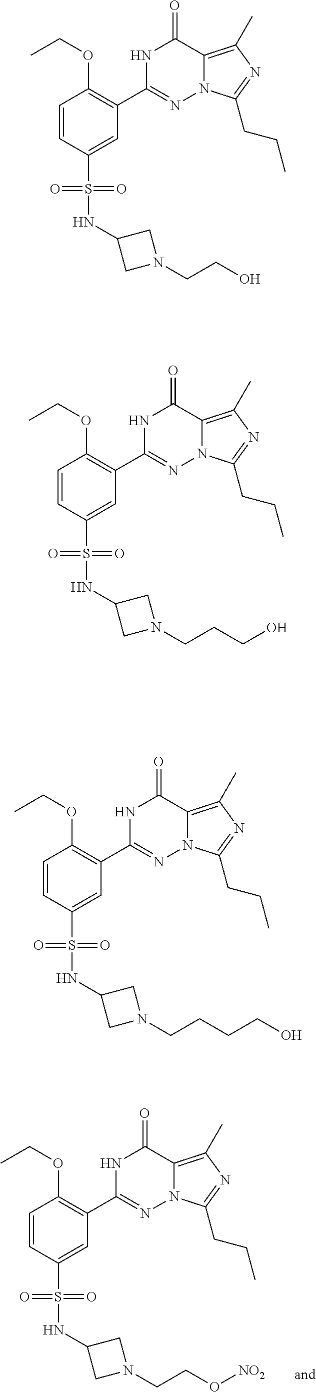

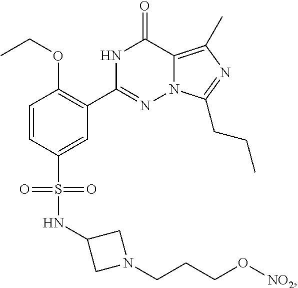 [SMILES: CCCc1nc(C)c2c(=O)[nH]c(-c3cc(S(=O)(=O)NC4CN(CCCO[N+](=O)[O-])C4)ccc3OCC)nn12]

or a pharmaceutically acceptable salt, a solvate, a hydrate, a prodrug, or a stereoisomer thereof.

In some embodiments of formula (I)-(Ia), R⁴ and R⁵ together with the nitrogen atom to which they are attached are cyclically linked to form a substituted azetidine. In some embodiments, R⁴ and R⁵ are cyclically linked to provide an azetidine substituted (e.g., at the 3-position) with optionally substituted (C₁-C₅)alkyl, optionally substituted (C₁-C₁₀)alkylene, optionally substituted (C₂-C₁₀)alkenyl, optionally substituted (C₂-C₁₀)alkynyl, or optionally substituted (C₁-C₅)alkoxy.

In some embodiments of formula (I)-(Ia), the compound is of formula (III):

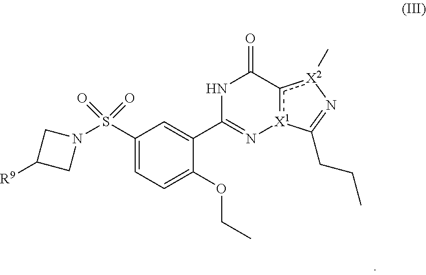

wherein:

R⁹ is selected from —O—NO₂, —NR¹⁰R¹¹, —OR¹², R⁹⁰, and R⁹¹—Z³—R⁹²;

R⁹⁰, R⁹¹ and R⁹² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, optionally substituted (C₁-C₅)alkoxy, optionally substituted (C₃-C₅)heterocycle-(C₁-C₅)alkyl, and optionally substituted (C₁-C₅)alkyl-(C₃-C₅)heterocycle-(C₁-C₅)alkyl, wherein the optional substituent is selected from —OH, —NH₂, and —O—NO₂;

Z³ is —CO₂—, —O—, —OCO—, —CONH—, —NHCO—, or —NH—; and

R¹⁰, R¹¹, and R¹² are independently H, optionally substituted (C₁-C₅)alkyl, or optionally substituted (C₁-C₅)alkyl-Z¹—(C₁-C₅)alkyl, wherein the optional substituent is selected from —OH, —NH₂, and —O—NO₂;

or R¹⁰ and R¹¹ together with the nitrogen atom to which they are attached are cyclically linked to form an optionally substituted heterocycle, wherein the optional substituent is selected from —OH, —O—NO₂, —CH₂OH, —CH₂CH₂OH, and —CH₂—O—NO₂.

In some embodiments, Z³ is —CO₂—, —O—, —OCO—, —CONH—, or —NH—.

In some embodiments, the compound of formula (III) is a compound of formula (IIIa):

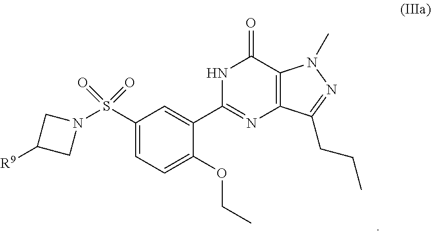

In some embodiments, the compound of formula (III) is a compound of formula (IIIb):

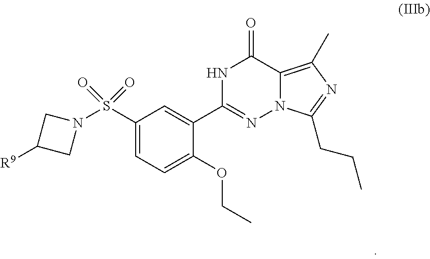

In some embodiments of formula (IIIa)-(IIIb), R⁹ is

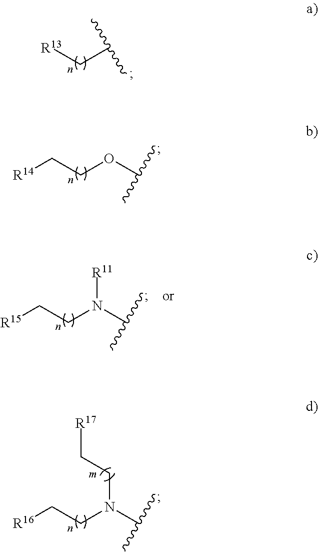

and wherein:

R¹¹ is —H or methyl;

R¹³, R¹⁴, R¹⁵, R¹⁶, and R¹⁷ are independently selected from —OH, —NH₂, and —O—NO₂; and

n and m are independently selected from 0, 1, 2, 3, 4, or 5.

In some embodiments of formula (IIIa)-(IIIb), R⁹ is

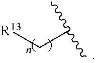

In some embodiments, R¹³ is —OH, or —O—NO₂. In some embodiments, R¹³ is —NH₂. In some embodiments, n is 0 to 4, such as 0 to 3. In some embodiments of formula (IIIa)-(IIIb), R⁹ is

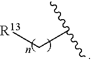

In some embodiments of formula (IIIa)-(IIIb), R⁹ is

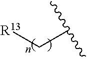

selected from:

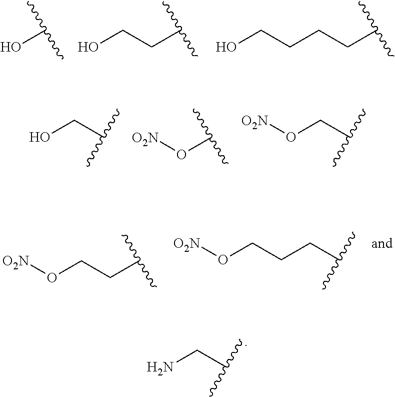

In some embodiments of formula (IIIa), the compound is selected from:

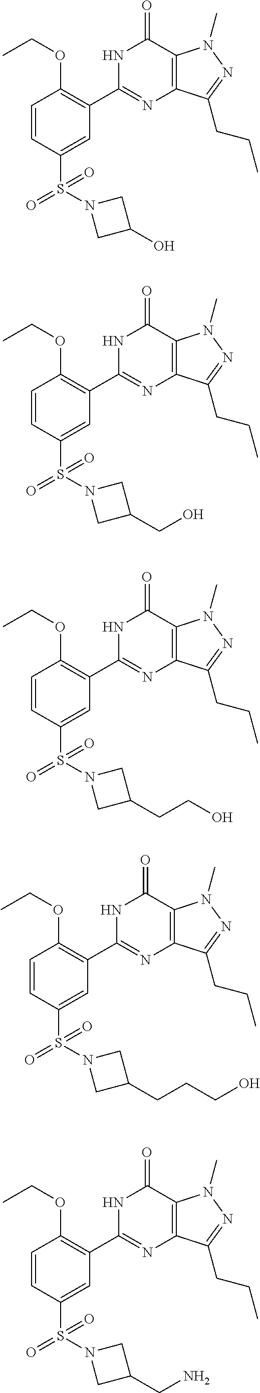

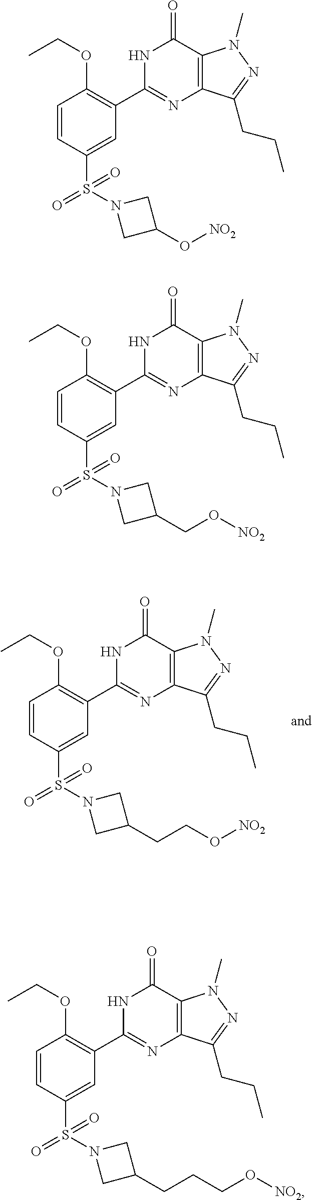

or a pharmaceutically acceptable salt, a solvate, a hydrate, a prodrug, or a stereoisomer thereof.

In some embodiments of formula (IIIa), R⁹ is

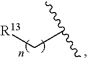

R¹³ is —OH, or —O—NO₂, and n is 0 to 4, such as 0 to 3.

In some embodiments, the compound of formula (IIIa) has the structure:

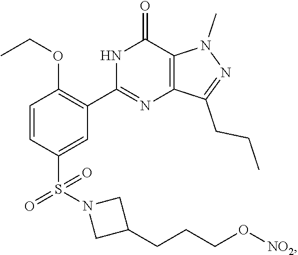 [SMILES: CCCc1nn(C)c2c(=O)[nH]c(-c3cc(S(=O)(=O)N4CC(CCCO[N+](=O)[O-])C4)ccc3OCC)nc12]

or a pharmaceutically acceptable salt, a solvate, a hydrate, a prodrug, or a stereoisomer thereof.

In some embodiments, the compound of formula (IIIa) has the structure:

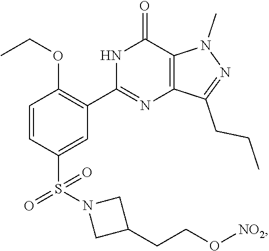 [SMILES: CCCc1nn(C)c2c(=O)[nH]c(-c3cc(S(=O)(=O)N4CC(CCO[N+](=O)[O-])C4)ccc3OCC)nc12]

or a pharmaceutically acceptable salt, a solvate, a hydrate, a prodrug, or a stereoisomer thereof.

In some embodiments, the compound of formula (IIIa) has the structure:

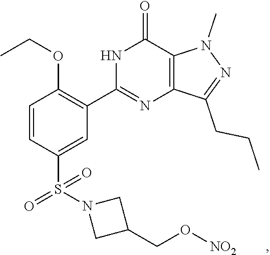 [SMILES: CCCc1nn(C)c2c(=O)[nH]c(-c3cc(S(=O)(=O)N4CC(CO[N+](=O)[O-])C4)ccc3OCC)nc12]

or a pharmaceutically acceptable salt, a solvate, a hydrate, a prodrug, or a stereoisomer thereof.

In some embodiments of formula (IIIb), R⁹ is

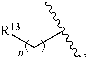

R¹³ is —OH, or —O—NO₂, and n is 0 to 4, such as 0 to 3.

In some embodiments of formula (IIIa)-(IIIb), R⁹ is

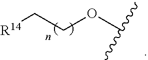

In some embodiments, R¹⁴ is —OH, or —O—NO₂. In some embodiments, n is 1 to 5, such as 1 to 4.

In some embodiments of formula (IIIa)-(IIIb), R⁹ is

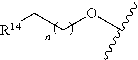

selected from:

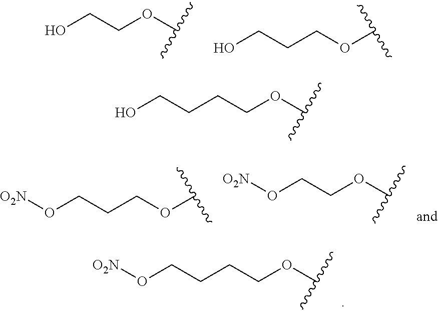 [SMILES: *OCCO ; *OCCCO ; *OCCCCO ; *OCCO[N+](=O)[O-] ; *OCCCO[N+](=O)[O-] ; *OCCCCO[N+](=O)[O-]]

In some embodiments of formula (IIIa), the compound is selected from:

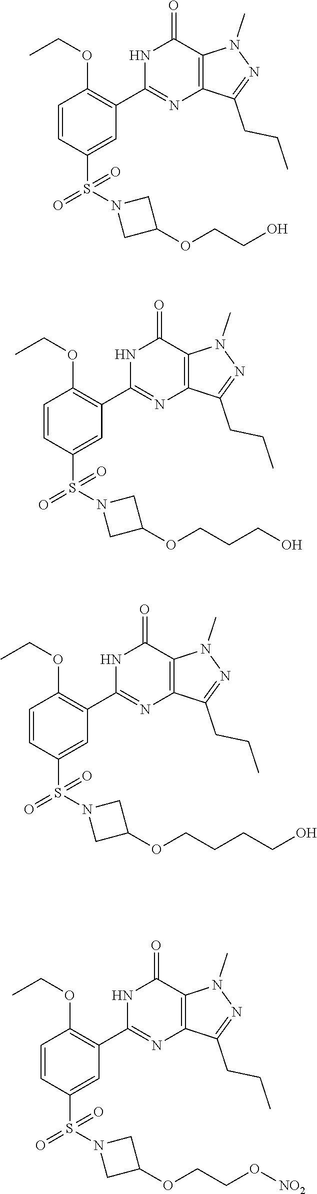

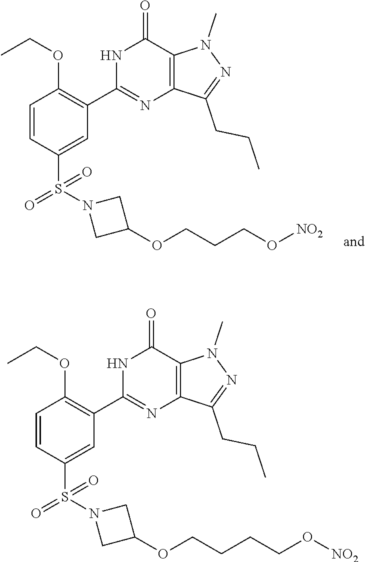 [SMILES: CCCc1nn(C)c2c(=O)[nH]c(-c3cc(S(=O)(=O)N4CC(OCCCO[N+](=O)[O-])C4)ccc3OCC)nc12 ; CCCc1nn(C)c2c(=O)[nH]c(-c3cc(S(=O)(=O)N4CC(OCCCCO[N+](=O)[O-])C4)ccc3OCC)nc12]

or a pharmaceutically acceptable salt, a solvate, a hydrate, a prodrug, or a stereoisomer thereof.

In some embodiments of formula (IIIa)-(IIIb), R⁹ is

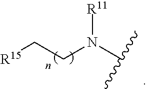

In some embodiments, R¹⁵ is —OH, or —O—NO₂. In some embodiments, n is 1 to 5, such as 1 to 4. In some embodiments, R¹¹ is —H. In some embodiments, R¹¹ is methyl.

In some embodiments of formula (IIIa)-(IIIb), R⁹ is

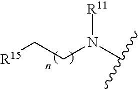

selected from:

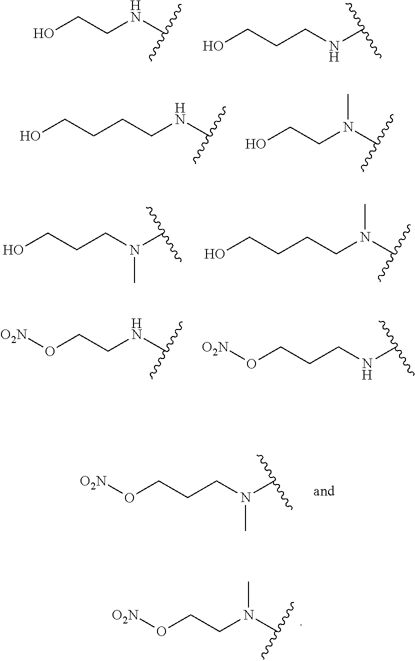

In some embodiments of formula (IIIa), the compound is selected from:

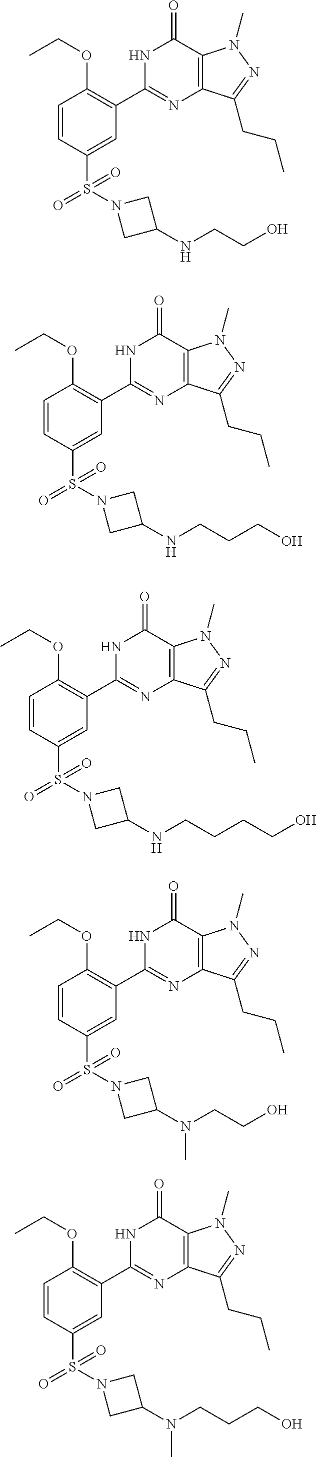

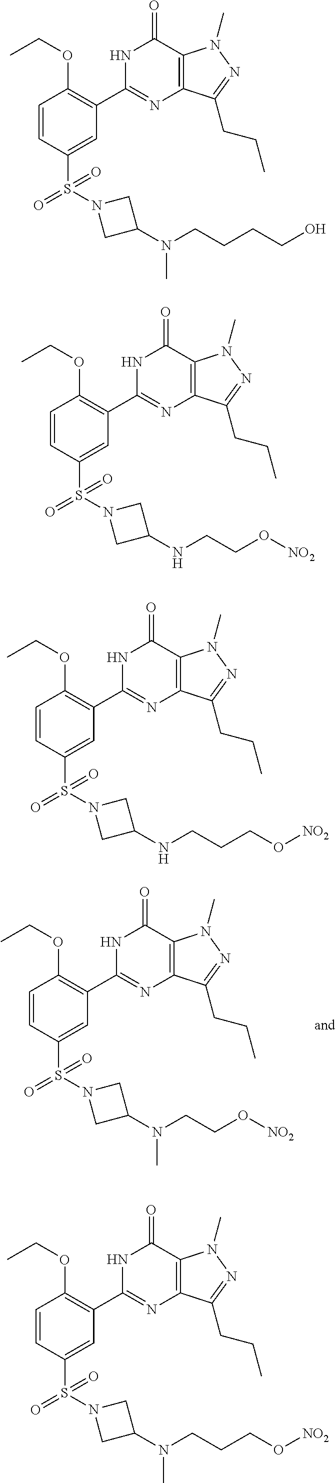

or a pharmaceutically acceptable salt, a solvate, a hydrate, a prodrug, or a stereoisomer thereof.

In some embodiments of formula (IIIa)-(IIIb), R⁹ is

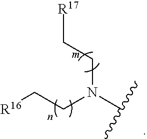

In some embodiments, R¹⁶ and R¹⁷ are independently —OH, or —O—NO₂. In some embodiments, n and m are independently 2 to 5, such as 2 to 4. In some embodiments, R¹⁶ and R¹⁷ are each —OH, or —O—NO₂. In some embodiments, n and m are each 2 to 5, such as 2 to 4.

In some embodiments of formula (IIIa)-(IIIb), R⁹ is

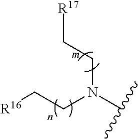

selected from:

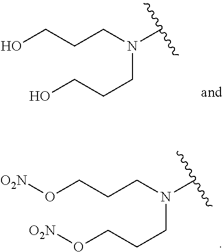 [SMILES: *N(CCCO)CCCO ; *N(CCCO[N+](=O)[O-])CCCO[N+](=O)[O-]]

In some embodiments of formula (IIIa), the compound is selected from:

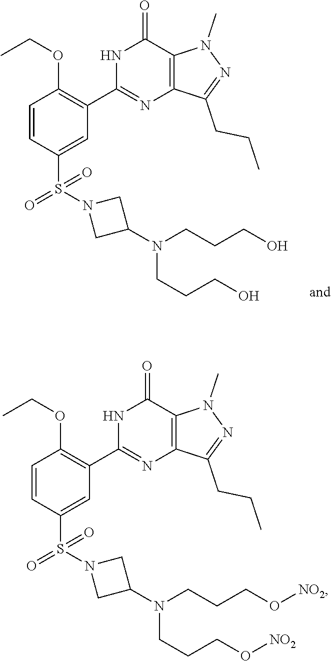 [SMILES: CCCc1nn(C)c2c(=O)[nH]c(-c3cc(S(=O)(=O)N4CC(N(CCCO)CCCO)C4)ccc3OCC)nc12 ; CCCc1nn(C)c2c(=O)[nH]c(-c3cc(S(=O)(=O)N4CC(N(CCCO[N+](=O)[O-])CCCO[N+](=O)[O-])C4)ccc3OCC)nc12]

or a pharmaceutically acceptable salt, a solvate, a hydrate, a prodrug, or a stereoisomer thereof.

In some embodiments of formula (IIIa)-(IIIb), R⁹ is

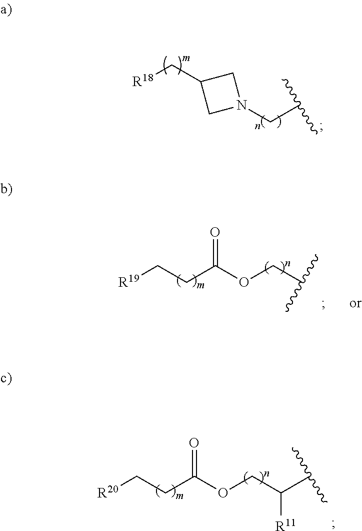

wherein:

R¹¹ is —H or methyl;

R¹⁸ is selected from —OH, —NH₂, and —O—NO₂;

R¹⁹ and R²⁰ are independently selected from —OH, —NH₂, —O—NO₂, and

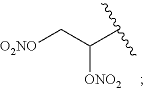

and

n and m are independently selected from 0, 1, 2, 3, 4, 5, and 6.

In some embodiments of formula (IIIa)-(IIIb), R⁹ is

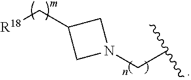

In some embodiments, R¹⁸ is selected from —OH, and —O—NO₂. In some embodiments, n is 0 to 2, such as 0 or 1. In some embodiments, m is 0 to 3, such as 0 to 2, e.g., 0, 1 or 2. In some embodiments, n is 0 to 2, and m is 0 to 3, such as 0 to 2.

In some embodiments of formula (IIIa)-(IIIb), R⁹ is

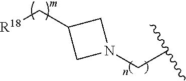

selected from:

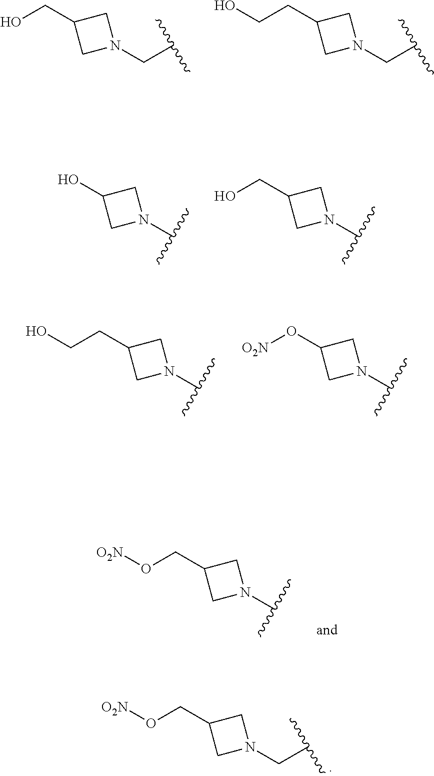

In some embodiments of formula (IIIa), the compound is selected from:

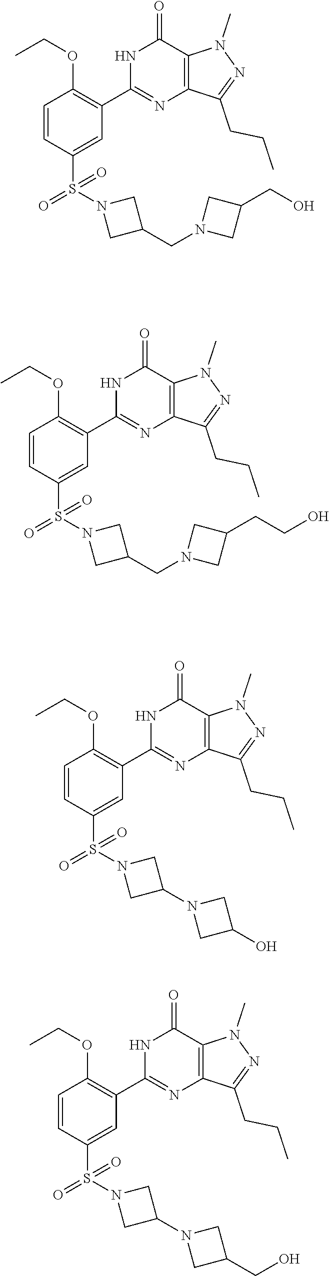

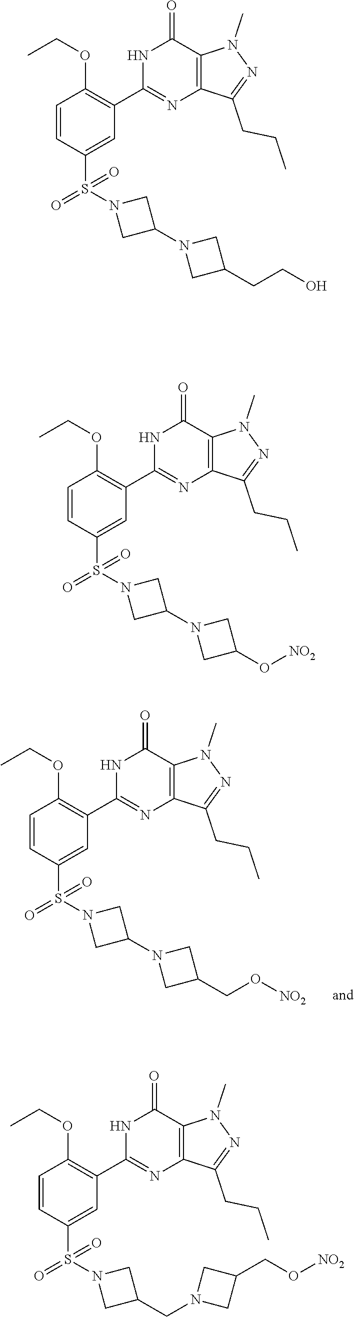

or a pharmaceutically acceptable salt, a solvate, a hydrate, a prodrug, or a stereoisomer thereof.

In some embodiments of formula (IIIa)-(IIIb), R⁹ is

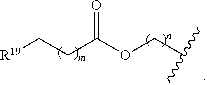

In some embodiments, R¹⁹ is selected from —OH, —O—NO₂ and

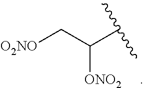

In some embodiments, n is 0 to 4, such as 1 to 3. In some embodiments, m is 0 to 4, such as 1 to 4. In some embodiments, n is 0 to 4 and m is 0 to 4.

In some embodiments of formula (IIIa)-(IIIb), R⁹ is

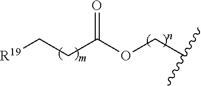

selected from:

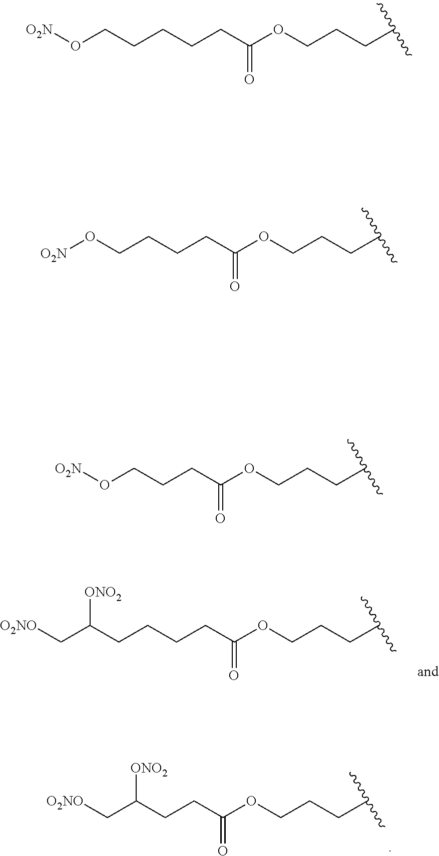

In some embodiments of formula (IIIa), the compound is selected from:

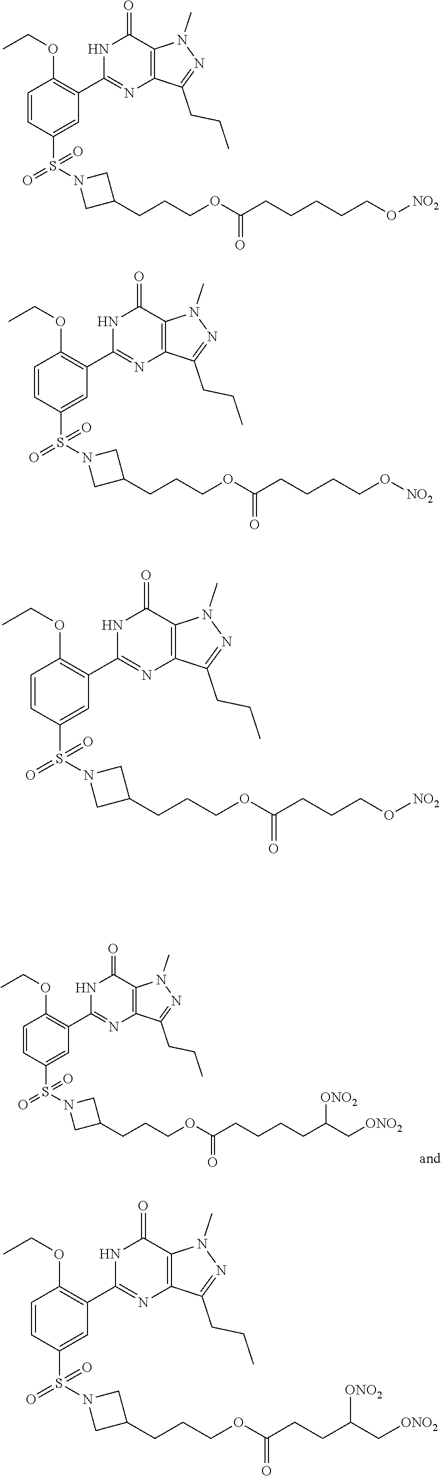

or a pharmaceutically acceptable salt, a solvate, a hydrate, a prodrug, or a stereoisomer thereof.

In some embodiments of formula (IIIa)-(IIIb), R⁹ is

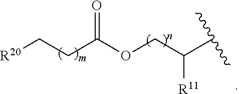

In some embodiments, R²⁰ is selected from —OH, —O—NO₂ and

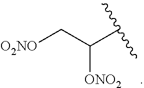

In some embodiments, n is 2 to 6, such as 2 to 4. In some embodiments, m is 0 to 5, such as 1 to 4. In some embodiments, n is 2 to 4 and m is 0 to 5. In some embodiments, R¹¹ is —H. In some embodiments, R¹¹ is methyl.

In some embodiments of formula (IIIa)-(IIIb), R⁹ is

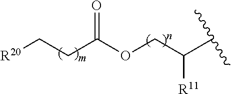

selected from:

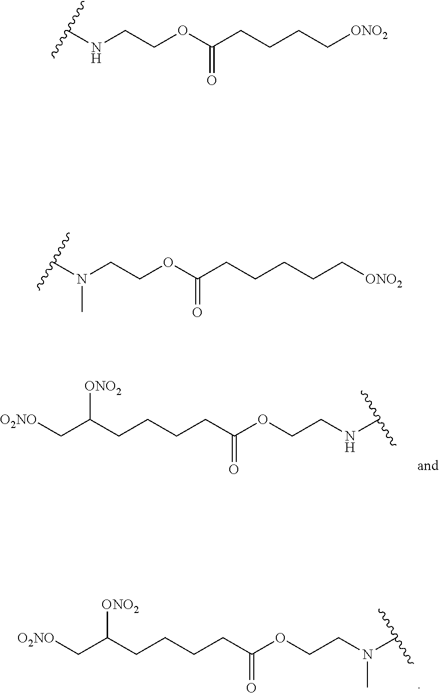

In some embodiments of formula (IIIa), the compound is selected from:

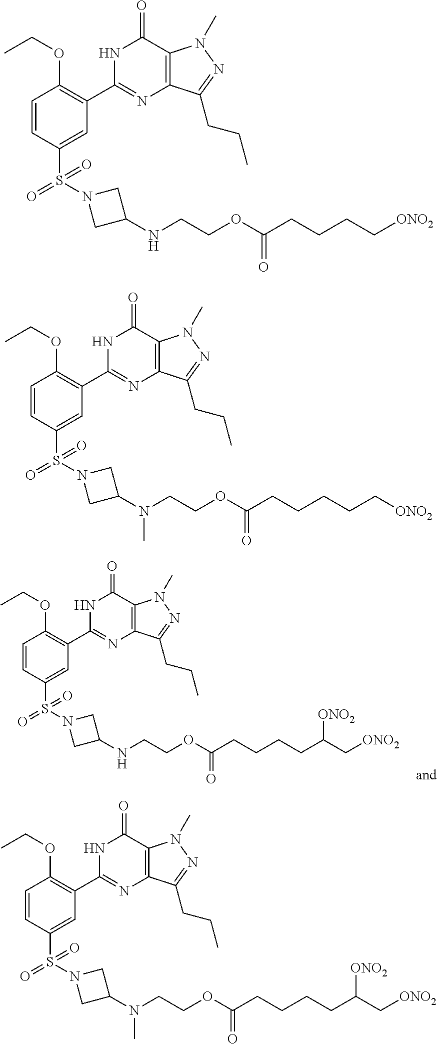

or a pharmaceutically acceptable salt, a solvate, a hydrate, a prodrug, or a stereoisomer thereof. In some embodiments of formula (IIIa)-(IIIb), R⁹ is

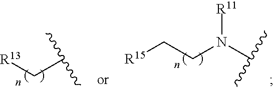

wherein:

R¹¹ is —H or methyl;

R¹³ and R¹⁵ are independently selected from —OH, —NH₂, and —O—NO₂; and

n and m are independently selected from 0, 1, 2, 3, 4 or 5.

In some embodiments of formula (IIIb), R⁹ is

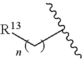

selected from:

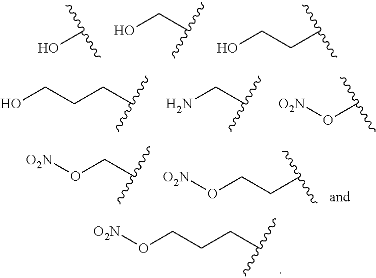

In some embodiments of formula (IIIb), the compound is selected from:

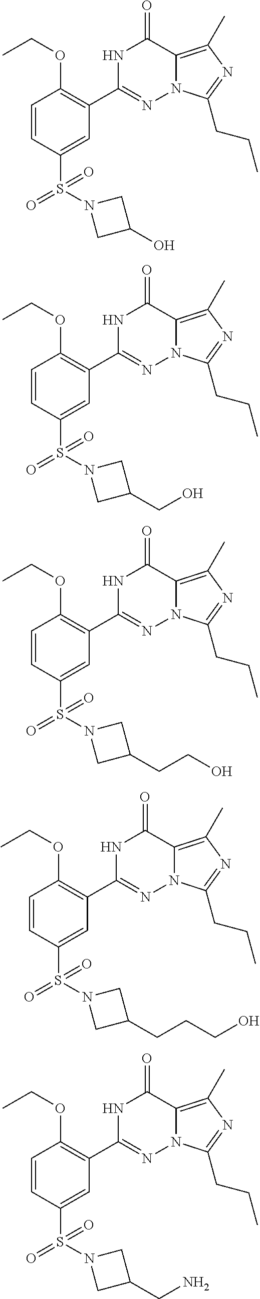

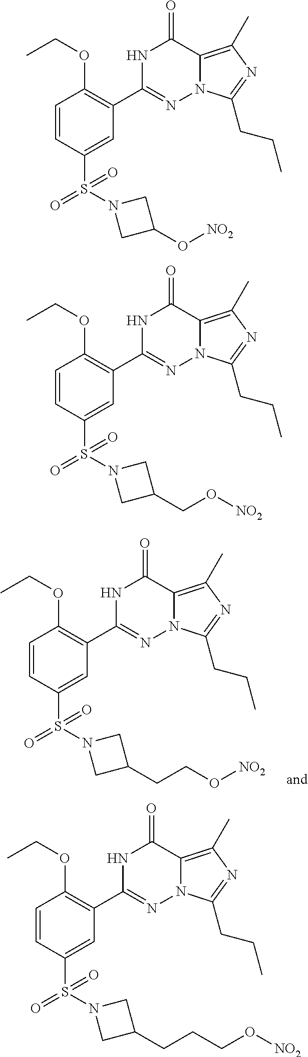

or a pharmaceutically acceptable salt, a solvate, a hydrate, a prodrug, or a stereoisomer thereof.

In some embodiments of formula (IIIb), R⁹ is

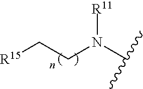

selected from:

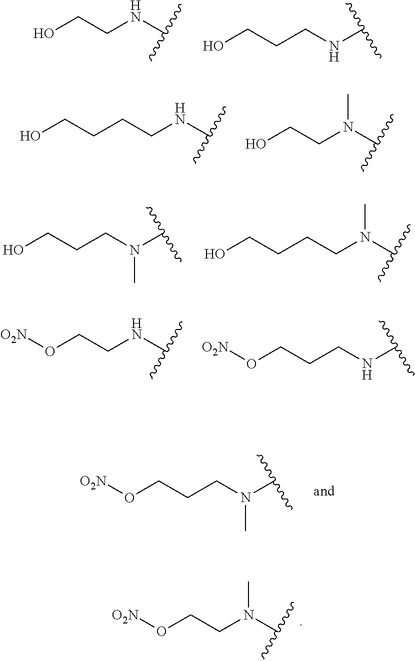

In some embodiments of formula (IIIb), the compound is selected from:

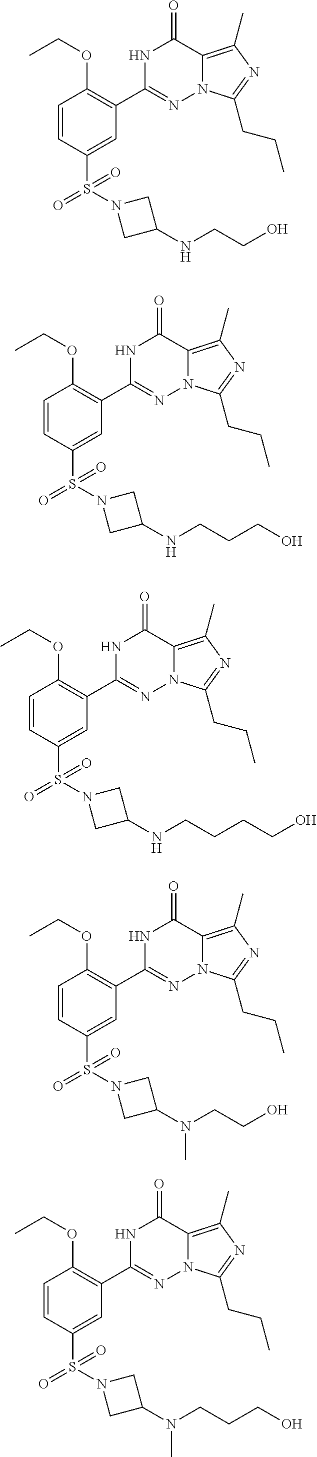

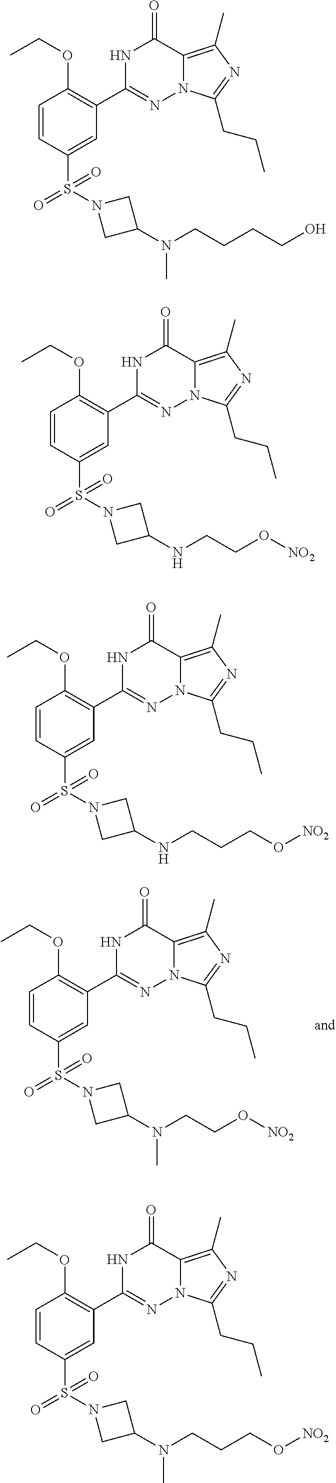

or a pharmaceutically acceptable salt, a solvate, a hydrate, a prodrug, or a stereoisomer thereof.

It is understood that all variations of salts, solvates, hydrates, prodrugs and/or stereoisomers of the compounds described herein and shown in Table 1 are meant to be encompassed by the present disclosure.

In some embodiments, the compound is represented by the structure of one of the compounds in Table 1. The present disclosure is meant to encompass, a compound of any one of Table 1, or a salt, a solvate, a hydrate, a prodrug, a single stereoisomer, a mixture of stereoisomers and/or an isotopically labelled form thereof.

TABLE 1
Exemplary Compounds

| Cmpd | | |
|---|---|---|
| No. | Structure | Name |
| 1 | 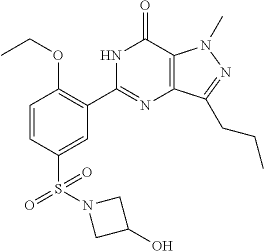 [SMILES: CCCc1nn(C)c2c(=O)[nH]c(-c3cc(S(=O)(=O)N4CC(O)C4)ccc3OCC)nc12] | 5-(2-ethoxy-5-((3-hydroxyazetidin-1- yl)sulfonyl)phenyl)-1-methyl-3- propyl-1,6-dihydro-7H-pyrazolo[4,3- d]pyrimidin-7-one |
| 2 | 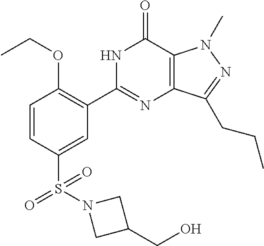 [SMILES: CCCc1nn(C)c2c(=O)[nH]c(-c3cc(S(=O)(=O)N4CC(CO)C4)ccc3OCC)nc12] | 5-(2-ethoxy-5-((3- (hydroxymethyl)azetidin-1- yl)sulfonyl)phenyl)-1-methyl-3- propyl-1,6-dihydro-7H-pyrazolo[4,3- d]pyrimidin-7-one |
| 3 | 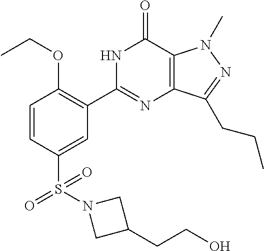 [SMILES: CCCc1nn(C)c2c(=O)[nH]c(-c3cc(S(=O)(=O)N4CC(CCO)C4)ccc3OCC)nc12] | 5-(2-ethoxy-5-((3-(2- hydroxyethyl)azetidin-1- yl)sulfonyl)phenyl)-1-methyl-3- propyl-1,6-dihydro-7H-pyrazolo[4,3- d]pyrimidin-7-one |
| 4 | 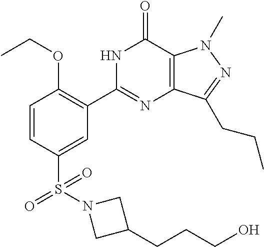 [SMILES: CCCc1nn(C)c2c(=O)[nH]c(-c3cc(S(=O)(=O)N4CC(CCCO)C4)ccc3OCC)nc12] | 5-(2-ethoxy-5-((3-(3- hydroxypropyl)azetidin-1- yl)sulfonyl)phenyl)-1-methyl-3- propyl-1,6-dihydro-7H-pyrazolo[4,3- d]pyrimidin-7-one |
| 5 | 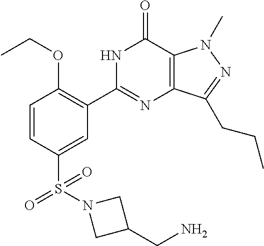 [SMILES: CCCc1nn(C)c2c(=O)[nH]c(-c3cc(S(=O)(=O)N4CC(CN)C4)ccc3OCC)nc12] | 5-(5-((3-(aminomethyl)azetidin-1- yl)sulfonyl)-2--ethoxyphenyl)-1- methyl-3-propyl-1,6-dihydro-7H- pyrazolo[4,3-d]pyrimidin-7-one |
| 6 | 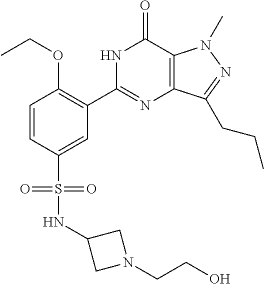 [SMILES: CCCc1nn(C)c2c(=O)[nH]c(-c3cc(S(=O)(=O)NC4CN(CCO)C4)ccc3OCC)nc12] | 4-ethoxy-N-(1-(2- hydroxyethyl)azetidin-3-yl)-3-(1- methyl-7-oxo-3-propyl-6,7-dihydro- 1H-pyrazolo[4,3-d]pyrimidin-5- yl)benzenesulfonamide |
| 7 | 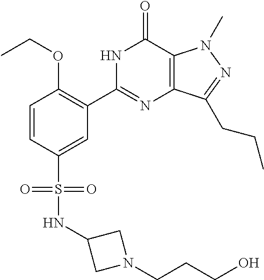 [SMILES: CCCc1nn(C)c2c(=O)[nH]c(-c3cc(S(=O)(=O)NC4CN(CCCO)C4)ccc3OCC)nc12] | 4-ethoxy-N-(1-(3- hydroxypropyl)azetidin-3-yl)-3-(1- methyl-7-oxo-3-propyl-6,7-dihydro- 1H-pyrazolo[4,3-d]pyrimidin-5- yl)benzenesulfonamide |
| 8 | 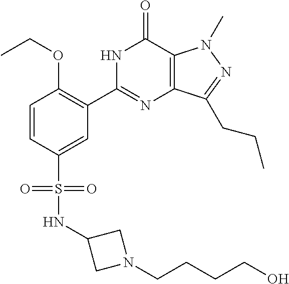 [SMILES: CCCc1nn(C)c2c(=O)[nH]c(-c3cc(S(=O)(=O)NC4CN(CCCCO)C4)ccc3OCC)nc12] | 4-ethoxy-N-(1-(4- hydroxybutyl)azetidin-3-yl)-3-(1- methyl-7-oxo-3-propyl-6,7-dihydro- 1H-pyrazolo[4,3-d]pyrimidin-5- yl)benzenesulfonamide |
| 9 | 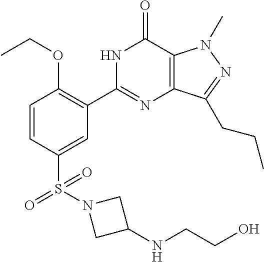 [SMILES: CCCc1nn(C)c2c(=O)[nH]c(-c3cc(S(=O)(=O)N4CC(NCCO)C4)ccc3OCC)nc12] | 5-(2-ethoxy-5-((3-((2- hydroxyethyl)amino)azetidin-1- yl)sulfonyl)phenyl)-1-methyl-3- propyl-1,6-dihydro-7H-pyrazolo[4,3- d]pyrimidin-7-one |
| 10 | 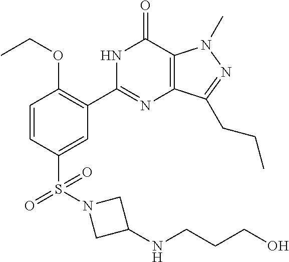 [SMILES: CCCc1nn(C)c2c(=O)[nH]c(-c3cc(S(=O)(=O)N4CC(NCCCO)C4)ccc3OCC)nc12] | 5-(2-ethoxy-5-((3-((3- hydroxypropyl)amino)azetidin-1- yl)sulfonyl)phenyl)-1-methyl-3- propyl-1,6-dihydro-7H-pyrazolo[4,3- d]pyrimidin-7-one |
| 11 | 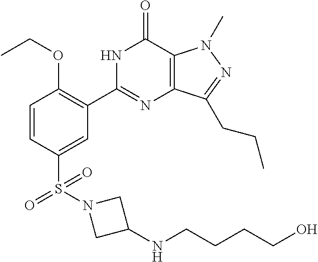 [SMILES: CCCc1nn(C)c2c(=O)[nH]c(-c3cc(S(=O)(=O)N4CC(NCCCCO)C4)ccc3OCC)nc12] | 5-(2-ethoxy-5-((3-((4- hydroxybutyl)amino)azetidin-1- yl)sulfonyl)phenyl)-1-methyl-3- propyl-1,6-dihydro-7H-pyrazolo[4,3- d]pyrimidin-7-one |
| 12 | 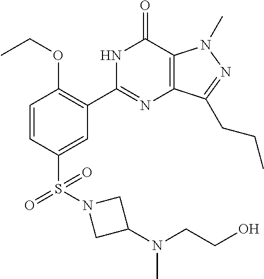 [SMILES: CCCc1nn(C)c2c(=O)[nH]c(-c3cc(S(=O)(=O)N4CC(N(C)CCO)C4)ccc3OCC)nc12] | 5-(2-ethoxy-5-((3-((2- hydroxyethyl)(methyl)amino) azetidin-1- yl)sulfonyl)phenyl)-1-methyl-3- propyl-1,6-dihydro-7H-pyrazolo[4,3- d]pyrimidin-7-one |
| 13 | 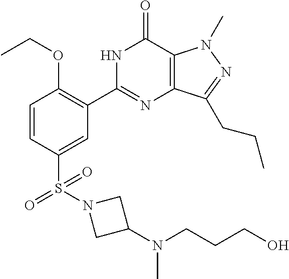 [SMILES: CCCc1nn(C)c2c(=O)[nH]c(-c3cc(S(=O)(=O)N4CC(N(C)CCCO)C4)ccc3OCC)nc12] | 5-(2-ethoxy-5-((3-((3- hydroxyethyl)(methyl)amino) azetidin-1- yl)sulfonyl)phenyl)-1-methyl-3- propyl-1,6-dihydro-7H-pyrazolo[4,3- d]pyrimidin-7-one |
| 14 | 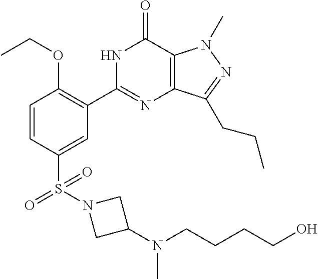 [SMILES: CCCc1nn(C)c2c(=O)[nH]c(-c3cc(S(=O)(=O)N4CC(N(C)CCCCO)C4)ccc3OCC)nc12] | 5-(2-ethoxy-5-((3-((4- hydroxybutyl)(methyl)amino) azetidin-1- yl)sulfonyl)phenyl)-1-methyl-3- propyl-1,6-dihydro-7H-pyrazolo[4,3- d]pyrimidin-7-one |
| 15 | 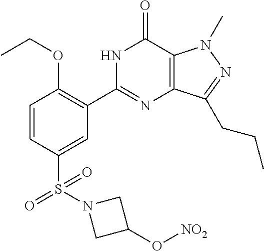 [SMILES: CCCc1nn(C)c2c(=O)[nH]c(-c3cc(S(=O)(=O)N4CC(O[N+](=O)[O-])C4)ccc3OCC)nc12] | 1-((4-ethoxy-3-(1-methyl-7-oxo-3- propyl-6,7-dihydro-1H-pyrazolo[4,3- d]pyrimidin-5- yl)phenyl)sulfonyl)azetidin-3-yl nitrate |
| 16 | 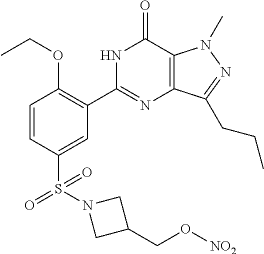 [SMILES: CCCc1nn(C)c2c(=O)[nH]c(-c3cc(S(=O)(=O)N4CC(CO[N+](=O)[O-])C4)ccc3OCC)nc12] | (1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)methyl nitrate |
| 17 | 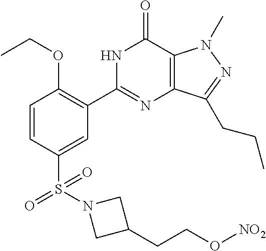 [SMILES: CCCc1nn(C)c2c(=O)[nH]c(-c3cc(S(=O)(=O)N4CC(CCO[N+](=O)[O-])C4)ccc3OCC)nc12] | 2-(1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)ethyl nitrate |
| 18 | 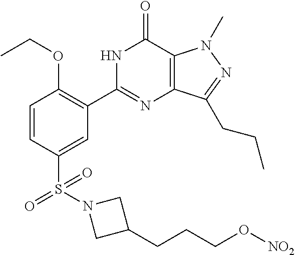 [SMILES: CCCc1nn(C)c2c(=O)[nH]c(-c3cc(S(=O)(=O)N4CC(CCCO[N+](=O)[O-])C4)ccc3OCC)nc12] | 3-(1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)propyl nitrate |
| 19 | 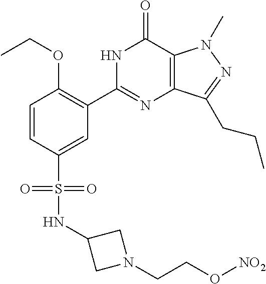 [SMILES: CCCc1nn(C)c2c(=O)[nH]c(-c3cc(S(=O)(=O)NC4CN(CCO[N+](=O)[O-])C4)ccc3OCC)nc12] | 2-(3-((4-ethoxy-3-(1-methyl-7-oxo-3- propyl-6,7-dihydro-1H-pyrazolo[4,3- d]pyrimidin-5- yl)phenyl)sulfonamido)azetidin-1- yl)ethyl nitrate |
| 20 | 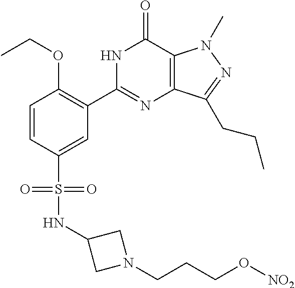 [SMILES: CCCc1nn(C)c2c(=O)[nH]c(-c3cc(S(=O)(=O)NC4CN(CCCO[N+](=O)[O-])C4)ccc3OCC)nc12] | 3-(3-((4-ethoxy-3-(1-methyl-7-oxo-3- propyl-6,7-dihydro-1H-pyrazolo[4,3- d]pyrimidin-5- yl)phenyl)sulfonamido)azetidin-1- yl)propyl nitrate |
| 21 | 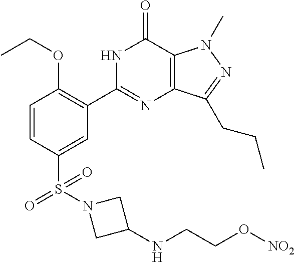 [SMILES: CCCc1nn(C)c2c(=O)[nH]c(-c3cc(S(=O)(=O)N4CC(NCCO[N+](=O)[O-])C4)ccc3OCC)nc12] | 2-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)amino)ethyl nitrate |
| 22 | 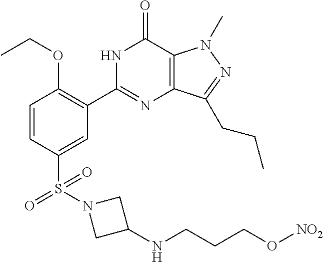 [SMILES: CCCc1nn(C)c2c(=O)[nH]c(-c3cc(S(=O)(=O)N4CC(NCCCO[N+](=O)[O-])C4)ccc3OCC)nc12] | 3-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)amino)propyl nitrate |
| 23 | 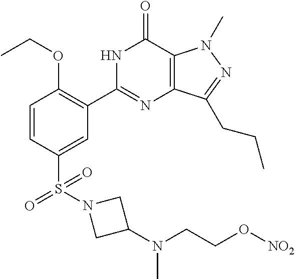 [SMILES: CCCc1nn(C)c2c(=O)[nH]c(-c3cc(S(=O)(=O)N4CC(N(C)CCO[N+](=O)[O-])C4)ccc3OCC)nc12] | 2-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)(methyl)amino)ethyl nitrate |
| 24 | 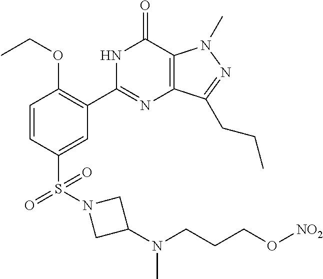 [SMILES: CCCc1nn(C)c2c(=O)[nH]c(-c3cc(S(=O)(=O)N4CC(N(C)CCCO[N+](=O)[O-])C4)ccc3OCC)nc12] | 3-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)(methyl)amino)propyl nitrate |
| 25 | 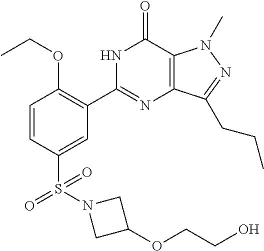 [SMILES: CCCc1nn(C)c2c(=O)[nH]c(-c3cc(S(=O)(=O)N4CC(OCCO)C4)ccc3OCC)nc12] | 5-(2-ethoxy-5-((3-(2- hydroxyethoxy)azetidin-1- yl)sulfonyl)phenyl)-1-methyl-3- propyl-1,6-dihydro-7H-pyrazolo[4,3- d]pyrimidin-7-one |
| 26 | 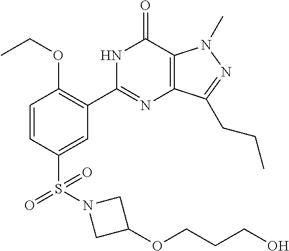 [SMILES: CCCc1nn(C)c2c(=O)[nH]c(-c3cc(S(=O)(=O)N4CC(OCCCO)C4)ccc3OCC)nc12] | 5-(2-ethoxy-5-((3-(3- hydroxypropoxy)azetidin-1- yl)sulfonyl)phenyl)-1-methyl-3- propyl-1,6-dihydro-7H-pyrazolo[4,3- d]pyrimidin-7-one |
| 27 | 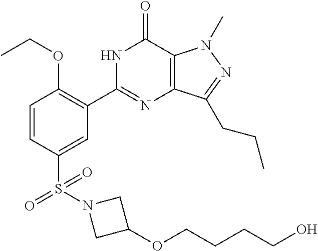 [SMILES: CCCc1nn(C)c2c(=O)[nH]c(-c3cc(S(=O)(=O)N4CC(OCCCCO)C4)ccc3OCC)nc12] | 5-(2-ethoxy-5-((3-(4- hydroxybutoxy)azetidin-1- yl)sulfonyl)phenyl)-1-methyl-3- propyl-1,6-dihydro-7H-pyrazolo[4,3- d]pyrimidin-7-one |
| 28 | 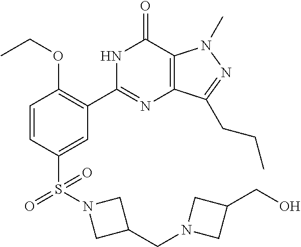 [SMILES: CCCc1nn(C)c2c(=O)[nH]c(-c3cc(S(=O)(=O)N4CC(CN5CC(CO)C5)C4)ccc3OCC)nc12] | 5-(2-ethoxy-5-((3-((3- (hydroxymethyl)azetidin-1- yl)methyl)azetidin-1- yl)sulfonyl)phenyl)-1-methyl-3- propyl-1,6-dihydro-7H-pyrazolo[4,3- d]pyrimidin-7-one |
| 29 | 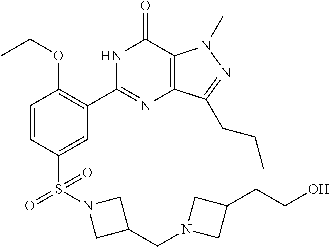 [SMILES: CCCc1nn(C)c2c(=O)[nH]c(-c3cc(S(=O)(=O)N4CC(CN5CC(CCO)C5)C4)ccc3OCC)nc12] | 5-(2-ethoxy-5-((3-((3-(2- hydroxyethyl)azetidin-1- yl)methyl)azetidin-1- yl)sulfonyl)phenyl)-1-methyl-3- propyl-1,6-dihydro-7H-pyrazolo[4,3- d]pyrimidin-7-one |
| 30 | 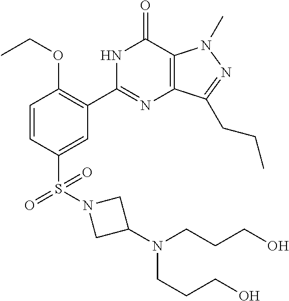 [SMILES: CCCc1nn(C)c2c(=O)[nH]c(-c3cc(S(=O)(=O)N4CC(N(CCCO)CCCO)C4)ccc3OCC)nc12] | 5-(5-((3-(bis(3- hydroxypropyl)amino)azetidin-1- yl)sulfonyl)-2-ethoxyphenyl)-1- methyl-3-propyl-1,6-dihydro-7H- pyrazolo[4,3-d]pyrimidin-7-one |
| 31 | 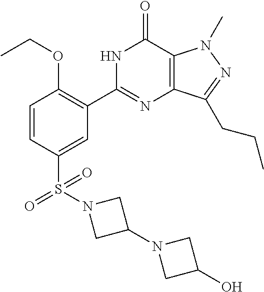 [SMILES: CCCc1nn(C)c2c(=O)[nH]c(-c3cc(S(=O)(=O)N4CC(N5CC(O)C5)C4)ccc3OCC)nc12] | 5-(2-ethoxy-5-((3-hydroxy-[1,3′- biazetidin]-1′-yl)sulfonyl)phenyl)-1- methyl-3-propyl-1,6-dihydro-7H- pyrazolo[4,3-d]pyrimidin-7-one |
| 32 | 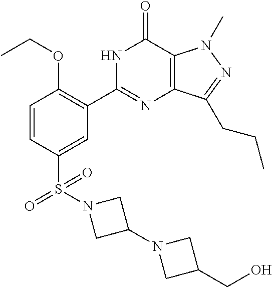 [SMILES: CCCc1nn(C)c2c(=O)[nH]c(-c3cc(S(=O)(=O)N4CC(N5CC(CO)C5)C4)ccc3OCC)nc12] | 5-(2-ethoxy-5-((3-(hydroxymethyl)- [1,3′-biazetidin]-1′- yl)sulfonyl)phenyl)-1-methyl-3- propyl-1,6-dihydro-7H-pyrazolo[4,3- d]pyrimidin-7-one |
| 33 | 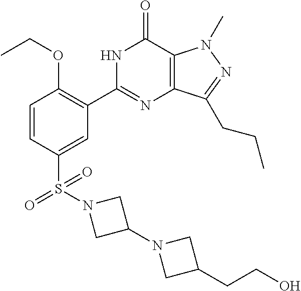 [SMILES: CCCc1nn(C)c2c(=O)[nH]c(-c3cc(S(=O)(=O)N4CC(N5CC(CCO)C5)C4)ccc3OCC)nc12] | 5-(2-ethoxy-5-((3-(2-hydroxyethyl)- [1,3′-biazetidin]-1′- yl)sulfonyl)phenyl)-1-methyl-3- propyl-1,6-dihydro-7H-pyrazolo[4,3- d]pyrimidin-7-one |
| 34 | 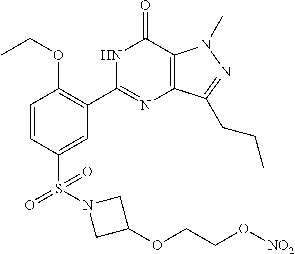 [SMILES: CCCc1nn(C)c2c(=O)[nH]c(-c3cc(S(=O)(=O)N4CC(OCCO[N+](=O)[O-])C4)ccc3OCC)nc12] | 2-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)oxy)ethyl nitrate |
| 35 | 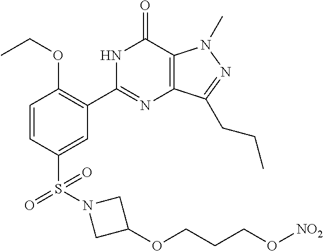 [SMILES: CCCc1nn(C)c2c(=O)[nH]c(-c3cc(S(=O)(=O)N4CC(OCCCO[N+](=O)[O-])C4)ccc3OCC)nc12] | 3-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)oxy)propyl nitrate |
| 36 | 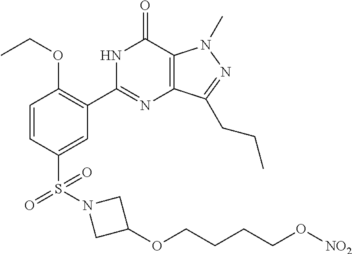 [SMILES: CCCc1nn(C)c2c(=O)[nH]c(-c3cc(S(=O)(=O)N4CC(OCCCCO[N+](=O)[O-])C4)ccc3OCC)nc12] | 4-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)oxy)butyl nitrate |
| 37 | 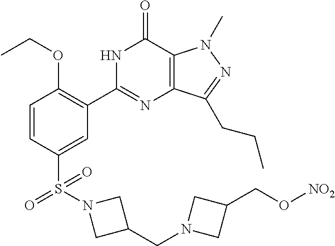 [SMILES: CCCc1nn(C)c2c(=O)[nH]c(-c3cc(S(=O)(=O)N4CC(CN5CC(CO[N+](=O)[O-])C5)C4)ccc3OCC)nc12] | (1-((1-((4-ethoxy-3-(1-methyl-7-oxo- 3-propyl-6,7-dihydro-1H- pyrazolo[4,3-d]pyrimidin-5- yl)phenyl)sulfonyl)azetidin-3- yl)methyl)azetidin-3-yl)methyl nitrate |
| 38 | 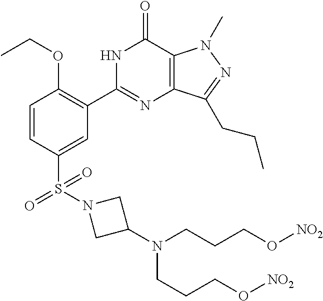 [SMILES: CCCc1nn(C)c2c(=O)[nH]c(-c3cc(S(=O)(=O)N4CC(N(CCCO[N+](=O)[O-])CCCO[N+](=O)[O-])C4)ccc3OCC)nc12] | ((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)azanediyl)bis(propane-3,1-diyl) dinitrate |
| 39 | 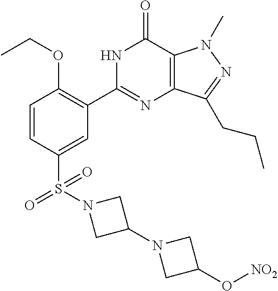 [SMILES: CCCc1nn(C)c2c(=O)[nH]c(-c3cc(S(=O)(=O)N4CC(N5CC(O[N+](=O)[O-])C5)C4)ccc3OCC)nc12] | 1′-((4-ethoxy-3-(1-methyl-7-oxo-3- propyl-6,7-dihydro-1H-pyrazolo[4,3- d]pyrimidin-5-yl)phenyl)sulfonyl)- [1,3′-biazetidin]-3-yl nitrate |
| 40 | 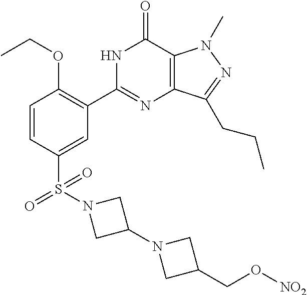 [SMILES: CCCc1nn(C)c2c(=O)[nH]c(-c3cc(S(=O)(=O)N4CC(N5CC(CO[N+](=O)[O-])C5)C4)ccc3OCC)nc12] | (1′-((4-ethoxy-3-(1-methyl-7-oxo-3- propyl-6,7-dihydro-1H-pyrazolo[4,3- d]pyrimidin-5-yl)phenyl)sulfonyl)- [1,3′-biazetidin]-3-yl)methyl nitrate |
| 41 | 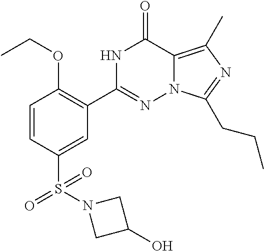 [SMILES: CCCc1nc(C)c2c(=O)[nH]c(-c3cc(S(=O)(=O)N4CC(O)C4)ccc3OCC)nn12] | 2-(2-ethoxy-5-((3-hydroxyazetidin-1- yl)sulfonyl)phenyl)-5-methyl-7- propylimidazo[5,1-f][1,2,4]triazin- 4(3H)-one |
| 42 | 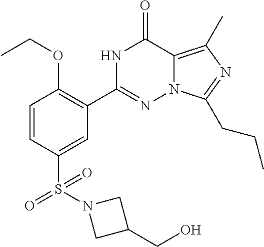 [SMILES: CCCc1nc(C)c2c(=O)[nH]c(-c3cc(S(=O)(=O)N4CC(CO)C4)ccc3OCC)nn12] | 2-(2-ethoxy-5-((3- (hydroxymethyl)azetidin-1- yl)sulfonyl)phenyl)-5-methyl-7- propylimidazo[5,1-f][1,2,4]triazin- 4(3H)-one |
| 43 | 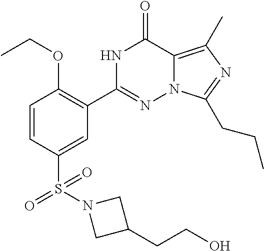 [SMILES: CCCc1nc(C)c2c(=O)[nH]c(-c3cc(S(=O)(=O)N4CC(CCO)C4)ccc3OCC)nn12] | 2-(2-ethoxy-5-((3-(2- hydroxyethyl)azetidin-1- yl)sulfonyl)phenyl)-5-methyl-7- propylimidazo[5,1-f][1,2,4]triazin- 4(3H)-one |
| 44 | 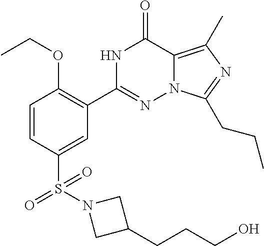 [SMILES: CCCc1nc(C)c2c(=O)[nH]c(-c3cc(S(=O)(=O)N4CC(CCCO)C4)ccc3OCC)nn12] | 2-(2-ethoxy-5-((3-(3- hydroxypropyl)azetidin-1- yl)sulfonyl)phenyl)-5-methyl-7- propylimidazo[5,1-f][1,2,4]triazin- 4(3H)-one |
| 45 | 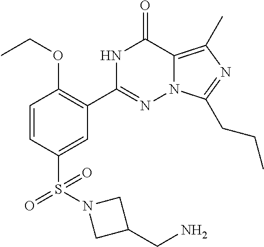 [SMILES: CCCc1nc(C)c2c(=O)[nH]c(-c3cc(S(=O)(=O)N4CC(CN)C4)ccc3OCC)nn12] | 2-(5-((3-(aminomethyl)azetidin-1- y])sulfonyl)-2-ethoxyphenyl)-5- methyl-7-propylimidazo[5,1- f][1,2,4]triazin-4(3H)-one |
| 46 | 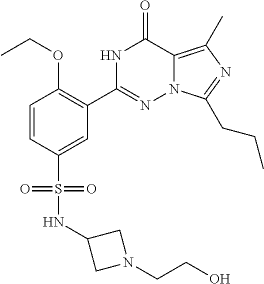 [SMILES: CCCc1nc(C)c2c(=O)[nH]c(-c3cc(S(=O)(=O)NC4CN(CCO)C4)ccc3OCC)nn12] | 4-ethoxy-N-(1-(2- hydroxyethyl)azetidin-3-yl)-3-(5- methyl-4-oxo-7-propyl-3,4- dihydroimidazo[5,1-f][1,2,4]triazin-2- yl)benzenesulfonamide |
| 47 | 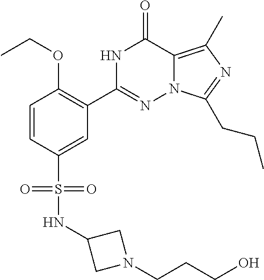 [SMILES: CCCc1nc(C)c2c(=O)[nH]c(-c3cc(S(=O)(=O)NC4CN(CCCO)C4)ccc3OCC)nn12] | 4-ethoxy-N-(1-(3- hydroxypropyl)azetidin-3-yl)-3-(5- methyl-4-oxo-7-propyl-3,4- dihydroimidazo[5,1-f][1,2,4]triazin-2- yl)benzenesulfonamide |
| 48 | 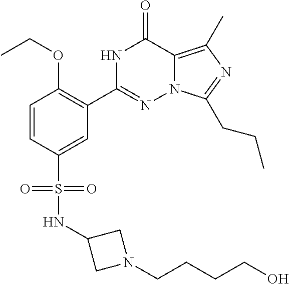 [SMILES: CCCc1nc(C)c2c(=O)[nH]c(-c3cc(S(=O)(=O)NC4CN(CCCCO)C4)ccc3OCC)nn12] | 4-ethoxy-N-(1-(4- hydroxybutyl)azetidin-3-yl)-3-(5- methyl-4-oxo-7-propyl-3,4- dihydroimidazo[5,1-f][1,2,4]triazin-2- yl)benzenesulfonamide |
| 49 | 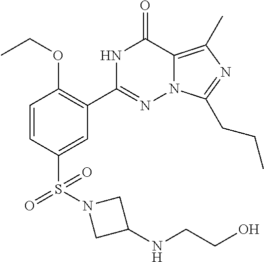 [SMILES: CCCc1nc(C)c2c(=O)[nH]c(-c3cc(S(=O)(=O)N4CC(NCCO)C4)ccc3OCC)nn12] | 2-(2-ethoxy-5-((3-((2- hydroxyethyl)amino)azetidin-1- yl)sulfonyl)phenyl)-5-methyl-7- propylimidazo[5,1-f][1,2,4]triazin- 4(3H)-one |
| 50 | 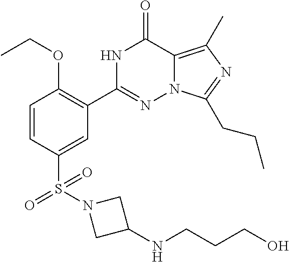 [SMILES: CCCc1nc(C)c2c(=O)[nH]c(-c3cc(S(=O)(=O)N4CC(NCCCO)C4)ccc3OCC)nn12] | 2-(2-ethoxy-5-((3-((3- hydroxypropyl)amino)azetidin-1- yl)sulfonyl)phenyl)-5-methyl-7- propylimidazo[5,1-f][1,2,4]triazin- 4(3H)-one |
| 51 | 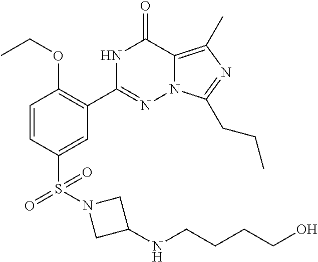 [SMILES: CCCc1nc(C)c2c(=O)[nH]c(-c3cc(S(=O)(=O)N4CC(NCCCCO)C4)ccc3OCC)nn12] | 2-(2-ethoxy-5-((3-((4- hydroxybutyl)amino)azetidin-1- yl)sulfonyl)phenyl)-5-methyl-7- propylimidazo[5,1-f][1,2,4]triazin- 4(3H)-one |
| 52 | 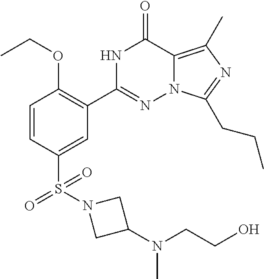 [SMILES: CCCc1nc(C)c2c(=O)[nH]c(-c3cc(S(=O)(=O)N4CC(N(C)CCO)C4)ccc3OCC)nn12] | 2-(2-ethoxy-5-((3-((2- hydroxyethyl)(methyl)amino)azetidin- 1-yl)sulfonyl)phenyl)-5-methyl-7- propylimidazo[5,1-f][1,2,4]triazin- 4(3H)-one |
| 53 | 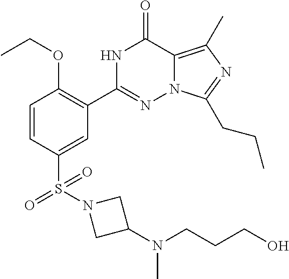 [SMILES: CCCc1nc(C)c2c(=O)[nH]c(-c3cc(S(=O)(=O)N4CC(N(C)CCCO)C4)ccc3OCC)nn12] | 2-(2-ethoxy-5-((3-((3- hydroxypropyl)(methyl)amino) azetidin-1- yl)sulfonyl)phenyl)-5-methyl-7- propylimidazo[5,1-f][1,2,4]triazin- 4(3H)-one |
| 54 | 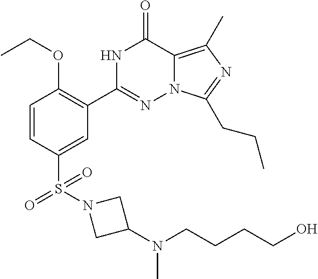 [SMILES: CCCc1nc(C)c2c(=O)[nH]c(-c3cc(S(=O)(=O)N4CC(N(C)CCCCO)C4)ccc3OCC)nn12] | 2-(2-ethoxy-5-((3-((4- hydroxybutyl)(methyl)amino)azetidin- 1-yl)sulfonyl)phenyl)-5-methyl-7- propylimidazo[5,1-f][1,2,4]triazin- 4(3H)-one |
| 55 | 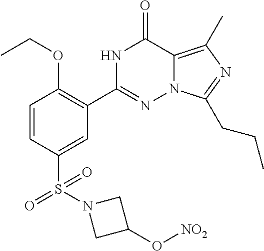 [SMILES: CCCc1nc(C)c2c(=O)[nH]c(-c3cc(S(=O)(=O)N4CC(O[N+](=O)[O-])C4)ccc3OCC)nn12] | 1-((4-ethoxy-3-(5-methyl-4-oxo-7- propyl-3,4-dihydroimidazo[5,1- f][1,2,4]triazin-2- yl)phenyl)sulfonyl)azetidin-3-yl nitrate |
| 56 | 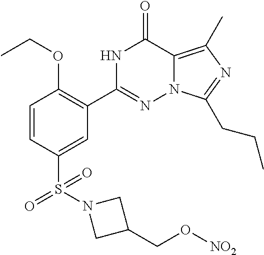 [SMILES: CCCc1nc(C)c2c(=O)[nH]c(-c3cc(S(=O)(=O)N4CC(CO[N+](=O)[O-])C4)ccc3OCC)nn12] | (1-((4-ethoxy-3-(5-methyl-4-oxo-7- propyl-3,4-dihydroimidazo[5,1- f][1,2,4]triazin-2- yl)phenyl)sulfonyl)azetidin-3- yl)methyl nitrate |
| 57 | 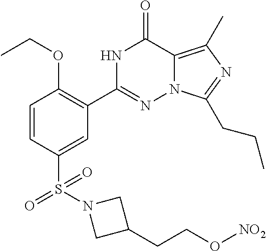 [SMILES: CCCc1nc(C)c2c(=O)[nH]c(-c3cc(S(=O)(=O)N4CC(CCO[N+](=O)[O-])C4)ccc3OCC)nn12] | 2-(1-((4-ethoxy-3-(5-methyl-4-oxo-7- propyl-3,4-dihydroimidazo[5,1- f][1,2,4]triazin-2- yl)phenyl)sulfonyl)azetidin-3-yl) ethyl nitrate |
| 58 | 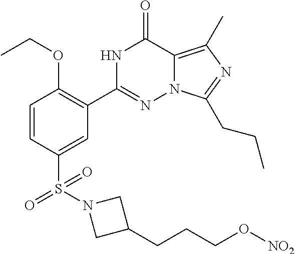 [SMILES: CCCc1nc(C)c2c(=O)[nH]c(-c3cc(S(=O)(=O)N4CC(CCCO[N+](=O)[O-])C4)ccc3OCC)nn12] | 3-(1-((4-ethoxy-3-(5-methyl-4-oxo-7- propyl-3,4-dihydroimidazo[5,1- f][1,2,4]triazin-2- yl)phenyl)sulfonyl)azetidin-3-yl) propyl nitrate |
| 59 | 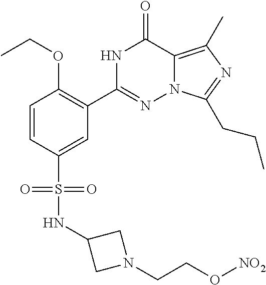 [SMILES: CCCc1nc(C)c2c(=O)[nH]c(-c3cc(S(=O)(=O)NC4CN(CCO[N+](=O)[O-])C4)ccc3OCC)nn12] | 2-(3-((4-ethoxy-3-(5-methyl-4-oxo-7- propyl-3,4-dihydroimidazo[5,1- f][1,2,4]triazin-2- yl)phenyl)sulfonamido)azetidin-1- yl)ethyl nitrate |
| 60 | 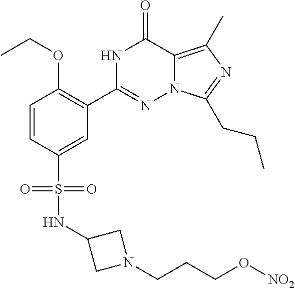 [SMILES: CCCc1nc(C)c2c(=O)[nH]c(-c3cc(S(=O)(=O)NC4CN(CCCO[N+](=O)[O-])C4)ccc3OCC)nn12] | 3-(3-((4-ethoxy-3-(5-methyl-4-oxo-7- propyl-3,4-dihydroimidazo[5,1- f][1,2,4]triazin-2- yl)phenyl)sulfonamido)azetidin-1- yl)propyl nitrate |
| 61 | 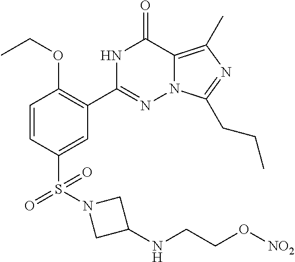 [SMILES: CCCc1nc(C)c2c(=O)[nH]c(-c3cc(S(=O)(=O)N4CC(NCCO[N+](=O)[O-])C4)ccc3OCC)nn12] | 2-((1-((4-ethoxy-3-(5-methyl-4-oxo-7- propyl-3,4-dihydroimidazo[5,1- f][1,2,4]triazin-2- yl)phenyl)sulfonyl)azetidin-3- yl)amino)ethyl nitrate |
| 62 | 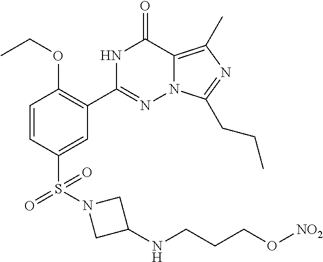 [SMILES: CCCc1nc(C)c2c(=O)[nH]c(-c3cc(S(=O)(=O)N4CC(NCCCO[N+](=O)[O-])C4)ccc3OCC)nn12] | 3-((1-((4-ethoxy-3-(5-methyl-4-oxo-7- propyl-3,4-dihydroimidazo[5,1- f][1,2,4]triazin-2- yl)phenyl)sulfonyl)azetidin-3- yl)amino)propyl nitrate |
| 63 | 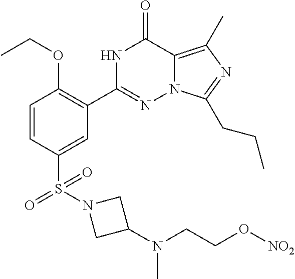 [SMILES: CCCc1nc(C)c2c(=O)[nH]c(-c3cc(S(=O)(=O)N4CC(N(C)CCO[N+](=O)[O-])C4)ccc3OCC)nn12] | 2-((1-((4-ethoxy-3-(5-methyl-4-oxo-7- propyl-3,4-dihydroimidazo[5,1- f][1,2,4]triazin-2- yl)phenyl)sulfonyl)azetidin-3- yl)(methyl)amino)ethyl nitrate |
| 64 | 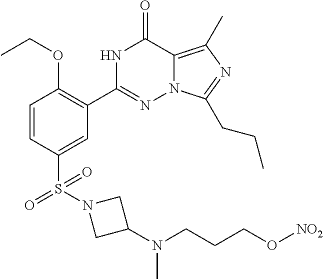 [SMILES: CCCc1nc(C)c2c(=O)[nH]c(-c3cc(S(=O)(=O)N4CC(N(C)CCCO[N+](=O)[O-])C4)ccc3OCC)nn12] | 3-((1-((4-ethoxy-3-(5-methyl-4-oxo-7- propyl-3,4-dihydroimidazo[5,1- f][1,2,4]triazin-2- yl)phenyl)sulfonyl)azetidin-3- yl)(methyl)amino)propyl nitrate |
| 65 | 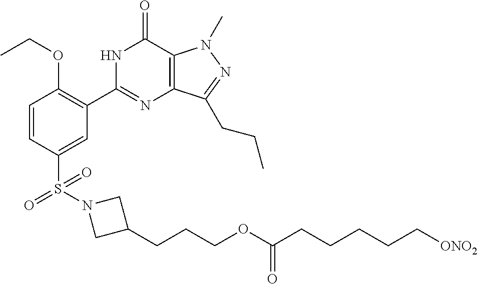 | 3-(1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)propyl 6-(nitrooxy)hexanoate |
| 66 | 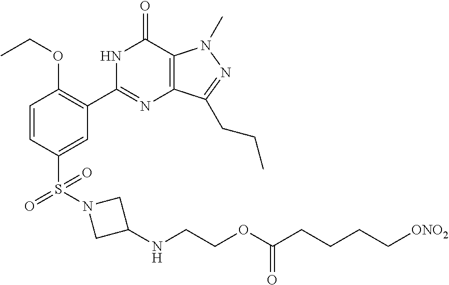 | 2-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)amino)ethyl 5-(nitrooxy)pentanoate |
| 67 | 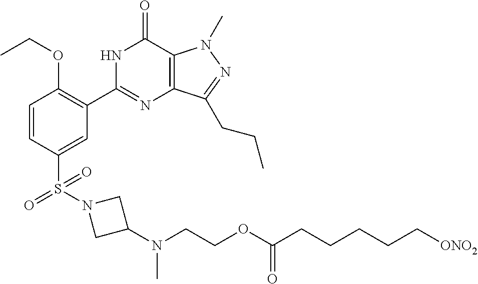 | 2-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)(methyl)amino)ethyl 6- (nitrooxy)hexanoate |
| 68 | 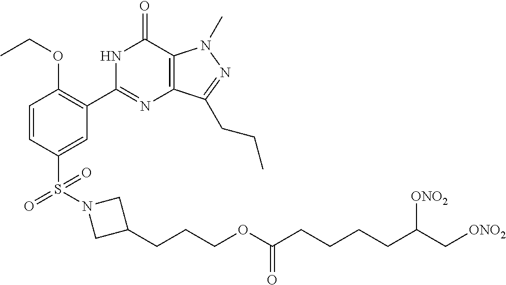 | 3-(1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)propyl 6,7-bis(nitrooxy)heptanoate |
| 69 | 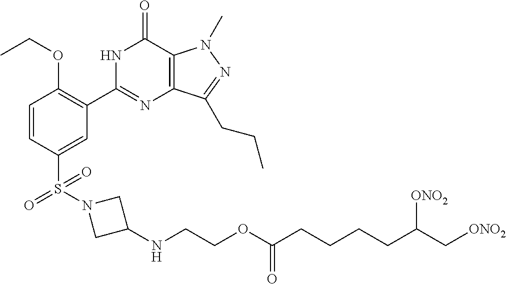 | 2-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)amino)ethyl 6,7- bis(nitrooxy)heptanoate |
| 70 | 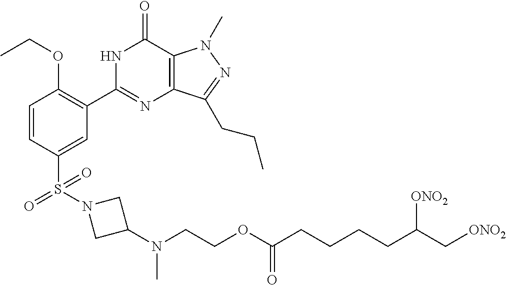 | 2-((1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)(methyl)amino)ethyl 6,7- bis(nitrooxy)heptanoate |
| 71 | 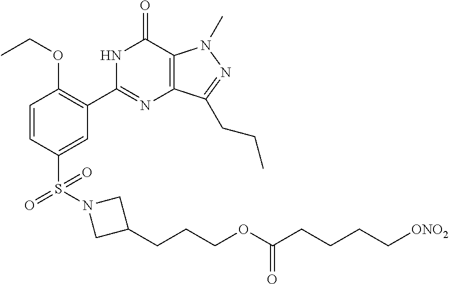 | 3-(1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)propyl 5-(nitrooxy)pentanoate |
| 72 | 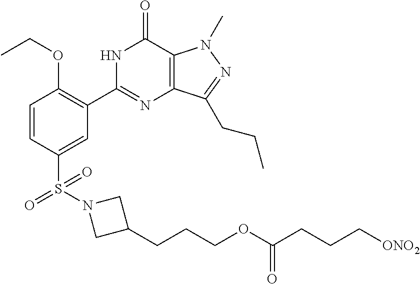 | 3-(1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)propyl 4-(nitrooxy)butanoate |
| 73 | 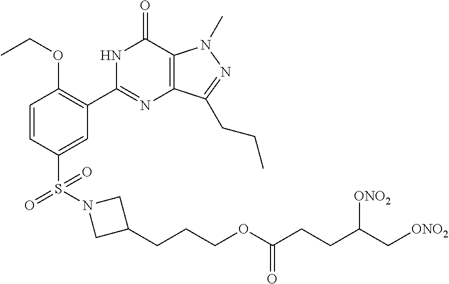 | 3-(1-((4-ethoxy-3-(1-methyl-7-oxo-3- propyl-6,7-dihydro-1H-pyrazolo[4,3- d]pyrimidin-5- yl)phenyl)sulfonyl)azetidin-3- yl)propyl 4,5-bis(nitrooxy)pentanoate |
| 74 | 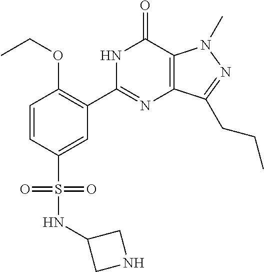 [SMILES: CCCc1nn(C)c2c(=O)[nH]c(-c3cc(S(=O)(=O)NC4CNC4)ccc3OCC)nc12] | N-(azetidin-3-yl)-4-ethoxy-3-(1- methyl-7-oxo-3-propyl-6,7-dihydro- 1H-pyrazolo[4,3-d]pyrirmdin-5- yl)benzenesulfonamide |
| 75 | 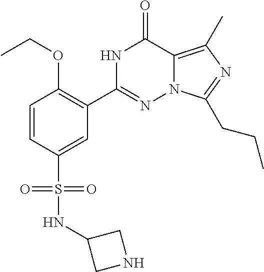 [SMILES: CCCc1nc(C)c2c(=O)[nH]c(-c3cc(S(=O)(=O)NC4CNC4)ccc3OCC)nn12] | N-(azetidin-3-yl)-4-ethoxy-3-(5- methyl-4-oxo-7-propyl-3,4- dihydroimidazo[5,1-f][1,2,4]triazin-2- yl)benzenesulfonamide |

### Isotopically Labelled Analogs

The present disclosure also encompasses isotopically-labeled compounds which are identical to those compounds as described herein, except that one or more atoms are replaced by an atom having an atomic mass or mass number different from the atomic mass or mass number usually found in nature (“isotopologues”). The compounds of the present disclosure may also contain unnatural proportions of atomic isotopes at one or more atoms that constituted such compounds. Examples of isotopes that can be incorporated into compounds described herein include isotopes of hydrogen, carbon, nitrogen, oxygen, phosphorus, fluorine and chlorine, such as ²H (“D”), ³H, ¹³C, ¹⁴C, ¹⁵N, ¹⁸O, ¹⁷O, ³¹P, ³²P, ³⁵S, ¹⁸F, and ³⁶Cl, respectively. For example, a compound described herein can have one or more H atoms replaced with deuterium.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

In some embodiments, certain isotopically-labeled compounds, such as those labeled with ³H and ¹⁴C, can be useful in compound and/or substrate tissue distribution assays. Tritiated (³H) and carbon-14 (¹⁴C) isotopes can be particularly preferred for their ease of preparation and detectability. Further, substitution with heavier isotopes such as deuterium can afford certain therapeutic advantages resulting from greater metabolic stability, such as increased in vivo half-life or reduced dosage requirements, and hence can be preferred in some circumstances. Isotopically-labeled compounds can generally be prepared by following procedures analogous to those disclosed herein, for example, in the Examples section, by substituting an isotopically-labeled reagent for a non-isotopically-labeled reagent.

In some embodiments, the compounds disclosed in the present disclosure are deuterated analogs of any of the compounds, or a salt thereof, as described herein. A deuterated analog of a compound of formula (I)-(IIIb) is a compound where one or more hydrogen atoms are substituted with a deuterium. In some embodiments, the deuterated analog is a compound of formula (I) that includes a deuterated R¹, R², R³, R⁴, R⁵, or R⁶ group.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

### Fluorinated Analogs

In some embodiments, the compounds disclosed in the present disclosure are fluorinated analogs of any of the compounds, or a salt thereof, as described herein. A fluorinated analog of a compound of formula (I)-(III) is a compound where one or more hydrogen atoms or substituents are substituted with a fluorine atom. In some embodiments, the fluorinated analog is a compound of formula (I) that includes a fluorinated R¹, R², R³, R⁴, R⁵, or R⁶ group. In some embodiments of a fluorinated analog of a compound of formula (I), the hydrogen atom of an aliphatic or an aromatic C—H bond is replaced by a fluorine atom. In some embodiments of a fluorinated analog of a compound of formula (I), at least one hydrogen of an optionally substituted aryl or an optionally substituted heteroaryl is replaced by a fluorine atom. In some embodiments of a fluorinated analog of a compound of formula (I), a hydroxyl substituent (—OH) or an amino substituent (—NH₂) is replaced by a fluorine atom. In some embodiments of a fluorinated analog of a compound, the compound includes one or more substituents independently selected from —F, —CF₃, —CF₂CF₃, —CHF₂, —OCF₃, —OCHF₂, and —OCF₂CF₃.

### Isomers

The term “compound”, as used herein, is meant to include all stereoisomers, geometric isomers, tautomers, and isotopes of the structures depicted.

The compounds herein described may have asymmetric centers, geometric centers (e.g., double bond), or both. All chiral, diastereomeric, racemic forms and all geometric isomeric forms of a structure are intended, unless the specific stereochemistry or isomeric form is specifically indicated. In some embodiments, the compounds described herein have one or more chiral centers. It is understood that if an absolute stereochemistry is not expressly indicated, then each chiral center may independently be of the R-configuration or the S-configuration or a mixture thereof. Thus, compounds described herein include enriched or resolved optical isomers at any or all asymmetric atoms as are apparent from the depictions. Racemic mixtures of R-enantiomer and S-enantiomer, and enantio-enriched stereoisomeric mixtures comprising of R- and S-enantiomers, as well as the individual optical isomers can be isolated or synthesized so as to be substantially free of their enantiomeric or diastereomeric partners, and these stereoisomers are all within the scope of the present technology.

Compounds of the present disclosure containing an asymmetrically substituted atom may be isolated in optically active or racemic forms. It is well known in the art how to prepare optically active forms, such as by resolution of racemic forms, by synthesis from optically active starting materials, or through use of chiral auxiliaries.

Geometric isomers, resulting from the arrangement of substituents around a carbon-carbon double bond or arrangement of substituents around a cycloalkyl or heterocyclic ring, can also exist in the compounds of the present disclosure. Geometric isomers of olefins, C═N double bonds, or other types of double bonds may be present in the compounds described herein, and all such stable isomers are included in the present disclosure. Specifically, cis and trans geometric isomers of the compounds of the present disclosure may also exist and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

### Salts and Other Forms

In some embodiments, the compounds described herein are present in a salt form. In some embodiments, the compounds are provided in the form of pharmaceutically acceptable salts.

Compounds included in the present compositions that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, chloride, 2,2,2-trifluoroacetate (TFA), and formate salts.

Compounds containing an amine functional group or a nitrogen-containing heteroaryl group may be basic in nature and may react with a variety of inorganic and organic acids to form the corresponding pharmaceutically acceptable salts. Inorganic acids commonly employed to form such salts include hydrochloric, and related inorganic acids. Organic acids commonly employed to form such salts include formic acid, and related organic acids. Such pharmaceutically acceptable salts thus include chloride, and related salts.

Other examples of salts include anions of the compounds of the present disclosure compounded with a suitable cation such as N⁺, NH₄⁺, and NW₄⁺ (where W can be a C₁-C₈ alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

The compounds described herein can be present in various forms including crystalline, powder and amorphous forms of those compounds, pharmaceutically acceptable salts, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including dimethylformamide (DMF), ether, and alcohol solvates, among others. The identification and preparation of any particular solvate is within the skill of the ordinary artisan of synthetic organic or medicinal chemistry.

In some embodiments, the compounds described herein are present in a solvate form. In some embodiments, the compounds described herein are present in a hydrate form when the solvent component of the solvate is water.

### Prodrugs

In some embodiments, the compounds described herein are present in a prodrug form. Any convenient prodrug forms of the subject compounds can be prepared, for example, according to the strategies and methods described by Rautio et al. (“Prodrugs: design and clinical applications”, Nature Reviews Drug Discovery 7, 255-270 (February 2008)).

### Compound Synthesis

Compounds of the present disclosure may be synthesized according to standard methods known in the art [see, e.g. Morrison and Boyd in “Organic Chemistry”, 6^{th} edition, Prentice Hall (1992)]. Some compounds and/or intermediates of the present disclosure may be commercially available, known in the literature, or readily obtainable by those skilled in the art using standard procedures. Some compounds of the present disclosure may be synthesized using schemes, examples, or intermediates described herein. Where the synthesis of a compound, intermediate or variant thereof is not fully described, those skilled in the art can recognize that the reaction time, number of equivalents of reagents and/or temperature may be modified from reactions described herein to prepare compounds presented or intermediates or variants thereof and that different work-up and/or purification techniques may be necessary or desirable to prepare such compounds, intermediates, or variants.

Synthesized compounds may be validated for proper structure by methods known to those skilled in the art, for example by nuclear magnetic resonance (NMR) spectroscopy and/or mass spectrometry.

### Compositions

Compounds of the present disclosure may be included in a composition that includes one or more such compounds and at least one excipient (e.g., a pharmaceutically acceptable excipient). Such compositions may include an inhibitor compound of PDE-5 and/or -6, or a NO-donating and PDE-5 and/or -6 inhibiting compound (e.g., as described herein).

The compounds described herein can find use in pharmaceutical compositions for administration to a subject in need thereof in a variety of therapeutic applications where inhibition of PDE-5 and/or -6 is desirable. In some embodiments, compounds of the present disclosure may be formulated as pharmaceutical compositions.

Accordingly, in a second aspect, the present disclosure provides pharmaceutical compositions comprising at least one compound described herein, a pharmaceutically acceptable salt thereof, or a prodrug thereof, and at least one pharmaceutically acceptable excipient. The phrase “pharmaceutically acceptable excipient,” refers any ingredient other than the inventive compounds described herein (for example, a vehicle capable of suspending or dissolving the active compound, or any other convenient pharmaceutically acceptable carriers, excipients or additives) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, dispensing, or dispersing agents, sweeteners, and waters of hydration. In some embodiments, the pharmaceutical composition comprises a compound as described herein, a pharmaceutically acceptable salt thereof, or a prodrug thereof in a therapeutically effective amount.

The pharmaceutical composition may be formulated according to any convenient methods, and may be prepared in various forms for oral administration such as tablets, pills, powders, capsules, syrups, emulsions and microemulsions, or in forms for non-oral administration such as eye drops or preparations for intramuscular, intravenous or subcutaneous administration. In one example, the pharmaceutical composition may be administered through the eyes in the form of eyedrops. In one example, the pharmaceutical composition may be an ophthalmic composition, such as an eye drop composition.

In some embodiments, the pharmaceutical compositions are formulated for oral delivery. In a case wherein the pharmaceutical composition is prepared in a form for oral administration, examples of additives or carriers which may be used include cellulose, calcium silicate, corn starch, lactose, sucrose, dextrose, calcium phosphate, magnesium stearate, stearic acid, stearate, talc, surfactant, suspending agent, emulsifier and diluent. Examples of additives or carriers which may be used in a case wherein the pharmaceutical composition of the present invention is prepared as an injection include water, saline solution, glucose aqueous solution, pseudosugar solution, alcohol, glycol, ether (e.g., polyethylene glycol 400), oil, fatty acid, fatty acid ester, glyceride, surfactants, suspending agents and emulsifiers.

In some embodiments, the pharmaceutical compositions are formulated for parenteral administration to a subject in need thereof. In some parenteral embodiments, the pharmaceutical compositions are formulated for intravenous administration to a subject in need thereof. In some parenteral embodiments, the pharmaceutical compositions are formulated for subcutaneous administration to a subject in need thereof.

In some embodiments, the pharmaceutical compositions are formulated for ophthalmic administration. In some embodiments, the pharmaceutical compositions are formulated for topical administration.

In a third aspect, the present disclosure provides an ophthalmic composition comprising a therapeutically effective amount of a compound or a pharmaceutically acceptable salt thereof as described herein, and a physiologically compatible ophthalmic vehicle.

In some embodiments of the ophthalmic composition, the composition is an aqueous solution. In some embodiments, the ophthalmic composition is an eye drop composition.

In the eye drop composition according to one example, an anionic polymer such as hyaluronic acid and carboxymethylcellulose or pharmaceutically acceptable salts of the same, or other substances which play a moisturizing and lubricating role in eye drops, may be included. In addition to these substances, pharmaceutically acceptable carriers may also be included. Examples of such pharmaceutically acceptable carriers include isotonic agents, buffers, stabilizers, pH modulators and solvents. Isotonic agents play a role of regulating the tonicity of eye drops, and common choices may be sodium chloride or potassium chloride. Buffers perform the function of regulating the acidity or alkalinity of the eye drops. Buffers commonly used in the preparation of eye drops include aminocaproic acid, dibasic sodium phosphate, and monobasic sodium phosphate. Stabilizers perform the function of stabilizing eye drops, and common stabilizers which may be used include disodium edetate and/or sodium perborate. pH regulators regulate the pH of an eye drop composition, and examples include hydrochloric acid and/or sodium hydroxide. As the solvent, sterilized distilled water or sterile water for injection may be used. The eye drop composition may be in the form of a liquid, a gel, or an ointment. The eye drop composition according to one example may be in the form of a liquid. The eye drop composition may include preservative agents and antimicrobials as needed.

In some embodiments, the ophthalmic compositions are formulated for ophthalmic administration. In some embodiments, the ophthalmic compositions are formulated for topical administration.

### Methods of Increasing Activity of Protein Kinase G (PKG)

Aspects of the present disclosure include methods of increasing or activating activity of PKG in a biological system or sample by contacting with compounds which exhibit dual functionality by: i) inhibiting PDE-5 and PDE-6 to increase activity of protein kinase G (PKG), and ii) activating soluble guanylate cyclase (sGC) via nitric oxide (NO) donation from a nitric oxide (NO) donor substituent group of the compound. In some embodiments, the compound is a cGMP-reliant PKG activator that includes a NO donor substituent group and simultaneously inhibits both PDE-5 and PDE-6.

In certain embodiments, the biological system or sample is in vitro. In another embodiment, the biological system or sample is in vivo. In some instances, the sample is a cellular sample.

Also provided are methods of using compounds that lack a NO donating substituent and exhibit potent inhibition activity of PDE-5 and/or PDE-6. In some embodiments, the compounds exhibit desirable activity by simultaneously inhibiting both PDE-5 and PDE-6.

“Protein kinase G (PKG)” is a serine/threonine specific protein which is activated by cGMP, and is also referred to as cGMP-dependent protein kinase, cGMP in cells is synthesized by guanylate cyclase (GC), and is broken down by phosphodiesterases PDEs). Further, 11 types of PDEs exist in the organs of the human body, with phosphodiesterases 2, 3 and 4 specific to cAMP while phosphodiesterases 5 and 6 are reported to act specifically on cGMP.

Soluble guanylate cyclase (sGC) is a receptor for nitric oxide (NO) and can be activated by a NO-donating composition to increase cyclic guanosine monophosphate (cGMP), which in turn increases the activity of protein kinase G (PKG). Nitric oxide is a physiological transmitter and plays a core role in regulating ocular pressure in healthy eyes and has blood vessel relaxing characteristics, nitric oxide (NO) refers to a compound in which nitrogen is oxygenated. Nitric oxide is basically a free radical, and includes unpaired electrons (indicated by the dot in .NO) within its chemical structure. Nitric oxide plays an important role in regulating blood pressure, neurotransmission, and maintaining homeostasis in the immune processes. For example, nitric oxide can increase guanylate cyclase (GC). Soluble guanylate cyclase (sGC) is a receptor for nitric oxide (NO) found in the cytoplasm. Soluble guanylate cyclase (sGC) is activated by a nitric oxide (NO) donor drug to increase cGMP, and accordingly increases activity of protein kinase G (PKG). Further, nitric oxide has vascular relaxation characteristics for which it is used as a therapeutic agent for cardiovascular disease, and is a physiological signal transmitter which plays a role in regulating ocular pressure in healthy eyes.

Phosphodiesterase 5 (PDE-5) and phosphodiesterase 6 (PDE-6) are phosphodiesterases and have 45 to 48% homologous base sequences. PDE-6, unlike other phosphodiesterases, is highly distributed in the cone cells of the retina and plays a core role in transmitting visual signals. Inhibition of PDE-5 and/or PDE-6 suppresses the decomposition of cGMP and activates guanylate cyclase (GC) enzyme, which can then lead to increased activity of PKG along with increasing the concentration of cGMP. Increasing the activity of PKG can then cause phosphorylation of numerous biologically important targets, relaxation of the smooth muscles, and increase in the flow of blood.

The present disclosure provides compounds having potent PDE-5 and PDE-6 inhibition activity. The compounds can be assessed using a variety of assays. For example, Table 3 of Example 5 in the experimental section shows the IC₅₀ values for exemplary compounds in in vitro inhibition assays of PDE-5A1 and PDE-6C, in comparison to compounds sildenafil and vardenafil. Sildenafil has lower PDE-6 selectivity compared to PDE-5.

Aspects of the present disclosure include methods of inhibiting PDE-5 and/or -6 using PDE-5 and/or -6 inhibitor compounds described herein. Such methods may include methods of inhibiting PDE-5 and/or -6 in biological systems by contacting such systems with PDE-5 and/or -6 inhibiting compounds (e.g., PDE-5 and/or -6 inhibitor compounds having structures according to any of those of Tables 1 or a pharmaceutically acceptable salt thereof). Biological systems may include, but are not limited to, cells, tissues, organs, bodily fluids, organisms, non-mammalian subjects, and mammalian subjects (e.g., humans).

In some embodiments, the method of inhibiting PDE-5 and/or -6 comprises contacting a biological system or sample comprising PDE-5 and/or -6 with an effective amount of any of the compounds or a pharmaceutically acceptable salt thereof as described herein, or a pharmaceutical composition as described herein to inhibit PDE-5 and/or -6. In certain embodiments, the biological system or sample is in vitro. In another embodiment, the biological system or sample is in vivo.

The PDE-5 and/or -6 inhibitors may inhibit the enzymatic activity of PDE-5 and/or -6 in a sample, e.g., as assessed by a PDE-5 and/or -6 enzymatic inhibition assay described in Example 5. PDE-5 and/or -6 inhibitors according to such methods may have IC₅₀ values for PDE-5 and/or -6 inhibition (e.g., as assessed by the assay of Example 12) of less than 1000 nM, such as 200 nM or less, or 20 nM or less. Biological systems may include subjects (e.g., human subjects).

In some embodiments of the method, the PDE-5 and/or -6 inhibitors (e.g. the compound of formula (I)) exhibit dual functionality. In some embodiment, the dual functionality of the compounds as describe herein are to inhibit PDE-5 and/or -6 and to serve as a nitric oxide (NO) donor.

In some embodiments, the present disclosure provides methods of inhibiting PDE-5 and/or -6 activity in a subject. In some cases, the percentage of PDE-5 and/or -6 activity inhibited in a subject may be at least 10%, at least 20%, at least 30%, at least 40%, at least 50%, at least 60%, at least 70%, at least 80%, at least, 85%, at least 90%, at least 95%, at least 96%, at least 97%, at least 98%, at least 99%, at least 99.5%, or at least 99.9%. In some cases, this level of inhibition and/or maximum inhibition of PDE-5 and/or -6 activity may be achieved by from about 1 hour after administration to about 3 hours after administration, from about 2 hours after administration to about 4 hours after administration, from about 3 hours after administration to about 10 hours after administration, from about 5 hours after administration to about 20 hours after administration, or from about 12 hours after administration to about 24 hours after administration. Inhibition of PDE-5 and/or -6 activity may continue throughout a period of at least 1 day, of at least 2 days, of at least 3 days, of at least 4 days, of at least 5 days, of at least 6 days, of at least 7 days, of at least 2 weeks, of at least 3 weeks, of at least 4 weeks, of at least 8 weeks, of at least 3 months, of at least 6 months, or at least 1 year. In some cases, this level of inhibition may be achieved through daily administration. Such daily administration may include administration for at least 2 days, for at least 3 days, for at least 4 days, for at least 5 days, for at least 6 days, for at least 7 days, for at least 2 weeks, for at least 3 weeks, for at least 4 weeks, for at least 2 months, for at least 4 months, for at least 6 months, for at least 1 year, or for at least 5 years. In some cases, subjects may be administered compounds or compositions of the present disclosure for the life of such subjects.

The compound according to one example may evenly and simultaneously inhibit PDE-5 and PDE-6. In some cases, the compound exhibits a substantial degree of PDE-6 enzyme inhibiting activity versus that for PDE 5, based on IC50 value, e.g., a relative inhibition activity where PDE-6 is inhibited in the range of 0.4 to 3.0 times compared to PDE 5. For example, the compound may inhibit PDE-6 at 0.5 times to 4.0 times the level of activity as compared to the compound's activity at PDE-5. In some embodiments, the inhibition of PDE-6 may be 0.4× to 3.0× as compared to PDE-5 by the compounds described herein. For example, the compounds as described herein may inhibit PDE-6 0.5× to 4.0× as compared to PDE-5. In some cases, compounds which exhibit high relative inhibition activity at PDE-6 as compared to PDE-5 find use in treatment of eye disease. PDE 6, unlike other phosphodiesterases, is highly distributed in the cone cells of the retina and can be associated with eye disease.

In some embodiments, present disclosure provides methods of modulating the protein kinase G (PKG) or a PKG-associated activity in a subject. In some cases, the percentage of PKG or a PKG-associated activity modulated in a subject may be at least 10%, at least 20%, at least 30%, at least 40%, at least 50%, at least 60%, at least 70%, at least 80%, at least, 85%, at least 90%, at least 95%, at least 96%, at least 97%, at least 98%, at least 99%, at least 99.5%, or at least 99.9%.

In some embodiments, compounds of the present disclosure may be used in assays to assess PDE-5 and/or -6 inhibition and/or modulation of PKG or PKG-associated biological activity. Some assays may include diagnostic assays. In some cases, compounds may be included in methods of drug discovery. In some embodiments, methods of the present disclosure include use of PDE-5 and/or -6 inhibiting compounds of the present disclosure to assess PDE-5 and/or -6 inhibition by other compounds. Such methods may include conjugating PDE-5 and/or -6 inhibiting compounds with one or more detectable labels (e.g., fluorescent dyes) and measuring PDE-5 and/or -6 dissociation (via detectable label detection) in the presence of the other compounds. The detectable labels may include fluorescent compounds.

### Therapeutic Indications

Aspects of the present disclosure include methods of treating therapeutic indications of interest using compounds and/or compositions disclosed herein. The term “therapeutic indication” refers to any symptom, condition, disorder, or disease that may be alleviated, stabilized, improved, cured, or otherwise addressed by some form of treatment or other therapeutic intervention (e.g., through PDE-5 and/or -6 inhibitor administration). Therapeutic indications associated with PDE-5 and/or -6 and/or PKG biological activity and/or dysfunction are referred to herein as “PDE-5 and/or -6-related indications.” In some embodiments, methods of the present disclosure may include treating PDE-5 and/or -6-related indications by administering compounds and/or compositions disclosed herein (e.g., PDE-5 and/or -6 inhibitor compounds).

The terms “treat,” “treatment,” and the like, refer to relief from or alleviation of pathological processes. In the context of the present disclosure insofar as it relates to any of the other conditions recited herein below, the terms “treat,” “treatment,” and the like mean to relieve or alleviate at least one symptom associated with such condition, or to slow or reverse the progression or anticipated progression of such condition.

Eye Diseases

The present disclosure provides a method of treating or preventing eye disease in a subject using the subject compounds as therapeutic agents, and compositions including the compounds.

The compounds and compositions of this disclosure according to one example may, in addition to inhibiting PDE 5, simultaneously inhibit PDE 6 which is highly expressed in the retina to exhibit superior therapeutic effect against eye disease. In some embodiments, the compounds and compositions according to one example may, in addition to inhibiting PDE 5 and PDE 6, include a nitric oxide (NO) donating substituent group which activates the nitric oxide (NO) receptor soluble guanylate cyclase (sGC) to increase cGMP, and in turn increases activity of protein kinase G (PKG) to exhibit superior therapeutic effect against eye disease.

In some embodiments, the compounds and compositions of the present disclosure are effective in lowering the intraocular pressure (IOP) of test subjects when dosed at various concentrations. Example 8 describes intraocular pressure (IOP) lowering studies with an exemplary compound 18 in comparison to control compounds in an ocular normotensive rabbit model, which indicates that the compounds of this disclosure are effective at lowering intraocular pressure (IOP). In another embodiment, the compounds and compositions of the present disclosure are able to significantly lower the IOP of test subjects after its administration. Example 9 describes the IOP lowering effect of exemplary compound of this disclosure (Compound 18) in in vivo ocular normotensive or hypertensive rabbit models. Example 9 indicates that Compound 18 was well tolerated and stable IOP lowering effects were observed in the ocular hypertensive rabbit model. In summary, these results indicate that the compounds of this disclosure would be effective in treating eye diseases characterized by an increase in IOP, such as glaucoma.

In some embodiments of the method, topical administration of the compound of this disclosure provides a IOP lowering effect (e.g., a decrease in IOP of greater than 20%, such as greater than 30%, greater than 40%, or greater than 50% as compared to a baseline IOP prior to therapy), where the IOP lowering effect is stable and maintained at least during the period of the dosage regimen stable, and in some cases for a period of time after the regimen is complete.

Eye diseases, which are the object of prevention or therapy using the compounds and pharmaceutical compositions, are diseases associated with the eye, and include, but are not limited to, diseases such as glaucoma, age-related macular degeneration (AMD), diabetic retinopathy (DR), xerophthalmia, cataracts, uveitis, ischemic retinopathy, optic neuropathy, diabetic macular edema (DME), senile cataracts, conjunctivitis, Stephensen-Johnson Syndrome, Sjogren's Syndrome, dry eye syndrome (DES) (also known as keratoconjunctivitis sicca (KCS) or keratitis sicca), trauma, and trauma of the eye due to eye surgery (eye surgery refers to all surgery that involves incisions in the eyeball, including glaucoma surgery, cataract surgery, retinal surgery, LASIK surgery and LASEK surgery). The eye diseases of interest may be diseases or conditions accompanying old age, diabetes, inflammation or cancer, etc., oxidative stress of the retinal pigment epithelium, damage induced by hypoxia, and diseases associated with reduced or increased ocular blood flow. In one example, the eye disease may be, but is not limited to, glaucoma, age-related macular degeneration (AMD), diabetic retinopathy (DR), xerophthalmia, dry eye syndrome (DES) (also known as keratoconjunctivitis sicca (KCS) or keratitis sicca), cataracts or uveitis.

“Glaucoma” is a representative eye disease which is caused by inability to regulate ocular pressure, which, if not treated appropriately, damages the optic nerve to cause loss of eyesight and permanent loss of vision. Depending on the presence or absence of pressure on the iridocorneal angle, glaucoma is categorized as primary open angle glaucoma or angle closure glaucoma. Whereas the range of normal pressure within the eyeball is reported to be 10 to 21 mmHg, in actual cases, glaucoma progresses and causes optic nerve damage even at pressures of less than 21 mmHg. The transparent liquid which supplies the eye with nutrients is called the aqueous, produced by the ciliary body and drained out through the meshwork. If the path through the meshwork is affected, intraocular pressure rises, and glaucoma is caused.

“Age-related macular degeneration (AMD)” is a disease where, with the progression of aging, the macula, which is the part of the eye where images of objects form, degenerates and causes deterioration of eyesight. Age-related macular degeneration is categorized as non-exudative AMD (dry) and exudative or neovascular AMD (wet). Non-exudative AMD occurs with functional anomalies in the retinal pigment epithelium due to aging of photoreceptors. Functional anomalies in the retinal pigment epithelium cause changes in the permeability of Bruch's Membrane, causing brown fat residue to accumulate on the retina and form a Drusen, which impedes the supply of nutrients from the choroid to the retina and causes secretion of vascular growth factor to form new abnormal blood vessels on the choroid.

“Diabetic retinopathy (DR)” is a complication of diabetes, which occurs when capillaries in the retina are damaged. The major categories of diabetic retinopathy are nonproliferative diabetic retinopathy and proliferative diabetic retinopathy. Nonproliferative diabetic retinopathy manifests with bleeding and edema at the macula which is at the center of the retina, and turns into the proliferative form if left untreated. Proliferative diabetic retinopathy involves generation of new abnormal blood vessels, causing bleeding which fills the vitreous body with blood and reduces eyesight. Fibrous tissue grows in the vitreous body, causing retinal detachment, etc. and ultimately complete loss of vision.

“Xerophthalmia” is a disease which occurs when anomalies occur in the tear film due to an imbalance due to shortage of tears or excessive evaporation of the same. Xerophthalmia is a syndrome involving foreign body sensation or irritation, etc. due to instability in the tear film due to a shortage of tears or excessive evaporation of tears from the tear film. More specifically, xerophthalmia involves cases where tear secretion is reduced, and cases of Stephenson-Johnson syndrome or pemphigoid accompanied by illnesses of the eyeball and auxiliary organs of the eyes, that is, anomalies, inflammation or skin disease in the eyelids, and vitamin A deficiency and Sjogren's Syndrome, which are cases accompanied by systemic illness. Also included are cases where the surface of the eyeball which lies exposed between the eyelids is damaged, causing irritation, foreign body sensation, and dryness, where, if damage to the cornea is severe, inflammation occurs on the surface of the eyeball. As the lesion progresses, the eyes may appear bloodshot. As for complications, mild impairment of vision may be followed by corneal ulcers, corneal perforation and secondary bacterial infection. Vision impairment becomes severe when corneal scarring and angiogenesis occur.

“Dry eye syndrome (DES)” also known as keratoconjunctivitis sicca (KCS), or keratitis sicca, is a multifactorial disease of the tears and the ocular surface that results in discomfort, visual disturbance, and tear film instability, and can lead to damage to the ocular surface.

The terms “individual” and “subject” are used interchangeably and refer to a subject requiring treatment of a disease. More specifically, what is referred to is a human or non-human primate, mouse, dog, cat, horse, cow, rabbit, rat, or other mammal.

In some embodiments, the method further comprising identifying a subject suffering from or at risk of eye disease.

In some embodiments, the method further comprising identifying an underlying disease or condition associated with the eye disease.

In some embodiments, the method includes administering to an eye of a subject a therapeutically effective amount of a compound as described herein.

In some embodiments, the method includes administering to an eye of a subject a therapeutically effective amount of a pharmaceutical composition (e.g., ophthalmic composition) including a compound as described herein. In some embodiments, the ophthalmic composition is an eye drop composition.

The recommended dose and frequency of administration of the eye drop composition according to one example may be 1 to 3 drops per administration with 5 to 6 administrations daily, adjusted appropriately according to symptoms. The administration dose for specific patients may vary depending on patient body weight, age, sex, health condition, administration intervals, times administered, and the severity of the illness.

In some embodiments, one or more symptoms of the eye disease are reduced or alleviated in the subject after administration of the ophthalmic composition.

In some embodiments, the ophthalmic composition is topically administered to the eye daily or as needed. In another embodiment, the ophthalmic composition is topically administered to the eye once a day. In another embodiment, the ophthalmic solution is topically administered to the eye two times or more daily. In certain embodiments, the ophthalmic composition is a solution.

In some embodiments, the method includes oral administration of the subject compound or composition. The administration dose may be administrated orally or non-orally depending on the purpose, in an amount effective at prevention or therapy in the individual or patient in question. When administering orally, the compound may be administered so that 0.01 to 1000 mg, more specifically 0.1 to 300 mg of the active agent is administered per 1 kg body weight, and when administering non-orally, the compound may be administered so that 0.01 to 100 mg, more specifically 0.1 to 50 mg of the active ingredient is administered per 1 kg body weight. The dose may be administered at one time or over multiple administrations. The administration dose for a specific individual or patient should be decided based on various related factors such as the body weight, age, sex, health, diet, administration intervals, method of administration and severity of the illness, and may be appropriately increased or reduced by an expert. The administration doses stated above are not intended to limit the scope of the present invention in any manner. A physician or veterinarian have ordinary skill in related art may readily decide and prescribe an effective required dose for the pharmaceutical composition. For example, a physician or veterinarian may, beginning at levels less than that required for achieving the target therapeutic effect, gradually increase the dose of the compound of the present invention in a pharmaceutical composition until the intended effect is achieved.

The compounds and compositions of the present disclosure may be administered alone, in combination with a compound according to another example of the present disclosure, or in simultaneous, separate or sequential concomitant administration with at least one other therapeutic agent, for example with other pharmaceutical active ingredients such as eye disease therapeutic agents, antibiotics, anti-inflammatory agents and anti-microbials.

### Definitions

Unless defined otherwise, all technical and scientific terms used herein have the same meaning as commonly understood by one of ordinary skill in the art to which this disclosure pertains.

It is understood that the definitions provided herein are not intended to be mutually exclusive. Accordingly, some chemical moieties may fall within the definition of more than one term.

The symbol “ [symbol] ” refers to a covalent bond that is a single or a double bond.

The term “Cₓ-C_{y}” when used in conjunction with a chemical moiety, such as alkyl, alkenyl, or alkynyl is meant to include groups that contain from x to y carbons in the chain. For example, the term “C₁-C₆ alkyl” refers to substituted or unsubstituted saturated hydrocarbon groups, including straight-chain alkyl and branched-chain alkyl groups that contain from 1 to 6 carbons. In some embodiments, the term “(Cₓ-C_{y})alkylene” refers to a substituted or unsubstituted alkylene chain with from x to y carbons in the alkylene chain. For example “(Cₓ-C_{y})alkylene may be selected from methylene, ethylene, propylene, butylene, pentylene, and hexylene, any one of which is optionally substituted.

The term “alkyl” refers to an unbranched or branched saturated hydrocarbon chain. In some embodiments, alkyl as used herein has 1 to 20 carbon atoms ((C₁-C₂₀)alkyl), 1 to 10 carbon atoms ((C₁-C₁₀)alkyl), 1 to 8 carbon atoms ((C₁-C₈)alkyl), 1 to 6 carbon atoms ((C₁-C₆)alkyl), 1 to 5 carbon atoms ((C₁-C₅)alkyl) or 1 to 3 carbon atoms ((C₁-C₅)alkyl). Examples include, but are not limited to, methyl, ethyl, n-propyl, isopropyl, n-butyl, sec-butyl, tert-butyl, n-pentyl, 2-pentyl, isopentyl, neopentyl, n-hexyl, 2-hexyl, 3-hexyl, and 3-methyl pentyl. When an alkyl residue having a specific number of carbons is named, all geometric isomers having that number of carbons may be encompassed. For example, “butyl” can include n-butyl, sec-butyl, isobutyl and t-butyl, and “propyl” can include n-propyl and isopropyl. Unless stated otherwise specifically in the specification, an alkyl chain is optionally substituted by one or more substituents such as those substituents described herein.

The term “alkoxy” refers to an unbranched or branched alkyl group attached to an oxygen atom (alkyl-O—). In some embodiments, alkoxy as used herein has 1 to 20 carbon atoms ((C₁-C₂₀)alkoxy), 1 to 10 carbon atoms ((C₁-C₁₀)alkoxy), 1 to 8 carbon atoms ((C₁-C₈)alkoxy), 1 to 6 carbon atoms ((C₁-C₆)alkoxy), 1 to 5 carbon atoms ((C₁-C₅)alkoxy) or 1 to 3 carbon atoms ((C₁-C₅)alkoxy). Examples include, but are not limited to, methoxy, ethoxy, n-propoxy, and butoxy. When an alkoxy residue having a specific number of carbons is named, all geometric isomers having that number of carbons may be encompassed, such as isopropoxy, isobutoxy, and t-butoxy. Unless stated otherwise specifically in the specification, an alkoxy chain is optionally substituted by one or more substituents such as those substituents described herein.

The term “alkylene” refers to a straight divalent hydrocarbon chain linking the rest of the molecule to a radical group, consisting solely of carbon and hydrogen, containing no unsaturation, and preferably having from 1 to 20 carbon atoms ((C₁-C₂₀)alkylene), 1 to 10 carbon atoms ((C₁-C₁₀)alkylene), 1 to 6 carbon atoms ((C₁-C₆)alkylene), or 1 to 5 carbon atoms ((C₁-C₅)alkylene). Examples include, but are not limited to, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Unless stated otherwise specifically in the specification, an alkylene chain is optionally substituted by one or more substituents such as those substituents described herein. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

The term “alkenyl” refers to an aliphatic hydrocarbon group containing at least one carbon-carbon double bond including straight-chain, branched-chain and cyclic alkenyl groups. In some embodiments, the alkenyl group has 2-10 carbon atoms (a C₂₋₁₀ alkenyl). In another embodiment, the alkenyl group has 2-4 carbon atoms in the chain (a C₂₋₄ alkenyl). Exemplary alkenyl groups include, but are not limited to, ethenyl, propenyl, n-butenyl, i-butenyl, 3-methylbut-2-enyl, n-pentenyl, heptenyl, octenyl, cyclohexyl-butenyl and decenyl. An alkylalkenyl is an alkyl group as defined herein bonded to an alkenyl group as defined herein. The alkenyl group can be unsubstituted or substituted through available carbon atoms with one or more groups defined hereinabove for alkyl

The term “alkynyl” refers to straight or branched monovalent hydrocarbyl groups having from 2 to 6 carbon atoms and preferably 2 to 3 carbon atoms and having at least 1 and preferably from 1 to 2 sites of acetylenic (C≡C—) unsaturation. Examples of such alkynyl groups include, but are not limited to, acetylenyl (C≡CH), and propargyl (CH₂C≡CH).

The term “aryl” refers to a monocyclic or polycyclic group having at least one hydrocarbon aromatic ring, wherein all of the ring atoms of the at least one hydrocarbon aromatic ring are carbon. Aryl may include groups with a single aromatic ring (e.g., phenyl) and multiple fused aromatic rings (e.g., naphthyl, anthryl). Aryl may further include groups with one or more aromatic hydrocarbon rings fused to one or more non-aromatic hydrocarbon rings (e.g., fluorenyl; 2,3-dihydro-1H-indene; 1,2,3,4-tetrahydronaphthalene). In certain embodiments, aryl includes groups with an aromatic hydrocarbon ring fused to a non-aromatic ring, wherein the non-aromatic ring comprises at least one ring heteroatom independently selected from the group consisting of N, O, and S. For example, in some embodiments, aryl includes groups with a phenyl ring fused to a non-aromatic ring, wherein the non-aromatic ring comprises at least one ring heteroatom independently selected from the group consisting of N, O, and S (e.g., chromane; thiochromane; 2,3-dihydrobenzofuran; indoline). In some embodiments, aryl as used herein has from 6 to 14 carbon atoms ((C₆-C₁₄)aryl), or 6 to 10 carbon atoms ((C₆-C₁₀)aryl). Where the aryl includes fused rings, the aryl may connect to one or more substituents or moieties of the formulae described herein through any atom of the fused ring for which valency permits.

The term “cycloalkyl” refers to a monocyclic or polycyclic saturated hydrocarbon. In some embodiments, cycloalkyl has 3 to 20 carbon atoms ((C₃-C₂₀)cycloalkyl), 3 to 8 carbon atoms ((C₃-C₈)cycloalkyl), 3 to 6 carbon atoms ((C₃-C₆)cycloalkyl), or 3 to 5 carbon atoms ((C₃-C₅)cycloalkyl). In some embodiments, cycloalkyl has 3 to 8 carbon atoms having single or multiple cyclic rings including fused, bridged, and spiro ring systems. Examples of suitable cycloalkyl groups include, but are not limited to, adamantyl, cyclopropyl, cyclobutyl, cyclopentyl, cyclooctyl, octahydropentalenyl, octahydro-1H-indene, decahydronaphthalene, cubane, bicyclo[3.1.0]hexane, and bicyclo[1.1.1]pentane, and the like.

The term “carbocycle” refers to a saturated, unsaturated or aromatic ring system in which each atom of the ring system is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl.

The term “heterocycle” refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems.

The term “heteroaryl” refers to an aromatic group of from 4 to 10 carbon atoms and 1 to 4 heteroatoms selected from the group consisting of oxygen, nitrogen and sulfur within the ring. Such heteroaryl groups can have a single ring (i.e., pyridinyl or furyl) or multiple condensed rings (i.e., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N oxide (N→O), sulfinyl, or sulfonyl moieties. Preferred heteroaryls include 5 or 6 membered heteroaryls such as pyridinyl, pyrrolyl, indolyl, thiophenyl, and furanyl.

The term “heteroalkyl” refers to an alkyl substituent in which one or more of the carbon atoms and any attached hydrogen atoms are independently replaced with the same or different heteroatomic group. For example, 1, 2, or 3 carbon atoms may be independently replaced with the same or different heteroatomic substituent.

The term “substituted” refers to moieties having substituents replacing a hydrogen on one or more carbons or substitutable heteroatoms, e.g., NH or NH₂, of a compound. It will be understood that “substitution” or “substituted with” includes the implicit proviso that such substitution is in accordance with permitted valence of the substituted atom and the substituent, and that the substitution results in a stable compound. For example, stable compounds include, but is not limited to, compounds which do not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. The term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants, unless specified otherwise.

When referring to compound features, the phrase “optionally substituted” may be used interchangeably with the phrase “unsubstituted or substituted” and refers to when a non-hydrogen substituent may or may not be present on a given atom or group, and, thus, the description includes structures where a non-hydrogen substituent is present and structures where a non-hydrogen substituent is not present. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible and/or inherently unstable.

It will also be understood by those skilled in the art that when “optionally substituted” is used, any part of the following term can be substituted.

The terms “linker”, “linkage” and “linking group” are used interchangeably and refer to a linking moiety that covalently connects two or more substituents. A linking moiety may connect two groups where the linker may be linear, branched, cyclic or a single atom. In some embodiments, the linker is divalent. In some embodiments, the linker is a branched linker. In some embodiments, the two or more substituents that are covalently connected by the linking moiety are optionally substituted alkyl or alkoxy groups. In some embodiments, the linkers are selected from —CO₂—, —O—, —OCO—, —CONH—, —NHCO—, and —NH—.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}N (R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2). In another exemplary embodiment, substituents include alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo, thioxo, cyano, nitro, imino, oximo, hydrazine, —R^{b}OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{a}, R^{b}, and R^{c} are independently selected from hydrogen, alkyl, cycloalkyl, cycloalkylalkyl, aryl, aralkyl, heterocycloalkyl, heterocycloalkylalkyl, heteroaryl, and heteroarylalkyl; and wherein each R^{a}, R^{b}, and R^{c}, valence permitting, may be optionally substituted with alkyl, alkenyl, alkynyl, halogen, haloalkyl, haloalkenyl, haloalkynyl, oxo, thioxo, cyano, nitro, imino, oximo, hydrazine, —R^{b}OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2).

The term “isomers” refers to two or more compounds comprising the same numbers and types of atoms, groups or components, but with different structural arrangement and connectivity of the atoms.

The term “tautomer” refers to one of two or more structural isomers which readily convert from one isomeric form to another and which exist in equilibrium.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are non-superimposable mirror images of one another.

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns, or (4) kinetic resolution using stereoselective chemical or enzymatic reagents. Racemic mixtures also can be resolved into their respective enantiomers by well-known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations. See, for example, Carreira and Kvaerno, Classics in Stereoselective Synthesis, Wiley-VCH: Weinheim, 2009.

The symbol=denotes a bond that may be a single, double or triple bond as described herein. Substituents around a carbon-carbon double bond are designated as being in the “Z” or “E” configuration, where the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituent on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compound wherein the substituents are disposed on both the same and opposite sides of the plane of the ring are designated “cis/trans.”

Singular articles such as “a,” “an” and “the” and similar referents in the context of describing the elements are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (i.e., “such as”) provided herein, is intended merely to better illuminate the embodiments and does not pose a limitation on the scope of the claims unless otherwise stated.

In some embodiments, where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remain operable. Moreover, two or more steps or actions can be conducted simultaneously.

A dash (“ [symbol] ”) symbol that is not between two letters or symbols refers to a point of bonding or attachment for a substituent. For example, —NH₂ is attached through the nitrogen atom.

The term “pharmaceutically acceptable salt” refers to a salt which is acceptable for administration to a subject. It is understood that such salts, with counter ions, will have acceptable mammalian safety for a given dosage regime. Such salts can also be derived from pharmaceutically acceptable inorganic or organic bases and from pharmaceutically acceptable inorganic or organic acids, and may comprise organic and inorganic counter ions. The neutral forms of the compounds described herein may be converted to the corresponding salt forms by contacting the compound with a base or acid and isolating the resulting salts.

The terms “pharmaceutically acceptable excipient,” “pharmaceutically acceptable diluent,” “pharmaceutically acceptable carrier,” and “pharmaceutically acceptable adjuvant” are used interchangeably and refer to an excipient, diluent, carrier, or adjuvant that is useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. The phrase “pharmaceutically acceptable excipient” includes both one and more than one such excipient, diluent, carrier, and/or adjuvant.

The term “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (i.e., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

### 5.2. Exemplary Embodiments

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

Notwithstanding the appended claims, aspects of the present disclosure are illustrated by the following clauses.

Clause 1. A method of treating an eye disease, the method comprising administering to an eye of a subject a therapeutically effective amount of a compound an ophthalmic composition comprising the compound, wherein the compound is of formula (I):

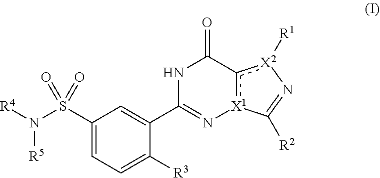

or a pharmaceutically acceptable salt, a solvate, a hydrate, a prodrug, or a stereoisomer thereof, wherein:

X¹ and X² are independently selected from N and C and at least one of X¹ and X² is N;

R¹ is —H, or optionally substituted (C₁-C₅)alkyl;

R² is optionally substituted (C₁-C₅)alkyl;

R³ is optionally substituted (C₁-C₅)alkoxy;

R⁴ is —H or optionally substituted (C₁-C₅)alkyl, and R⁵ is a 4-membered carbocycle or heterocycle ring that is substituted with one or more R⁶,

or R⁴ and R⁵ together with the nitrogen atom to which they are attached are cyclically linked to form a 4-membered heterocycle that is substituted with one or more R⁶; and

and each R⁶ is independently selected from —OH, —O—NO₂, optionally substituted (C₁-C₅)alkyl, optionally substituted (C₁-C₁₀)alkylene, optionally substituted (C₂-C₁₀) alkenyl, optionally substituted (C₂-C₁₀)alkynyl, optionally substituted (C₁-C₅)alkoxy, optionally substituted (C₃-C₅)heterocycle, optionally substituted (C₁-C₅)alkyl-(C₃-C₅)heterocycle-, optionally substituted (C₃-C₅)heterocycle-(C₁-C₅)alkyl-, optionally substituted (C₁-C₅)alkyl-Z¹—(C₁-C₅)alkyl-, optionally substituted (C₁-C₅)alkyl-Z¹—(C₁-C₅)alkoxy-, optionally substituted (C₁-C₁₀)alkyl-NR¹—, optionally substituted (C₁-C₁₀)alkyl-Z¹—(C₁-C₅)alkyl-NR¹—, optionally substituted (C₁-C₁₀)alkoxy-Z¹—(C₁-C₅)alkyl-NR¹—, substituted (C₁-C₅)alkyl-(C₃-C₅)heterocycle-(C₁-C₅)alkyl-, substituted linear linker, and substituted branched linker, wherein Z¹ is —CO₂—, —O—, —OCO—, —CONH—, —NHCO—, or —NH—, and the substituents of each R⁶ are independently selected from —O—NO₂, —ONO, —OH, —NH₂, —COOH, halogen, (C₁-C₃)alkoxy and (C₁-C₃)alkyl;

wherein at least one R⁶ is substituted with —O—NO₂, —ONO, —OH or —NH₂.

Clause 2. The method of clause 1, wherein the subject has an eye disease.

Clause 3. The method of clause 1 or 2, wherein the eye disease is selected from glaucoma, age-related macular degeneration (AMD), diabetic retinopathy (DR), xerophthalmia, cataracts, uveitis, ischemic retinopathy, optic neuropathy, diabetic macular edema (DME), senile cataracts, conjunctivitis, Stevens-Johnson Syndrome, Sjogren's Syndrome, dry eye syndrome (DES) (also known as keratoconjunctivitis sicca (KCS) or keratitis sicca), trauma, and trauma of the eye due to eye surgery.

Clause 4. The method of clause 3, wherein the eye disease is glaucoma.

Clause 5. The method of clause 3, wherein the eye disease is AMD.

Clause 6. The method of clause 3, wherein the eye disease is dry AMD.

Clause 7. The method of clause 4 or 5, further comprising identifying the subject as suffering from glaucoma or AMD.

Clause 8. The method of any one of clauses 1 to 7, wherein the ophthalmic composition comprises a physiologically compatible ophthalmic vehicle.

Clause 9. The method of clause 8, wherein the ophthalmic composition is an eye drop composition.

Clause 10. The method of any one of clauses 1 to 9, wherein the compound or composition is topically administered to the eye daily or as needed.

Clause 11. The method of clause 10, wherein the compound or composition is topically Clause administered to the eye once a day.

Clause 12. The method of clause 10, wherein the compound or composition is topically administered to the eye two times or more daily.

Clause 13. A method of treating a PDE-5 and/or -6-related indication, the method comprising administering to a subject in need thereof a therapeutically effective amount of a compound of formula (I):

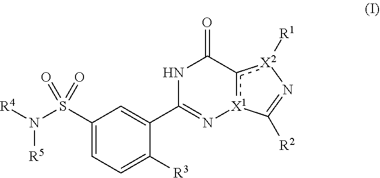

or a pharmaceutically acceptable salt, a solvate, a hydrate, a prodrug, or a stereoisomer thereof, wherein:

X¹ and X² are independently selected from N and C and at least one of X¹ and X² is N;

R¹ is —H, or optionally substituted (C₁-C₅)alkyl;

R² is optionally substituted (C₁-C₅)alkyl;

R³ is optionally substituted (C₁-C₅)alkoxy;

R⁴ is —H or optionally substituted (C₁-C₅)alkyl, and R⁵ is a 4-membered carbocycle or heterocycle ring that is substituted with one or more R⁶,

or R⁴ and R⁵ together with the nitrogen atom to which they are attached are cyclically linked to form a 4-membered heterocycle that is substituted with one or more R⁶; and

and each R⁶ is independently selected from —OH, —O—NO₂, optionally substituted (C₁-C₅)alkyl, optionally substituted (C₁-C₁₀)alkylene, optionally substituted (C₂-C₁₀) alkenyl, optionally substituted (C₂-C₁₀)alkynyl, optionally substituted (C₁-C₅)alkoxy, optionally substituted (C₃-C₅)heterocycle, optionally substituted (C₁-C₅)alkyl-(C₃-C₅)heterocycle-, optionally substituted (C₃-C₅)heterocycle-(C₁-C₅)alkyl-, optionally substituted (C₁-C₅)alkyl-Z¹—(C₁-C₅)alkyl-, optionally substituted (C₁-C₅)alkyl-Z¹—(C₁-C₅)alkoxy-, optionally substituted (C₁-C₁₀)alkyl-NR¹—, optionally substituted (C₁-C₁₀)alkyl-Z¹—(C₁-C₅)alkyl-NR¹—, optionally substituted (C₁-C₁₀)alkoxy-Z¹—(C₁-C₅)alkyl-NR¹—, substituted (C₁-C₅)alkyl-(C₃-C₅)heterocycle-(C₁-C₅)alkyl-, substituted linear linker, and substituted branched linker, wherein Z¹ is —CO₂—, —O—, —OCO—, —CONH—, —NHCO—, or —NH—, and the substituents of each R⁶ are independently selected from —O—NO₂, —ONO, —OH, —NH₂, —COOH, halogen, (C₁-C₃)alkoxy and (C₁-C₃)alkyl;

wherein at least one R⁶ is substituted with —O—NO₂, —ONO, —OH or —NH₂.

Clause 14. The method of clause 13, wherein the method comprises modulating the PKG signaling pathway via inhibiting PDE-5 and/or -6.

Clause 15. The method of clause 14, wherein the modulating activates PKG.

Clause 16. The method of any one of clauses 1 to 15, wherein in formula (I) at least one R⁶ is substituted with —O—NO₂.

Clause 17. The method of any one of clauses 1 to 16, wherein R¹ is (C₁-C₅)alkyl.

Clause 18. The method of any one of clauses 1 to 17, wherein R¹ is methyl.

Clause 19. The method of any one of clauses 1 to 18, wherein R² is n-propyl.

Clause 20. The method of any one of clauses 1 to 19, wherein R³ is ethoxy.

Clause 21. The method of clause 20, wherein the compound is of formula (Ia):

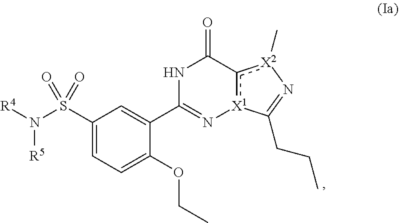

or a pharmaceutically acceptable salt, a solvate, a hydrate, a prodrug, or a stereoisomer thereof.

Clause 22. The method of any one of clauses 1 to 21, wherein R⁴ is —H and R⁵ is substituted azetidine.

Clause 23. The method of any one of clauses 1 to 22, wherein R⁴ and R⁵ together with the nitrogen atom to which they are attached are cyclically linked to form substituted azetidine.

Clause 24. The method of any one of clauses 1 to 23, wherein X¹ is N and X² is C.

Clause 25. The method of any one of clauses 1 to 23, wherein X¹ is C and X² is N.

Clause 26. The method of clause 22, wherein the compound is of formula (II):

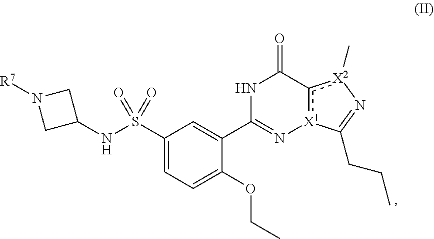

wherein:

R⁷ is selected from —H, R⁷⁰, and R⁷¹—Z²—R⁷²;

R⁷⁰, R⁷¹ and R⁷² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, and optionally substituted (C₁-C₅)alkoxy, wherein the optional substituent is selected from —OH, —NH₂, and —O—NO₂; and

Z² is —CO₂—, —O—, —OCO—, —CONH—, —NHCO—, or —NH—.

Clause 27. The method of clause 26, wherein the compound is of formula (IIa):

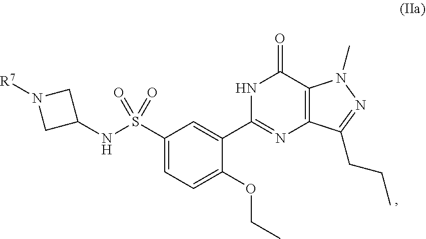

or a pharmaceutically acceptable salt, a solvate, a hydrate, a prodrug, or a stereoisomer thereof.

Clause 28. The method of clause 27, wherein:

### R⁷ is

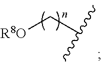

R⁸ is —H or —NO₂; and
n is 1, 2, 3, 4, or 5.

Clause 29. The method of clause 28, wherein the compound is selected from:

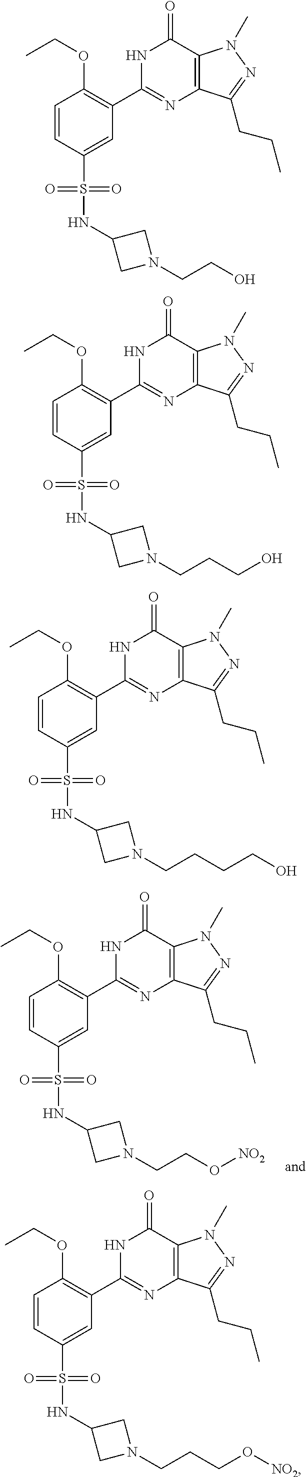

or a pharmaceutically acceptable salt, a solvate, a hydrate, a prodrug, or a stereoisomer thereof.

Clause 30. The method of clause 26, wherein the compound is of formula (IIb):

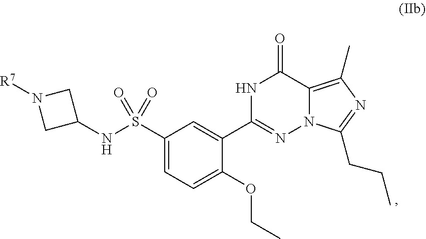

or a pharmaceutically acceptable salt, a solvate, a hydrate, a prodrug, or a stereoisomer thereof, wherein:

R⁷ is selected from —H, R⁷⁰, and R⁷¹—Z²—R⁷²;

R⁷⁰, R⁷¹ and R⁷² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, and optionally substituted (C₁-C₅)alkoxy, wherein the optional substituent is selected from —OH, —NH₂, and —O—NO₂; and

Z² is —CO₂—, —O—, —OCO—, —CONH—, —NHCO—, or —NH—.

Clause 31. The method of clause 30, wherein:

R⁷ is

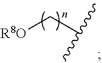

R⁸ is —H or —NO₂; and

n is 1, 2, 3, 4, or 5.

Clause 32. The method of clause 31, wherein the compound is selected from:

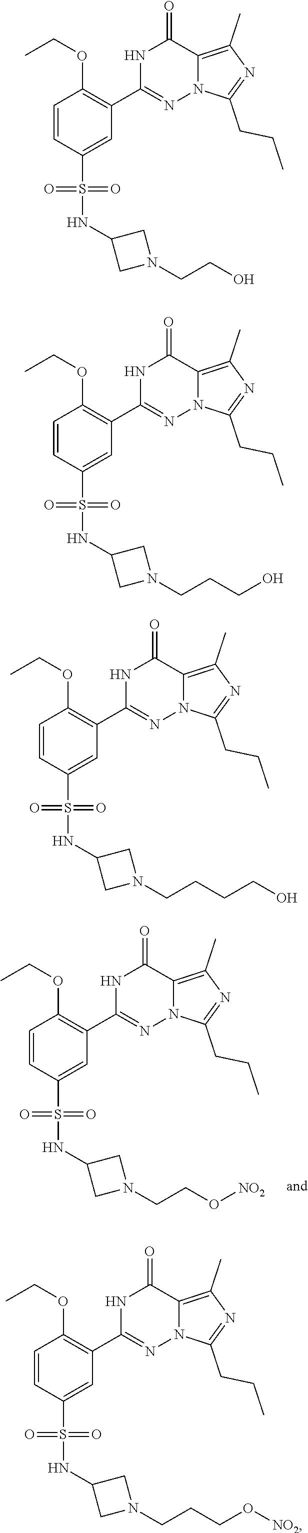

or a pharmaceutically acceptable salt thereof, a solvate, a hydrate, a prodrug, or a stereoisomer.

Clause 33. The method of clause 23, wherein the compound is of formula (III):

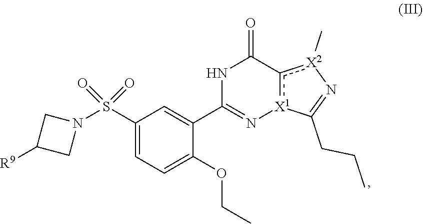

or a pharmaceutically acceptable salt, a solvate, a hydrate, a prodrug, or a stereoisomer thereof, wherein:

R⁹ is selected from —O—NO₂, —NR¹⁰R¹¹, —OR¹², R⁹⁰, and R⁹¹—Z³—R⁹²;

R⁹⁰, R⁹¹ and R⁹² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, optionally substituted (C₁-C₅)alkoxy, optionally substituted (C₃-C₅)heterocycle-(C₁-C₅)alkyl-, and optionally substituted (C₁-C₅)alkyl-(C₃-C₅)heterocycle-(C₁-C₅)alkyl-, wherein the optional substituent is selected from —OH, —NH₂, and —O—NO₂;

Z³ is —CO₂—, —O—, —OCO—, —CONH—, —NHCO—, or —NH—; and

R¹⁰, R¹¹, and R¹² are independently H, optionally substituted (C₁-C₅)alkyl, or optionally substituted (C₁-C₅)alkyl-Z¹—(C₁-C₅)alkyl, wherein the optional substituent is selected from —OH, —NH₂, and —O—NO₂;

or R¹⁰ and R¹¹ together with the nitrogen atom to which they are attached are cyclically linked to form an optionally substituted heterocycle, wherein the optional substituent is selected from —OH, —O—NO₂, —CH₂OH, —CH₂CH₂OH, and —CH₂ONO₂.

Clause 34. The method of clause 33, wherein the compound is of formula (IIIa):

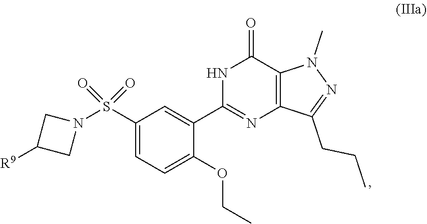

or a pharmaceutically acceptable salt, a solvate, a hydrate, a prodrug, or a stereoisomer thereof.

Clause 35. The method of clause 34, wherein R⁹ is

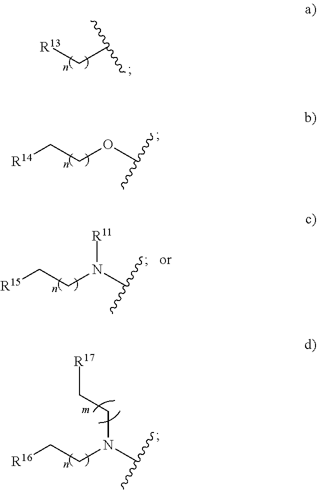

and wherein:

R¹¹ is H or methyl;

R¹³, R¹⁴, R¹⁵, R¹⁶, and R¹⁷ are independently selected from —OH, —NH₂, and —O—NO₂; and

n and m are independently selected from 0, 1, 2, 3, 4, or 5.

Clause 36. The method of clause 35, wherein R⁹ is

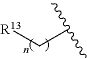

selected from:

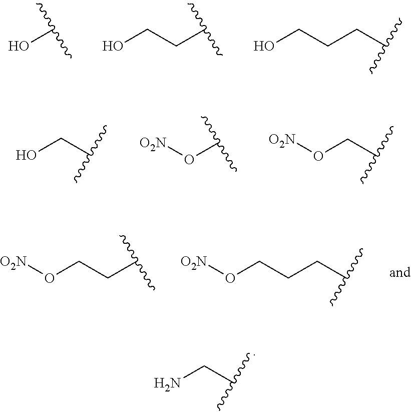

Clause 37. The method of clause 36, wherein the compound is selected from:

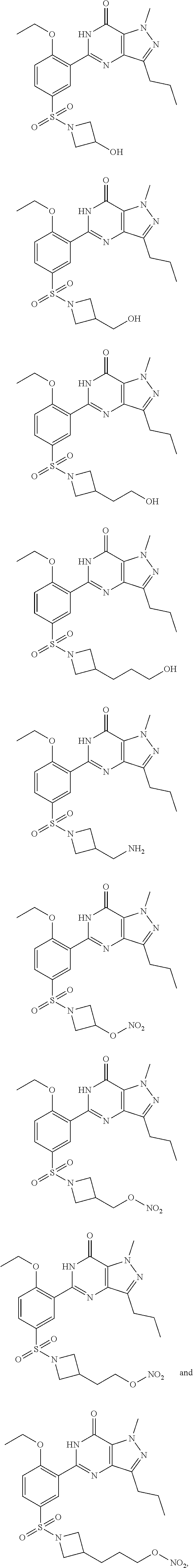

or a pharmaceutically acceptable salt, a solvate, a hydrate, a prodrug, or a stereoisomer thereof.

Clause 38. The method of clause 35, wherein R⁹ is

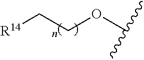

selected from:

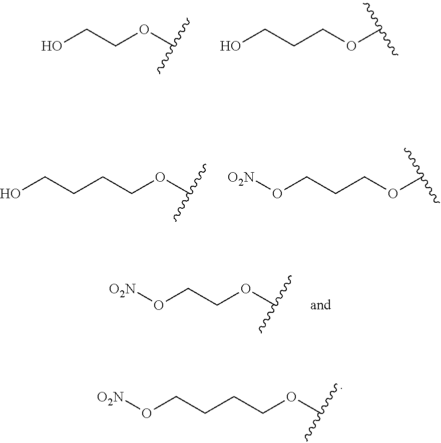 [SMILES: *OCCO ; *OCCCO ; *OCCCCO ; *OCCCO[N+](=O)[O-] ; *OCCO[N+](=O)[O-] ; *OCCCCO[N+](=O)[O-]]

Clause 39. The method of clause 38, wherein the compound is selected from:

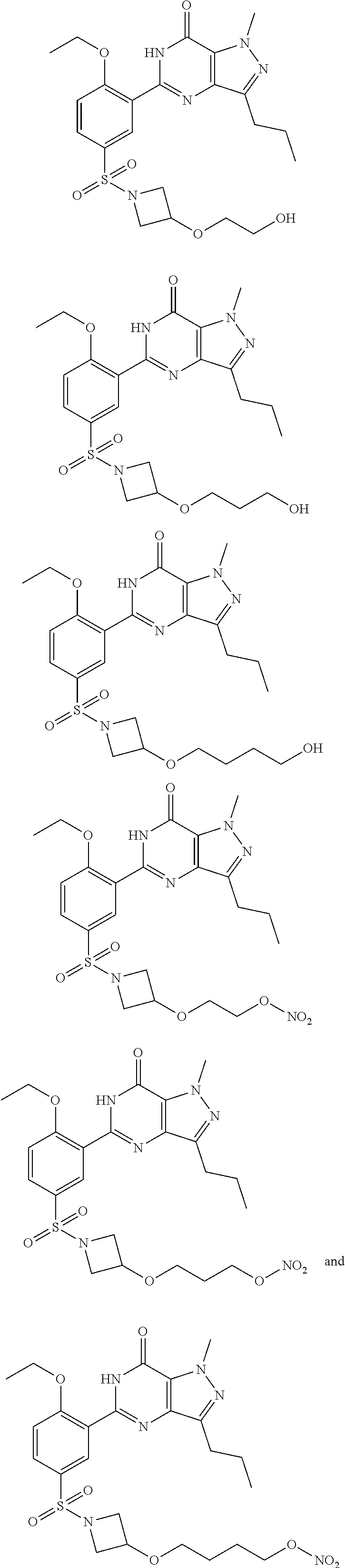

or a pharmaceutically acceptable salt, a solvate, a hydrate, a prodrug, or a stereoisomer thereof.

Clause 40. The method of clause 35, wherein R⁹ is

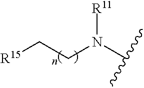

selected from:

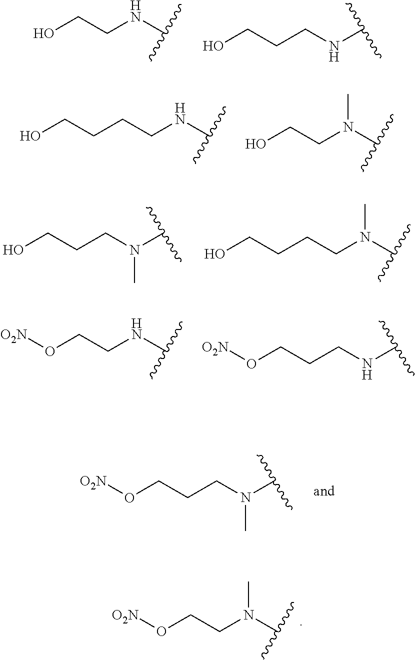

Clause 41. The method of clause 40, wherein the compound is selected from:

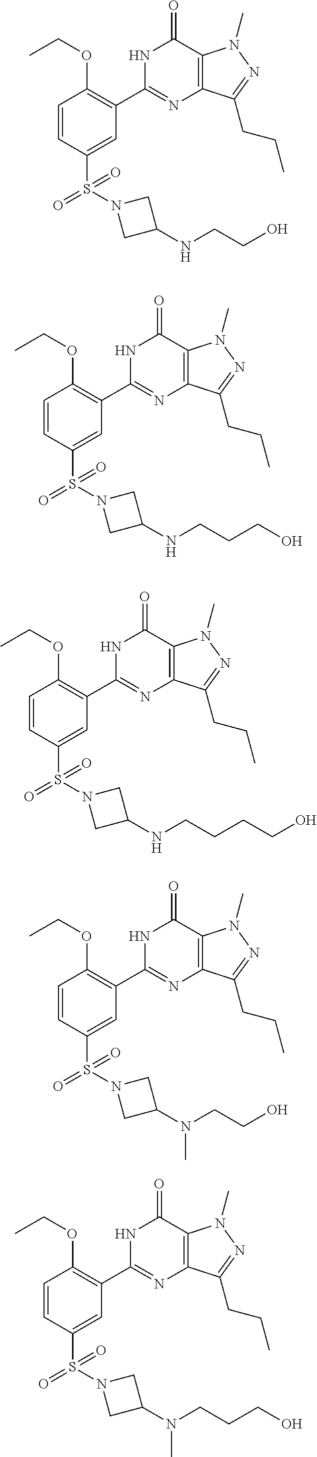

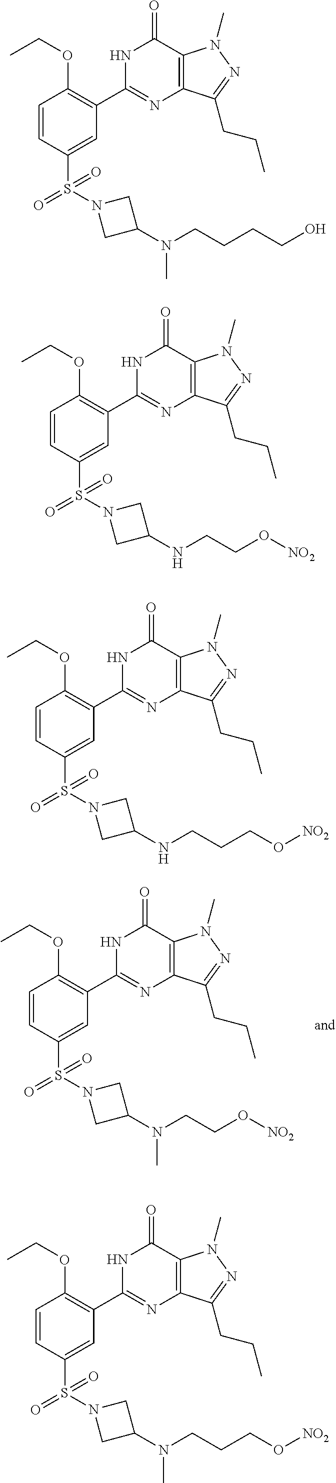

or a pharmaceutically acceptable salt, a solvate, a hydrate, a prodrug, or a stereoisomer thereof.

Clause 42. The method of clause 35, wherein R⁹ is

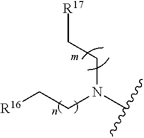

selected from:

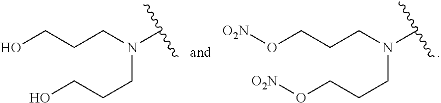 [SMILES: *N(CCCO)CCCO ; *N(CCCO[N+](=O)[O-])CCCO[N+](=O)[O-]]

Clause 43. The method of clause 42, wherein the compound is selected from:

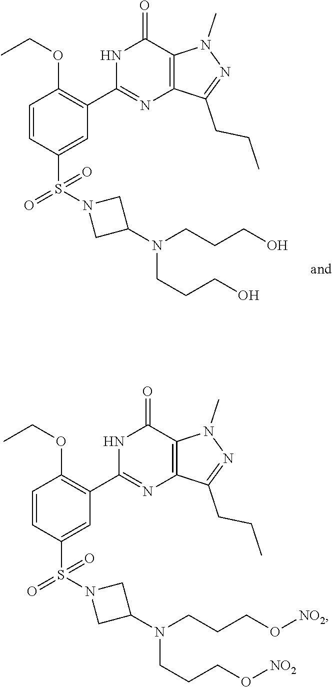 [SMILES: CCCc1nn(C)c2c(=O)[nH]c(-c3cc(S(=O)(=O)N4CC(N(CCCO)CCCO)C4)ccc3OCC)nc12 ; CCCc1nn(C)c2c(=O)[nH]c(-c3cc(S(=O)(=O)N4CC(N(CCCO[N+](=O)[O-])CCCO[N+](=O)[O-])C4)ccc3OCC)nc12]

or a pharmaceutically acceptable salt, a solvate, a hydrate, a prodrug, or a stereoisomer thereof.

Clause 44. The method of clause 34, wherein R⁹ is

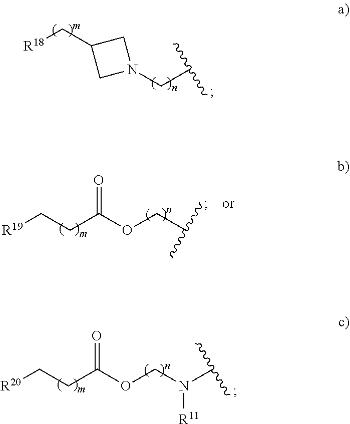

wherein:

R¹¹ is —H or -methyl;

R¹⁸ is selected from —OH, —NH₂, and —O—NO₂;
R¹⁹ and R²⁰ are independently selected from —OH, —NH₂, —O—NO₂, and

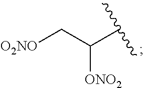

and
n and m are independently selected from 0, 1, 2, 3, 4, 5 or 6.

Clause 45. The method of clause 44, wherein R⁹ is

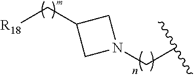

selected from:

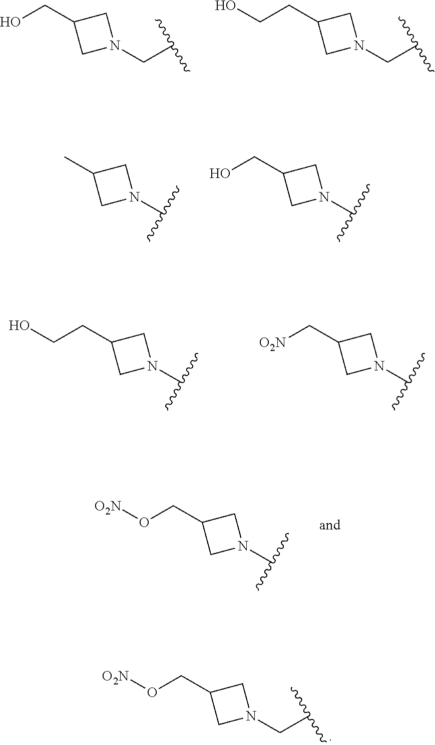

Clause 46. The method of clause 45, wherein the compound is selected from:

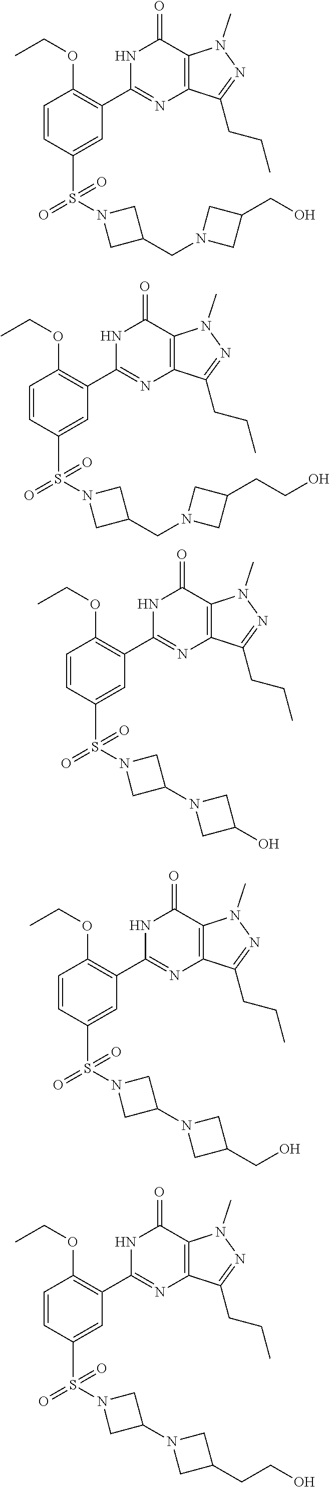

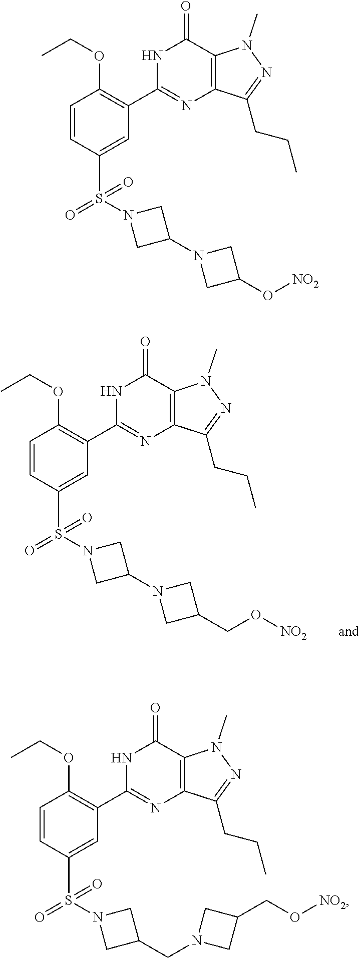 [SMILES: CCCc1nn(C)c2c(=O)[nH]c(-c3cc(S(=O)(=O)N4CC(N5CC(O[N+](=O)[O-])C5)C4)ccc3OCC)nc12 ; CCCc1nn(C)c2c(=O)[nH]c(-c3cc(S(=O)(=O)N4CC(N5CC(CO[N+](=O)[O-])C5)C4)ccc3OCC)nc12 ; CCCc1nn(C)c2c(=O)[nH]c(-c3cc(S(=O)(=O)N4CC(CN5CC(CO[N+](=O)[O-])C5)C4)ccc3OCC)nc12]

or a pharmaceutically acceptable salt, a solvate, a hydrate, a prodrug, or a stereoisomer thereof.

Clause 47. The method of clause 44, wherein R⁹ is

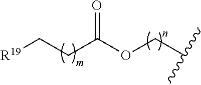

selected from:

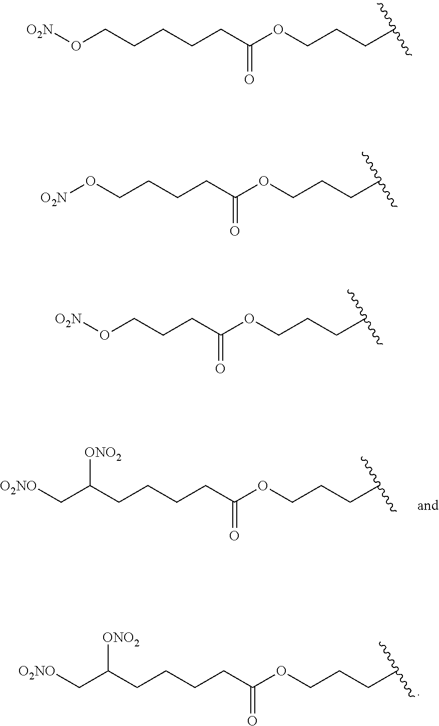

Clause 48. The method of clause 47, wherein the compound is selected from:

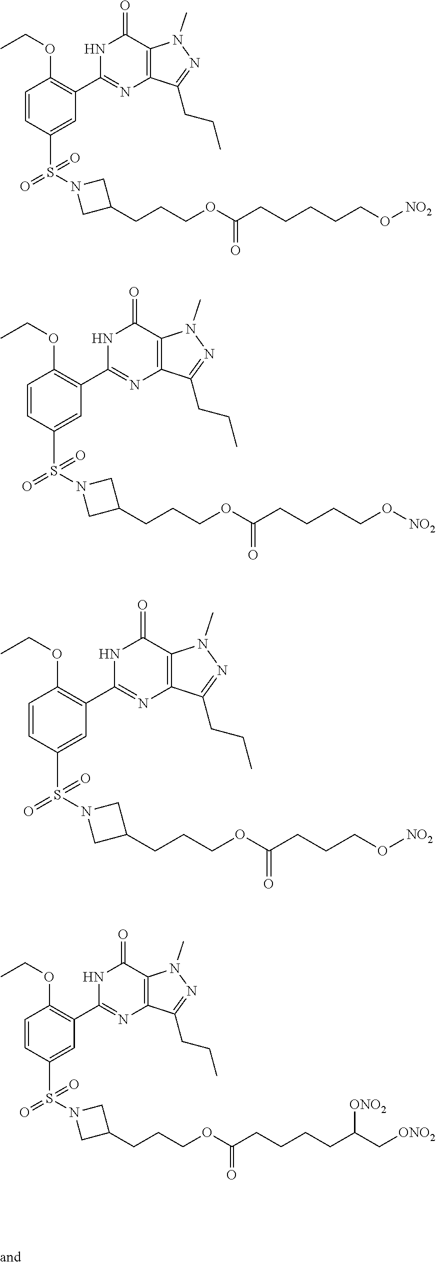

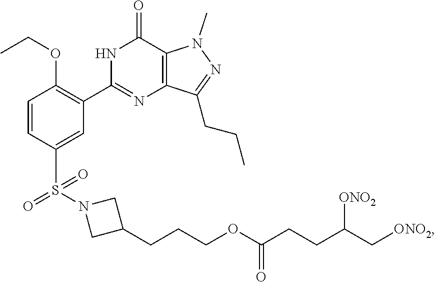

or a pharmaceutically acceptable salt, a solvate, a hydrate, a prodrug, or a stereoisomer thereof.

Clause 49. The method of clause 44, wherein R⁹ is

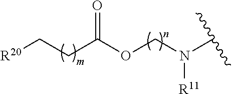

selected from:

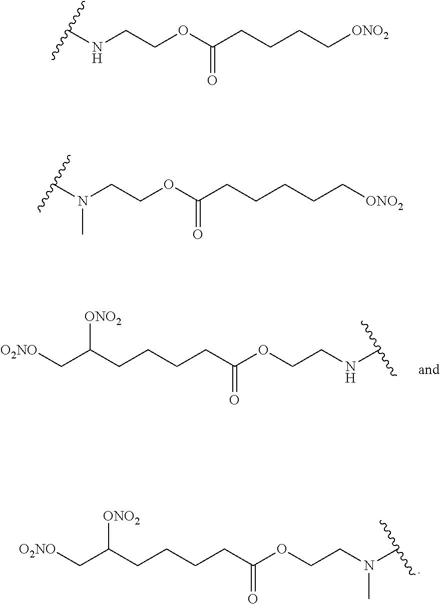

Clause 50. The method of clause 49, wherein the compound is selected from:

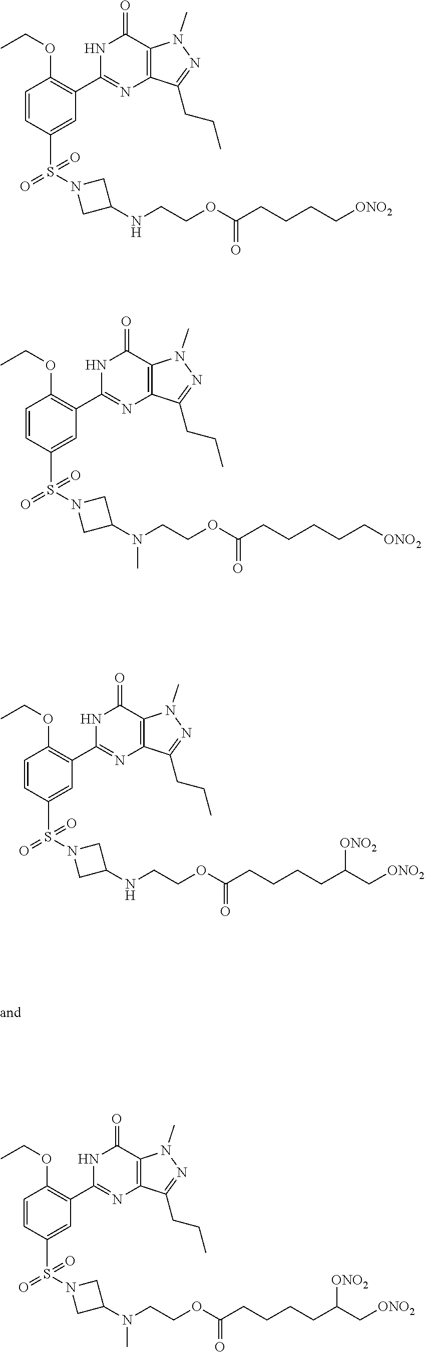

or a pharmaceutically acceptable salt, a solvate, a hydrate, a prodrug, or a stereoisomer thereof.

Clause 51. The method of clause 33, wherein the compound is of formula (IIIb):

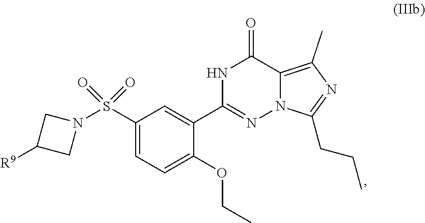

wherein:

R⁹ is selected from —O—NO₂, —NR¹⁰R¹¹, —OR¹², R⁹⁰, and R⁹¹—Z³—R⁹²;

R⁹⁰, R⁹¹ and R⁹² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, optionally substituted (C₁-C₅)alkoxy, and optionally substituted (C₃-C₅)heterocycle-(C₁-C₅)alkyl, wherein the optional substituent is selected from —OH, —NH₂, and —O—NO₂;

Z³ is —CO₂—, —O—, —OCO—, —CONH—, —NHCO—, or —NH—; and

R¹⁰, R¹¹, and R¹² are independently H, optionally substituted (C₁-C₅)alkyl, or optionally substituted (C₁-C₅)alkyl-Z¹—(C₁-C₅)alkyl, wherein the optional substituent is selected from —OH, —NH₂, and —O—NO₂;

or R¹⁰ and R¹¹ together with the nitrogen atom to which they are attached are cyclically linked to form an optionally substituted heterocycle, wherein the optional substituent is selected from —OH, —O—NO₂, —CH₂OH, —CH₂CH₂OH, and —CH₂O—NO₂.

Clause 52. The method of clause 51, wherein R⁹ is

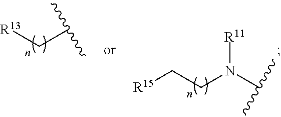

and wherein:

R¹¹ is H or methyl;

R¹³ and R¹⁵ are independently selected from —OH, —NH₂, and —O—NO₂; and
n is 0, 1, 2, 3, 4, or 5.

Clause 53. The method of clause 52, wherein R⁹ is

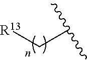

selected from:

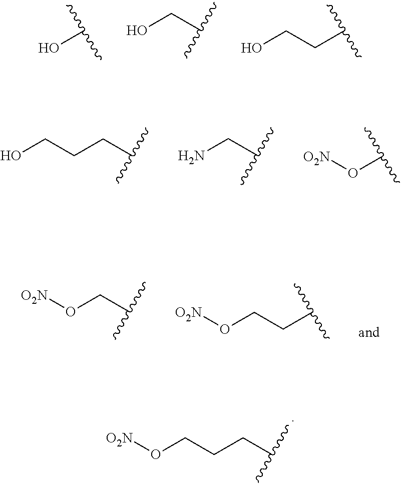

Clause 54. The method of clause 53, wherein the compound is selected from:

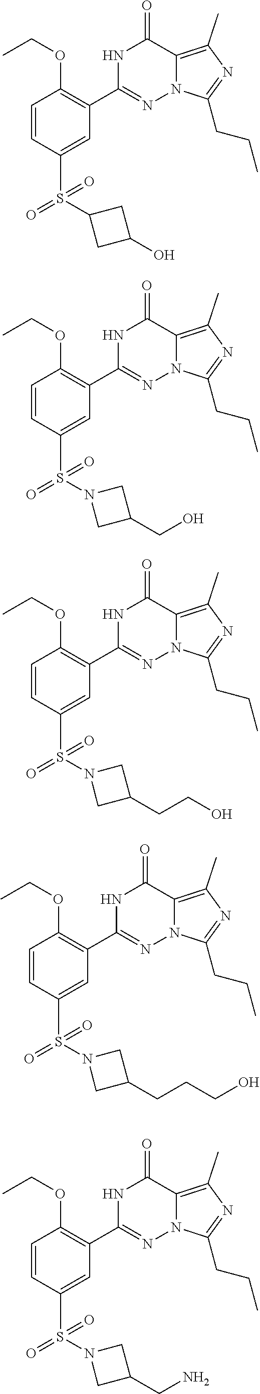

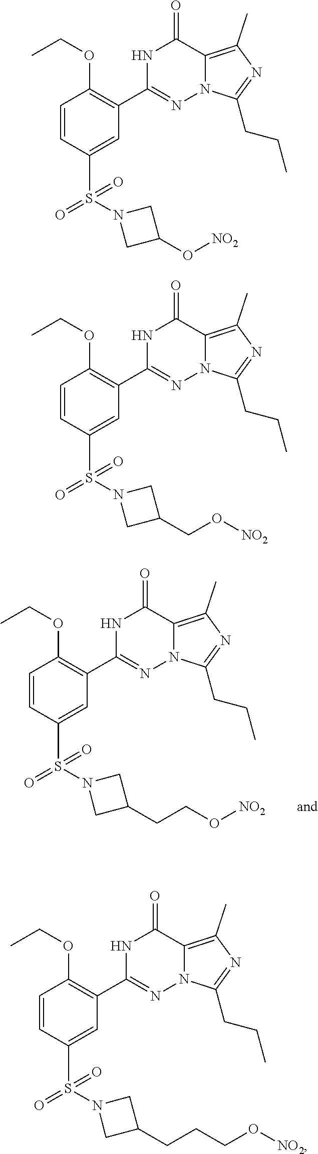

or a pharmaceutically acceptable salt, a solvate, a hydrate, a prodrug, or a stereoisomer thereof.

Clause 55. The method of clause 51, wherein R⁹ is

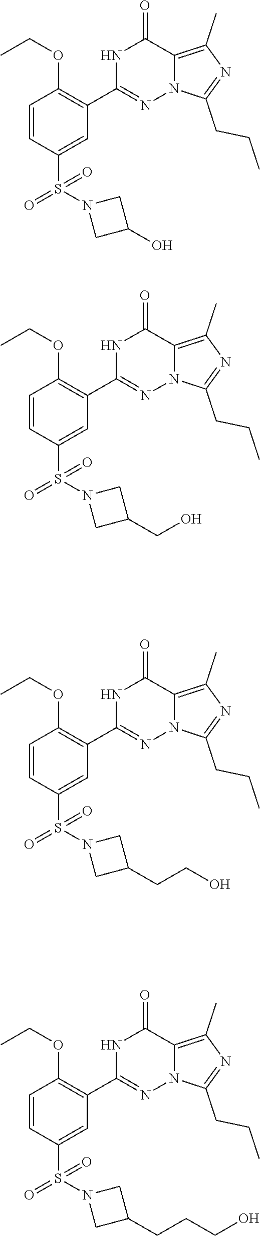

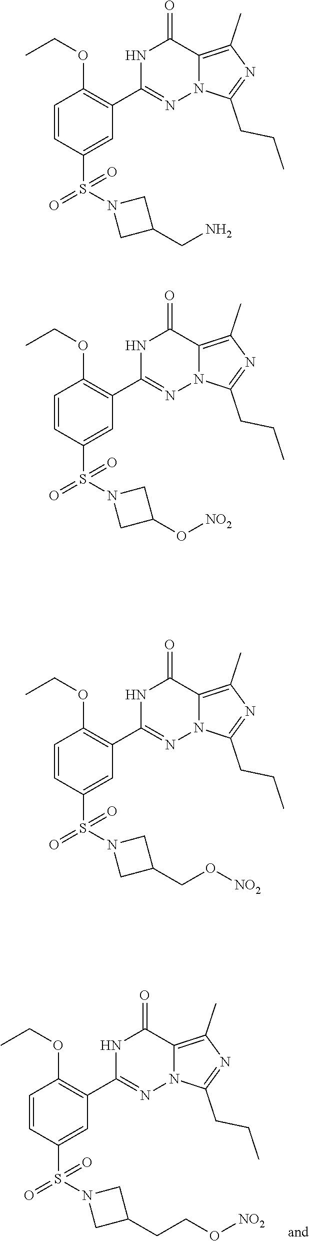

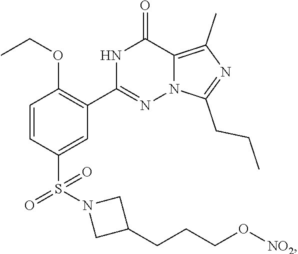 [SMILES: CCCc1nc(C)c2c(=O)[nH]c(-c3cc(S(=O)(=O)N4CC(CCCO[N+](=O)[O-])C4)ccc3OCC)nn12]

selected from:

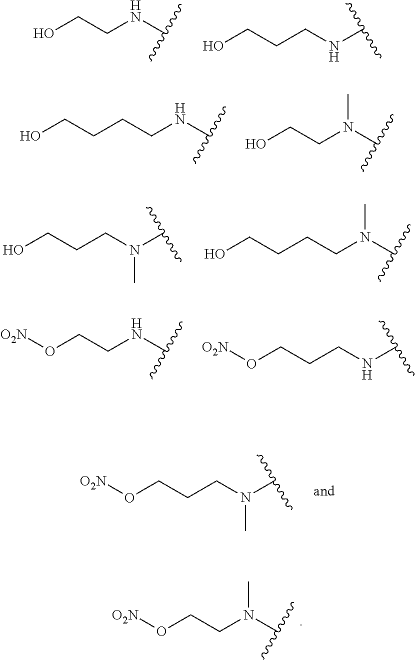

Clause 56. The method of clause 55, wherein the compound is selected from:

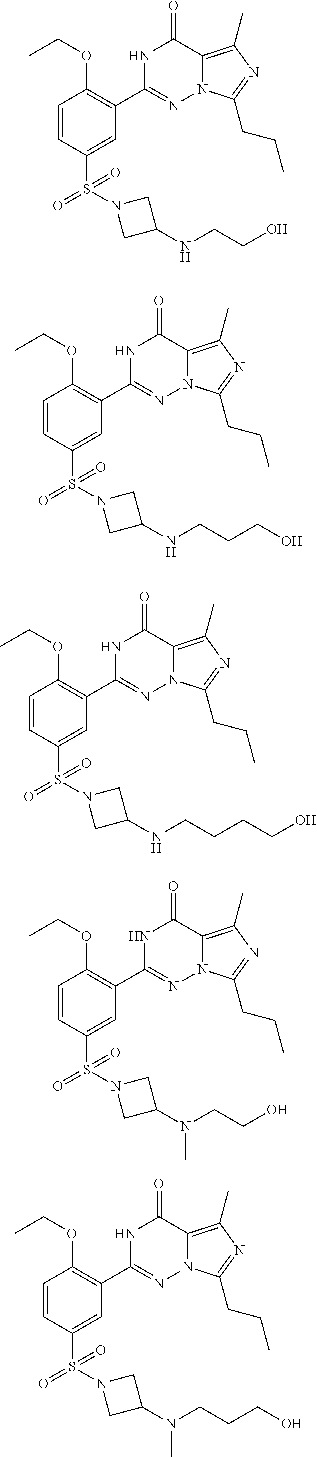

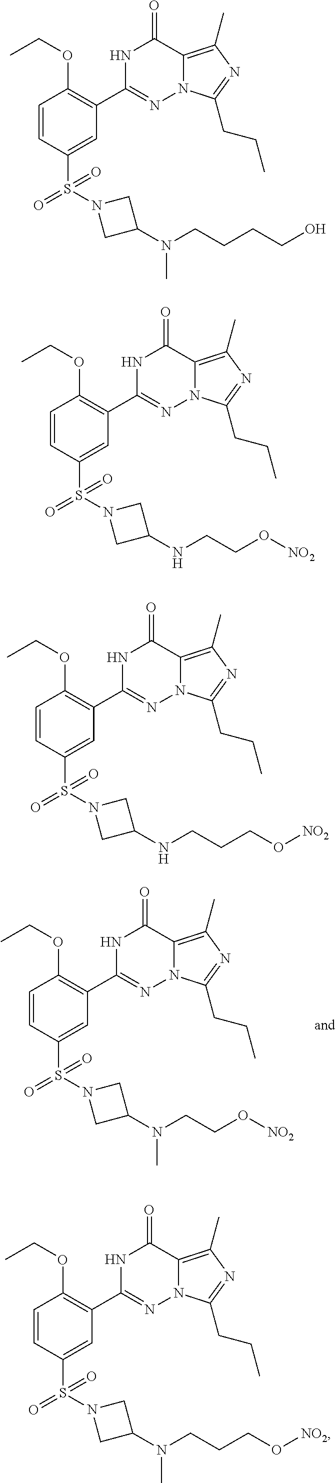

or a pharmaceutically acceptable salt, a solvate, a hydrate, a prodrug, or a stereoisomer thereof.

Clause 57. The method of any one of clauses 1 to 16, wherein the compound is any one of the compounds of Table 1, or a pharmaceutically acceptable salt, a solvate, a hydrate, a prodrug, or a stereoisomer thereof.

Clause 58. A compound, wherein the compound is selected from compounds 26 to 73 of Table 1, or a pharmaceutically acceptable salt, a solvate, a hydrate, a prodrug, or a stereoisomer thereof.

Clause 59. A pharmaceutical composition, comprising:

a therapeutically effective amount of a compound of clause 58, or a pharmaceutically acceptable salt, a solvate, a hydrate, a prodrug, or a stereoisomer thereof; and a pharmaceutically acceptable excipient.

Clause 60. A compound for use in treating an eye disease, wherein the compound is of formula (I):

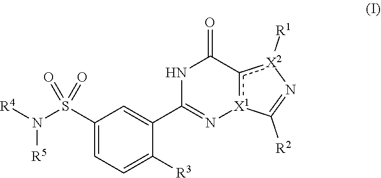

or a pharmaceutically acceptable salt, a solvate, a hydrate, a prodrug, or a stereoisomer thereof, wherein:

X¹ and X² are independently selected from N and C and at least one of X¹ and X² is N;

R¹ is —H, or optionally substituted (C₁-C₅)alkyl;

R² is optionally substituted (C₁-C₅)alkyl;

R³ is optionally substituted (C₁-C₅)alkoxy;

R⁴ is —H or optionally substituted (C₁-C₅)alkyl, and R⁵ is a 4-membered carbocycle or heterocycle ring that is substituted with one or more R⁶,

or R⁴ and R⁵ together with the nitrogen atom to which they are attached are cyclically linked to form a 4-membered heterocycle that is substituted with one or more R⁶; and

and each R⁶ is independently selected from —OH, —O—NO₂, optionally substituted (C₁-C₅)alkyl, optionally substituted (C₁-C₁₀)alkylene, optionally substituted (C₂-C₁₀) alkenyl, optionally substituted (C₂-C₁₀)alkynyl, optionally substituted (C₁-C₅)alkoxy, optionally substituted (C₃-C₅)heterocycle, optionally substituted (C₁-C₅)alkyl-(C₃-C₅)heterocycle-, optionally substituted (C₃-C₅)heterocycle-(C₁-C₅)alkyl-, optionally substituted (C₁-C₅)alkyl-Z¹—(C₁-C₅)alkyl-, optionally substituted (C₁-C₅)alkyl-Z¹—(C₁-C₅)alkoxy-, optionally substituted (C₁-C₁₀)alkyl-NR¹—, optionally substituted (C₁-C₁₀)alkyl-Z¹—(C₁-C₅)alkyl-NR¹—, optionally substituted (C₁-C₁₀)alkoxy-Z¹—(C₁-C₅)alkyl-NR¹—, substituted (C₁-C₅)alkyl-(C₃-C₅)heterocycle-(C₁-C₅)alkyl-, substituted linear linker, and substituted branched linker, wherein Z¹ is —CO₂—, —O—, —OCO—, —CONH—, —NHCO—, or —NH—, and the substituents of each R⁶ are independently selected from —O—NO₂, —ONO, —OH, —NH₂, —COOH, halogen, (C₁-C₃)alkoxy and (C₁-C₃)alkyl;

wherein at least one R⁶ is substituted with —O—NO₂, —ONO, —OH or —NH₂.

Clause 61. The compound for use according to clause 60, wherein the eye disease is glaucoma.

Clause 62. A pharmaceutical composition for use in treating an eye disease, wherein the composition is according to clause 59.

Clause 63. The pharmaceutical composition for use according to clause 62, wherein the eye disease is glaucoma.

Clause 64. A compound for use in treating a PDE-5 and/or -6-related indication, wherein the compound is of formula (I):

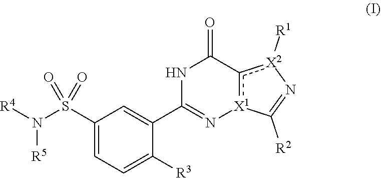

or a pharmaceutically acceptable salt, a solvate, a hydrate, a prodrug, or a stereoisomer thereof, wherein:

X¹ and X² are independently selected from N and C and at least one of X¹ and X² is N;

R¹ is —H, or optionally substituted (C₁-C₅)alkyl;

R² is optionally substituted (C₁-C₅)alkyl;

R³ is optionally substituted (C₁-C₅)alkoxy;

R⁴ is —H or optionally substituted (C₁-C₅)alkyl, and R⁵ is a 4-membered carbocycle or heterocycle ring that is substituted with one or more R⁶,

or R⁴ and R⁵ together with the nitrogen atom to which they are attached are cyclically linked to form a 4-membered heterocycle that is substituted with one or more R⁶; and

and each R⁶ is independently selected from —OH, —O—NO₂, optionally substituted (C₁-C₅)alkyl, optionally substituted (C₁-C₁₀)alkylene, optionally substituted (C₂-C₁₀) alkenyl, optionally substituted (C₂-C₁₀)alkynyl, optionally substituted (C₁-C₅)alkoxy, optionally substituted (C₃-C₅)heterocycle, optionally substituted (C₁-C₅)alkyl-(C₃-C₅)heterocycle-, optionally substituted (C₃-C₅)heterocycle-(C₁-C₅)alkyl-, optionally substituted (C₁-C₅)alkyl-Z¹—(C₁-C₅)alkyl-, optionally substituted (C₁-C₅)alkyl-Z¹—(C₁-C₅)alkoxy-, optionally substituted (C₁-C₁₀)alkyl-NR¹—, optionally substituted (C₁-C₁₀)alkyl-Z¹—(C₁-C₅)alkyl-NR¹—, optionally substituted (C₁-C₁₀)alkoxy-Z¹—(C₁-C₅)alkyl-NR¹—, substituted (C₁-C₅)alkyl-(C₃-C₅)heterocycle-(C₁-C₅)alkyl-, substituted linear linker, and substituted branched linker, wherein Z¹ is —CO₂—, —O—, —OCO—, —CONH—, —NHCO—, or —NH—, and the substituents of each R⁶ are independently selected from —O—NO₂, —ONO, —OH, —NH₂, —COOH, halogen, (C₁-C₃)alkoxy and (C₁-C₃)alkyl;

wherein at least one R⁶ is substituted with —O—NO₂, —ONO, —OH or —NH₂.

Clause 65. A pharmaceutical composition for use in treating a PDE-5 and/or -6-related indication, wherein the composition is according to clause 59.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### 6. EXAMPLES

The following examples are offered to illustrate the present disclosure and are not to be construed in any way as limiting the scope of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

Unless otherwise stated, all temperatures are in degrees Celsius. Efforts have been made to ensure accuracy with respect to numbers used (e.g., amounts, temperatures, etc.), but some experimental errors and deviation should be allowed for.

If an abbreviation is not defined, it has its generally accepted meaning.

### General Synthetic Methods

Final compounds were confirmed by high-performance liquid chromatography/mass spectrometry (HPLC/MS) analysis and determined to be >90% pure by weight. ¹H and ¹³C nuclear magnetic resonance (NMR) spectra were recorded in CDCl₃ (residual internal standard CHCl₃=δ 7.26), dimethyl sulfoxide (DMSO)-d₆ (residual internal standard CD₃SOCD₂H=δ 2.50), methanol-d₄ (residual internal standard CD₂HOD=δ 3.20), or acetone-d6 (residual internal standard CD₃COCD₂H=δ 2.05). The chemical shifts (δ) reported are given in parts per million (ppm) and the coupling constants (J) are in Hertz (Hz). The spin multiplicities are reported as s=singlet, bs=broad singlet, bm=broad multiplet, d=doublet, t=triplet, q=quartet, p=pentuplet, dd=doublet of doublet, ddd=doublet of doublet of doublet, dt=doublet of triplet, td=triplet of doublet, tt=triplet of triplet, and m=multiplet.

HPLC-MS analysis was carried out with gradient elution. Medium pressure liquid chromatography (MPLC) was performed with silica gel columns in both the normal phase and reverse phase.

### Example 1—Preparation of Substituted Azetidine-Linked dihydro-1H-pyrazolo[4,3-d]pyrimidine Compounds

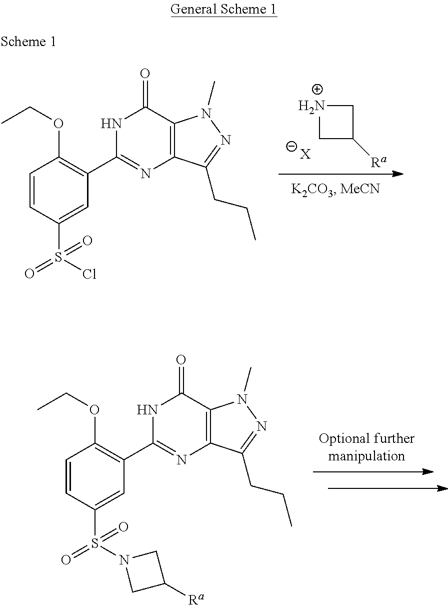

### Synthesis of Compound 1

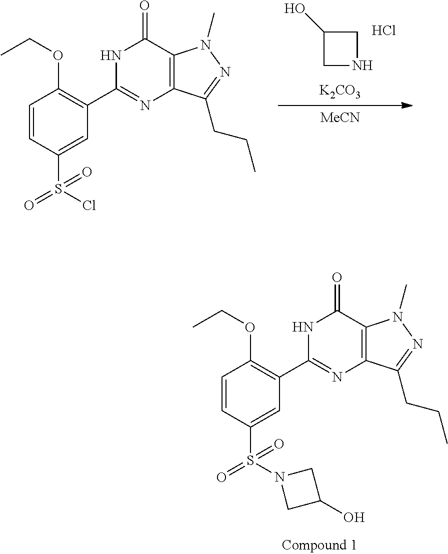

To a solution of 4-ethoxy-3-(1-methyl-7-oxo-3-propyl-6,7-dihydro-1H-pyrazolo[4,3-d]pyrimidin-5-yl) benzenesulfonyl chloride (350 mg, 851.84 umol) and azetidin-3-ol hydrochloride (139.98 mg, 1.28 mmol) in MeCN (15 mL) was added K₂CO₃ (353.19 mg, 2.56 mmol), the reaction mixture was stirred at 25° C. for 16 h. Filtered and evaporated under reduced pressure, the residue was purified by prep-HPLC to afford compound 1, 5-(2-ethoxy-5-((3-hydroxyazetidin-1-yl)sulfonyl)phenyl)-1-methyl-3-propyl-1,6-dihydro-7H-pyrazolo[4,3-d] pyrimidin-7-one (350 mg, 91.81% yield) as a white solid. ¹H NMR (400 MHz, DMSO-d₆) δ 12.23 (s, 1H), 7.92-7.88 (m, 2H), 7.41 (d, 1H), 5.77 (d, 1H), 4.31-4.20 (m, 3H), 4.16 (s, 3H), 3.90-3.86 (m, 2H), 3.38-3.35 (m, 2H), 2.78 (t, 2H), 1.77-1.71 (m, 2H), 1.34 (t, 3H), 0.93 (t, 3H); MS: (m/z)=448.3 (M+1, ESI+); HRMS: 448.1652.

### Synthesis of Compound 2

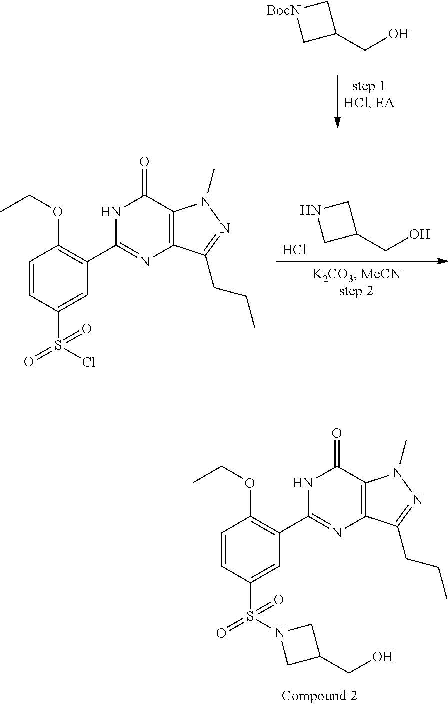

### Step 1:

A mixture of tert-butyl 3-(hydroxymethyl)azetidine-1-carboxylate (300 mg, 1.60 mmol) was dissolved in 3M HCl in EA (3M, 5 mL) was stirred at 25° C. for 2 h. The reaction mixture was evaporated under reduced pressure to afford azetidin-3-ylmethanol hydrochloride (195 mg, 98.48% yield) as a colorless oil. MS: m/z=88.13 (M+1, ESI+).

### Step 2:

To a solution of 4-ethoxy-3-(1-methyl-7-oxo-3-propyl-6,7-dihydro-1H-pyrazolo[4,3-d]pyrimidin-5-yl) benzenesulfonyl chloride (432.22 mg, 1.05 mmol) and azetidin-3-ylmethanol hydrochloride (195 mg, 1.58 mmol) in MeCN (10 mL) was added K₂CO₃ (436.15 mg, 3.16 mmol), the reaction mixture was stirred at 100° C. for 4 h. Filtered and evaporated under reduced pressure, the residue was purified by prep-HPLC to afford compound 2, 5-(2-ethoxy-5-((3-(hydroxymethyl)azetidin-1-yl)sulfonyl)phenyl)-1-methyl-3-propyl-1,6-dihydro-7H-pyrazolo[4,3-d]pyrimidin-7-one (125 mg, 25.75% yield) as a white solid. ¹H NMR (400 MHz, DMSO-d₆) δ 12.23 (s, 1H), 7.93-7.89 (m, 2H), 7.40 (d, 1H), 4.68 (t, 1H), 4.22 (m, 2H), 4.16 (s, 3H), 3.72 (t, 2H), 3.47-3.44 (m, 2H), 3.31-3.28 (m, 3H), 2.79 (t, 2H), 1.77-1.71 (m, 2H), 1.34 (t, 3H), 0.93 (t, 3H); MS: m/z=462.3 (M+1, ESI+); HRMS: 462.1805.

### Synthesis of Compound 3

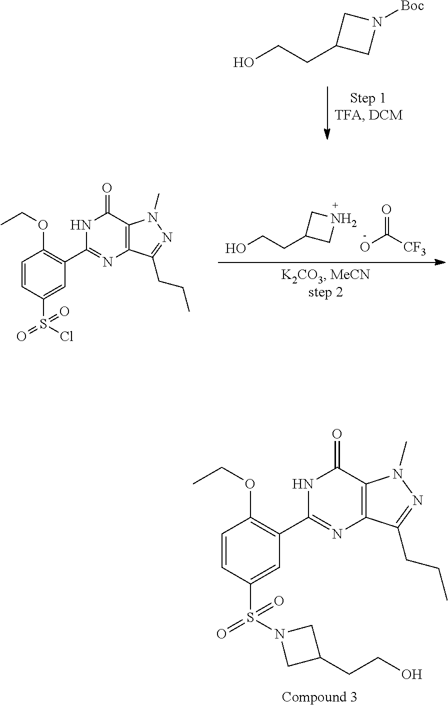

### Step 1:

To a suspension of tert-butyl 3-(2-hydroxyethyl)azetidine-1-carboxylate (2.0 g, 9.94 mmol) in DCM (30 mL) was added trifluoroacetic acid (TFA) (5.67 g, 49.69 mmol), the reaction mixture was stirred at 25° C. for 5 h. The resulting solution was evaporated to afford 2-(azetidin-3-yl)ethan-1-ol; 2,2,2-trifluoroacetate salt (2.0 g, 93.98% yield) as a yellow oil. MS: m/z=102.4 (M+1, ESI+).

### Step 2:

To a solution of 4-ethoxy-3-(1-methyl-7-oxo-3-propyl-6,7-dihydro-1H-pyrazolo[4,3-d]pyrimidin-5-yl) benzenesulfonyl chloride (600.00 mg, 1.46 mmol) and 2-(azetidin-3-yl)ethan-1-ol; 2,2,2-trifluoroacetate salt (375.29 mg, 1.75 mmol) in MeCN (15 mL) was added K₂CO₃ (605.46 mg, 4.38 mmol), the reaction mixture was stirred at 80° C. for 3 h. Filtered and evaporated under reduced pressure, the residue was purified by prep-HPLC to afford compound 3, 5-(2-ethoxy-5-((3-(2-hydroxyethyl)azetidin-1-yl)sulfonyl)phenyl)-1-methyl-3-propyl-1,6-dihydro-7H-pyrazolo[4,3-d]pyrimidin-7-one (380 mg, 54.72% yield) as a white solid. ¹H NMR (400 MHz, DMSO-d₆) δ 12.25 (s, 1H), 7.92-7.88 (m, 2H), 7.40 (d, 1H), 4.37 (bs, 1H), 4.24 (q, 2H), 4.16 (s, 3H), 3.80 (t, 2H), 3.38-3.34 (m, 2H), 3.29-3.26 (m, 2H), 2.78 (t, 2H), 2.49-2.46 (m, 1H), 1.77-1.71 (m, 2H), 1.43 (q, 2H), 1.35 (t, 3H), 0.93 (t, 3H); MS: m/z=476.2 (M+1, ESI+); HRMS: 476.1963.

### Synthesis of Compound 4

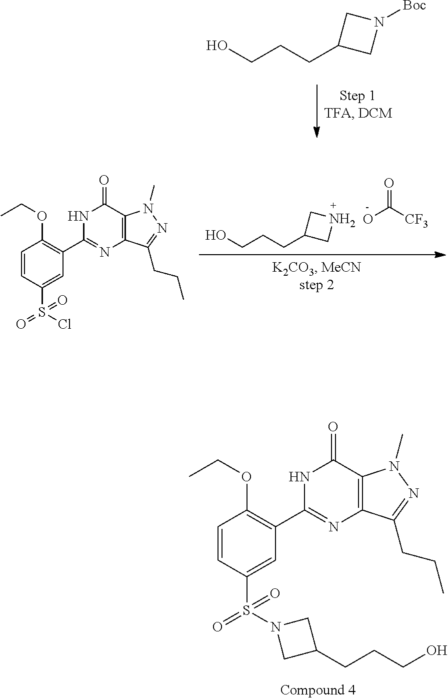

### Step 1:

To a solution of tert-butyl 3-(3-hydroxypropyl)azetidine-1-carboxylate (382 mg, 1.70 mmol) in dichloromethane (DCM) (10 mL) was added TFA (2.17 g, 19.04 mmol), the reaction mixture was stirred at 25° C. for 3 h. The resulting solution was evaporated to afford 3-(azetidin-3-yl)propan-1-ol; 2,2,2-trifluoroacetate salt (218 mg, crude) as a yellow oil. MS: m/z=116.1 (M+1, ESI+).

### Step 2:

To a solution of 4-ethoxy-3-(1-methyl-7-oxo-3-propyl-6,7-dihydro-1H-pyrazolo[4,3-d]pyrimidin-5-yl) benzenesulfonyl chloride (500 mg, 1.22 mmol) and 3-(azetidin-3-yl)propan-1-ol; 2,2,2-trifluoroacetate salt (140 mg, 1.83 mmol) in MeCN (15 mL) was added K₂CO₃ (505 mg, 3.65 mmol), the reaction mixture was stirred at 25° C. for 16 h. Filtered and evaporated under reduced pressure, the residue was purified by prep-HPLC to afford compound 4, 5-(2-ethoxy-5-((3-(3-hydroxypropyl)azetidin-1-yl)sulfonyl)phenyl)-1-methyl-3-propyl-1,6-dihydro-7H-pyrazolo[4,3-d]pyrimidin-7-one (450 mg, 75.5% yield) as a white solid. ¹H NMR (400 MHz, DMSO-d₆) δ 11.13 (bs, 1H), 7.92-7.89 (m, 2H), 7.40 (d, 1H), 4.23 (q, 2H), 4.16 (s, 3H), 3.79 (t, 2H), 3.32-3.27 (m, 4H), 2.78 (t, 2H), 2.41-2.34 (m, 1H), 1.77-1.69 (m, 2H), 1.36-1.19 (m, 7H), 0.93 (t, 3H); MS: m/z=490.3 (M+1, ESI+); HRMS: 490.2120.

### Synthesis of Compound 5

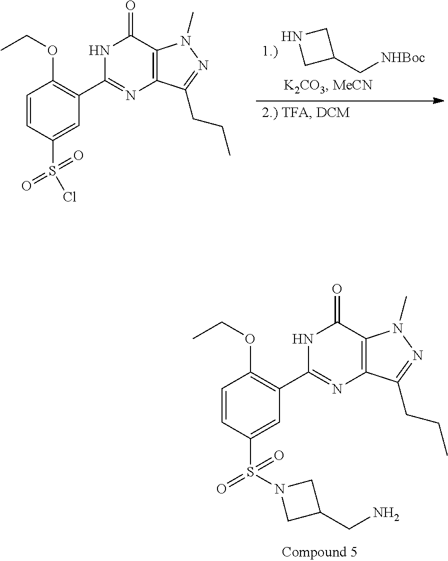

### Step 1:

To a solution of 4-ethoxy-3-(1-methyl-7-oxo-3-propyl-6,7-dihydro-1H-pyrazolo[4,3-d]pyrimidin-5-yl) benzenesulfonyl chloride (492 mg, 1.20 mmol) and tert-butyl (azetidin-3-ylmethyl)carbamate (186 mg, 999 umol) in MeCN (10 mL) was added K₂CO₃ (414 mg, 3.00 mmol), the resulting mixture was stirred at 100° C. for 4 h. Filtered and evaporated under reduced pressure, the residue was purified by prep-HPLC to afford tert-butyl ((1-((4-ethoxy-3-(1-methyl-7-oxo-3-propyl-6,7-dihydro-1H-pyrazolo[4,3-d] pyrimidin-5-yl)phenyl) sulfonyl)azetidin-3-yl)methyl)carbamate (400 mg, 71.44% yield) as a white solid. MS: m/z=561.2 (M+1, ESI+).

### Step 2:

A mixture of tert-butyl ((1-((4-ethoxy-3-(1-methyl-7-oxo-3-propyl-6,7-dihydro-1H-pyrazolo[4,3-d] pyrimidin-5-yl)phenyl)sulfonyl)azetidin-3-yl)methyl)carbamate (400 mg, 713 umol) in DCM (5 mL) was added TFA (411 mg, 3.61 mmol), the reaction mixture was stirred at 25° C. for 2 h. The reaction mixture was evaporated under reduced pressure, the residue was purified by prep-HPLC to afford 5-(5-((3-(aminomethyl)azetidin-1-yl)sulfonyl)-2-ethoxyphenyl)-1-methyl-3-propyl-1,6-dihydro-7H-pyrazolo[4,3-d] pyrimidin-7-one (207 mg, 63.00% yield) as a white solid. ¹H NMR (400 MHz, DMSO-d₆) δ 7.92-7.89 (m, 2H), 7.41-7.39 (d, 1H), 5.62 (bs, 2H), 4.22 (q, 2H), 4.16 (s, 3H), 3.72 (t, 2H), 3.45-3.42 (m, 2H), 2.78 (t, 2H), 2.50-2.49 (m, 2H), 2.39-2.34 (m, 11H), 1.77-1.69 (m, 2H), 1.35 (t, 3H), 0.93 (t, 3H); MS: m/z=462.1 (M+1, ESI+); HRMS: 461.1965.

### Synthesis of Compound 9

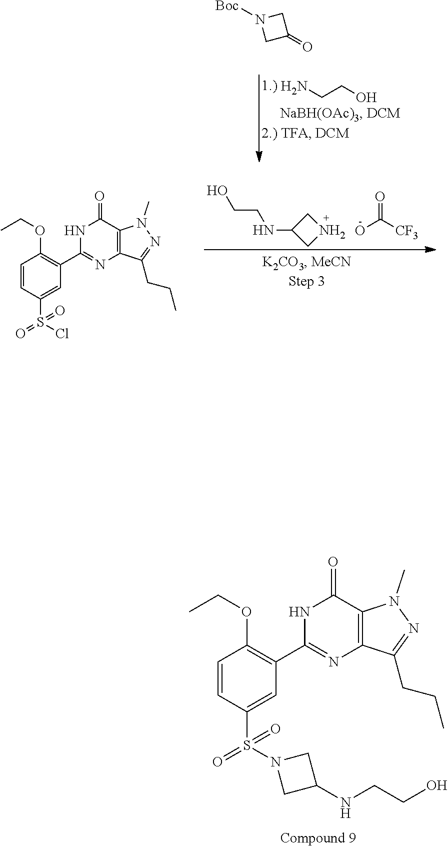

### Step 1:

To a solution of tert-butyl 3-oxoazetidine-1-carboxylate (4.00 g, 23.37 mmol) and 2-aminoethan-1-ol (1.86 g, 30.38 mmol) in DCM (50 mL) was added NaBH(OAc)₃ (7.43 g, 35.05 mmol), the reaction mixture was stirred at 25° C. for 16 h. The reaction mixture was poured into water (100 mL) and extracted with DCM (20 mL×3). The combined organic layers were dried over Na₂SO₄ and concentrated under reduced pressure, the residue was purified by column chromatography to afford tert-butyl 3-((2-hydroxyethyl)amino)azetidine-1-carboxylate (4.00 g, 79% yield) as a yellow oil. MS: m/z=217.2 (M+1, ESI+).

### Step 2:

To a solution of tert-butyl 3-((2-hydroxyethyl)amino)azetidine-1-carboxylate (4.00 g, 18.49 mmol) in DCM (20 mL) was added TFA (21.09 g, 184.95 mmol), the reaction mixture was stirred at 25° C. for 16 h. The reaction mixture was evaporated under reduced pressure to afford 2-(azetidin-3-ylamino)ethan-1-ol; 2,2,2-trifluoroacetate salt (2.15 g, crude) as a colorless oil. MS: m/z=117.3 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (70 mg, 1.70 mmol) and 2-(azetidin-3-ylamino)ethan-1-ol; 2,2,2-trifluoroacetate salt (836 mg, 7.20 mmol) in MeCN (20 mL) was added K₂CO₃ (2.35 g, 17.04 mmol), the reaction mixture was stirred at 25° C. for 16 h. Filtered and evaporated under reduced pressure, the residue was purified by prep-HPLC to afford compound 9, 5-(2-ethoxy-5-((3-((2-hydroxyethyl)amino)azetidin-1-yl)sulfonyl) phenyl)-1-methyl-3-propyl-1,6-dihydro-7H-pyrazolo[4,3-d]pyrimidin-7-one (500 mg, 59% yield) as a white solid. ¹H NMR (400 MHz, DMSO-d₆) δ 11.21 (bs, 1H), 7.93-7.89 (m, 2H), 7.40 (d, 1H), 4.44 (bs, 1H), 4.22 (q, 2H), 4.16 (s, 3H), 3.83-3.82 (m, 2H), 3.40-3.32 (m, 6H), 2.78 (t, 2H), 2.39 (t, 2H), 1.77-1.71 (m, 2H), 1.35 (t, 3H), 0.94 (t, 3H); MS: m/z=491.1 (M+1, ESI+); HRMS: 491.2072.

### Synthesis of Compound 10

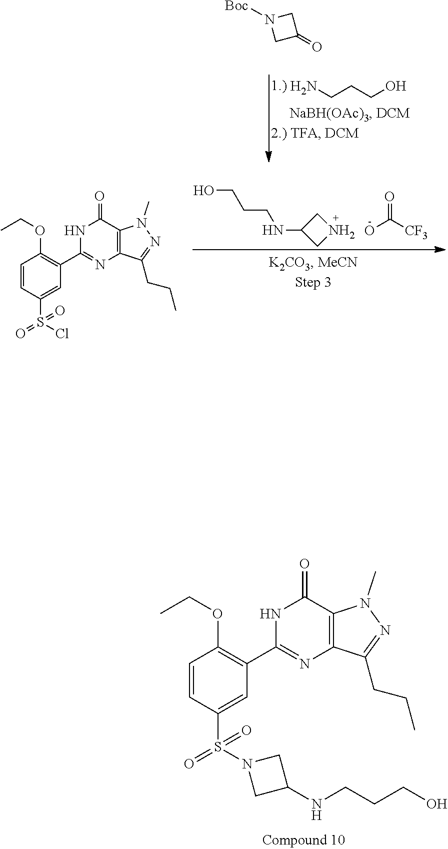 [SMILES: CC(C)(C)OC(=O)N1CC(=O)C1 ; NCCCO ; CCCc1nn(C)c2c(=O)[nH]c(-c3cc(S(=O)(=O)Cl)ccc3OCC)nc12 ; OCCCNC1CNC1 ; O=C(O)C(F)(F)F ; CCCc1nn(C)c2c(=O)[nH]c(-c3cc(S(=O)(=O)N4CC(NCCCO)C4)ccc3OCC)nc12]

### Step 1:

To a solution of tert-butyl 3-oxoazetidine-1-carboxylate (2.5 g, 14.60 mmol) and 3-aminopropan-1-ol (1.10 g, 14.60 mmol) in DCM (40 mL) was added NaBH(OAc)₃ (4.64 g, 21.91 mmol), the reaction mixture was stirred at 25° C. for 16 h. The reaction mixture was poured into water (200 mL) and extracted with DCM (50 mL×3). The combined organic layers were dried over Na₂SO₄ and concentrated under reduced pressure, the residue was purified by column chromatography to afford tert-butyl 3-((3-hydroxypropyl)amino) azetidine-1-carboxylate (3.0 g, 89.20% yield) as a yellow oil. MS: m/z=231.2 (M+1, ESI+). ¹H NMR (400 MHz, Methanol-d₄) δ 4.23-4.04 (m, 2H), 3.90-3.76 (m, 2H), 3.67-3.62 (m, 2H), 3.32-3.28 (m, 1H), 2.89-2.81 (m, 2H), 1.84-1.76 (m, 2H), 1.44 (s, 9H).

### Step 2:

To a solution of tert-butyl 3-((3-hydroxypropyl)amino)azetidine-1-carboxylate (3.0 g, 13.03 mmol) in DCM (20 mL) was added TFA (7.43 g, 65.13 mmol), the reaction mixture was stirred at 25° C. for 16 h. The resulting solution was evaporated to afford 3-(azetidin-3-ylamino)propan-1-ol; 2,2,2-trifluoroacetate salt (2.8 g, 88.38% yield) as a yellow oil. MS: m/z=131.2 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (500 mg, 1.22 mmol) and 3-(azetidin-3-ylamino)propan-1-ol; 2,2,2-trifluoroacetate salt (355 mg, 1.46 mmol) in MeCN (15 mL) was added K₂CO₃ (505 mg, 3.65 mmol), the reaction mixture was stirred at 80° C. for 2 h. Filtered and evaporated under reduced pressure, the residue was purified by prep-HPLC to afford compound 10, 5-(2-ethoxy-5-((3-((3-hydroxypropyl)amino)azetidin-1-yl)sulfonyl)phenyl)-1-methy-3-propyl-1,6-dihydro-7H-pyrazolo[4,3-d]pyrimidin-7-one (300 mg, 48.86% yield) as a white solid. ¹H NMR (400 MHz, DMSO-d₆) δ 12.22 (bs, 1H), 7.93-7.88 (m, 2H), 7.40 (d, 1H), 4.34 (bs, 1H), 4.24 (q, 2H), 4.16 (s, 3H), 3.83-3.80 (m, 2H), 3.41-3.34 (m, 5H), 2.78 (t, 2H), 2.34 (t, 2H), 2.05 (bs, 1H), 1.77-1.71 (m, 2H), 1.43-1.38 (m, 2H), 1.35 (t, 3H), 0.93 (t, 3H); MS: m/z=505.3 (M+1, ESI+); HRMS: 505.2228.

### Synthesis of Compound 11

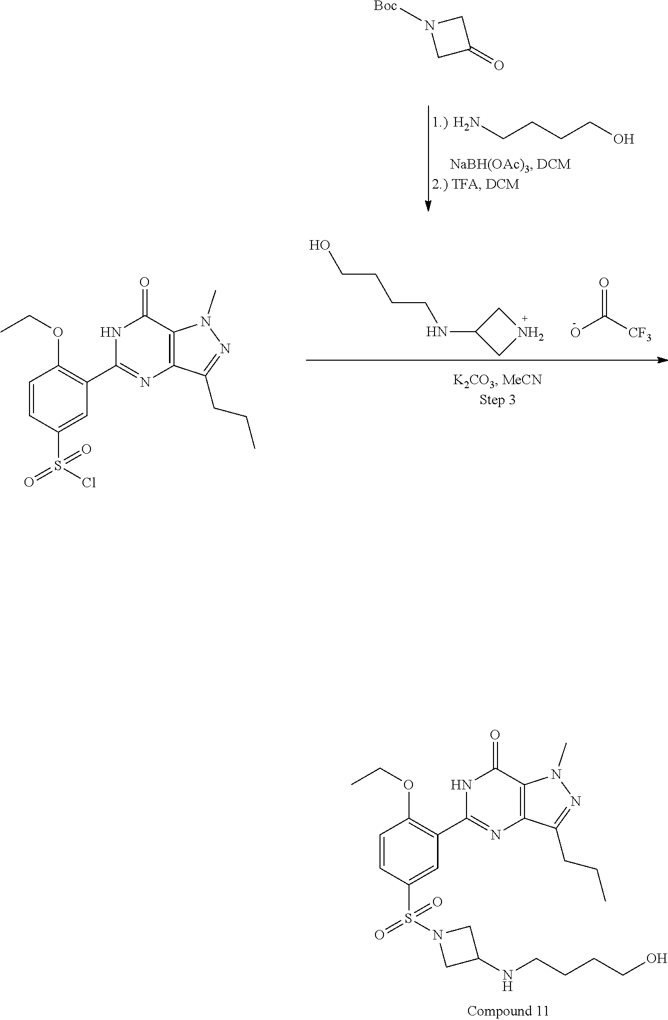 [SMILES: CC(C)(C)OC(=O)N1CC(=O)C1 ; NCCCCO ; CCCc1nn(C)c2c(=O)[nH]c(-c3cc(S(=O)(=O)Cl)ccc3OCC)nc12 ; OCCCCNC1CNC1 ; O=C(O)C(F)(F)F ; CCCc1nn(C)c2c(=O)[nH]c(-c3cc(S(=O)(=O)N4CC(NCCCCO)C4)ccc3OCC)nc12]

### Step 1:

To a solution of tert-butyl 3-oxoazetidine-1-carboxylate (2.0 g, 11.68 mmol) and 4-aminobutan-1-ol (1.25 g, 14.02 mmol) in DCM (20 mL) was added NaBH(OAc)₃ (3.71 g, 17.52 mmol), the reaction mixture was stirred at 25° C. for 16 h. The reaction mixture was poured into water (200 mL) and extracted with DCM (50 mL×3). The combined organic layers were dried over Na₂SO₄ and concentrated under reduced pressure, the residue was purified by column chromatography to afford tert-butyl 3-((4-hydroxybutyl)amino)azetidine-1-carboxylate (2.3 g, 80.58% yield) as a yellow oil. MS: m/z=245.3 (M+1, ESI+).

### Step 2:

To a solution of tert-butyl 3-((4-hydroxybutyl)amino)azetidine-1-carboxylate (2.3 g, 9.41 mmol) in DCM (5 mL) was added TFA (5 mL), the reaction mixture was stirred at 25° C. for 16 h. The resulting solution was evaporated to afford 4-(azetidin-3-ylamino)butan-1-ol; 2,2,2-trifluoroacetate salt (1.3 g, 95.76% yield) as a yellow oil. MS: m/z=145.3 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (500 mg, 1.22 mmol) and 4-(azetidin-3-ylamino)butan-1-ol; 2,2,2-trifluoroacetate salt (351 mg, 2.43 mmol) in tetrahydrofuran (THF) (10 mL) was added TEA (369 mg, 3.65 mmol), the reaction mixture was stirred at 25° C. for 2 h. Filtered and evaporated under reduced pressure, the residue was purified by prep-HPLC to afford compound 11, 5-(2-ethoxy-5-((3-((4-hydroxybutyl)amino)azetidin-1-yl)sulfonyl)phenyl)-1-methyl-3-propyl-1,6-dihydro-7H-pyrazolo[4,3-d]pyrimidin-7-one (550 mg, 87.15% yield) as a white solid. ¹H NMR (400 MHz, DMSO-d₆) δ 7.92-7.88 (m, 2H), 7.39 (d, 1H), 4.23 (q, 2H), 4.16 (s, 3H), 3.81-3.80 (m, 2H), 3.41-3.31 (m, 5H), 2.77 (t, 2H), 2.29-2.26 (m, 2H), 2.05 (bs, 1H), 1.77-1.71 (m, 2H), 1.36-1.28 (m, 7H), 0.93 (t, 3H); MS: m/z=519.3 (M+1, ESI+); HRMS: 519.2383.

### Synthesis of Compound 12

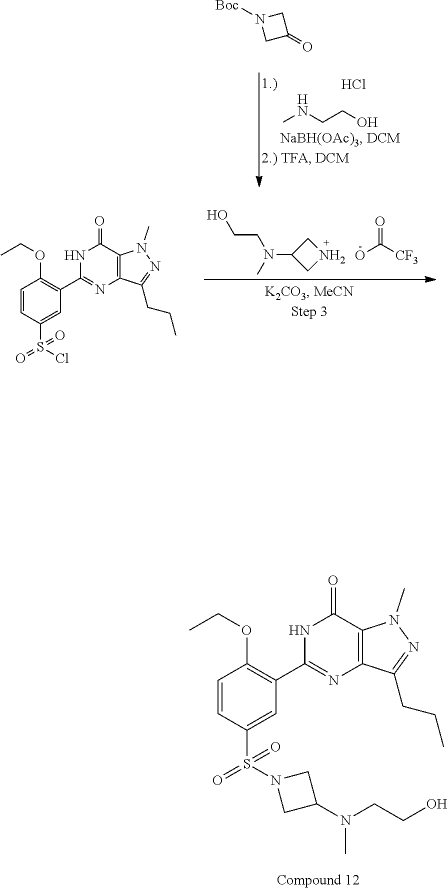

### Step 1:

To a solution of 2-(methylamino)ethan-1-ol hydrochloride (830 mg, 11.05 mmol) and tert-butyl 3-oxoazetidine-1-carboxylate (2.08 g, 12.16 mmol) in DCM (20 mL) was added NaBH(OAc)₃ (3.51 g, 16.58 mmol), the reaction mixture was stirred at 25° C. for 16 h. The reaction mixture was poured into water (200 mL) and extracted with DCM (50 mL×3). The combined organic layers were dried over Na₂SO₄ and concentrated under reduced pressure, the residue was purified by column chromatography to afford tert-butyl 3-((2-hydroxyethyl)(methyl)amino)azetidine-1-carboxylate (2 g, 78.74% yield) as a white solid. MS: m/z=231.3 (M+1, ESI+).

### Step 2:

To a solution of tert-butyl 3-((2-hydroxyethyl)(methyl)amino)azetidine-1-carboxylate (2 g, 8.68 mmol) in DCM (40 mL) was added TFA (9.90 g, 86.84 mmol), the reaction mixture was stirred at 25° C. for 16 h. The resulting solution was evaporated to afford 2-(azetidin-3-yl(methyl)amino)ethan-1-ol; 2,2,2-trifluoroacetate salt (1 g, 88.45% yield) as a yellow oil. MS: m/z=131.3 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (316 mg, 768 umol) and 2-(azetidin-3-yl(methyl)amino)ethan-1-ol; 2,2,2-trifluoroacetate salt (100 mg, 768 umol) in MeCN (6 mL) was added K₂CO₃ (318 mg, 2.30 mmol), the reaction mixture was stirred at 100° C. for 6 h. Filtered and evaporated under reduced pressure, the residue was purified by prep-HPLC to afford 5-(2-ethoxy-5-((3-((2-hydroxyethyl)(methyl)amino)azetidin-1-yl)sulfonyl)phenyl)-1-methyl-3-propyl-1,6-dihydro-7H-pyrazolo[4,3-d]pyrimidin-7-one (85 mg, 22.38% yield) as a white solid. ¹H NMR (400 MHz, DMSO-d₆) δ 11.96 (bs, 1H), 7.94-7.90 (m, 2H), 7.40 (d, 1H), 4.42 (bs, 1H), 4.24-4.20 (m, 2H), 4.17 (s, 3H), 3.75 (t, 2H), 3.49 (t, 2H), 3.35-3.34 (m, 2H), 3.25-3.21 (m, 1H), 2.78 (t, 2H), 2.20 (t, 2H), 1.95 (s, 3H), 1.77-1.71 (m, 2H), 1.35 (t, 3H), 0.93 (t, 3H); MS: m/z=505.4 (M+1, ESI+); HRMS: 505.2229.

### Synthesis of Compound 13

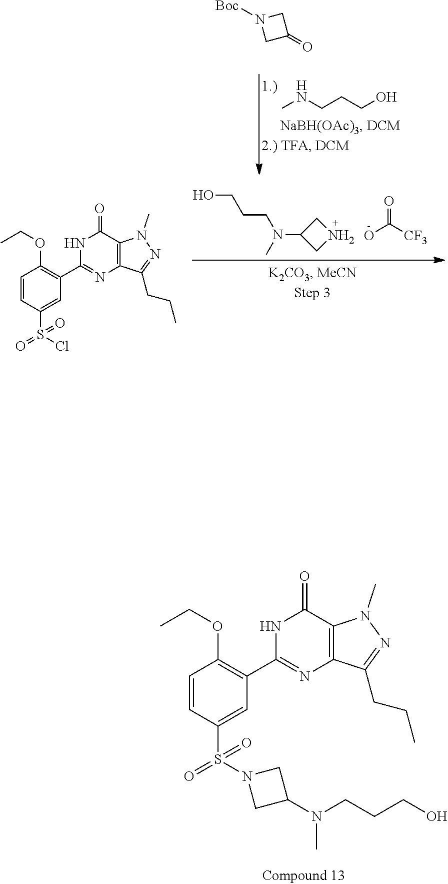

### Step 1:

To a solution of 3-(methylamino)propan-1-ol (2 g, 22.44 mmol) and tert-butyl 3-oxoazetidine-1-carboxylate (3.84 g, 22.44 mmol) in DCM (50 mL) was added NaBH(OAc)₃ (4.76 g, 22.44 mmol), the reaction mixture was stirred at 25° C. for 16 h. The reaction mixture was poured into water (200 mL) and extracted with DCM (50 mL×3). The combined organic layers were dried over Na₂SO₄ and concentrated under reduced pressure, the residue was purified by column chromatography to afford tert-butyl 3-((3-hydroxypropyl)(methyl)amino)azetidine-1-carboxylate (5 g, 91.20% yield) as a white solid. MS: m/z=245.3 (M+1, ESI+).

### Step 2:

To a solution of tert-butyl 3-((3-hydroxypropyl)(methyl)amino)azetidine-1-carboxylate (5 g, 20.46 mmol) in DCM (50 mL) was added TFA (2.32 g, 20.46 mmol), the reaction mixture was stirred at 25° C. for 16 h. The resulting solution was evaporated to afford 3-(azetidin-3-yl(methyl)amino)propan-1-ol; 2,2,2-trifluoroacetate salt 2 g, 67.77% yield) as a yellow oil. MS: m/z=145.3 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (712 mg, 1.73 mmol) and 3-(azetidin-3-yl(methyl)amino)propan-1-ol; 2,2,2-trifluoroacetate salt (500 mg, 3.47 mmol) in MeCN (20 mL) was added K₂CO₃ (958 mg, 6.93 mmol), the reaction mixture was stirred at 80° C. for 4 h. Filtered and evaporated under reduced pressure, the residue was purified by prep-HPLC to afford compound 13, 5-(2-ethoxy-5-((3-((3-hydroxypropyl)(methyl)amino)azetidin-1-yl)sulfonyl) phenyl)-1-methyl-3-propyl-1,6-dihydro-7H-pyrazolo[4,3-d]pyrimidin-7-one (110 mg, 12.24% yield) as a white solid. ¹H NMR (400 MHz, DMSO-d₆) δ 12.25 (s, 1H), 7.95-7.90 (m, 2H), 7.41 (d, 1H), 4.37 (bs, 1H), 4.24 (q, 2H), 4.17 (s, 3H), 3.77 (t, 2H), 3.48 (t, 2H), 3.31-3.28 (m, 2H), 3.14-3.10 (m, 1H), 2.78 (t, 2H), 2.11 (t, 2H), 1.88 (s, 3H), 1.77-1.72 (m, 2H), 1.44-1.34 (m, 5H), 0.94 (t, 3H); MS: m/z=519.3 (M+1, ESI+); HRMS: 519.2381.

### Synthesis of Compound 14

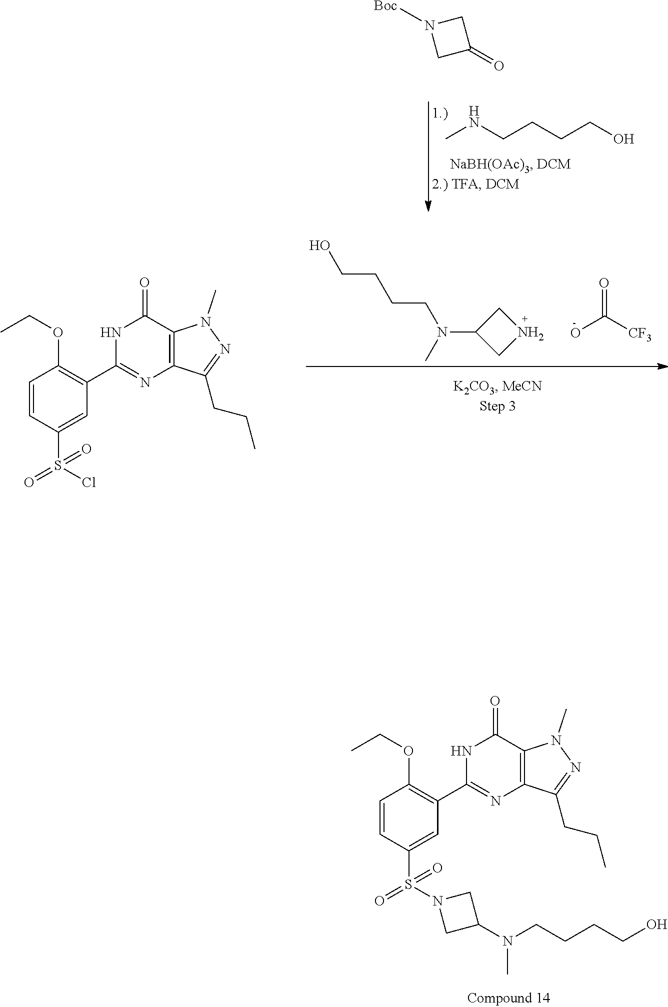

### Step 1:

To a solution of 4-(methylamino)butan-1-ol hydrochloride (400 mg, 3.88 mmol) and tert-butyl 3-oxoazetidine-1-carboxylate (664 mg, 3.88 mmol) in DCM (20 mL) was added NaBH(OAc)₃ (1.23 g, 5.82 mmol), the reaction mixture was stirred at 25° C. for 16 h. The reaction mixture was poured into water (200 mL) and extracted with DCM (50 mL×3). The combined organic layers were dried over Na₂SO₄ and concentrated under reduced pressure, the residue was purified by column chromatography to afford tert-butyl 3-((4-hydroxybutyl)(methyl)amino)azetidine-1-carboxylate (700 mg, 69.88% yield) as a white solid. MS: m/z=259.2 (M+1, ESI+).

### Step 2:

To a solution of tert-butyl 3-((4-hydroxybutyl)(methyl)amino)azetidine-1-carboxylate (500 mg, 1.94 mmol) in DCM (40 mL) was added TFA (2.21 g, 19.35 mmol), the reaction mixture was stirred at 25° C. for 16 h. The resulting solution was evaporated to afford 4-(azetidin-3-yl (methyl)amino)butan-1-ol; 2,2,2-trifluoroacetate salt (260 mg, 84.90% yield) as a yellow oil. MS: m/z=159.2 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (300 mg, 730 umol) and 4-(azetidin-3-yl(methyl)amino)butan-1-ol; 2,2,2-trifluoroacetate salt (116 mg, 730 umol) in THF (6 mL) was added TEA (369 mg, 3.65 mmol), the reaction mixture was stirred at 25° C. for 0.5 h. Filtered and evaporated under reduced pressure, the residue was purified by prep-HPLC to afford compound 14, 5-(2-ethoxy-5-((3-((4-hydroxybutyl)(methyl)amino)azetidin-1-yl)sulfonyl)phenyl)-1-methyl-3-propyl-1,6-dihydro-7H-pyrazolo[4,3-d] pyrimidin-7-one (135 mg, 34.71% yield) as a white solid. ¹H NMR (400 MHz, DMSO-d₆) δ 12.24 (s, 1H), 7.97-7.92 (m, 2H), 7.42 (d, 1H), 4.44 (bs, 1H), 4.25 (q, 2H), 4.17 (s, 3H), 3.91-3.90 (m, 5H), 3.38-3.35 (m, 3H), 2.81-2.77 (m, 4H), 2.41-2.40 (m, 2H), 1.78-1.72 (m, 2H), 1.49-1.48 (m, 2H), 1.37-1.34 (m, 5H), 0.94 (t, 3H); MS: m/z=533.3 (M+1, ESI+); HRMS: 533.2539.

### Synthesis of Compound 15

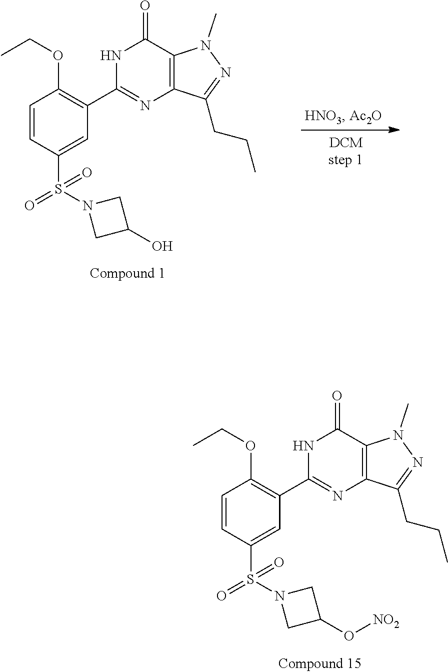

To a solution of compound 1, 5-(2-ethoxy-5-((3-hydroxyazetidin-1-yl)sulfonyl)phenyl)-1-methyl-3-propyl-1,6-dihydro-7H-pyrazolo[4,3-d]pyrimidin-7-one (180 mg, 402 umol) in DCM (10 mL) was added HNO₃ (117 mg, 1.21 mmol) and Ac₂O (213 mg, 2.01 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 15, 1-((4-ethoxy-3-(1-methyl-7-oxo-3-propyl-6,7-dihydro-1H-pyrazolo[4,3-d]pyrimidin-5-yl)phenyl)sulfonyl) azetidin-3-yl nitrate (75 mg, 37.8% yield) as a white solid. ¹H NMR (400 MHz, DMSO-d₆) δ 12.27 (s, 1H), 7.97-7.94 (m, 2H), 7.42-7.40 (m, 1H), 5.43-5.37 (m, 1H), 4.24 (q, 2H), 4.17-4.13 (m, 5H), 3.91-3.88 (m, 2H), 2.77 (t, 2H), 1.76-1.69 (m, 2H), 1.34 (t, 3H), 0.93 (t, 3H); MS: m/z=493.1 (M+1, ESI+); HRMS: 493.1503.

### Synthesis of Compound 16

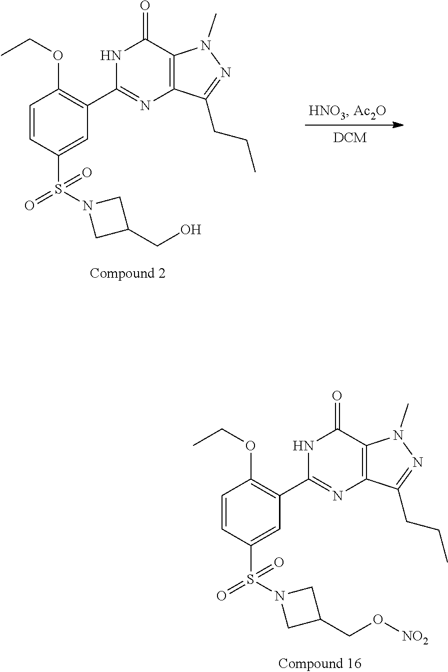

To a solution of compound 2, 5-(2-ethoxy-5-((3-(hydroxymethyl)azetidin-1-yl)sulfonyl)phenyl)-1-methyl-3-propyl-1,6-dihydro-7H-pyrazolo[4,3-d]pyrimidin-7-one (461 mg, 999 umol) in DCM (10 mL) was added HNO₃ (189 mg, 3 mmol) and Ac₂O (318 mg, 3 mmol), the resulting mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 16, (1-((4-ethoxy-3-(1-methyl-7-oxo-3-propyl-6,7-dihydro-1H-pyrazolo[4,3-d]pyrimidin-5-yl)phenyl) sulfonyl)azetidin-3-yl)methyl nitrate (220 mg, 43.31% yield) as a wbite solid. ¹H NMR (400 MHz, DMSO-d₆) δ 12.16 (bs, 1H), 7.94-7.91 (m, 2H), 7.41 (d, 1H), 4.50 (d, 2H), 4.24 (q, 2H), 4.16 (s, 3H), 3.82 (t, 2H), 3.61-3.57 (m, 2H), 2.81-2.76 (m, 3H), 1.76-1.71 (m, 2H), 1.35 (t, 3H), 0.93 (t, 3H); MS: m/z=507.1 (M+1, ESI+); HRMS: 507.1659.

### Synthesis of Compound 17

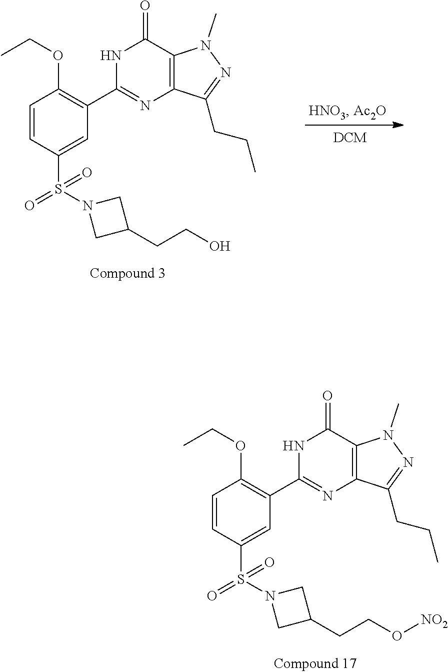

To a solution of compound 3, 5-(2-ethoxy-5-((3-(2-hydroxyethyl)azetidin-1-yl)sulfonyl)phenyl)-1-methyl-3-propyl-1,6-dihydro-7H-pyrazolo[4,3-d]pyrimidin-7-one (210 mg, 442 umol) in DCM (8 mL) was added HNO₃ (83 mg, 1.32 mmol) and Ac₂O (140 mg, 1.32 mmol), the resulting mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 17, 2-(1-((4-ethoxy-3-(1-methyl-7-oxo-3-propyl-6,7-dihydro-1H-pyrazolo [4,3-d]pyrimidin-5-yl)phenyl) sulfonyl)azetidin-3-yl)ethyl nitrate (110 mg, 47.85% yield) as a white solid. ¹H NMR (400 MHz, DMSO-d₆) δ 12.24 (s, 1H), 7.92-7.89 (m, 2H), 7.41 (d, 1H), 4.42-4.39 (m, 2H), 4.22 (q, 2H), 4.16 (s, 3H), 3.81 (t, 2H), 3.40 (t, 2H), 2.77 (t, 2H), 2.61-2.54 (m, 1H), 1.76-1.72 (m, 4H), 1.34 (t, 3H), 0.93 (t, 3H); MS: m/z=521.4 (M+1, ESI+); HRMS: 521.1815.

### Synthesis of Compound 18

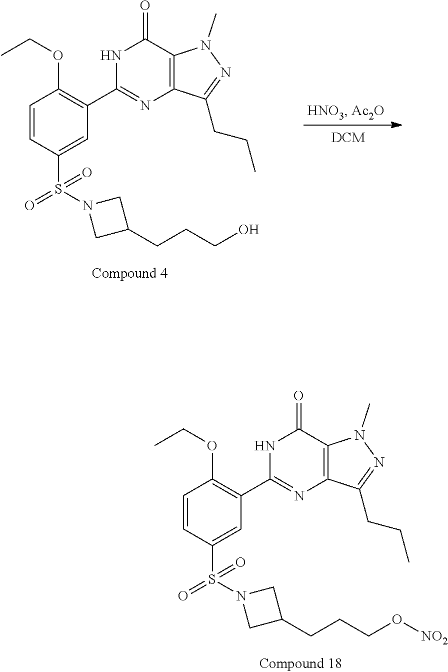

To a solution of compound 4, 5-(2-ethoxy-5-((3-(3-hydroxypropyl)azetidin-1-yl)sulfonyl)phenyl)-1-methyl-3-propyl-1,6-dihydro-7H-pyrazolo[4,3-d]pyrimidin-7-one (260 mg, 531 umol) in DCM (10 mL) was added HNO₃ (154 mg, 1.59 mmol) and Ac₂O (102 mg, 1.59 mmol), the resulting mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 18, 3-(1-((4-ethoxy-3-(1-methyl-7-oxo-3-propyl-6,7-dihydro-1H-pyrazolo[4,3-d]pyrimidin-5-yl) phenyl)sulfonyl)azetidin-3-yl)propyl nitrate (80 mg, 28% yield) as a white solid. ¹H NMR (400 MHz, DMSO-d₆) δ 12.23 (s, 1H), 7.93-7.89 (m, 2H), 7.40 (d, 1H), 4.42 (t, 2H), 4.22 (q, 2H), 4.16 (s, 3H), 3.79 (t, 2H), 3.36-3.32 (m, 2H), 2.77 (t, 2H), 2.44-2.37 (m, 1H), 1.79-1.69 (m, 2H), 1.54-1.47 (m, 2H), 1.41-1.33 (m, 5H), 0.93 (t, 3H); MS: m/z=535.3 (M+1, ESI+); HRMS: 535.1972.

### Synthesis of Compound 21

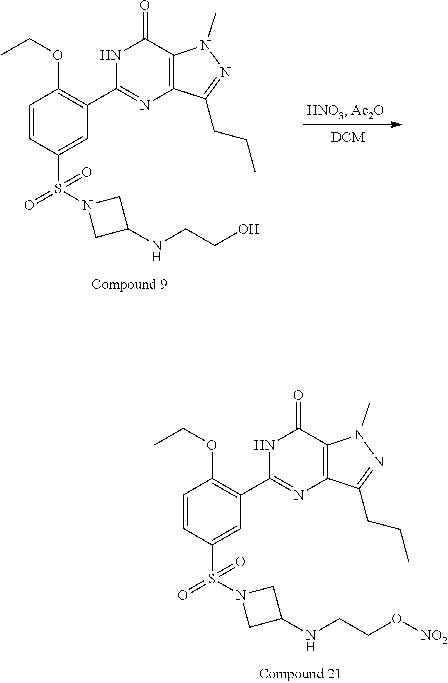

To a solution of 5-(2-ethoxy-5-((3-((2-hydroxyethyl)amino)azetidin-1-yl)sulfonyl)phenyl)-1-methyl-3-propyl-1,6-dihydro-7H-pyrazolo[4,3-d]pyrimidin-7-one (250 mg, 509 umol) in DCM (10 mL) was added HNO₃ (142 mg, 1.53 mmol) and Ac₂O (156 mg, 1.53 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 21, 2-((1-((4-ethoxy-3-(1-methyl-7-oxo-3-propyl-6,7-dihydro-1H-pyrazolo[4,3-d]pyrimidin-5-yl) phenyl)sulfonyl)azetidin-3-yl)amino)ethyl nitrate (80 mg, 28% yield) as a white solid. ¹H NMR (400 MHz, DMSO-d₆) δ 12.21 (bs, 1H), 7.94-7.90 (m, 2H), 7.40 (d, 1H), 4.47-4.45 (m, 2H), 4.26-4.21 (m, 2H), 4.17 (s, 3H), 3.84-3.82 (m, 2H), 3.45-3.43 (m, 3H), 3.33 (bs, 1H), 2.80-2.71 (m, 4H), 1.77-1.72 (m, 2H), 1.35 (t, 3H), 0.94 (t, 3H); MS: m/z=536.2 (M+1, ESI+); HRMS: 536.1923.

### Synthesis of Compound 22

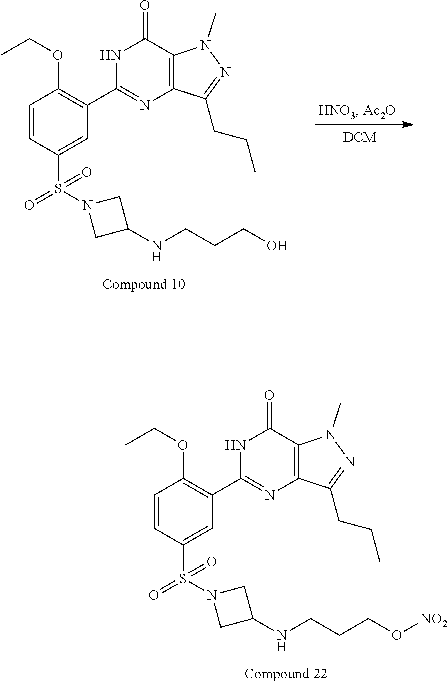

To a solution of 5-(2-ethoxy-5-((3-((3-hydroxypropyl)amino)azetidin-1-yl)sulfonyl)phenyl)-1-methyl-3-propyl-1,6-dihydro-7H-pyrazolo[4,3-d]pyrimidin-7-one (180 mg, 357 umol) in DCM (8 mL) was added HNO₃ (104 mg, 1.07 mmol) and Ac₂O (113 mg, 1.07 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 22, 3-((1-((4-ethoxy-3-(1-methyl-7-oxo-3-propyl-6,7-dihydro-1H-pyrazolo[4,3-d]pyrimidin-5-yl) phenyl)sulfonyl)azetidin-3-yl)amino)propyl nitrate (53 mg, 27.03% yield) as a white solid. ¹H NMR (400 MHz, DMSO-d₆) δ 12.27 (bs, 1H), 7.99-7.95 (m, 2H), 7.45 (d, 1H), 4.53 (t, 2H), 4.29 (q, 2H), 4.22 (s, 3H), 3.88-3.86 (m, 2H), 3.47-3.42 (m, 3H), 2.83 (t, 2H), 2.46-2.43 (m, 3H), 1.82-1.71 (m, 4H), 1.40 (t, 3H), 0.99 (t, 3H); MS: m/z=550.3 (M+1, ESI+), HRMS: 550.2081.

### Synthesis of Compound 23

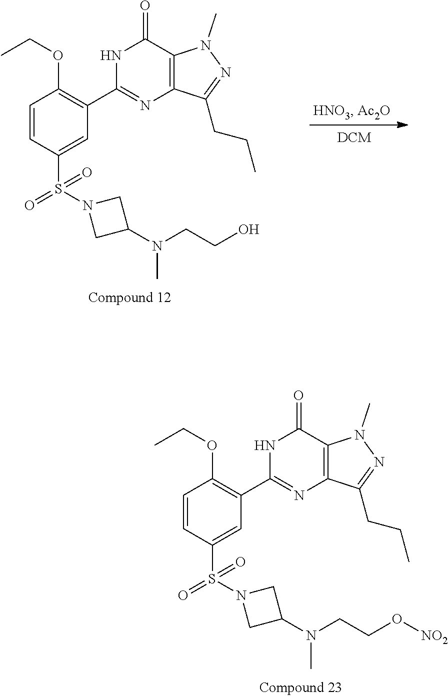

To a solution of 5-(2-ethoxy-5-((3-((2-hydroxyethyl)(methyl)amino)azetidin-1-yl)sulfonyl)phenyl)-1-methyl-3-propyl-1,6-dihydro-7H-pyrazolo[4,3-d]pyrimidin-7-one (400 mg, 817 umol) in DCM (10 mL) was added HNO₃ (257 mg, 4.09 mmol) and Ac₂O (433 mg, 4.09 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 23, 2-((1-((4-ethoxy-3-(1-methyl-7-oxo-3-propyl-6,7-dihydro-1H-pyrazolo[4,3-d] pyrimidin-5-yl)phenyl)sulfonyl)azetidin-3-yl)(methyl)amino)ethyl nitrate (35 mg, 8.01% yield) as a white solid. ¹H NMR (400 MHz, DMSO-d₆) δ 11.44 (bs, 1H), 7.96-7.91 (m, 2H), 7.41 (d, 1H), 4.48-4.46 (m, 2H), 4.24-4.21 (m, 2H), 4.17 (s, 3H), 3.79-3.76 (m, 2H), 3.53-3.49 (m, 2H), 3.33-3.28 (m, 2H), 2.78 (t, 2H), 2.51-2.50 (m, 1H), 2.00 (s, 3H), 1.77-1.72 (m, 2H), 1.36 (t, 3H), 0.94 (t, 3H); MS: m/z=550.3 (M+1, ESI+); HRMS: 550.2076.

### Synthesis of Compound 24

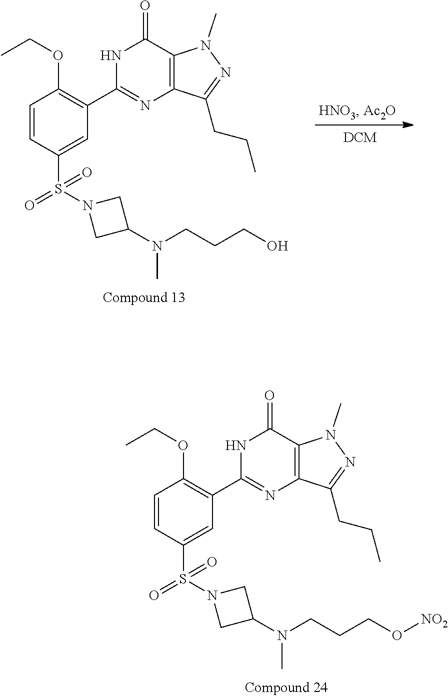

To a solution of 5-(2-ethoxy-5-((3-((3-hydroxypropyl)(methyl)amino)azetidin-1-yl)sulfonyl)phenyl)-1-methyl-3-propyl-1,6-dihydro-7H-pyrazolo[4,3-d]pyrimidin-7-one (400 mg, 771 umol) in DCM (20 mL) was added HNO₃ (583 mg, 9.26 mmol) and Ac₂O (236 mg, 2.31 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 24, 3-((1-((4-ethoxy-3-(1-methyl-7-oxo-3-propyl-6,7-dihydro-1H-pyrazolo[4,3-d] pyrimidin-5-yl)phenyl)sulfonyl)azetidin-3-yl)(methyl)amino)propyl nitrate (100 mg, 23% yield) as a white solid. ¹H NMR (400 MHz, DMSO-d₆) δ 12.24 (bs, 1H), 7.97 (d, 1H), 7.92 (dd, 1H), 7.41 (d, 1H), 4.40 (t, 2H), 4.24 (q, 2H), 4.17 (t, 3H), 3.80 (t, 2H), 3.47 (t, 2H), 3.17-3.14 (m, 1H), 2.78 (t, 2H), 2.51 (t, 2H), 1.91 (s, 3H), 1.78-1.65 (m, 4H), 1.36 (t, 3H), 0.94 (t, 3H); MS: m/z=564.3 (M+1, ESI+); HRMS: 564.2233

### Synthesis of Compound 25

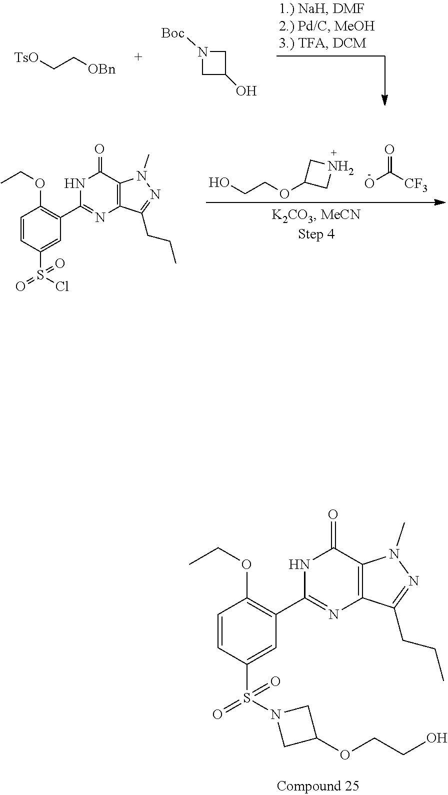 [SMILES: Cc1ccc(S(=O)(=O)OCCOCc2ccccc2)cc1 ; CC(C)(C)OC(=O)N1CC(O)C1 ; CCCc1nn(C)c2c(=O)[nH]c(-c3cc(S(=O)(=O)Cl)ccc3OCC)nc12 ; OCCOC1CNC1 ; O=C(O)C(F)(F)F ; CCCc1nn(C)c2c(=O)[nH]c(-c3cc(S(=O)(=O)N4CC(OCCO)C4)ccc3OCC)nc12]

### Step 1:

To a solution of tert-butyl 3-hydroxyazetidine-1-carboxylate (1.13 g, 6.53 mmol) in DMF (20 mL) was added NaH (203.65 mg, 8.49 mmol) at 0° C. in portions, the reaction mixture was stirred at 25° C. for 1 h. Then 2-(benzyloxy)ethyl 4-methylbenzenesulfonate (2 g, 6.53 mmol) was added and stirred at 25° C. for 16 h. The resulting solution was quenched by saturated NH₄Cl (50 mL), extracted with EA (50 mL×3), washed with water (50 mL) and brine (50 mL), dried over Na₂SO₄ and concentrated. The residue was purified by column chromatography to afford tert-butyl 3-(2-(benzyloxy)ethoxy)azetidine-1-carboxylate (1.3 g, 64.79% yield) as a yellow oil. MS: m/z=330.2 (M+23, ESI+).

### Step 2:

To a solution of tert-butyl 3-(2-(benzyloxy)ethoxy)azetidine-1-carboxylate (1.3 g, 4.23 mmol) in MeOH (20 mL) was added Pd/C (300 mg), the reaction mixture was stirred at 25° C. for 16 h under H₂. The resulting solution was filtered and evaporated to afford tert-butyl 3-(2-hydroxyethoxy) azetidine-1-carboxylate (700 mg, 76.18% yield) as a yellow oil. MS: m/z=218.2 (M+23, ESI+). ¹H NMR (400 MHz, DMSO-d₆) δ 4.65 (t, 1H), 4.24 (tt, 1H), 4.03-3.93 (m, 2H), 3.65 (dd, 2H), 3.48 (q, 2H), 3.36 (dd, 2H), 1.37 (s, 9H).

### Step 3:

To a solution of tert-butyl 3-(2-hydroxyethoxy)azetidine-1-carboxylate (700 mg, 3.22 mmol) in DCM (10 mL) was added TFA (1.84 g, 16.11 mmol), the reaction mixture was stirred at 25° C. for 5 h. The resulting solution was evaporated to afford 2-(azetidin-3-yloxy)ethan-1-ol; 2,2,2-trifluoroacetate salt (650 mg, 87.65% yield) as a yellow oil. MS: m/z=118.3 (M+1, ESI+).

### Step 4:

To a solution of 4-ethoxy-3-(1-methyl-7-oxo-3-propyl-6,7-dihydro-1H-pyrazolo[4,3-d]pyrimidin-5-yl) benzenesulfonyl chloride (572.62 mg, 1.39 mmol) and 2-(azetidin-3-yloxy)ethan-1-ol; 2,2,2-trifluoroacetate salt (400 mg, 1.74 mmol) in MeCN (20 mL) was added K₂CO₃ (720.57 mg, 5.21 mmol), the reaction mixture was stirred at 80° C. for 2 h. The resulting solution was evaporated and purified by prep-HPLC to afford compound 25, 5-(2-ethoxy-5-((3-(2-hydroxyethoxy)azetidin-1-yl)sulfonyl)phenyl)-1-methyl-3-propyl-1,6-dihydro-7H-pyrazolo[4,3-d] pyrimidin-7-one (500 mg, 58.41% yield) as a white solid. ¹H NMR (400 MHz, DMSO-d₆) δ 12.14 (bs, 1H), 7.92-7.87 (m, 2H), 7.39 (d, 1H), 4.24-4.16 (m, 6H), 3.92 (t, 2H), 3.50-3.47 (m, 2H), 3.41-3.38 (m, 2H), 3.31-3.29 (m, 2H), 2.77 (t, 2H), 1.77-1.71 (m, 2H), 1.34 (t, 3H), 0.94 (t, 3H); MS: m/z=492.3 (M+1, ESI+); HRMS: 492.1913.

### Synthesis of Compound 26

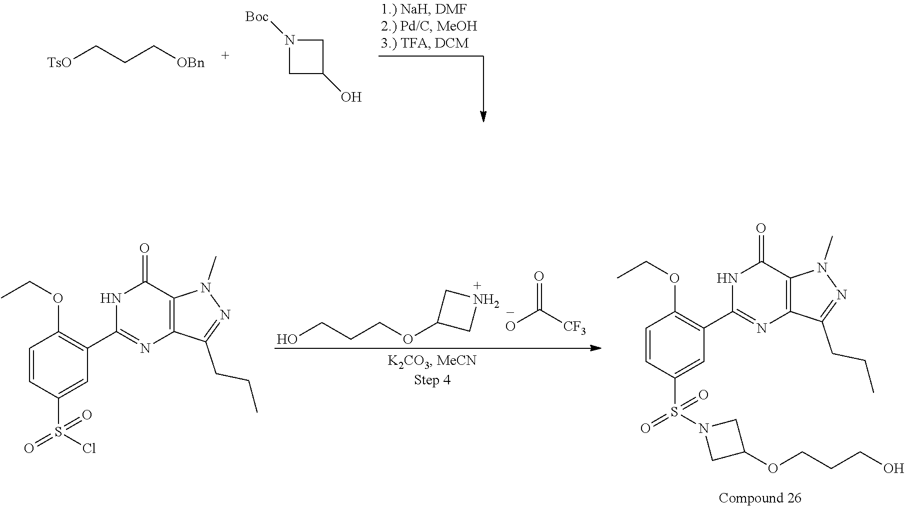

### Step 1:

To a solution of tert-butyl 3-hydroxyazetidine-1-carboxylate (703 mg, 4.06 mmol) in DMF (20 mL) was added NaH (243.42 mg, 6.09 mmol) at 0° C. in portions, the reaction mixture was stirred at 25° C. for 1 h. Then 3-(benzyloxy)propyl 4-methylbenzenesulfonate (1.3 g, 4.06 mmol) was added and stirred at 25° C. for 16 h. The resulting solution was quenched by saturated NH₄Cl (50 mL), extracted with EA (50 mL×3), washed with water (50 mL) and brine (50 mL), dried over Na₂SO₄ and concentrated. The residue was purified by column chromatography to afford tert-butyl 3-(3-(benzyloxy)propoxy)azetidine-1-carboxylate (0.9 g, 69.01% yield) as a yellow oil. MS: m/z=322.2 (M+1, ESI+).

### Step 2:

To a solution of tert-butyl 3-(3-(benzyloxy)propoxy)azetidine-1-carboxylate (900 mg, 2.80 mmol) in MeOH (20 mL) was added Pd/C (400 mg), the reaction mixture was stirred at 25° C. for 16 h under H₂. The resulting solution was filtered and evaporated to afford tert-butyl 3-(3-hydroxypropoxy)azetidine-1-carboxylate (500 mg, 77.20% yield) as a yellow oil. MS: m/z=272.1 (M+41, ESI+).

### Step 3:

To a solution of tert-butyl 3-(3-hydroxypropoxy)azetidine-1-carboxylate (500 mg, 2.16 mmol) in DCM (10 mL) was added TFA (2.46 g, 21.62 mmol), the reaction mixture was stirred at 25° C. for 16 h. The resulting solution was evaporated to afford 3-(azetidin-3-yloxy)propan-1-ol; 2,2,2-trifluoroacetate salt (240 mg, 84.64% yield) as a yellow oil. MS: m/z=132.3 (M+1, ESI+).

### Step 4:

To a solution of 4-ethoxy-3-(1-methyl-7-oxo-3-propyl-6,7-dihydro-1H-pyrazolo[4,3-d]pyrimidin-5-yl) benzenesulfonyl chloride (500 mg, 1.22 mmol) and 3-(azetidin-3-yloxy)propan-1-ol; 2,2,2-trifluoroacetate salt (240 mg, 1.83 mmol) in MeCN (20 mL) was added K₂CO₃ (2.52 g, 18.25 mmol), the reaction mixture was stirred at 80° C. for 2 h. The resulting solution was evaporated and purified by prep-HPLC to afford compound 26, 5-(2-ethoxy-5-((3-(3-hydroxypropoxy)azetidin-1-yl)sulfonyl)phenyl)-1-methyl-3-propyl-1,6-dihydro-7H-pyrazolo[4,3-d]pyrimidin-7-one (400 mg, 65.01% yield) as a white solid. ¹H NMR (400 MHz, DMSO-d₆) δ 7.91-7.88 (m, 2H), 7.39 (d, 1H), 4.24-4.08 (m, 6H), 3.95-3.91 (m, 2H), 3.48-3.45 (m, 2H), 3.31-3.27 (m, 4H), 2.79-2.73 (m, 2H), 1.76-1.71 (m, 2H), 1.53-1.50 (m, 2H), 1.35-1.32 (m, 3H), 0.93 (t, 3H); MS: m/z=506.1 (M+1, ESI+); HRMS: 506.2071.

### Synthesis of Compound 27

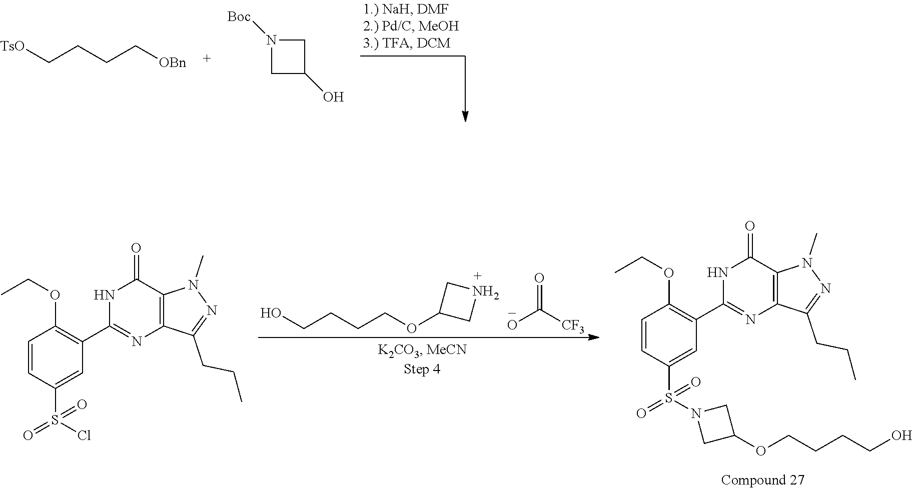

### Step 1:

To a solution of tert-butyl 3-hydroxyazetidine-1-carboxylate (800 mg, 4.62 mmol) in DMF (20 mL) was added NaH (276 mg, 6.91 mmol) at 0° C. in portions, the reaction mixture was stirred at 25° C. for 1 h. Then 4-(benzyloxy)butyl 4-methylbenzenesulfonate (1.54 g, 4.60 mmol) was added and stirred at 25° C. for 16 h. The resulting solution was quenched by saturated NH₄Cl (50 mL), extracted with EA (50 mL×3), washed with water (50 mL) and brine (50 mL), dried over Na₂SO₄ and concentrated. The residue was purified by column chromatography to afford tert-butyl 3-(4-(benzyloxy)butoxy)azetidine-1-carboxylate (1.1 g, 71.21% yield) as a yellow oil. MS: m/z=358.1 (M+23, ESI+).

### Step 2:

To a solution of tert-butyl 3-(4-(benzyloxy)butoxy)azetidine-1-carboxylate (1.1 g, 3.28 mmol) in MeOH (20 mL) was added Pd/C (400 mg), the reaction mixture was stirred at 25° C. for 16 h under H2. The resulting solution was filtered and evaporated to afford tert-butyl 3-(4-hydroxybutoxy)azetidine-1-carboxylate (800 mg, 91.16% yield) as a yellow oil. MS: m/z=268.2 (M+23, ESI+).

### Step 3:

To a solution of tert-butyl 3-(4-hydroxybutoxy)azetidine-1-carboxylate (800 mg, 3.26 mmol) in DCM (20 mL) was added TFA (3.72 g, 32.61 mmol), the reaction mixture was stirred at 25° C. for 16 h. The resulting solution was evaporated to afford 4-(azetidin-3-yloxy)butan-1-ol; 2,2,2-trifluoroacetate salt (370 mg, 78.14% yield) as a yellow oil. MS: m/z=146.1 (M+1, ESI+).

### Step 4:

To a solution of 4-ethoxy-3-(1-methyl-7-oxo-3-propyl-6,7-dihydro-1H-pyrazolo[4,3-d]pyrimidin-5-yl) benzenesulfonyl chloride (500 mg, 1.22 mmol) and 4-(azetidin-3-yloxy)butan-1-ol; 2,2,2-trifluoroacetate salt (265 mg, 1.83 mmol l) in MeCN (20 mL) was added K₂CO₃ (2.52 g, 18.25 mmol), the reaction mixture was stirred at 80° C. for 2 h. The resulting solution was evaporated and purified by prep-HPLC to afford compound 27, 5-(2-ethoxy-5-((3-(4-hydroxybutoxy)azetidin-1-yl)sulfonyl)phenyl)-1-methyl-3-propyl-1,6-dihydro-7H-pyrazolo[4,3-d]pyrimidin-7-one (580 mg, 91.72% yield) as a white solid. ¹H NMR (400 MHz, DMSO-d₆) δ 12.23 (bs, 1H), 7.91-7.87 (m, 2H), 7.39 (d, 1H), 4.44 (bs, 1H), 4.22 (q, 2H), 4.17 (s, 3H), 4.15-4.08 (m, 1H), 3.93 (t, 2H), 3.46-3.43 (m, 2H), 3.31-3.28 (m, 2H), 3.21 (t, 2H), 2.76 (t, 2H), 1.76-1.69 (m, 2H), 1.40-1.26 (m, 7H), 0.93 (t, 3H); MS: m/z=520.2 (M+1, ESI+); HRMS: 520.2227.

### Synthesis of Compound 28

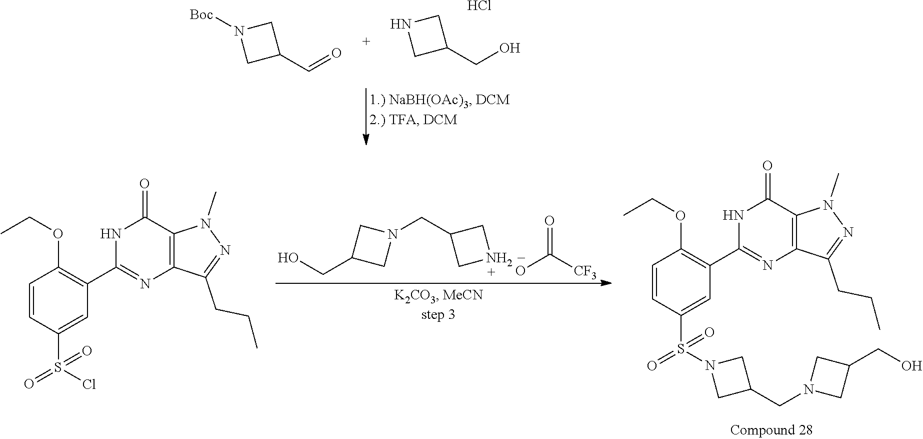

### Step 1:

To a solution of tert-butyl 3-formylazetidine-1-carboxylate (380 mg, 2.05 mmol) and azetidin-3-ylmethanol hydrochloride (233 mg, 2.67 mmol) in DCM (10 mL) was added NaBH(OAc)₃ (521 mg, 2.46 mmol), the reaction mixture was stirred at 25° C. for 16 h. The reaction mixture was poured into water (100 mL) and extracted with DCM (20 mL×3). The combined organic layers were dried over Na₂SO₄ and concentrated under reduced pressure, the residue was purified by column chromatography to afford tert-butyl 3-((3-(hydroxymethyl)azetidin-1-yl)methyl)azetidine-1-carboxylate (400 mg, 60% yield) as a white solid. MS: m/z=257.3 (M+1, ESI+).

### Step 2:

To a solution of tert-butyl 3-((3-(hydroxymethyl)azetidin-1-yl)methyl)azetidine-1-carboxylate (400 mg, 1.56 mmol) in DCM (5 mL) was added TFA (1.78 g, 15.6 mmol), the reaction mixture was stirred at 25° C. for 4 h. The reaction mixture was evaporated under reduced pressure to afford (1-(azetidin-3-ylmethyl)azetidin-3-yl)methanol; 2,2,2-trifluoroacetate salt (273 mg, crude) as a colorless oil. MS: m/z=157.4 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (600 mg, 1.46 mmol) and (1-(azetidin-3-ylmethyl)azetidin-3-yl) methanol; 2,2,2-trifluoroacetate salt (273 mg, 1.75 mmol) in MeCN (10 mL) was added K₂CO₃ (2.02 g, 14.6 mmol), the reaction mixture was stirred at 25° C. for 2 h. Filtered and evaporated under reduced pressure, the residue was purified by prep-HPLC to afford compound 28, 5-(2-ethoxy-5-((3-((3-(hydroxymethyl)azetidin-1-yl)methyl)azetidin-1-yl) sulfonyl)phenyl)-1-methyl-3-propyl-1,6-dihydro-7H-pyrazolo[4,3-d] pyrimidin-7-one (400 mg, 76% yield) as a white solid. ¹H NMR (400 MHz, DMSO-d₆) δ 12.17 (bs, 1H), 7.93-7.89 (m, 2H), 7.41 (d, 1H), 4.52 (bs, 1H), 4.25 (q, 2H), 4.17 (s, 3H), 3.74 (t, 2H), 3.38 (d, 2H), 3.35-3.32 (m, 2H), 3.05 (t, 2H), 2.79 (t, 2H), 2.71 (t, 2H), 2.37-2.32 (m, 2H), 2.24 (d, 2H), 1.77-1.72 (m, 2H), 1.35 (t, 3H), 0.94 (t, 3H); MS: m/z=531.3 (M+1, ESI+); HRMS: 531.2388.

### Synthesis of Compound 29

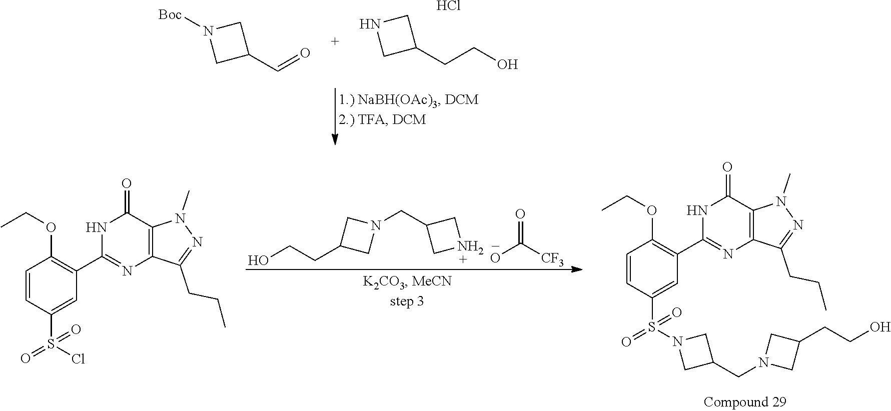

### Step 1:

To a solution of tert-butyl 3-formylazetidine-1-carboxylate (350 mg, 1.89 mmol) and 2-(azetidin-3-yl)than-1-ol; 2,2,2-trifluoroacetate salt (248 mg, 2.46 mmol) in DCM (10 mL) was added NaBH(OAc)₃ (480 mg, 2.27 mmol), the reaction mixture was stirred at 25° C. for 16 h. The reaction mixture was poured into water (100 mL) and extracted with DCM (20 mL×3). The combined organic layers were dried over Na₂SO₄ and concentrated under reduced pressure, the residue was purified by column chromatography to afford tert-butyl 3-((3-(2-hydroxyethyl)azetidin-1-yl)methyl)azetidine-1-carboxylate (285 mg, 48% yield) as a white solid. MS: m/z=271.3 (M+1, ESI+).

### Step 2:

To a solution of tert-butyl 3-((3-(2-hydroxyethyl)azetidin-1-yl)methyl)azetidine-1-carboxylate (280 mg, 1.04 mmol) in DCM (5 mL) was added TFA (1.18 g, 10.36 mmol), the reaction mixture was stirred at 25° C. for 4 h. The reaction mixture was evaporated under reduced pressure to afford 2-(1-(azetidin-3-ylmethyl)azetidin-3-yl)ethan-1-ol; 2,2,2-trifluoroacetate salt (199 mg, crude) as a colorless oil. MS: m/z=171.4 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (400 mg, 974 umol) and 2-(1-(azetidin-3-ylmethyl)azetidin-3-yl)ethan-1-ol; 2,2,2-trifluoroacetate salt (199 mg, 1.17 mmol) in MeCN (10 mL) was added K₂CO₃ (1.34 g, 9.73 mmol), the reaction mixture was stirred at 25° C. for 2 h. Filtered and evaporated under reduced pressure, the residue was purified by prep-HPLC to afford compound 29, 5-(2-ethoxy-5-((3-((3-(2-hydroxyethyl)azetidin-1-yl)methyl)azetidin-1-yl)sulfonyl)phenyl)-1-methyl-3-propyl-1,6-dihydro-7H-pyrazolo[4,3-d]pyrimidin-7-one (190 mg, 28% yield) as a white solid. ¹H NMR (400 MHz, DMSO-d₆) δ 12.22 (bs, 1H), 7.93-7.88 (m, 2H), 7.41 (d, 1H), 4.37 (bs, 1H), 4.24 (q, 2H), 4.17 (s, 3H), 3.74 (t, 2H), 3.36-3.32 (m, 2H), 3.26 (t, 2H), 3.18 (t, 2H), 2.78 (t, 2H), 2.57 (t, 2H), 2.35-2.22 (m, 4H), 1.77-1.72 (m, 2H), 1.54 (q, 2H), 1.35 (t, 3H), 0.94 (t, 3H); MS: m/z=545.4 (M+1, ESI+); HRMS: 545.2544.

### Synthesis of Compound 30

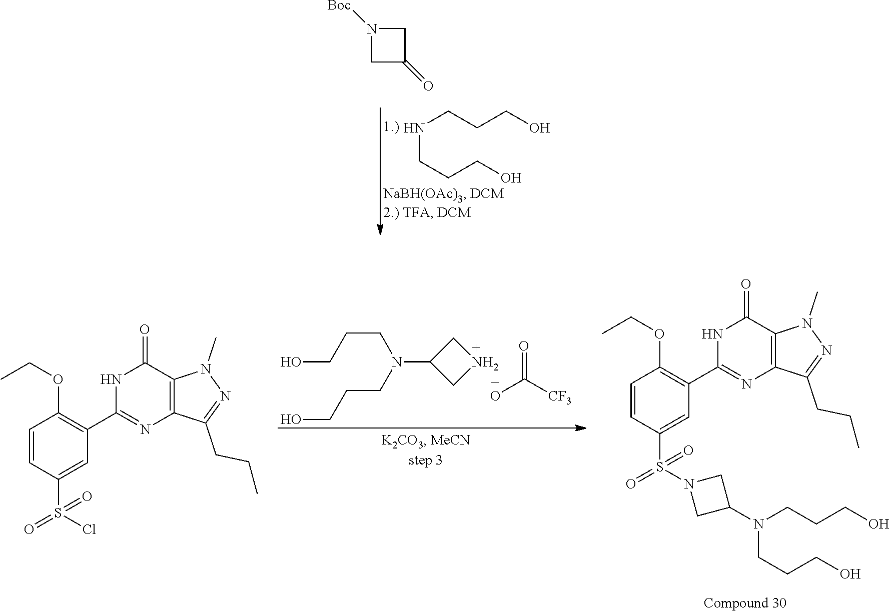

### Step 1:

A mixture of tert-butyl 3-oxoazetidine-1-carboxylate (5.78 g, 33.79 mmol) and 3,3′-azanediylbis(propan-1-ol) (1.8 g, 13.51 mmol) in DCM (40 mL) was stirred at 25° C. for 3 h, then NaBH(OAc)₃ (5.73 g, 27.03 mmol) was added to the above solution and stirred at 25° C. for 72 h. The reaction mixture was poured into water (100 mL) and extracted with DCM (20 mL×3). The combined organic layers were dried over Na₂SO₄ and concentrated under reduced pressure, the residue was purified by column chromatography to afford tert-butyl 3-(bis(3-hydroxypropyl)amino)azetidine-1-carboxylate (370 mg, 7.70% yield) as a yellow oil. MS: m/z=289.3 (M+1, ESI+).

### Step 2:

To a solution of tert-butyl 3-(bis(3-hydroxypropyl)amino)azetidine-1-carboxylate (370 mg, 1.28 mmol) in DCM (8 mL) was added TFA (1.46 g, 12.83 mmol), the reaction mixture was stirred at 25° C. for 16 h. The reaction mixture was evaporated under reduced pressure to afford 3,3′-(azetidin-3-ylazanediyl) bis(propan-1-ol); 2,2,2-trifluoroacetate salt (170 mg, crude) as a colorless oil. MS: m/z=189.3 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (550 mg, 1.34 mmol) and 3,3′-(azetidin-3-ylazanediyl)bis(propan-1-ol); 2,2,2-trifluoroacetate salt (252 mg, 1.34 mmol) in MeCN (20 mL) was added K₂CO₃ (1.85 g, 13.39 mmol), the reaction mixture was stirred at 70° C. for 2 h. Filtered and evaporated under reduced pressure, the residue was purified by prep-HPLC to afford compound 30, 5-(5-((3-(bis(3-hydroxypropyl)amino)azetidin-1-yl)sulfonyl)-2-ethoxyphenyl)-1-methyl-3-propyl-1,6-dihydro-7H-pyrazolo[4,3-d]pyrimidin-7-one (400 mg, 53.11% yield) as a white solid. ¹H NMR (400 MHz, DMSO-d₆) δ 12.21 (bs, 1H), 7.93-7.90 (m, 2H), 7.41 (d, 1H), 4.36 (bs, 2H), 4.26-4.21 (m, 2H), 4.17 (s, 3H), 3.80-3.76 (m, 2H), 3.53-3.41 (m, 3H), 3.29-3.26 (m, 4H), 2.78 (t, 2H), 2.25-2.21 (m, 4H), 1.77-1.71 (m, 2H), 1.37-1.34 (m, 7H), 0.94 (t, 3H); MS: m/z=563.2 (M+1, ESI+); HRMS: 563.2649.

### Synthesis of Compound 31

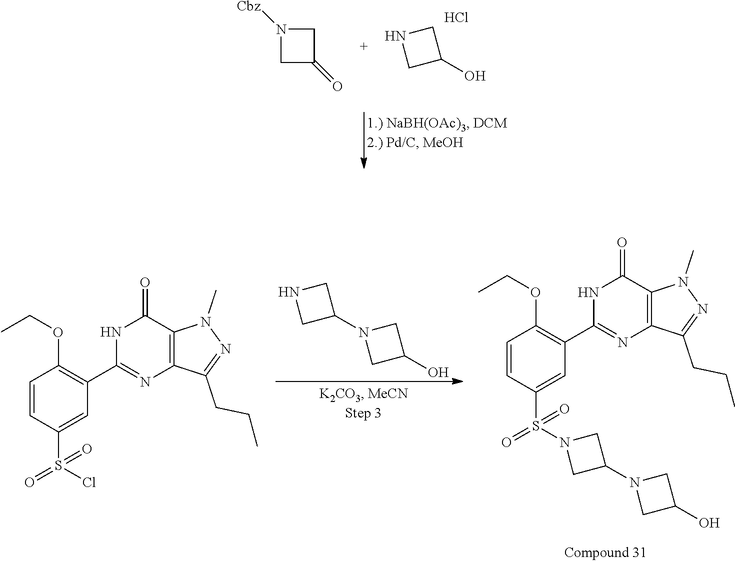 [SMILES: O=C1CN(C(=O)OCc2ccccc2)C1 ; OC1CNC1 ; Cl ; CCCc1nn(C)c2c(=O)[nH]c(-c3cc(S(=O)(=O)Cl)ccc3OCC)nc12 ; OC1CN(C2CNC2)C1 ; CCCc1nn(C)c2c(=O)[nH]c(-c3cc(S(=O)(=O)N4CC(N5CC(O)C5)C4)ccc3OCC)nc12]

### Step 1:

To a solution of benzyl 3-oxoazetidine-1-carboxylate (2.5 g, 12.18 mmol) and azetidin-3-ol hydrochloride (1.20 g, 10.96 mmol) in DCM (10 mL) was added NaBH(OAc)₃ (3.87 g, 18.27 mmol), the reaction mixture was stirred at 25° C. for 24 h. The reaction mixture was poured into water (100 mL) and extracted with DCM (20 mL×3). The combined organic layers were dried over Na₂SO₄ and concentrated under reduced pressure, the residue was purified by column chromatography to afford benzyl 3-hydroxy-[1,3′-biazetidine]-1′-carboxylate (1.8 g, 56.33% yield) as a yellow oil. MS: m/z=263.2 (M+1, ESI+). ¹H NMR (400 MHz, CDCl₃) δ 7.38-7.30 (m, 5H), 5.30 (s, 1H), 5.09 (s, 2H), 4.53-4.47 (m, 1H), 4.11-4.04 (m, 2H), 3.92-3.86 (m, 2H), 3.79-3.59 (m, 5H).

### Step 2:

To a solution of benzyl 3-hydroxy-[1,3′-biazetidine]-1′-carboxylate (1.8 g, 6.86 mmol) in MeOH (30 mL) was added Pd/C (500 mg) and stirred at 25° C. for 16 h under H₂. Filtered and concentrated to afford [1,3′-biazetidin]-3-ol (700 mg, 79.61% yield) as a yellow oil. MS: m/z=129.3 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (770 mg, 1.87 mmol) and [1,3′-biazetidin]-3-ol (300 mg, 2.34 mmol) in MeCN (15 mL) was added K₂CO₃ (970 mg, 7.02 mmol), the reaction mixture was stirred at 80° C. for 2 h. Filtered and evaporated under reduced pressure, the residue was purified by prep-HPLC to afford compound 31, 5-(2-ethoxy-5-((3-hydroxy-[1,3′-biazetidin]-1′-yl)sulfonyl)phenyl)-1-methyl-3-propyl-1,6-dihydro-7H-pyrazolo [4,3-d]pyrimidin-7-one (480 mg, 40.80% yield) as a white solid. ¹H NMR (400 MHz, DMSO-d₆) δ 12.25 (bs, 1H), 7.92-7.88 (m, 2H), 7.40 (d, 1H), 5.32 (bs, 1H), 4.25-4.20 (m, 2H), 4.17 (s, 3H), 4.10-4.07 (m, 1H), 3.71 (t, 2H), 3.49-3.46 (m, 2H), 3.31-3.25 (m, 3H), 2.77 (t, 2H), 2.63-2.61 (m, 2H), 1.77-1.72 (m, 2H), 1.35 (t, 3H), 0.94 (t, 3H); MS: m/z=503.2 (M+1, ESI+); HRMS: 503.2073.

### Synthesis of Compound 32

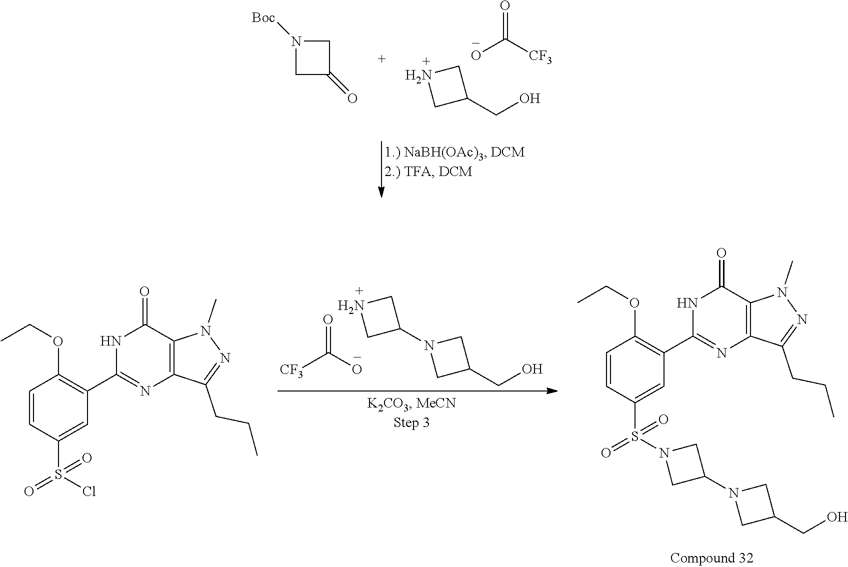

### Step 1:

To a solution of tert-butyl 3-oxoazetidine-1-carboxylate (1.5 g, 8.76 mmol) and azetidin-3-ylmethanol; 2,2,2-trifluoroacetate salt (332 mg, 3.81 mmol) in DCM (30 mL) was added NaBH(OAc)₃ (1.86 g, 8.76 mmol), the reaction mixture was stirred at 25° C. for 24 h. The reaction mixture was poured into water (100 mL) and extracted with DCM (20 mL×3). The combined organic layers were dried over Na₂SO₄ and concentrated under reduced pressure, the residue was purified by column chromatography to afford tert-butyl 3-(hydroxymethyl)-[1,3′-biazetidine]-1′-carboxylate (1.1 g, 51.81% yield) as a yellow oil. MS: m/z=243.3 (M+1, ESI+). ¹H NMR (400 MHz, Methanol-d₄) δ 4.16-4.04 (m, 3H), 3.96-3.86 (m, 2H), 3.85-3.78 (m, 2H), 3.74-3.62 (m, 4H), 2.96-2.78 (m, 1H), 1.43 (s, 9H).

### Step 2:

To a solution of tert-butyl 3-(hydroxymethyl)-[1,3′-biazetidine]-1′-carboxylate (1.1 g, 4.54 mmol) in DCM (20 mL) was added TFA (2.59 g, 22.70 mmol), the reaction mixture was stirred at 25° C. for 16 h. The resulting solution was evaporated to afford [1,3′-biazetidin]-3-ylmethanol; 2,2,2-trifluoroacetate salt (950 mg, 82% yield) as a yellow oil. MS: m/z=143.3 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (500 mg, 1.22 mmol) and [1,3′-biazetidin]-3-ylmethanol; 2,2,2-trifluoroacetate salt (373 mg, 1.46 mmol) in MeCN (10 mL) was added K₂CO₃ (505 mg, 3.65 mmol), the reaction mixture was stirred at 80° C. for 2 h. Filtered and evaporated under reduced pressure, the residue was purified by prep-HPLC to afford compound 32, 5-(2-ethoxy-5-((3-(hydroxymethyl)-[1,3′-biazetidin]-1′-yl)sulfonyl) phenyl)-1-methyl-3-propyl-1,6-dihydro-7H-pyrazolo[4,3-d]pyrimidin-7-one (300 mg, 47.72% yield) as a white solid. ¹H NMR (400 MHz, DMSO-d₆) δ 12.26 (bs, 1H), 7.94-7.90 (m, 2H), 7.41 (d, 1H), 4.58 (bs, 1H), 4.24 (q, 2H), 4.17 (s, 3H), 3.70 (t, 2H), 3.51-3.47 (m, 2H), 3.79-3.75 (m, 2H), 3.28-3.25 (m, 1H), 3.00 (t, 2H), 2.78 (t, 2H), 2.67 (t, 2H), 2.35-2.32 (m, 1H), 1.77-1.72 (m, 2H), 1.35 (t, 3H), 0.94 (t, 3H); MS: m/z=517.4 (M+1, ESI+); HRMS: 517.2230.

### Synthesis of Compound 33

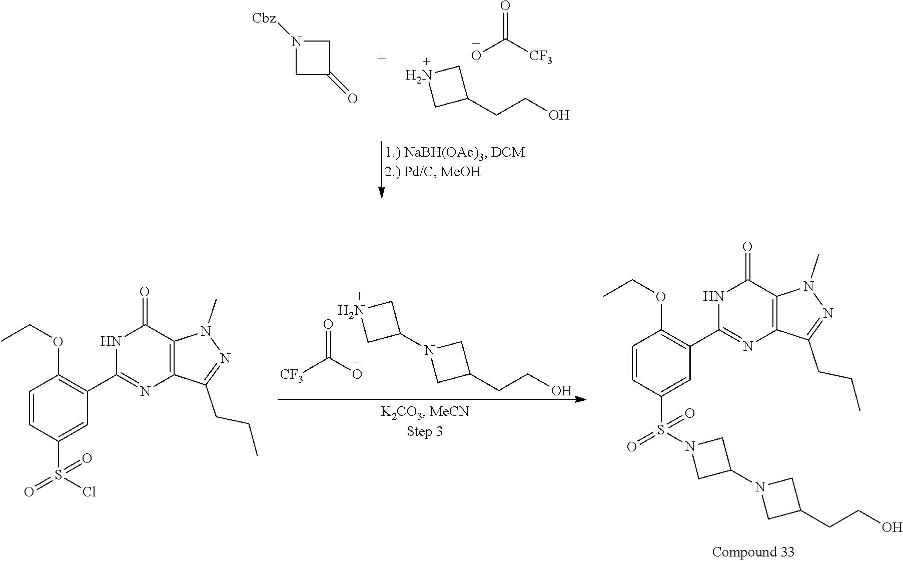

### Step 1:

To a solution of benzyl 3-oxoazetidine-1-carboxylate (1.5 g, 7.31 mmol) and 2-(azetidin-3-yl)ethan-1-ol; 2,2,2-trifluoroacetate salt (1.25 g, 5.85 mmol) in DCM (30 mL) was added NaBH(OAc)₃ (1.86 g, 8.77 mmol), the reaction mixture was stirred at 25° C. for 24 h. The reaction mixture was poured into water (100 mL) and extracted with DCM (20 mL×3). The combined organic layers were dried over Na₂SO₄ and concentrated under reduced pressure, the residue was purified by column chromatography to afford benzyl 3-(2-hydroxyethyl)-[1,3′-biazetidine]-1′-carboxylate (1.0 g, 47.12% yield) as a yellow oil. MS: m/z=291.3 (M+1, ESI+). ¹H NMR (400 MHz, CDCl₃) δ 7.38-7.31 (m, 5H), 5.09 (s, 2H), 4.10-4.05 (m, 2H), 3.92-3.89 (m, 2H), 3.74-3.58 (m, 7H), 2.84-2.75 (m, 1H), 1.85-1.80 (m, 2H).

### Step 2:

To a solution of benzyl 3-(2-hydroxyethyl)-[1,3′-biazetidine]-1′-carboxylate (1.0 g, 3.44 mmol) in MeOH (20 mL) was added Pd/C (300 mg) and stirred at 25° C. for 16 h under H₂. Filtered and concentrated to afford 2-([1,3′-biazetidin]-3-yl)ethan-1-ol (450 mg, 83.64% yield) as a yellow oil. MS: m/z=157.2 (M+1, ESI+).

### Step 3:

To a solution of 4-ethoxy-3-(1-methyl-7-oxo-3-propyl-6,7-dihydro-1H-pyrazolo[4,3-d]pyrimidin-5-yl) benzenesulfonyl chloride (630 mg, 1.54 mmol) and 2-([1,3′-biazetidin]-3-yl)ethan-1-ol (300 mg, 1.92 mmol) in MeCN (15 mL) was added K₂CO₃ (796 mg, 5.76 mmol), the reaction mixture was stirred at 80° C. for 2 h. Filtered and evaporated under reduced pressure, the residue was purified by prep-HPLC to afford compound 33, 5-(2-ethoxy-5-((3-(2-hydroxyethyl)-[1,3′-biazetidin]-1′-yl)sulfonyl)phenyl)-1-methyl-3-propyl-1,6-dihydro-7H-pyrazolo[4,3-d]pyrimidin-7-one (410 mg, 40.24% yield) as a white solid. ¹H NMR (400 MHz, DMSO-d₆) δ 12.28 (bs, 1H), 7.95-7.89 (m, 2H), 7.41 (d, 1H), 4.39 (bs, 1H), 4.24 (q, 2H), 4.17 (s, 3H), 3.70 (t, 2H), 3.47-3.44 (m, 2H), 3.28-3.24 (m, 3H), 3.06 (t, 2H), 2.78 (t, 2H), 2.49-2.46 (m, 2H), 2.32-2.27 (m, 1H), 1.77-1.70 (m, 2H), 1.52-1.47 (m, 2H), 1.35 (t, 3H), 0.94 (t, 3H); MS: m/z=531.2 (M+1, ESI+); HRMS: 531.2387.

### Synthesis of Compound 34

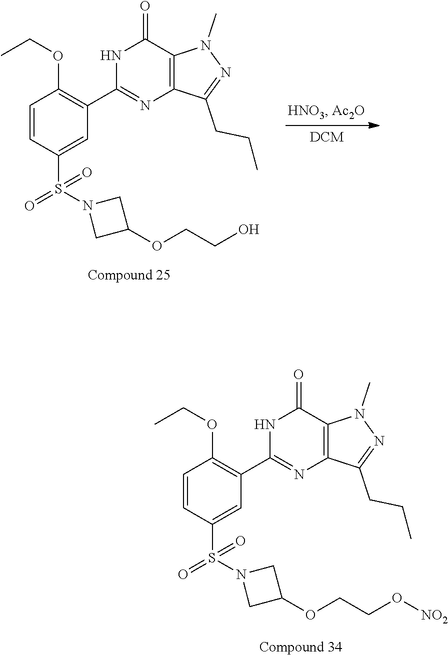

To a solution of compound 25, 5-(2-ethoxy-5-((3-(2-hydroxyethoxy)azetidin-1-yl)sulfonyl)phenyl)-1-methyl-3-propyl-1,6-dihydro-7H-pyrazolo[4,3-d]pyrimidin-7-one (250 mg, 508 umol) in DCM (10 mL) was added HNO₃ (141 mg, 1.52 mmol) and Ac₂O (161 mg, 1.52 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 34, 2-((1-((4-ethoxy-3-(l-methyl-7-oxo-3-propyl-6,7-dihydro-1H-pyrazolo[4,3-d]pyrimidin-5-yl)phenyl) sulfonyl)azetidin-3-yl)oxy) ethyl nitrate (120 mg, 43.98% yield) as a white solid. ¹H NMR (400 MHz, DMSO-d₆) δ 12.24 (bs, 1H), 7.92-7.87 (m, 2H), 7.40 (d, 1H), 4.59-4.56 (m, 2H), 4.24-4.19 (m, 3H), 4.17 (s, 3H), 3.95-3.92 (m, 2H), 3.62-3.60 (m, 2H), 3.54-3.50 (m, 2H), 2.78 (t, 2H), 1.79-1.70 (m, 2H), 1.35 (t, 3H), 0.94 (t, 3H); MS: m/z=537.1 (M+1, ESI+); HRMS: 537.1766.

### Synthesis of Compound 35

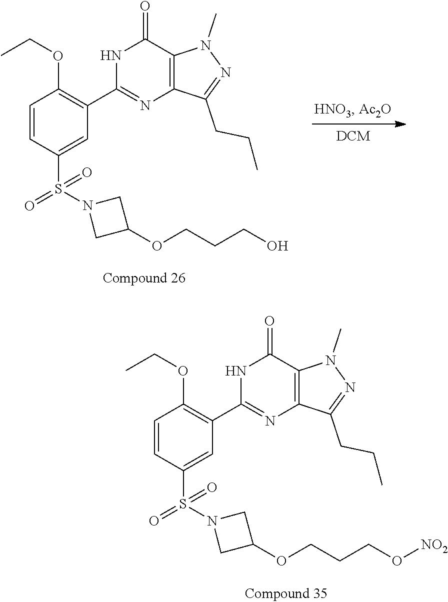 [SMILES: CCCc1nn(C)c2c(=O)[nH]c(-c3cc(S(=O)(=O)N4CC(OCCCO)C4)ccc3OCC)nc12 ; CCCc1nn(C)c2c(=O)[nH]c(-c3cc(S(=O)(=O)N4CC(OCCCO[N+](=O)[O-])C4)ccc3OCC)nc12]

To a solution of compound 26, 5-(2-ethoxy-5-((3-(3-hydroxypropoxy)azetidin-1-yl)sulfonyl)phenyl)-1-methyl-3-propyl-1,6-dihydro-7H-pyrazolo[4,3-d]pyrimidin-7-one (200 mg, 407 umol) in DCM (20 mL) was added HNO₃ (77 mg, 1.22 mmol) and Ac₂O (125 mg, 1.22 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 35, 3-((1-((4-ethoxy-3-(1-methyl-7-oxo-3-propyl-6,7-dihydro-1H-pyrazolo[4,3-d]pyrimidin-5-yl)phenyl) sulfonyl)azetidin-3-yl)oxy)propyl nitrate (140 mg, 62.5% yield) as a white solid. ¹H NMR (400 MHz, DMSO-d₆) δ 12.02 (bs, 1H), 7.94-7.90 (m, 2H), 7.40 (d, 1H), 4.45 (t, 2H), 4.24 (q, 2H), 4.16 (s, 3H), 4.14-4.12 (m, 1H), 3.96-3.93 (m, 2H), 3.51-3.47 (m, 2H), 3.34-3.32 (m, 2H), 2.78 (t, 2H), 1.84-1.70 (m, 4H), 1.35 (t, 3H), 0.93 (t, 3H); MS: m/z=551.3 (M+1, ESI+); HRMS: 551.1920.

### Synthesis of Compound 36

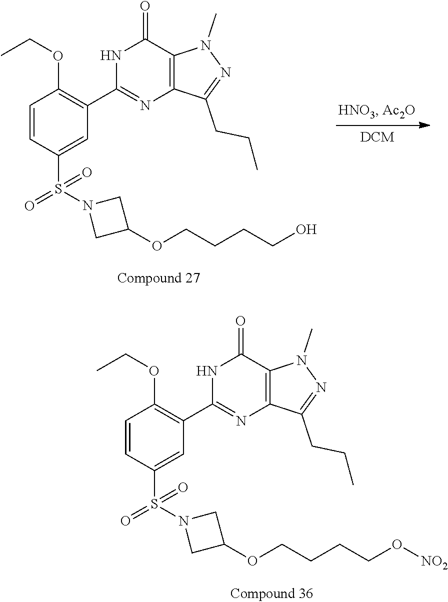 [SMILES: CCCc1nn(C)c2c(=O)[nH]c(-c3cc(S(=O)(=O)N4CC(OCCCCO)C4)ccc3OCC)nc12 ; CCCc1nn(C)c2c(=O)[nH]c(-c3cc(S(=O)(=O)N4CC(OCCCCO[N+](=O)[O-])C4)ccc3OCC)nc12]

To a solution of compound 27, 5-(2-ethoxy-5-((3-(4-hydroxybutoxy)azetidin-1-yl)sulfonyl)phenyl)-1-methyl-3-propyl-1,6-dihydro-7H-pyrazolo[4,3-d]pyrimidin-7-one (400 mg, 791 umol) in DCM (20 mL) was added HNO₃ (150 mg, 2.37 mmol) and Ac₂O (242 mg, 2.37 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 36, 4-((1-((4-ethoxy-3-(1-methyl-7-oxo-3-propyl-6,7-dihydro-1H-pyrazolo[4,3-d]pyrimidin-5-yl)phenyl) sulfonyl)azetidin-3-yl)oxy)butyl nitrate (220 mg, 39.4% yield) as a white solid. ¹H NMR (400 MHz, DMSO-d₆) δ 11.95 (bs, 1H), 7.94-7.89 (m, 2H), 7.40 (d, 1H), 4.44 (t, 2H), 4.23 (q, 2H), 4.16 (s, 3H), 4.14-4.11 (m, 1H), 3.96-3.93 (m, 2H), 3.49-3.46 (m, 2H), 3.26 (t, 2H), 2.77 (t, 2H), 1.77-1.71 (m, 2H), 1.59-1.54 (m, 2H), 1.49-1.44 (m, 2H), 1.34 (t, 3H), 0.93 (t, 3H); MS: m/z=565.1 (M+1, ESI+); HRMS: 565.2078.

### Synthesis of Compound 37

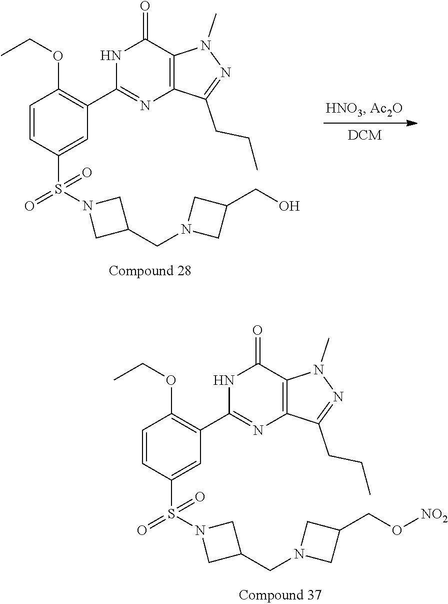 [SMILES: CCCc1nn(C)c2c(=O)[nH]c(-c3cc(S(=O)(=O)N4CC(CN5CC(CO)C5)C4)ccc3OCC)nc12 ; CCCc1nn(C)c2c(=O)[nH]c(-c3cc(S(=O)(=O)N4CC(CN5CC(CO[N+](=O)[O-])C5)C4)ccc3OCC)nc12]

To a solution of compound 28, 5-(2-ethoxy-5-((3-((3-(hydroxymethyl)azetidin-1-yl)methyl)azetidin-1-yl)sulfonyl) phenyl)-1-methyl-3-propyl-1,6-dihydro-7H-pyrazolo[4,3-d]pyrimidin-7-one (300 mg, 565 umol) in DCM (10 mL) was added HNO₃ (164 mg, 1.70 mmol) and Ac₂O (173 mg, 1.70 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 37, (1-((1-((4-ethoxy-3-(1-methyl-7-oxo-3-propyl-6,7-dihydro-1H-pyrazolo[4,3-d]pyrimidin-5-yl)phenyl) sulfonyl)azetidin-3-yl)methyl)azetidin-3-yl)methyl nitrate (80 mg, 28% yield) as a white solid. ¹H NMR (400 MHz, DMSO-d₆) δ 12.24 (bs, 1H), 7.92-7.87 (m, 2H), 7.40 (d, 1H), 4.56 (d, 2H), 4.24 (q, 2H), 4.16 (s, 3H), 3.74 (t, 2H), 3.36-3.33 (m, 2H), 3.15 (t, 2H), 2.83-2.75 (m, 4H), 2.65-2.59 (m, 1H), 2.40-2.32 (m, 1H), 2.29-2.27 (m, 2H), 1.79-1.69 (m, 2H), 1.35 (t, 3H), 0.93 (t, 3H); MS: m/z=576.5 (M+1, ESI+); HRMS: 576.2238.

### Synthesis of Compound 38

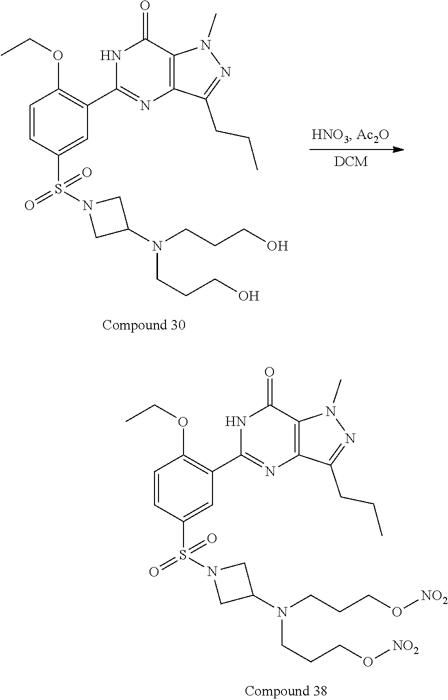 [SMILES: CCCc1nn(C)c2c(=O)[nH]c(-c3cc(S(=O)(=O)N4CC(N(CCCO)CCCO)C4)ccc3OCC)nc12 ; CCCc1nn(C)c2c(=O)[nH]c(-c3cc(S(=O)(=O)N4CC(N(CCCO[N+](=O)[O-])CCCO[N+](=O)[O-])C4)ccc3OCC)nc12]

To a solution of compound 30, 5-(5-((3-(bis(3-hydroxypropyl)amino)azetidin-1-yl)sulfonyl)-2-ethoxy phenyl)-1-methyl-3-propyl-1,6-dihydro-7H-pyrazolo[4,3-d]pyrimidin-7-one (200 mg, 355 umol) in DCM (10 mL) was added HNO₃ (224 mg, 3.55 mmol) and Ac₂O (363 mg, 3.55 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 38, ((1-((4-ethoxy-3-(1-methyl-7-oxo-3-propyl-6,7-dihydro-1H-pyrazolo[4,3-d]pyrimidin-5-yl)phenyl)sulfonyl)azetidin-3-yl)azanediyl)bis(propane-3,1-diyl)dinitrate (150 mg, 64.66% yield) as a white solid. ¹H NMR (400 MHz, DMSO-d₆) δ 12.23 (bs, 1H), 7.97 (d, 1H), 7.92 (dd, 2H), 7.40 (d, 1H), 4.40-4.37 (m, 4H), 4.25-4.20 (m, 2H), 4.16 (s, 3H), 3.82-3.80 (m, 2H), 3.49-3.46 (m, 3H), 2.77 (t, 2H), 2.30-2.26 (m, 4H), 1.77-1.62 (m, 6H), 1.36 (t, 3H), 0.93 (t, 3H); MS: m/z=653.3 (M+1, ESI+); HRMS: 653.2347.

### Synthesis of Compound 39

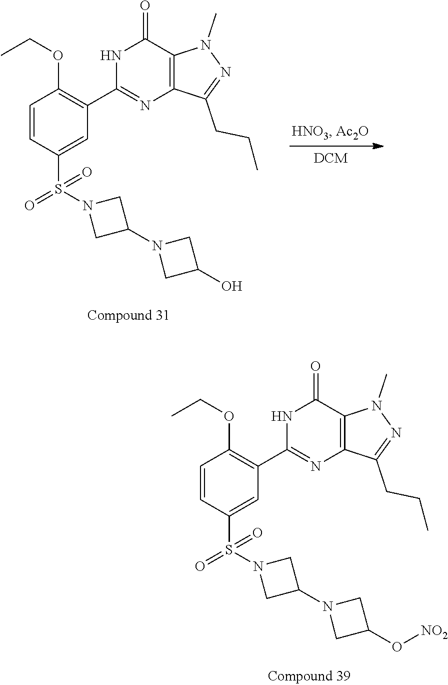

To a solution of compound 31, 5-(2-ethoxy-5-((3-hydroxy-[1,3′-biazetidin]-1′-yl)sulfonyl)phenyl)-1-methyl-3-propyl-1,6-dihydro-7H-pyrazolo[4,3-d]pyrimidin-7-one (310 mg, 617 umol) in DCM (10 mL) was added HNO₃ (117 mg, 1.85 mmol) and Ac₂O (196 mg, 1.85 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 39, 1′-((4-ethoxy-3-(1-methyl-7-oxo-3-propyl-6,7-dihydro-1H-pyrazolo[4,3-d]pyrimidin-5-yl)phenyl)sulfonyl)-[1,3′-biazetidin]-3-yl nitrate (76 mg, 22.50% yield) as a white solid. ¹H NMR (400 MHz, DMSO-d₆) δ 12.25 (bs, 1H), 7.94-7.90 (m, 2H), 7.41 (d, 1H), 5.29-5.27 (m, 1H), 4.25-4.20 (m, 2H), 4.17 (s, 3H), 3.75-3.72 (m, 2H), 3.51-3.36 (m, 5H), 3.07-3.04 (m, 2H), 2.78 (t, 2H), 1.77-1.72 (m, 2H), 1.35 (t, 3H), 0.93 (t, 3H); MS: m/z=548.2 (M+1, ESI+); HRMS: 548.1923.

### Synthesis of Compound 40

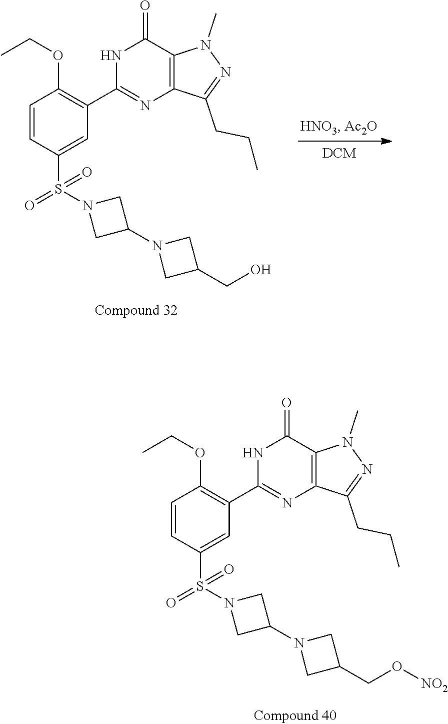

To a solution of compound 32, 5-(2-ethoxy-5-((3-(hydroxymethyl)-[1,3′-biazetidin]-1′-yl)sulfonyl)phenyl)-1-methyl-3-propyl-1,6-dihydro-7H-pyrazolo[4,3-d]pyrimidin-7-one (200 mg, 387 umol) in DCM (8 mL) was added HNO₃ (73 mg, 1.16 mmol) and Ac₂O (123 mg, 1.16 mmol), the reaction mixture was stirred at 25° C. for 16 h. The resulting solution was poured into water (50 mL) and extracted with DCM (20 mL×3). The combined organic lay ers were dried over Na₂SO₄ and concentrated under reduced pressure, the residue was purified by Prep-HPLC to afford compound 40, (1′-((4-ethoxy-3-(1-methyl-7-oxo-3-propyl-6,7-dihydro-1H-pyrazolo[4,3-d]pyrimidin-5-yl)phenyl)sulfonyl)-[1,3′-biazetidin]-3-yl)methyl nitrate (55 mg, 25.30% yield) as a white solid. ¹H NMR (400 MHz, DMSO-d₆) δ 12.28 (bs, 1H), 7.93-7.90 (m, 2H), 7.41 (d, 1H), 4.55 (d, 2H), 4.25-4.17 (m, 5H), 3.71 (t, 2H), 3.51-3.48 (m, 2H), 3.31-3.28 (m, 1H), 3.08 (t, 2H), 2.79-2.73 (m, 4H), 2.65-2.60 (m, 1H), 1.77-1.69 (m, 2H), 1.35 (t, 3H), 0.95-0.91 (m, 3H); MS: m/z=562.2 (M+1, ESI+); HRMS: 562.2081.

### Synthesis of Compound 65

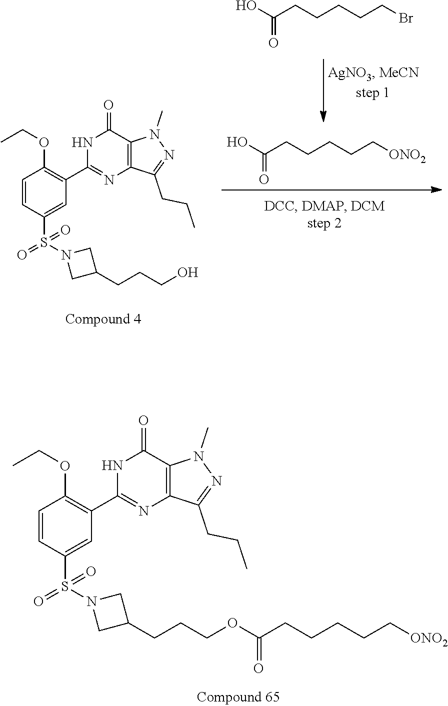

### Step 1:

To a solution of 6-bromohexanoic acid (4 g, 20.51 mmol) in MeCN (150 mL) was added AgNO₃ (13.93 g, 82.03 mmol), the reaction mixture was stirred at 90° C. for 16 h. Cooled to room temperature and filtered, the filtrate was evaporated under reduce pressure and the residue was purified by column chromatography to afford 6-(nitrooxy)hexanoic acid (2.8 g, 77.07% yield) as a light yellow oil. MS: m/z=178.1 (M+1, ESI+)

### Step 2:

To a solution of compound 4, 5-(2-ethoxy-5-((3-(3-hydroxypropyl)azetidin-1-yl)sulfonyl)phenyl)-1-methyl-3-propyl-1,6-dihydro-7H-pyrazolo[4,3-d]pyrimidin-7-one (200 mg, 408.51 umol) and 6-(nitrooxy) hexanoic acid (109 mg, 613 umol) in DCM (15 mL) was added DCC (101 mg, 490 umol) and DMAP (50 mg, 409 umol), the reaction mixture was stirred at 25° C. for 16 h. The reaction mixture was poured into water (100 mL) and extracted with DCM (20 mL×3). The organic layer was washed with brine (100 mL×2), dried over Na₂SO₄ and concentrated. The residue was purified by prep-HPLC to afford compound 65, 3-(1-((4-ethoxy-3-(1-methyl-7-oxo-3-propyl-6,7-dihydro-1H-pyrazolo[4,3-d] pyrimidin-5-yl)phenyl)sulfonyl)azetidin-3-yl)propyl 6-(nitrooxy)hexanoate (120 mg, 45.28% yield) as a white solid. MS: m/z=649.3 (M+1, ESI+). ¹H NMR (400 MHz, DMSO-d₆) δ 12.24 (bs, 1H), 7.93-7.89 (m, 2H), 7.41 (d, 1H), 4.47 (t, 2H), 4.24 (q, 2H), 4.16 (s, 3H), 3.90 (t, 2H), 3.80 (t, 2H), 3.34-3.31 (m, 1H), 2.77 (t, 2H), 2.42-2.35 (m, 1H), 2.23 (t, 2H), 1.79-1.69 (m, 2H), 1.65-1.57 (m, 2H), 1.52-1.47 (m, 2H), 1.45-1.23 (m, 10H), 0.93 (t, 3H); MS: m/z=649.3 (M+1, ESI+); HRMS: 649.2654.

### Synthesis of Compound 66

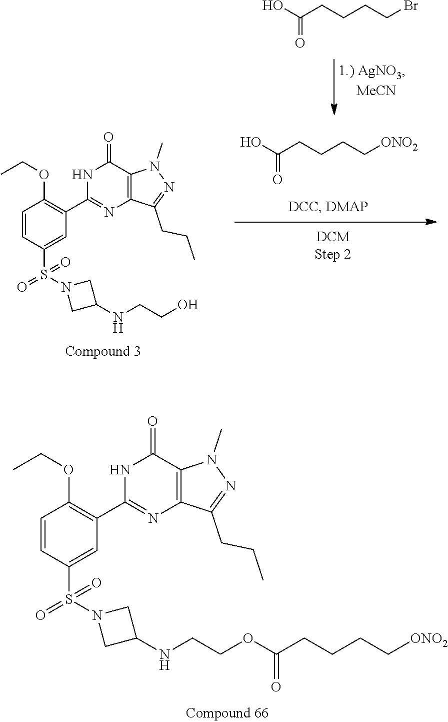

### Step 1:

To a solution of 5-bromopentanoic acid (3 g, 16.57 mmol) in MeCN (30 mL) was added AgNO₃ (4.22 g, 24.86 mmol), the reaction mixture was stirred at 70° C. for 16 h. Cooled to room temperature and filtered, the filtrate was evaporated under reduce pressure and the residue was purified by column chromatography to afford 5-(nitrooxy)pentanoic acid (2.6 g, 96.18% yield) as a light-yellow oil. MS: m/z=164.1 (M+1, ESI+).

### Step 2:

To a solution of compound 3, 5-(2-ethoxy-5-((3-((2-hydroxyethyl)amino)azetidin-1-yl)sulfonyl)phenyl)-1-methyl-3-propyl-1,6-dihydro-7H-pyrazolo[4,3-d]pyrimidin-7-one (1.1 g, 2.24 mmol) and 5-(nitrooxy) pentanoic acid (549 mg, 3.36 mmol) in DCM (20 mL) was added DCC (555 mg, 2.69 mmol) and DMAP (274 mg, 2.24 mmol), the reaction mixture was stirred at 25° C. for 16 h. The reaction mixture was poured into water (100 mL) and extracted with DCM (20 mL×3). The organic layer was washed with brine (100 mL×2), dried over Na₂SO₄ and concentrated. The residue was purified by prep-HPLC to afford compound 66, 2-((1-((4-ethoxy-3-(1-methyl-7-oxo-3-propyl-6,7-dihydro-1H-pyrazolo[4,3-d]pyrimidin-5-yl)phenyl)sulfonyl)azetidin-3-yl)amino)ethyl 5-(nitrooxy)pentanoate (187 mg, 13.07% yield) as a white solid. ¹H NMR (400 MHz, DMSO-d₆) δ 12.21 (bs, 1H), 7.94-7.89 (m, 2H), 7.40 (d, 1H), 4.48 (t, 2H), 4.24 (q, 2H), 4.16 (s, 3H), 3.92 (t, 2H), 3.84-3.82 (m, 2H), 3.41-3.38 (m, 3H), 2.78 (t, 2H), 2.57 (t, 2H), 2.31 (t, 2H), 1.77-1.71 (m, 2H), 1.66-1.53 (m, 4H), 1.35 (t, 3H), 0.93 (t, 3H); MS: m/z=636.3 (M+1, ESI+); HRMS: 636.2451.

### Synthesis of Compound 67

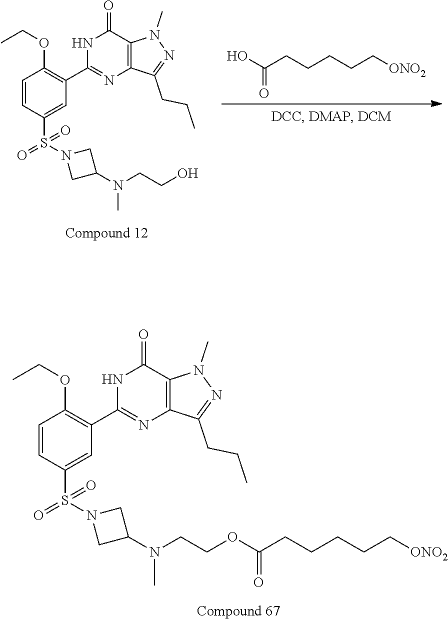

To a solution of compound 12, 5-(2-ethoxy-5-((3-((2-hydroxyethyl)(methyl)amino)azetidin-1-yl)sulfonyl)phenyl)-1-methyl-3-propyl-1,6-dihydro-7H-pyrazolo[4,3-d]pyrimidin-7-one (250 mg, 495.44 umol) and 6-(nitrooxy)hexanoic acid (132 mg, 743 umol) in DCM (20 mL) was added DCC (123 mg, 595 umol) and DMAP (61 mg, 495 umol), the reaction mixture was stirred at 25° C. for 16 h. The reaction mixture was poured into water (100 mL) and extracted with DCM (20 mL×3). The organic layer was washed with brine (100 mL×2), dried over Na₂SO₄ and concentrated. The residue was purified by prep-HPLC to afford compound 67, 2-((1-((4-ethoxy-3-(1-methyl-7-oxo-3-propyl-6,7-dihydro-1H-pyrazolo[4,3-d]pyrimidin-5-yl)phenyl)sulfonyl)azetidin-3-yl)(methyl)amino)ethyl 6-(nitrooxy) hexanoate (120 mg, 36.49% yield) as a white solid. ¹H NMR (400 MHz, DMSO-d₆) δ 12.25 (bs, 1H), 7.96-7.91 (m, 2H), 7.41 (d, 1H), 4.46 (t, 2H), 4.23 (q, 2H), 4.17 (s, 3H), 3.96 (t, 2H), 3.78 (t, 2H), 3.49 (t, 2H), 3.28-3.24 (m, 1H), 2.78 (t, 2H), 2.40-2.37 (m, 2H), 2.22 (t, 2H), 1.98 (s, 3H), 1.78-1.72 (m, 2H), 1.64-1.57 (m, 2H), 1.52-1.44 (m, 2H), 1.36 (t, 3H), 1.32-1.23 (m, 2H), 0.94 (t, 3H); MS: m/z=664.3 (M+1, ESI+); HRMS: 664.2762.

### Synthesis of Compound 68

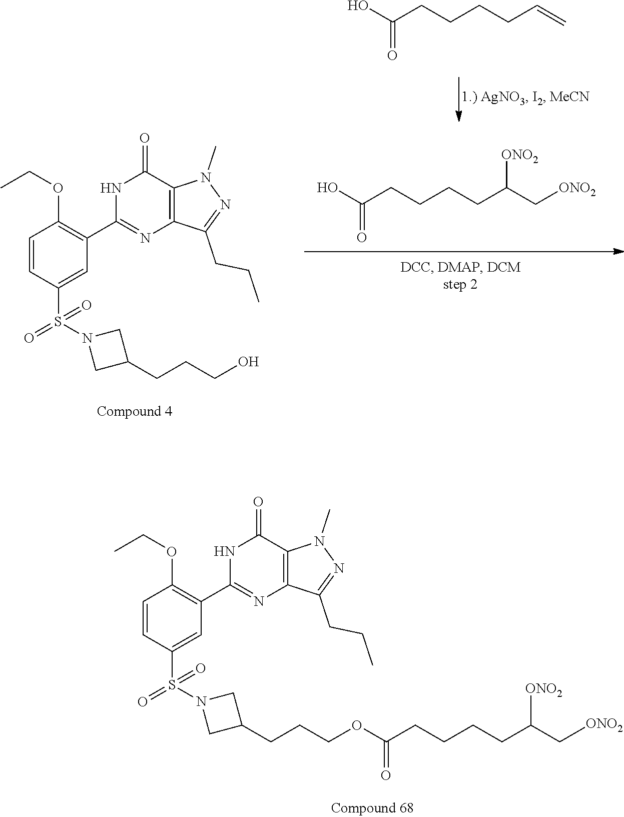

### Step 1:

To a solution of hept-6-enoic acid (1 g, 7.80 mmol) and AgNO₃ (3.98 g, 23.41 mmol) in MeCN (70 mL) was added I₂ (1.98 g, 7.80 mmol), the reaction mixture was stirred at 80° C. for 16 h. The resulting mixture was filtered and the filtrate was concentrated under reduced pressure, the residue was dissolved in EA (30 mL), washed with brine (20 mL×3), dried over Na₂SO₄ and concentrated under reduced pressure to afford 6,7-bis(nitrooxy)heptanoic acid (1.75 g, 88.94% yield) as a light yellow oil. MS: m/z=253.1 (M+1, ESI+)

### Step 2:

To a solution of compound 4, 5-(2-ethoxy-5-((3-(3-hydroxypropyl)azetidin-1-yl)sulfonyl)phenyl)-1-methyl-3-propyl-1,6-dihydro-7H-pyrazolo[4,3-d]pyrimidin-7-one (400 mg, 817.02 umol) and 6,7-bis(nitrooxy)heptanoic acid (309 mg, 1.23 mmol) in DCM (20 mL) was added DCC (202 mg, 980 umol) and DMAP (100 mg, 817 umol), the reaction mixture was stirred at 25° C. for 16 h. The reaction mixture was poured into water (100 mL) and extracted with DCM (20 mL×3). The organic layer was washed with brine (100 mL×2), dried over Na₂SO₄ and concentrated. The residue was purified by prep-HPLC to afford compound 68, 3-(1-((4-ethoxy-3-(1-methyl-7-oxo-3-propyl-6,7-dihydro-1H-pyrazolo[4,3-d]pyrimidin-5-yl)phenyl)sulfonyl)azetidin-3-yl)propyl 6,7-bis(nitrooxy)heptanoate (170 mg, 28.75% yield) as a white solid. ¹H NMR (400 MHz, DMSO-d₆) δ 12.24 (bs, 1H), 7.94-7.89 (m, 2H), 7.41 (d, 1H), 5.39-5.37 (m, 1H), 4.90 (dd, 1H), 4.68 (dd, 1H), 4.23 (q, 2H), 4.17 (s, 3H), 3.90 (t, 2H), 3.80 (t, 2H), 3.35-3.32 (m, 2H), 2.78 (t, 2H), 2.41-2.37 (m, 1H), 2.25 (t, 2H), 1.77-1.65 (m, 4H), 1.53-1.49 (m, 2H), 1.46-1.30 (m, 9H), 0.93 (t, 3H); MS: m/z=724.2 (M+1, ESI+); HRMS: 724.2604.

### Synthesis of Compound 69

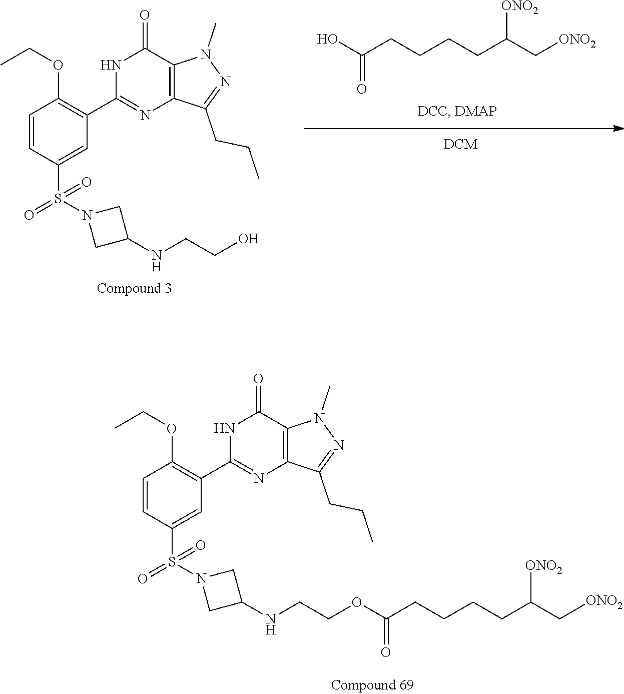

To a solution of compound 3, 5-(2-ethoxy-5-((3-((2-hydroxyethyl)amino)azetidin-1-yl)sulfonyl)phenyl)-1-methyl-3-propyl-1,6-dihydro-7H-pyrazolo[4,3-d]pyrimidin-7-one (1 g, 2.04 mmol) and 6,7-bis(nitrooxy) heptanoic acid (515 mg, 2.04 mmol) in DCM (20 mL) was added DCC (421 mg, 2.04 mmol) and DMAP (249 mg, 2.04 mmol), the reaction mixture was stirred at 25° C. for 16 h. The reaction mixture was poured into water (100 mL) and extracted with DCM (20 mL×3). The organic layer was washed with brine (100 mL×2), dried over Na₂SO₄ and concentrated. The residue was purified by prep-HPLC to afford compound 69, 2-((1-((4-ethoxy-3-(1-methyl-7-oxo-3-propyl-6,7-dihydro-1H-pyrazolo[4,3-d]pyrimidin-5-yl)phenyl)sulfonyl)azetidin-3-yl)amino)ethyl 6,7-bis(nitrooxy) heptanoate (580 mg, 39.19% yield) as a white solid. ¹H NMR (400 MHz, DMSO-d₆) δ 12.21 (bs, 1H), 7.93-7.88 (m, 2H), 7.40 (d, 1H), 5.39-5.36 (m, 1H), 4.91 (dd, 1H), 4.67 (dd, 1H), 4.23 (q, 2H), 4.16 (s, 3H), 3.91 (t, 2H), 3.84-3.81 (m, 2H), 3.41-3.38 (m, 3H), 2.78 (t, 2H), 2.57-2.55 (m, 2H), 2.26 (t, 2H), 1.77-1.65 (m, 4H), 1.53-1.46 (m, 2H), 1.38-1.23 (m, 5H), 0.93 (t, 3H); MS: m/z=725.3 (M+1, ESI+); HRMS: 725.2557.

### Synthesis of Compound 70

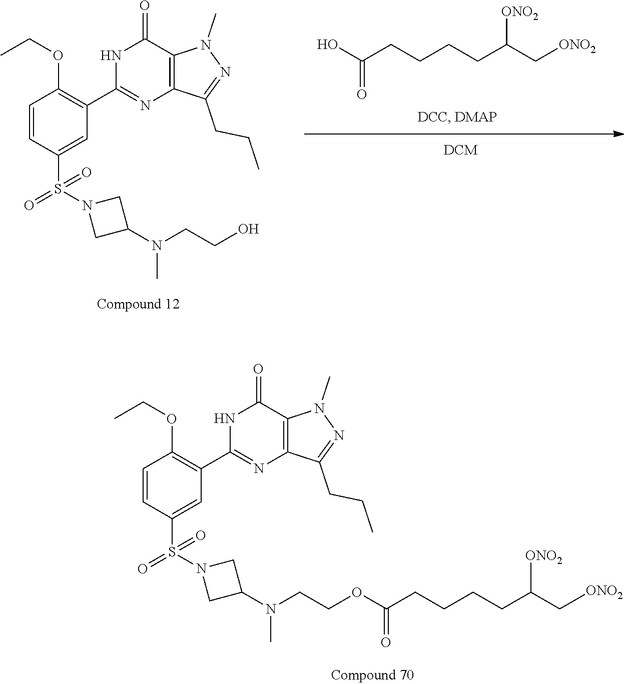

To a solution of compound 12, 5-(2-ethoxy-5-((3-((2-hydroxyethyl)(methyl)amino)azetidin-1-yl)sulfonyl)phenyl)-1-methyl-3-propyl-1,6-dihydro-7H-pyrazolo[4,3-d]pyrimidin-7-one (400 mg, 793 umol) and 6,7-bis(nitrooxy) heptanoic acid (300 mg, 1.19 mmol) in DCM (20 mL) was added DCC (196 mg, 951 umol) and DMAP (97 mg, 793 umol), the reaction mixture was stirred at 25° C. for 16 h. The reaction mixture was poured into water (100 mL) and extracted with DCM (20 mL×3). The organic layer was washed with brine (100 mL×2), dried over Na₂SO₄ and concentrated. The residue was purified by prep-HPLC to afford compound 70, 2-((1-((4-ethoxy-3-(1-methyl-7-oxo-3-propyl-6,7-dihydro-1H-pyrazolo[4,3-d]pyrimidin-5-yl)phenyl)sulfonyl)azetidin-3-yl)(methyl)amino)ethyl 6,7-bis(nitrooxy) heptanoate (200 mg, 34.15% yield) as a white solid. ¹H NMR (400 MHz, DMSO-d₆) δ 12.24 (bs, 1H), 7.95-7.90 (m, 2H), 7.40 (d, 1H), 5.39-5.36 (m, 1H), 4.90 (dd, 1H), 4.67 (dd, 1H), 4.23 (q, 2H), 4.16 (s, 3H), 3.96 (t, 2H), 3.77 (t, 2H), 3.49 (t, 2H), 3.27-3.24 (m, 1H), 2.78 (t, 2H), 2.38 (t, 2H), 2.24 (t, 2H), 1.97 (s, 3H), 1.77-1.64 (m, 4H), 1.50-1.44 (m, 2H), 1.37-1.31 (m, 5H), 0.93 (t, 3H); MS: m/z=739.2 (M+1, ESI+); HRMS: 739.2718.

### Synthesis of Compound 71

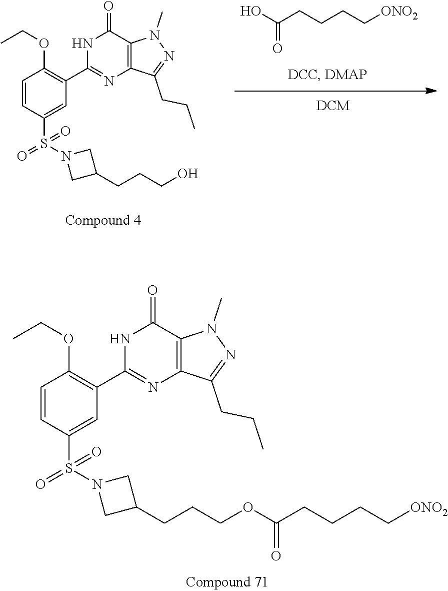

To a solution of compound 4, 5-(2-ethoxy-5-((3-(3-hydroxypropyl)azetidin-1-yl)sulfonyl)phenyl)-1-methyl-3-propyl-1,6-dihydro-7H-pyrazolo[4,3-d]pyrimidin-7-one (300 mg, 613 umol) and 5-(nitrooxy) pentanoic acid (200 mg, 1.23 mmol) in DCM (20 mL) was added DCC (152 mg, 735 umol) and DMAP (75 mg, 613 umol), the reaction mixture was stirred at 25° C. for 16 h. The reaction mixture was poured into water (100 mL) and extracted with DCM (20 mL×3). The organic layer was washed with brine (100 mL×2), dried over Na₂SO₄ and concentrated. The residue was purified by prep-HPLC to afford compound 71, 3-(1-((4-ethoxy-3-(1-methyl-7-oxo-3-propyl-6,7-dihydro-1H-pyrazolo[4,3-d] pyrimidin-5-yl)phenyl)sulfonyl)azetidin-3-yl)propyl 5-(nitrooxy)pentanoate (200 mg, 34.15% yield) as a white solid. ¹H NMR (400 MHz, DMSO-d₆) δ 12.24 (bs, 1H), 7.94-7.89 (m, 2H), 7.41 (d, 1H), 4.48 (t, 2H), 4.23 (q, 2H), 4.17 (s, 3H), 3.91 (t, 2H), 3.80 (t, 2H), 3.35-3.31 (m, 1H), 2.78 (t, 2H), 2.41-2.27 (m, 3H), 1.77-1.67 (m, 2H), 1.63-1.52 (m, 5H), 1.42-1.30 (m, 7H), 0.93 (t, 3H); MS: m/z=635.3 (M+1, ESI+); HRMS: 635.2491.

### Synthesis of Compound 72

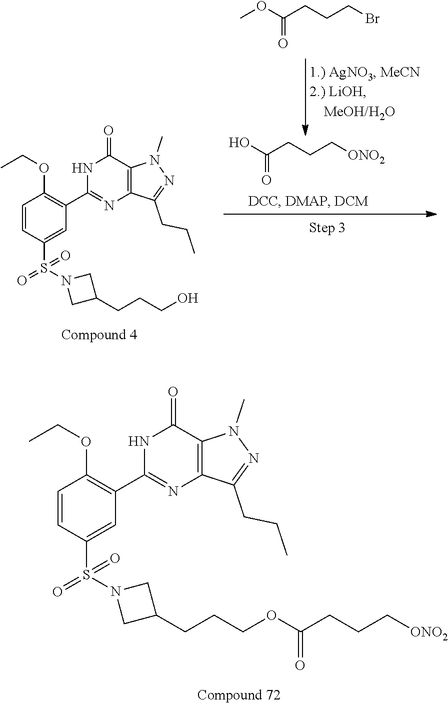

### Step 1:

To a solution of methyl 4-bromobutanoate (1.7 g, 9.39 mmol) in MeCN (80 mL) was added AgNO₃ (3.19 g, 18.78 mmol), the reaction mixture was stirred at 85° C. for 16 h. The resulting mixture was filtered and the filtrate was concentrated under reduced pressure to afford methyl 4-(nitrooxy) butanoate (1.25 g, 81.60% yield) as a light yellow oil.

### Step 2:

To a solution of methyl 4-(nitrooxy)butanoate (1.25 g, 7.66 mmol) in MeOH (10 mL) and H₂O (5 mL) was added LiOH (966 mg, 23 mmol), the reaction mixture was stirred at 25° C. for 16 h. After the reaction was completely finished, 2N HCl was added to adjust pH to 5˜6 and the excess of solvent was removed under reduced pressure to afford 4-(nitrooxy)butanoic acid (800 mg, crude) as a light yellow oil. MS: m/z=150.1 (M+1, ESI+).

### Step 3:

To a solution of compound 4, 5-(2-ethoxy-5-((3-(3-hydroxypropyl)azetidin-1-yl)sulfonyl)phenyl)-1-methyl-3-propyl-1,6-dihydro-7H-pyrazolo[4,3-d]pyrimidin-7-one (200 mg, 409 umol) and 4-(nitrooxy) butanoic acid (91 mg, 613 umol) in DCM (15 mL) was added DCC (101 mg, 490 umol) and DMAP (50 mg, 409 umol), the reaction mixture was stirred at 25° C. for 16 h. The reaction mixture was poured into water (100 mL) and extracted with DCM (20 mL×3). The organic layer was washed with brine (100 mL×2), dried over Na₂SO₄ and concentrated. The residue was purified by prep-HPLC to afford compound 72, 3-(1-((4-ethoxy-3-(1-methyl-7-oxo-3-propyl-6,7-dihydro-1H-pyrazolo[4,3-d] pyrimidin-5-yl)phenyl)sulfonyl)azetidin-3-yl)propyl 4-(nitrooxy)butanoate (110 mg, 43.38% yield) as a white solid. ¹H NMR (400 MHz, DMSO-d₆) δ 12.25 (bs, 1H), 7.95-7.90 (m, 2H), 7.41 (d, 1H), 4.50 (t, 2H), 4.24 (q, 2H), 4.17 (s, 3H), 3.93 (t, 2H), 3.81 (t, 2H), 3.36-3.32 (m, 2H), 2.78 (t, 2H), 2.39-2.35 (m, 3H), 1.92-1.87 (m, 2H), 1.77-1.72 (m, 2H), 1.43-1.33 (m, 7H), 0.94 (t, 3H); MS: m/z=621.1 (M+1, ESI+); HRMS: 621.2341.

### Synthesis of Compound 73

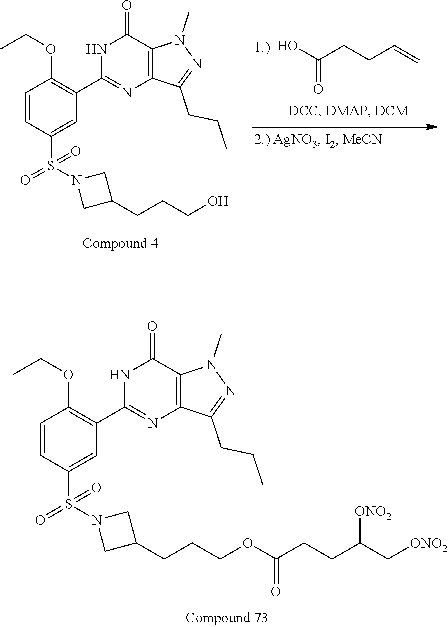

### Step 1:

To a solution of compound 4, 5-(2-ethoxy-5-((3-(3-hydroxypropyl)azetidin-1-yl)sulfonyl)phenyl)-1-methyl-3-propyl-1,6-dihydro-7H-pyrazolo[4,3-d]pyrimidin-7-one (1 g, 2.04 mmol) and pent-4-enoic acid (245 mg, 2.45 mmol) in DCM (20 mL) was added DCC (506 mg, 2.45 mmol) and DMAP (250 mg, 2.04 mmol), the reaction mixture was stirred at 25° C. for 16 h. The reaction mixture was poured into water (100 mL) and extracted with DCM (20 mL×3). The organic layer was washed with brine (100 mL×2), dried over Na₂SO₄ and concentrated. The residue was purified by column chromatography to afford 3-(1-((4-ethoxy-3-(1-methyl-7-oxo-3-propyl-6,7-dihydro-1H-pyrazolo [4,3-d]pyrimidin-5-yl)phenyl)sulfonyl)azetidin-3-yl)propyl pent-4-enoate (1.1 g, 60.2% purity) as a white solid. MS: m/z=572.3 (M+1, ESI+)

### Step 2:

To a solution of 3-(1-((4-ethoxy-3-(1-methyl-7-oxo-3-propyl-6,7-dihydro-1H-pyrazolo[4,3-d] pyrimidin-5-yl)phenyl)sulfonyl)azetidin-3-yl)propyl pent-4-enoate (1.1 g, 1.92 mmol) and AgNO₃ (1.96 g, 11.52 mmol) in MeCN (40 mL) was added 12 (488 mg, 1.92 mmol), the reaction mixture was stirred at 80° C. for 16 h. Cooled to room temperature, the resulting mixture was filtered and the filtrate was concentrated under reduced pressure. The residue was purified by prep-HPLC to afford compound 73, 3-(1-((4-ethoxy-3-(1-methyl-7-oxo-3-propyl-6,7-dihydro-1H-pyrazolo[4,3-d]pyrimidin-5-yl) phenyl)sulfonyl)azetidin-3-yl)propyl 4,5-bis(nitrooxy)pentanoate (107 mg, 7.99% yield) as a white solid. ¹H NMR (400 MHz, DMSO-d₆) δ 12.25 (bs, 1H), 7.92-7.89 (m, 2H), 7.41 (d, 1H), 5.45-5.43 (m, 1H), 4.92 (dd, 1H), 4.70 (dd, 1H), 4.23 (q, 2H), 4.16 (s, 3H), 3.92 (t, 2H), 3.80 (t, 2H), 3.35-3.32 (m, 2H), 2.77 (t, 2H), 2.47-2.37 (m, 3H), 2.00-1.89 (m, 2H), 1.77-1.71 (m, 2H), 1.43-1.33 (m, 7H), 0.93 (t, 3H); MS: m/z=696.2 (M+1, ESI+); HRMS: 696.2291.

### Example 2—Preparation of Substituted Amino-Azetidine-Linked dihydro-1H-pyrazolo[4,3-d]pyrimidine Compounds

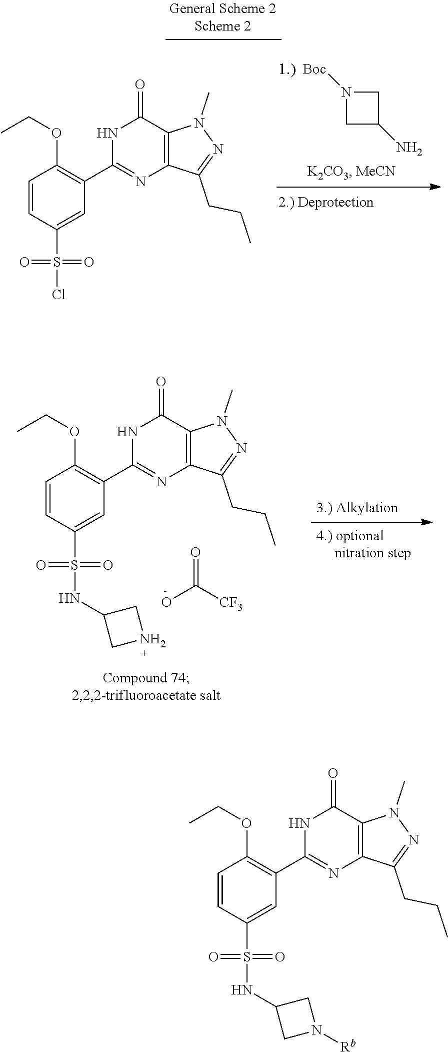

### Synthesis of Compound 6

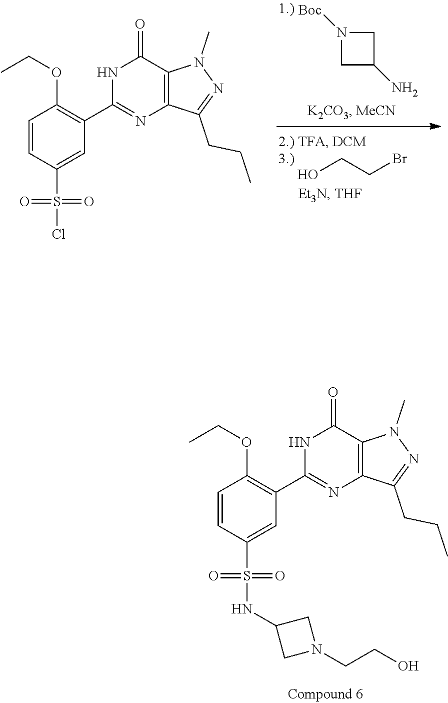

### Step 1:

To a solution of 4-ethoxy-3-(1-methyl-7-oxo-3-propyl-6,7-dihydro-1H-pyrazolo[4,3-d]pyrimidin-5-yl) benzenesulfonyl chloride (4.29 g, 10.45 mmol) and tert-butyl 3-aminoazetidine-1-carboxylate (2 g, 11.61 mmol) in MeCN (100 mL) was added K₂CO₃ (4.81 g, 34.84 mmol), the reaction mixture was stirred at 100° C. for 5 h. The reaction mixture was poured into water (200 mL), extracted with EA (50 mL×3), washed by brine (50 mL×3), dried over Na₂SO₄ and concentrated, the residue was purified by column chromatography to afford tert-butyl 3-((4-ethoxy-3-(1-methyl-7-oxo-3-propyl-6,7-dihydro-1H-pyrazolo [4,3-d]pyrimidin-5-yl)phenyl)sulfonamido)azetidine-1-carboxylate (5 g, 78.77% yield) as a white solid. MS: m/z=547.6 (M+1, ESI+).

### Step 2:

A mixture of tert-butyl 3-((4-ethoxy-3-(1-methyl-7-oxo-3-propyl-6,7-dihydro-1H-pyrazolo[4,3-d] pyrimidin-5-yl)phenyl)sulfonamido)azetidine-1-carboxylate (5 g, 9.15 mmol) in DCM (100 mL) was added TFA (10.43 g, 91.47 mmol) and stirred at 25° C. for 16 h. The reaction mixture was evaporated under reduced pressure to afford compound 74; 2,2,2-trifluoroacetate salt, N-(azetidin-3-yl)-4-ethoxy-3-(1-methyl-7-oxo-3-propyl-6,7-dihydro-1H-pyrazolo[4,3-d]pyrimidin-5-yl)benzenesulfonamide; 2,2,2-trifluoroacetate salt (4 g, 97.94% yield) as a yellow oil. MS: m/z=447.5 (M+1, ESI+).

### Step 3:

To a solution of compound 74; 2,2,2-trifluoroacetate salt, N-(azetidin-3-yl)-4-ethoxy-3-(1-methyl-7-oxo-3-propyl-6,7-dihydro-1H-pyrazolo [4,3-d] pyrimidin-5-yl)benzenesulfonamide; 2,2,2-trifluoroacetate salt (500 mg, 1.12 mmol) and 2-bromoethan-1-ol (420 mg, 3.36 mmol) in THF (10 mL) was added TEA (567 mg, 5.60 mmol), the reaction mixture was stirred at 80° C. for 24 h. The reaction mixture was poured into water (50 mL), extracted with EA (20 mL×3), washed by brine (30 mL×3), dried over Na₂SO₄ and concentrated, the residue was purified by prep-HPLC to afford compound 6, 4-ethoxy-N-(1-(2-hydroxyethyl)azetidin-3-yl)-3-(1-methyl-7-oxo-3-propyl-6,7-dihydro-1H-pyrazolo[4,3-d]pyrimidin-5-yl)benzenesulfonamide (95 mg, 17.29% yield) as a white solid. ¹H NMR (400 MHz, DMSO-d₆) δ 12.20 (bs, 1H), 8.22-8.20 (m, 1H), 7.93-7.86 (m, 2H), 7.32 (d, 1H), 4.41 (bs, 1H), 4.22-4.16 (m, 5H), 3.77-3.76 (m, 11H), 3.43-3.40 (m, 2H), 3.28-3.27 (m, 2H), 2.80-2.77 (m, 4H), 2.43-2.41 (m, 2H), 1.79-1.72 (m, 2H), 1.33 (t, 3H), 0.94 (t, 3H); MS: m/z=491.5 (M+1, ESI+); HRMS: 491.2072.

### Synthesis of Compound 7

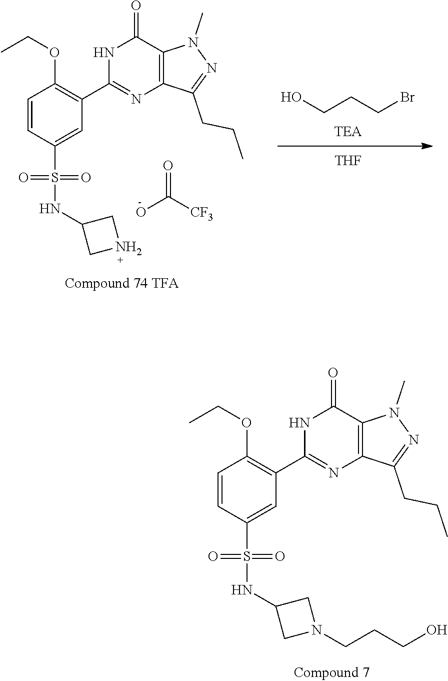

To a solution of compound 74; 2,2,2-trifluoroacetate salt, N-(azetidin-3-yl)-4-ethoxy-3-(1-methyl-7-oxo-3-propyl-6,7-dihydro-1H-pyrazolo [4,3-d] pyrimidin-5-yl)benzenesulfonamide; 2,2,2-trifluoroacetate salt (500 mg, 1.12 mmol) and 3-bromopropan-1-ol (467 mg, 3.36 mmol) in THF (10 mL) was added TEA (567 mg, 5.60 mmol), the reaction mixture was stirred at 80° C. for 24 h. The reaction mixture was poured into water (50 mL), extracted with EA (20 mL×3), washed by brine (30 mL×3), dried over Na₂SO₄ and concentrated, the residue was purified by prep-HPLC to afford compound 7, 4-ethoxy-N-(1-(3-hydroxypropyl)azetidin-3-yl)-3-(1-methyl-7-oxo-3-propyl-6,7-dihydro-1H-pyrazolo[4,3-d]pyrimidin-5-yl)benzenesulfonamide (200 mg, 35.4% yield) as a white solid. ¹H NMR (400 MHz, DMSO-d₆) δ 12.20 (bs, 1H), 8.16 (bs, 1H), 7.94-7.86 (m, 2H), 7.32 (d, 1H), 4.38 (bs, 1H), 4.22-4.17 (m, 5H), 3.73-3.72 (m, 1H), 3.34-3.32 (m, 4H), 2.78 (t, 2H), 2.63-2.60 (m, 2H), 2.33-2.30 (m, 2H), 1.78-1.72 (m, 2H), 1.36-1.32 (m, 5H), 0.94 (t, 3H); MS: m/z=506.6 (M+1, ESI+); HRMS: 505.2228.

### Synthesis of Compound 8

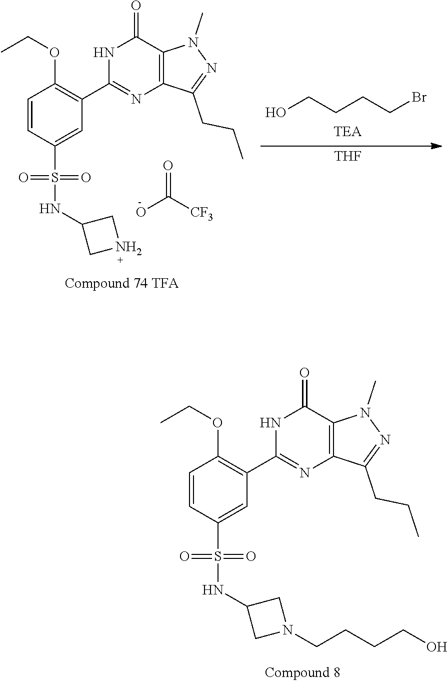

To a solution of compound 74; 2,2,2-trifluoroacetate salt, N-(azetidin-3-yl)-4-ethoxy-3-(1-methyl-7-oxo-3-propyl-6,7-dihydro-1H-pyrazolo [4,3-d] pyrimidin-5-yl)benzenesulfonamide; 2,2,2-trifluoroacetate salt (500 mg, 1.12 mmol) and 4-bromobutan-1-ol (514 mg, 3.36 mmol) in THF (10 mL) was added TEA (567 mg, 5.60 mmol), the reaction mixture was stirred at 80° C. for 24 h. The reaction mixture was poured into water (50 mL), extracted with EA (20 mL×3), washed by brine (30 mL×3), dried over Na₂SO₄ and concentrated, the residue was purified by prep-HPLC to afford compound 8, 4-ethoxy-N-(1-(4-hydroxybutyl)azetidin-3-yl)-3-(1-methyl-7-oxo-3-propyl-6,7-dihydro-1H-pyrazolo[4,3-d] pyrimidin-5-yl)benzenesulfonamide (53 mg, 9.13% yield) as a white solid. ¹H NMR (400 MHz, DMSO-d₆) δ 12.20 (bs, 1H), 8.16 (bs, 1H), 7.94 (d, 1H), 7.86 (dd, 1H), 7.32 (d, 1H), 4.38 (bs, 1H), 4.22-4.17 (m, 5H), 3.73-3.71 (m, 1H), 3.34-3.31 (m, 4H), 2.78 (t, 2H), 2.73-2.65 (m, 2H), 2.33-2.30 (m, 2H), 1.78-1.72 (m, 2H), 1.36-1.32 (m, 5H), 1.25-1.20 (m, 2H), 0.94 (t, 3H); MS: m/z=519.6 (M+1, ESI+); HRMS: 519.2385.

### Synthesis of Compound 19

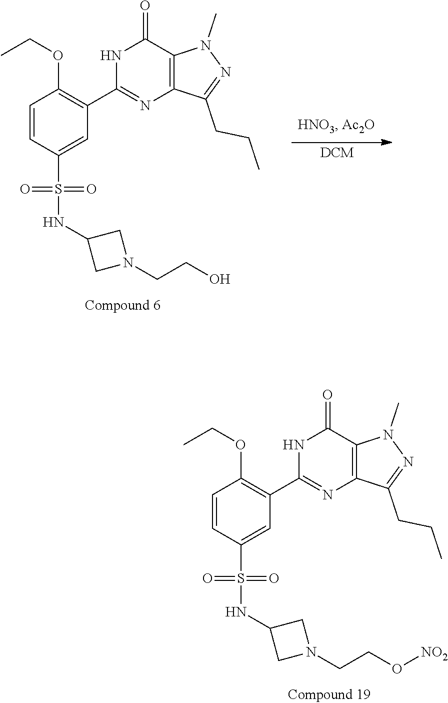

To a solution compound 6, of 4-ethoxy-N-(1-(2-hydroxyethyl)azetidin-3-yl)-3-(1-methyl-7-oxo-3-propyl-6,7-dihydro-1H-pyrazolo[4,3-d]pyrimidin-5-yl)benzenesulfonamide (300 mg, 611 umol) in DCM (6 mL) was added HNO₃ (193 mg, 3.06 mmol) and Ac₂O (324 mg, 3.06 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 19, 2-(3-((4-ethoxy-3-(1-methyl-7-oxo-3-propyl-6,7-dihydro-1H-pyrazolo[4,3-d]pyrimidin-5-yl) phenyl)sulfonamido)azetidin-1-yl)ethyl nitrate (22 mg, 6.54% yield) as a white solid. ¹H NMR (400 MHz, DMSO-d₆) δ 12.04 (bs, 1H), 7.93 (bs, 1H), 7.93 (d, 1H), 7.85 (dd, 1H), 7.32 (d, 1H), 4.41-4.39 (m, 2H), 4.21-4.16 (m, 5H), 3.77-3.73 (m, 1H), 3.39 (t, 2H), 2.80-2.73 (m, 4H), 2.63-2.61 (m, 2H), 1.77-1.71 (m, 2H), 1.33 (t, 3H), 0.94 (t, 3H); MS: m/z=536.5 (M+1, ESI+); HRMS: 536.1919.

### Synthesis of Compound 20

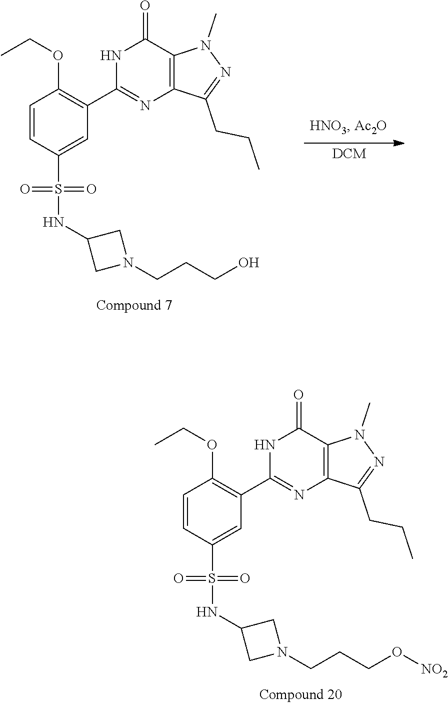

To a solution compound 7, of 4-ethoxy-N-(1-(3-hydroxypropyl)azetidin-3-yl)-3-(1-methyl-7-oxo-3-propyl-6,7-dihydro-1H-pyrazolo[4,3-d]pyrimidin-5-yl)benzenesulfonamide (245 mg, 486 umol) in DCM (10 mL) was added HNO₃ (153 mg, 2.43 mmol) and Ac₂O (248 mg, 2.43 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 20, 3-(3-((4-ethoxy-3-(1-methyl-7-oxo-3-propyl-6,7-dihydro-1H-pyrazolo[4,3-d]pyrimidin-5-yl) phenyl)sulfonamido)azetidin-1-yl)propyl nitrate (40 mg, 14.7% yield) as a white solid. ¹H NMR (400 MHz, DMSO-d₆) δ 12.21 (bs, 1H), 8.19-8.17 (m, 1H), 7.93-7.85 (m, 2H), 7.32 (d, 1H), 4.46 (t, 2H), 4.21-4.16 (m, 5H), 3.77-3.72 (m, 1H), 3.34-3.32 (m, 2H), 2.78 (t, 2H), 2.63-2.61 (m, 2H), 2.35-2.33 (m, 2H), 1.77-1.71 (m, 2H), 1.60-1.57 (m, 2H), 1.33 (t, 3H), 0.93 (t, 3H); MS: m/z=550.6 (M+1, ESI+); HRMS: 550.2083.

### Example 3—Preparation of Substituted Azetidine-Linked imidazo[5,1-f][1,2,4]triazin-4(3H)-one Compounds

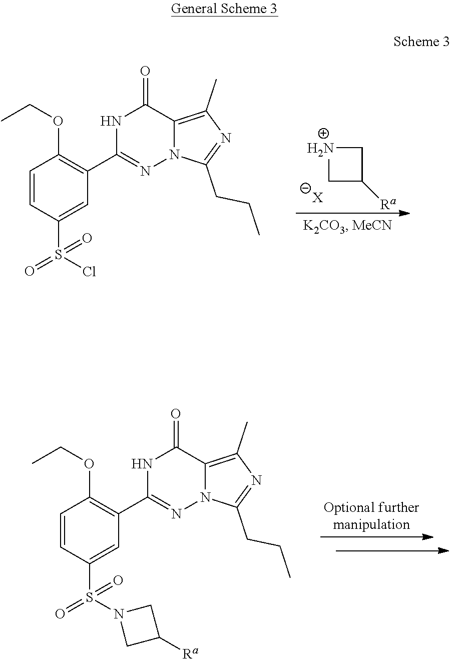

### Synthesis of Compound 41

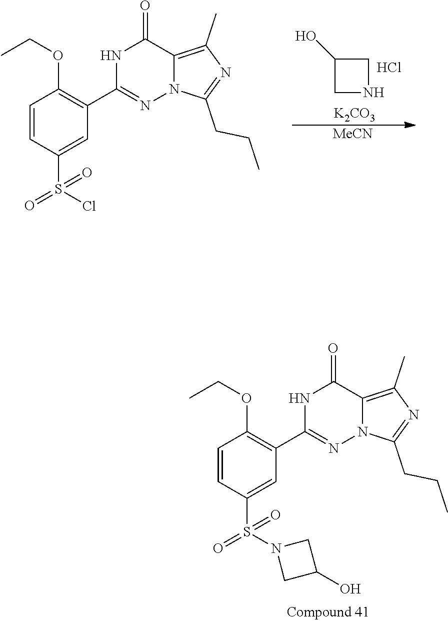 [SMILES: "MeCN" ; CCCc1nc(C)c2c(=O)[nH]c(-c3cc(S(=O)(=O)Cl)ccc3OCC)nn12 ; "K2CO3" ; OC1CNC1 ; Cl ; CCCc1nc(C)c2c(=O)[nH]c(-c3cc(S(=O)(=O)N4CC(O)C4)ccc3OCC)nn12]

To a solution of 4-ethoxy-3-(5-methyl-4-oxo-7-propyl-3,4-dihydroimidazo[5,1-f][1,2,4]triazin-2-yl) benzenesulfonyl chloride (500 mg, 1.22 umol) and azetidin-3-ol hydrochloride (200 mg, 1.83 mmol) in MeCN (40 mL) was added K₂CO₃ (589 mg, 4.26 mmol), the reaction mixture was stirred at 25° C. for 16 h. Filtered and evaporated under reduced pressure, the residue was purified by prep-HPLC to afford compound 41, 2-(2-ethoxy-5-((3-hydroxyazetidin-1-yl)sulfonyl)phenyl)-5-methyl-7-propylimidazo[5,1-f][1,2,4]triazin-4 (3H)-one (400 mg, 73.45% yield) as a white solid. ¹H NMR (400 MHz, DMSO-d₆) δ 11.73 (bs, 1H), 7.95-7.90 (m, 2H), 7.42 (d, 1H), 5.79 (bd, 1H), 4.31-4.28 (m, 1H), 4.24 (q, 2H), 3.91-3.87 (m, 2H), 3.39-3.35 (m, 2H), 2.83 (t, 2H), 2.48 (s, 3H), 1.76-1.71 (m, 2H), 1.34 (t, 3H), 0.92 (t, 3H); MS: m/z=448.3 (M+1, ESI+); HRMS: 448.1650.

### Synthesis of Compound 42

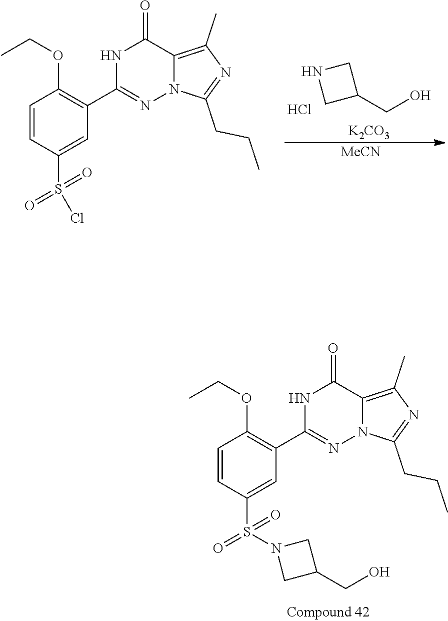

To a solution of 4-ethoxy-3-(5-methyl-4-oxo-7-propyl-3,4-dihydroimidazo[5,1-f][1,2,4]triazin-2-yl) benzenesulfonyl chloride (656 mg, 1.60 mmol) and azetidin-3-ylmethanol hydrochloride (139 mg, 1.60 mmol) in MeCN (10 mL) was added K₂CO₃ (662 mg, 4.79 mmol), the reaction mixture was stirred at 100° C. for 4 h. Filtered and evaporated under reduced pressure, the residue was purified by prep-HPLC to afford compound 42, 2-(2-ethoxy-5-((3-(hydroxymethyl)azetidin-1-yl)sulfonyl)phenyl)-5-methyl-7-propylimidazo[5,1-f][1,2,4]triazin-4(3H)-one (320 mg, 43.46% yield) as a white solid. ¹H NMR (400 MHz, DMSO-d₆) δ 11.57 (bs, 1H), 7.95-7.91 (m, 2H), 7.41 (d, 1H), 4.69 (bs, 1H), 4.23 (q, 2H), 3.73 (t, 2H), 3.48-3.44 (m, 2H), 3.31-3.29 (m, 2H), 2.83 (t, 2H), 2.48 (s, 3H), 1.78-1.69 (m, 2H), 1.34 (t, 3H), 0.92 (t, 3H); MS: m/z=462.3 (M+1, ESI+); HRMS: 462.1805.

### Synthesis of Compound 43

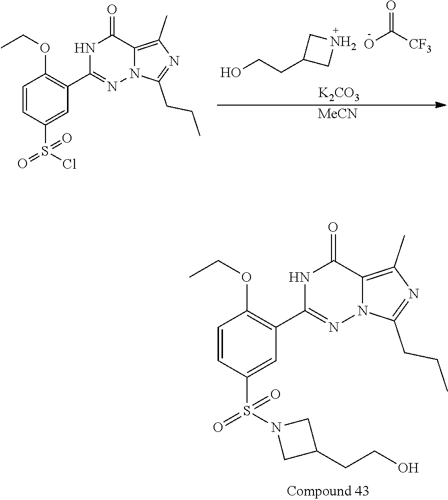

To a solution of 4-ethoxy-3-(5-methyl-4-oxo-7-propyl-3,4-dihydroimidazo[5,1-f][1,2,4]triazin-2-yl) benzenesulfonyl chloride (800 mg, 1.95 mmol) and 2-(azetidin-3-yl)ethan-1-ol; 2,2,2-trifluoroacetate salt (1.25 g, 5.84 mmol) in MeCN (20 mL) was added K₂CO₃ (807 mg, 5.84 mmol), the reaction mixture was stirred at 100° C. for 3 h. Filtered and evaporated under reduced pressure, the residue was purified by prep-HPLC to afford compound 43, 2-(2-ethoxy-5-((3-(2-hydroxyethyl)azetidin-1-yl)sulfonyl)phenyl)-5-methyl-7-propylimidazo[5,1-f][1,2,4]triazin-4(3H)-one (425 mg, 45.90% yield) as a white solid. ¹H NMR (400 MHz, DMSO-d₆) δ 11.43 (bs, 1H), 7.94-7.91 (m, 2H), 7.41 (d, 11H), 4.37 (bs, 1H), 4.23 (q, 2H), 3.80 (t, 2H), 3.38-3.35 (m, 2H), 3.29-3.25 (m, 2H), 2.82 (t, 2H), 2.48 (s, 31H), 1.77-1.68 (m, 2H), 1.45-1.38 (m, 2H), 1.34 (t, 31H), 0.92 (t, 3H); MS: m/z=476.2 (M+1, ESI+); HRMS: 476.1964.

### Synthesis of Compound 44

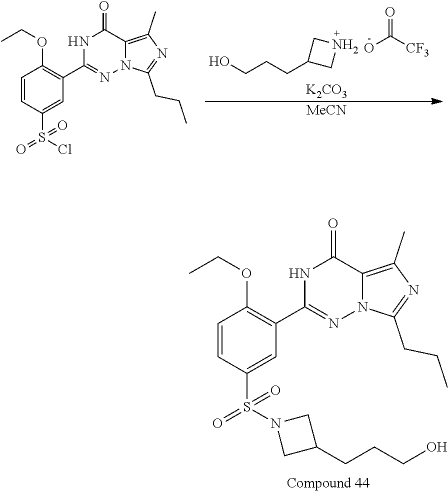

To a solution of 4-ethoxy-3-(5-methyl-4-oxo-7-propyl-3,4-dihydroimidazo[5,1-f][1,2,4]triazin-2-yl) benzenesulfonyl chloride (600 mg, 1.46 mmol) and 3-(azetidin-3-yl)propan-1-ol; 2,2,2-trifluoroacetate salt (202 mg, 1.75 mmol) in MeCN (10 mL) was added K₂CO₃ (2.02 g, 14.60 mmol), the reaction mixture was stirred at 25° C. for 3 h. Filtered and evaporated under reduced pressure, the residue was purified by prep-HPLC to afford compound 44, 2-(2-ethoxy-5-((3-(3-hydroxypropyl)azetidin-1-yl)sulfonyl)phenyl)-5-methyl-7-propylimidazo [5,1-f][1,2,4]triazin-4(3H)-one (400 mg, 55% yield) as a white solid. ¹H NMR (400 MHz, DMSO-d₆) δ 11.42 (bs, 1H), 7.95-7.92 (m, 2H), 7.42 (d, 1H), 4.23 (q, 2H), 3.80 (t, 2H), 3.32-3.27 (m, 4H), 2.83 (t, 2H), 2.48 (s, 3H), 2.39-2.36 (m, 1H), 1.76-1.69 (m, 2H), 1.36-1.21 (m, 7H), 0.92 (t, 3H); MS: m/z=490.3 (M+1, ESI+); HRMS: 490.2121.

### Synthesis of Compound 45

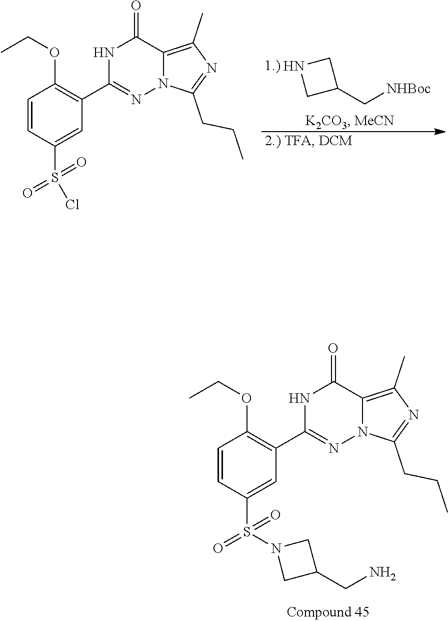

### Step 1:

To a solution of 4-ethoxy-3-(5-methyl-4-oxo-7-propyl-3,4-dihydroimidazo[5,1-f][1,2,4]triazin-2-yl) benzenesulfonyl chloride (492 mg, 1.20 mmol) and tert-butyl (azetidin-3-ylmethyl)carbamate (186 mg, 999 umol) in MeCN (10 mL) was added K₂CO₃ (414 mg, 3.00 mmol), the resulting mixture was stirred at 100° C. for 4 h. Filtered and evaporated under reduced pressure, the residue was purified by prep-HPLC to afford tert-butyl ((1-((4-ethoxy-3-(5-methyl-4-oxo-7-propyl-3,4-dihydro imidazo[5,1-f][1,2,4] triazin-2-yl)phenyl)sulfonyl)azetidin-3-yl)methyl)carbamate (450 mg, 80.37% yield) as a white solid. MS: m/z=561.3 (M+1, ESI+).

### Step 2:

A mixture of tert-butyl ((1-((4-ethoxy-3-(5-methyl-4-oxo-7-propyl-3,4-dihydroimidazo[5,1-f][1,2,4] triazin-2-yl)phenyl)sulfonyl)azetidin-3-yl)methyl)carbamate (450 mg, 713 umol) in DCM (5 mL) was added TFA (411 mg, 3.61 mmol), the reaction mixture was stirred at 25° C. for 2 h. The reaction mixture was evaporated under reduced pressure, the residue was purified by prep-HPLC to afford compound 45, 2-(5-((3-(aminomethyl)azetidin-1-yl)sulfonyl)-2-ethoxyphenyl)-5-methyl-7-propylimidazo[5,1-f][1,2,4]triazin-4 (3H)-one (181 mg, 49.05% yield) as a white solid. ¹H NMR (400 MHz, DMSO-d₆) δ 7.95-7.90 (m, 2H), 7.41 (d, 1H), 4.23 (q, 2H), 4.08 (bs, 2H), 3.73 (t, 2H), 3.47-3.43 (m, 2H), 2.83 (t, 2H), 2.53-2.48 (m, 5H), 2.40-2.33 (m, 1H), 1.78-1.69 (m, 2H), 1.34 (t, 3H), 0.92 (t, 3H); MS: m/z=462.2 (M+1, ESI+); HRMS: 461.1968.

### Synthesis of Compound 49

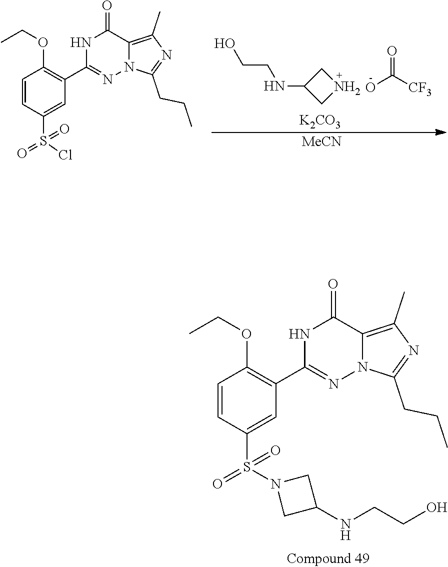

To a solution of 4-ethoxy-3-(5-methyl-4-oxo-7-propyl-3,4-dihydroimidazo[5,1-f][1,2,4]triazin-2-yl) benzenesulfonyl chloride (1.00 g, 2.43 mmol) and 2-(azetidin-3-ylamino)ethan-1-ol; 2,2,2-trifluoroacetate salt (1.13 g, 9.74 mmol) in MeCN (30 mL) was added K₂CO₃ (3.32 g, 24.3 mmol), the reaction mixture was stirred at 25° C. for 2 h. Filtered and evaporated under reduced pressure, the residue was purified by prep-HPLC to afford compound 49, 2-(2-ethoxy-5-((3-((2-hydroxyethyl)amino)azetidin-1-yl)sulfonyl) phenyl)-5-methyl-7-propylimidazo[5,1-f][1,2,4]triazin-4(3H)-one (600 mg, 50% yield) as a white solid. ¹H NMR (400 MHz, DMSO-d₆) δ 11.74 (bs, 1H), 7.95-7.93 (m, 2H), 7.41 (d, 1H), 4.46 (bs, 1H), 4.24 (q, 2H), 3.83-3.82 (m, 2H), 3.41-3.40 (m, 3H), 3.34-3.31 (m, 3H), 2.83 (t, 2H), 2.49 (s, 3H), 2.440 (t, 2H), 1.77-1.71 (m, 2H), 1.34 (t, 3H), 0.92 (t, 3H); MS: m/z=491.1 (M+1, ESI+); HRMS: 491.2074.

### Synthesis of Compound 50

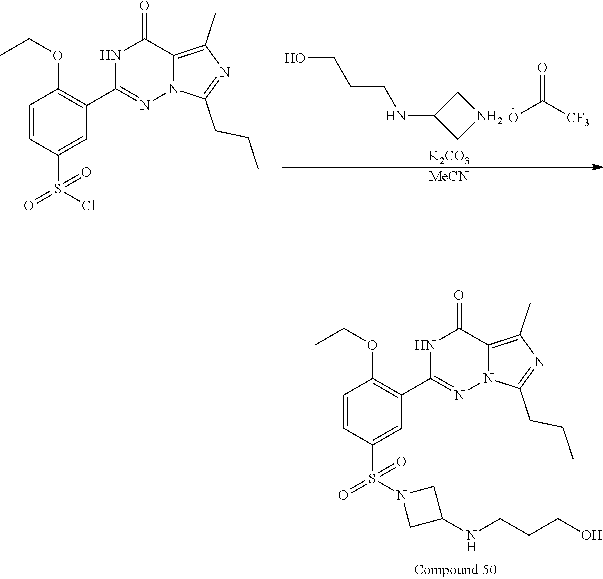

To a solution of 4-ethoxy-3-(5-methyl-4-oxo-7-propyl-3,4-dihydroimidazo[5,1-f][1,2,4]triazin-2-yl) benzenesulfonyl chloride (500 mg, 1.22 mmol) and 3-(azetidin-3-ylamino)propan-1-ol; 2,2,2-trifluoroacetate salt (355 mg, 1.46 mmol) in MeCN (15 mL) was added K₂CO₃ (505 mg, 3.65 mmol), the reaction mixture was stirred at 80° C. for 2 h. Filtered and evaporated under reduced pressure, the residue was purified by prep-HPLC to afford compound 50, 2-(2-ethoxy-5-((3-((3-hydroxypropyl)amino)azetidin-1-yl) sulfonyl)phenyl)-5-methyl-7-propylimidazo[5,1-f][1,2,4]triazin-4(3H)-one (310 mg, 50.48% yield) as a white solid. ¹H NMR (400 MHz, DMSO-d₆) δ 11.74 (bs, 1H), 7.95-7.92 (m, 2H), 7.41 (d, 1H), 4.24 (q, 2H), 3.83 (t, 2H), 3.44-3.35 (m, 5H), 2.83 (t, 2H), 2.49 (s, 3H), 2.35 (t, 2H), 1.76-1.71 (m, 2H), 1.44-1.41 (m, 2H), 1.34 (s, 3H), 0.92 (t, 3H); MS: m/z=505.3 (M+1, ESI+); HRMS: 505.2231.

### Synthesis of Compound 51

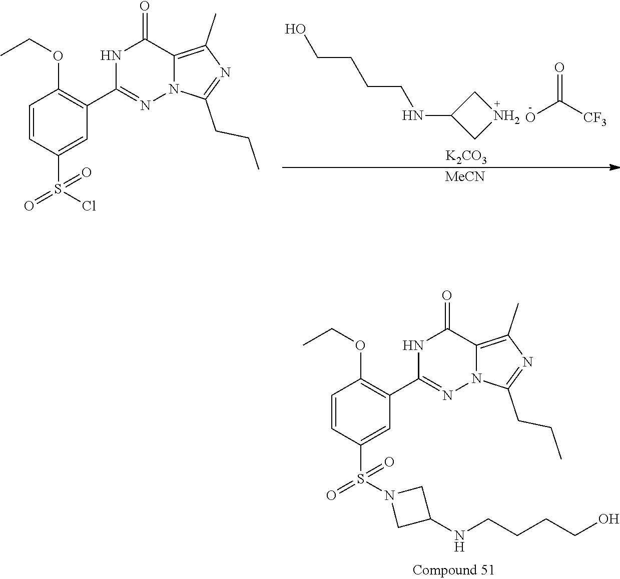

To a solution of 4-ethoxy-3-(5-methyl-4-oxo-7-propyl-3,4-dihydroimidazo[5,1-f][1,2,4]triazin-2-yl) benzenesulfonyl chloride (500 mg, 1.22 mmol) and 4-(azetidin-3-ylamino)butan-1-ol; 2,2,2-trifluoroacetate salt (351 mg, 2.43 mmol) in MeCN (10 mL) was added K₂CO₃ (505 mg, 3.65 mmol), the reaction mixture was stirred at 25° C. for 2 h. Filtered and evaporated under reduced pressure, the residue was purified by prep-HPLC to afford compound 51, 2-(2-ethoxy-5-((3-((4-hydroxybutyl)amino)azetidin-1-yl)sulfonyl)phenyl)-5-methyl-7-propylimidazo[5,1-f][1,2,4]triazin-4(3H)-one (500 mg, 79.22% yield) as a white solid. ¹H NMR (400 MHz, DMSO-d₆) δ 11.63 (bs, 1H), 7.94-7.91 (m, 2H), 7.41 (d, 1H), 4.25-4.20 (m, 2H), 3.82-3.80 (m, 2H), 3.42-3.30 (m, 6H), 2.82 (t, 2H), 2.48 (s, 3H), 2.29-2.25 (m, 2H), 1.76-1.70 (m, 2H), 1.35-1.28 (m, 7H), 0.92 (t, 3H); MS: m/z=519.3 (M+1, ESI+); HRMS: 519.2382.

### Synthesis of Compound 52

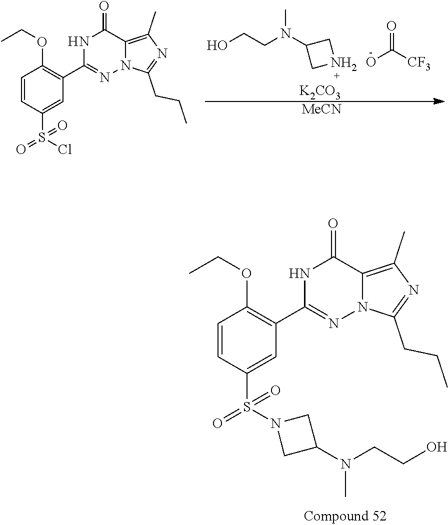

To a solution of 4-ethoxy-3-(5-methyl-4-oxo-7-propyl-3,4-dihydroimidazo[5,1-f][1,2,4]triazin-2-yl) benzenesulfonyl chloride (945 mg, 2.30 mmol) and 2-(azetidin-3-yl(methyl)amino)ethan-1-ol; 2,2,2-trifluoroacetate salt (300 mg, 2.30 mmol) in MeCN (20 mL) was added K₂CO₃ (955 mg, 6.91 mmol), the reaction mixture was stirred at 100° C. for 6 h. Filtered and evaporated under reduced pressure, the residue was purified by prep-HPLC to afford compound 52, 2-(2-ethoxy-5-((3-((2-hydroxyethyl)(methyl)amino)azetidin-1-yl)sulfonyl)phenyl)-5-methyl-7-propylimidazo[5,1-f][1,2,4]triazin-4(3H)-one (158 mg, 13.61% yield) as a white solid. ¹H NMR (400 MHz, DMSO-d₆) δ 11.73 (bs, 1H), 7.96-7.93 (m, 2H), 7.42 (d, 1H), 4.39 (bs, 1H), 4.24 (q, 2H), 3.76 (t, 2H), 3.50 (t, 2H), 3.33-3.31 (m, 2H), 3.25-3.22 (m, 1H), 2.83 (t, 2H), 2.49 (s, 3H), 2.21 (t, 2H), 1.95 (s, 3H), 1.76-1.71 (m, 2H), 1.34 (t, 3H), 0.92 (t, 3H); MS: m/z=505.1 (M+1, ESI+); HRMS: 505.2226.

### Synthesis of Compound 53

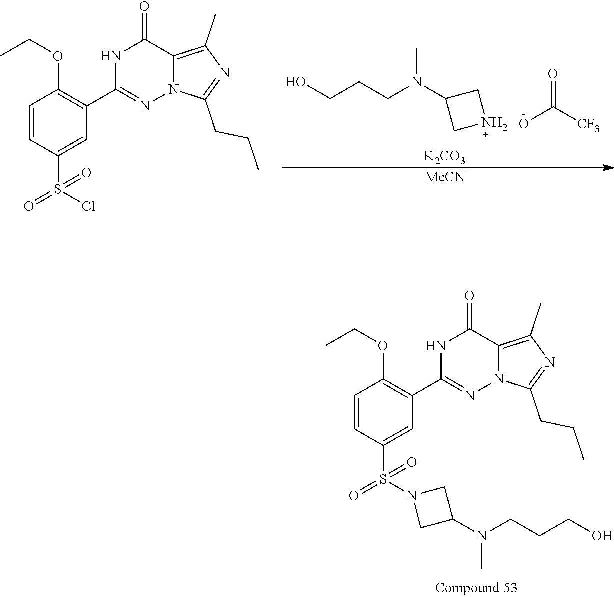

To a solution of 4-ethoxy-3-(5-methyl-4-oxo-7-propyl-3,4-dihydroimidazo[5,1-f][1,2,4]triazin-2-yl) benzenesulfonyl chloride (712 mg, 1.73 mmol) and 3-(azetidin-3-yl(methyl)amino)propan-1-ol; 2,2,2-trifluoroacetate salt (500 mg, 3.47 mmol) in MeCN (20 mL) was added K₂CO₃ (958 mg, 6.93 mmol), the reaction mixture was stirred at 80° C. for 4 h. Filtered and evaporated under reduced pressure, the residue was purified by prep-HPLC to afford compound 53, 2-(2-ethoxy-5-((3-((3-hydroxypropyl)(methyl)amino)azetidin-1-yl)sulfonyl)phenyl)-5-methyl-7-propylimidazo[5,1-f][1,2,4]triazin-4(3H)-one (63 mg, 7.01% yield) as a white solid. ¹H NMR (400 MHz, DMSO-d₆) δ 11.73 (bs, 1H), 7.97-7.94 (m, 2H), 7.42 (d, 1H), 4.34 (bs, 1H), 4.24 (q, 2H), 3.77 (t, 2H), 3.49 (t, 2H), 3.33-3.30 (m, 2H), 3.15-3.11 (m, 1H), 2.83 (t, 2H), 2.49 (s, 3H), 2.12 (t, 2H), 1.89 (s, 3H), 1.76-1.71 (m, 2H), 1.44-1.39 (m, 2H), 1.35 (t, 3H), 0.92 (t, 3H); MS: m/z=519.3 (M+1, ESI+); HRMS: 519.2385.

### Synthesis of Compound 54

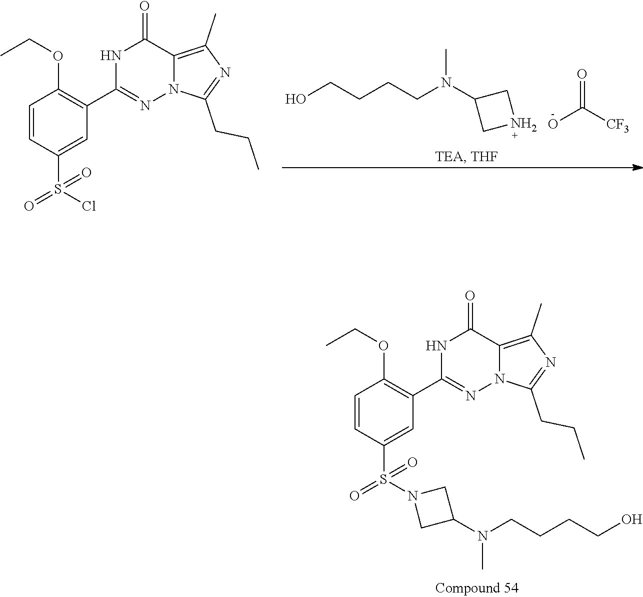

To a solution of 4-ethoxy-3-(5-methyl-4-oxo-7-propyl-3,4-dihydroimidazo[5,1-f][1,2,4]triazin-2-yl) benzenesulfonyl chloride (300 mg, 730 umol) and 4-(azetidin-3-yl(methyl)amino)butan-1-ol; 2,2,2-trifluoroacetate salt (116 mg, 730 umol) in THF (30 mL) was added TEA (369 mg, 3.65 mmol), the reaction mixture was stirred at 25° C. for 0.5 h. Filtered and evaporated under reduced pressure, the residue was purified by prep-HPLC to afford compound 54, 2-(2-ethoxy-5-((3-((4-hydroxybutyl)(methyl)amino) azetidin-1-yl)sulfonyl)phenyl)-5-methyl-7-propylimidazo[5,1-f][1,2,4]triazin-4(3H)-one (135 mg, 34.71% yield) as a white solid. ¹H NMR (400 MHz, DMSO-d₆) δ 11.74 (bs, 1H), 7.96-7.92 (m, 2H), 7.42 (d, 1H), 4.38 (bs, 1H), 4.24 (q, 2H), 3.78 (t, 2H), 3.48-3.46 (m, 2H), 3.32-3.31 (m, 2H), 3.14-3.12 (m, 1H), 2.83 (t, 2H), 2.48 (s, 3H), 2.04-2.02 (m, 2H), 1.89-1.87 (m, 3H), 1.78-1.69 (m, 2H), 1.36-1.27 (m, 7H), 0.92 (t, 3H); MS: m/z=533.3 (M+1, ESI+); HRMS: 533.2544.

### Synthesis of Compound 55

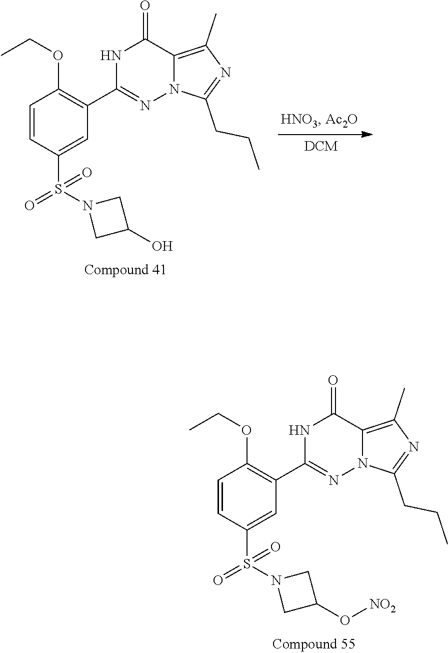 [SMILES: CCCc1nc(C)c2c(=O)[nH]c(-c3cc(S(=O)(=O)N4CC(O)C4)ccc3OCC)nn12 ; CCCc1nc(C)c2c(=O)[nH]c(-c3cc(S(=O)(=O)N4CC(O[N+](=O)[O-])C4)ccc3OCC)nn12]

To a solution of compound 41, 2-(2-ethoxy-5-((3-hydroxyazetidin-1-yl)sulfonyl)phenyl)-5-methyl-7-propylimidazo [5,1-f][1,2,4]triazin-4(3H)-one (250 mg, 559 umol) in DCM (10 mL) was added HNO₃ (162 mg, 1.68 mmol) and Ac₂O (296 mg, 2.79 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 55, 1-((4-ethoxy-3-(5-methyl-4-oxo-7-propyl-3,4-dihydroimidazo[5,1-f][1,2,4]triazin-2-yl)phenyl) sulfonyl)azetidin-3-yl nitrate (130 mg, 47.25% yield) as a white solid. ¹H NMR (400 MHz, DMSO-d₆) δ 11.75 (bs, 1H), 8.01-7.96 (m, 2H), 7.42 (d, 1H), 5.40-5.36 (m, 1H), 4.24 (q, 2H), 4.18-4.14 (m, 2H), 3.92-3.88 (dd, 2H), 2.82 (t, 2H), 2.48 (s, 3H), 1.76-1.68 (m, 2H), 1.34 (t, 3H), 0.92 (t, 3H); MS: m/z=493.1 (M+1, ESI+); HRMS: 493.1501.

### Synthesis of Compound 56

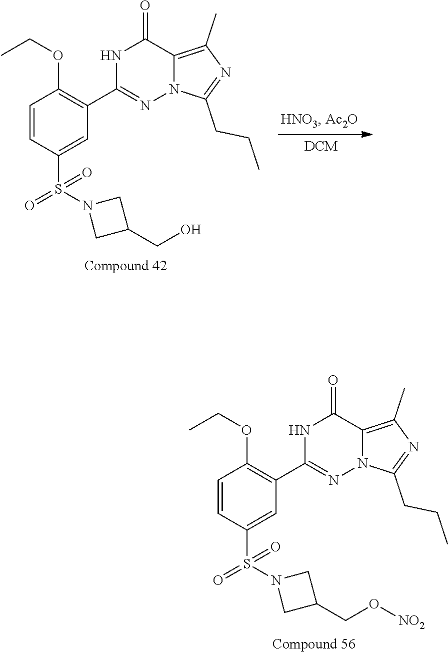 [SMILES: CCCc1nc(C)c2c(=O)[nH]c(-c3cc(S(=O)(=O)N4CC(CO)C4)ccc3OCC)nn12 ; CCCc1nc(C)c2c(=O)[nH]c(-c3cc(S(=O)(=O)N4CC(CO[N+](=O)[O-])C4)ccc3OCC)nn12]

To a solution of compound 42, 2-(2-ethoxy-5-((3-(hydroxymethyl)azetidin-1-yl)sulfonyl)phenyl)-5-methyl-7-propyl imidazo[5,1-f][1,2,4]triazin-4(3H)-one (220 mg, 477 umol) in DCM (10 mL) was added HNO₃ (90 mg, 1.43 mmol) and Ac₂O (152 mg, 1.43 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 56, (1-((4-ethoxy-3-(5-methyl-4-oxo-7-propyl-3,4-dihydroimidazo[5,1-f][1,2,4]triazin-2-yl)phenyl)sulfonyl)azetidin-3-yl)methyl nitrate (97 mg, 40.17% yield) as a white solid. ¹H NMR (400 MHz, DMSO-d₆) δ 11.56 (bs, 1H), 7.97-7.93 (m, 2H), 7.42 (d, 1H), 4.49 (d, 2H), 4.24 (q, 2H), 3.83 (t, 2H), 3.61-3.58 (m, 2H), 2.84-2.77 (m, 3H), 2.48 (s, 3H), 1.76-1.70 (m, 2H), 1.34 (t, 3H), 0.92 (t, 3H); MS: m/z=507.1 (M+1, ESI+); HRMS: 507.1659.

### Synthesis of Compound 57

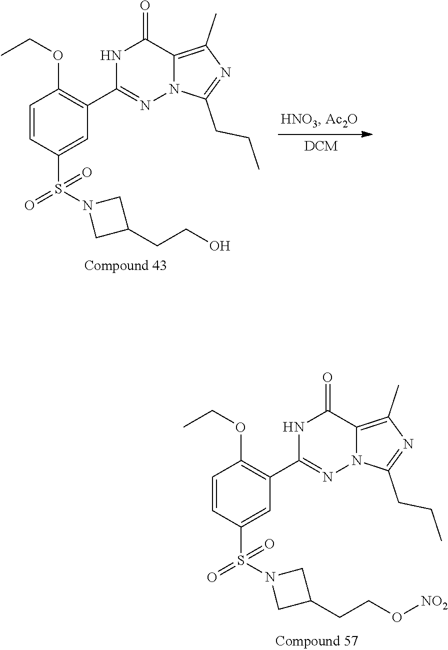 [SMILES: CCCc1nc(C)c2c(=O)[nH]c(-c3cc(S(=O)(=O)N4CC(CCO)C4)ccc3OCC)nn12 ; CCCc1nc(C)c2c(=O)[nH]c(-c3cc(S(=O)(=O)N4CC(CCO[N+](=O)[O-])C4)ccc3OCC)nn12]

To a solution of compound 43, 2-(2-ethoxy-5-((3-(2-hydroxyethyl)azetidin-1-yl)sulfonyl)phenyl)-5-methyl-7-propyl imidazo[5,1-f][1,2,4]triazin-4(3H)-one (220 mg, 463 umol) in DCM (8 mL) was added HNO₃ (63 mg, 1.39 mmol) and Ac₂O (147 mg, 1.39 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 57, 2-(1-((4-ethoxy-3-(5-methyl-4-oxo-7-propyl-3,4-dihydroimidazo[5,1-f][1,2,4]triazin-2-yl)phenyl)sulfonyl)azetidin-3-yl)ethyl nitrate (108 mg, 44.85% yield) as a white solid. ¹H NMR (400 MHz, DMSO-d₆) δ 10.53 (bs, 1H), 7.95-7.92 (m, 2H), 7.42 (d, 1H), 4.41 (t, 2H), 4.24 (q, 2H), 3.82 (t, 2H), 3.43-3.39 (m, 2H), 2.82 (t, 2H), 2.48 (s, 3H), 1.78-1.68 (m, 4H), 1.34 (t, 3H), 0.91 (t, 3H); MS: m/z=521.3 (M+1, ESI+); HRMS: 521.1815.

### Synthesis of Compound 58

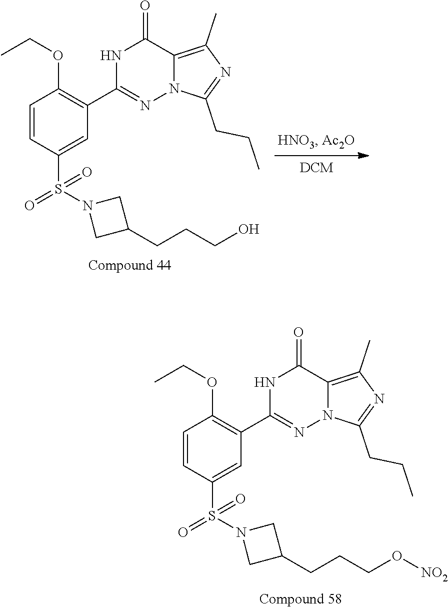

To a solution of compound 44, 2-(2-ethoxy-5-((3-(3-hydroxypropyl)azetidin-1-yl)sulfonyl)phenyl)-5-methyl-7-propyl imidazo[5,1-f][1,2,4]triazin-4(3H)-one (260 mg, 531 umol) in DCM (10 mL) was added HNO₃ (154 mg, 1.59 mmol) and Ac₂O (102 mg, 1.59 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 58, 3-(1-((4-ethoxy-3-(5-methyl-4-oxo-7-propyl-3,4-dihydroimidazo[5,1-f][1,2,4]triazin-2-yl)phenyl)sulfonyl)azetidin-3-yl)propyl nitrate (80 mg, 28% yield) as a white solid. ¹H NMR (400 MHz, DMSO-d₆) δ 11.41 (bs, 1H), 7.95-7.92 (m, 2H), 7.42 (d, 1H), 4.42 (t, 2H), 4.24 (q, 2H), 3.80 (t, 2H), 3.37-3.32 (m, 2H), 2.82 (t, 2H), 2.48 (s, 3H), 2.44-2.36 (m, 1H), 1.78-1.69 (m, 2H), 1.54-1.47 (m, 2H), 1.40-1.32 (m, 5H), 0.92 (t, 3H); MS: m/z=535.3 (M+1, ESI+); HRMS: 535.1972.

### Synthesis of Compound 61

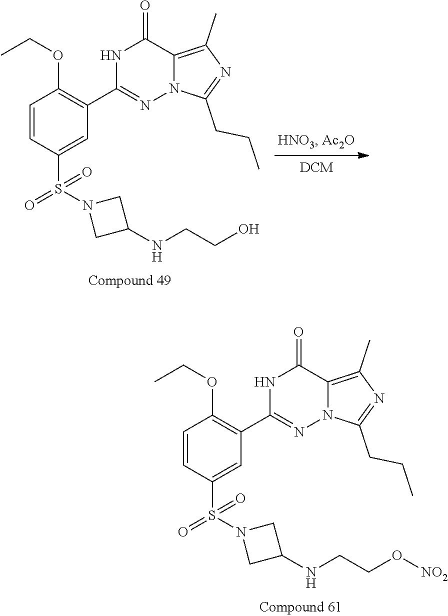

To a solution of compound 49, 2-(2-ethoxy-5-((3-((2-hydroxyethyl)amino)azetidin-1-yl)sulfonyl)phenyl)-5-methyl-7-propylimidazo[5,1-f][1,2,4]triazin-4(3H)-one (200 mg, 408 umol) in DCM (10 mL) was added HNO₃ (77 mg, 1.22 mol) and Ac₂O (124 mg, 1.22 mol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 61, 2-((1-((4-ethoxy-3-(5-methyl-4-oxo-7-propyl-3,4-dihydroimidazo[5,1-f] [1,2,4]triazin-2-yl)phenyl)sulfonyl)azetidin-3-yl)amino)ethyl nitrate (50 mg, 22% yield) as a white solid. ¹H NMR (400 MHz, DMSO-d₆) δ 11.74 (bs, 1H), 7.95-7.93 (m, 2H), 7.42 (d, 1H), 4.48-4.45 (m, 2H), 4.24 (q, 2H), 3.84-3.82 (m, 2H), 3.46-3.44 (m, 3H), 3.32-3.31 (m, 1H), 2.85-2.82 (m, 2H), 2.73-2.72 (m, 2H), 2.49 (s, 3H), 1.77-1.71 (m, 2H), 1.36-1.33 (m, 3H), 0.92 (t, 3H); MS: m/z=536.1 (M+1, ESI+); HRMS: 536.1920.

### Synthesis of Compound 62

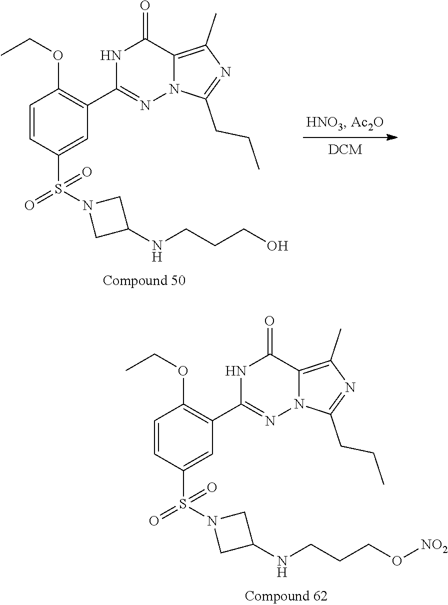 [SMILES: CCCc1nc(C)c2c(=O)[nH]c(-c3cc(S(=O)(=O)N4CC(NCCCO)C4)ccc3OCC)nn12 ; CCCc1nc(C)c2c(=O)[nH]c(-c3cc(S(=O)(=O)N4CC(NCCCO[N+](=O)[O-])C4)ccc3OCC)nn12]

To a solution of compound 50, 2-(2-ethoxy-5-((3-((3-hydroxypropyl)amino)azetidin-1-yl)sulfonyl)phenyl)-5-methyl-7-propylimidazo[5,1-f][1,2,4]triazin-4(3H)-one (200 mg, 396 umol) in DCM (8 mL) was added HNO₃ (115 mg, 1.19 mol) and Ac₂O (126 mg, 1.19 mol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 62, 3-((1-((4-ethoxy-3-(5-methyl-4-oxo-7-propyl-3,4-dihydroimidazo[5,1-f] [1,2,4]triazin-2-yl)phenyl)sulfonyl)azetidin-3-yl)amino) propyl nitrate (95 mg, 43.61% yield) as a white solid. ¹H NMR (400 MHz, DMSO-d₆) δ 11.79 (bs, 1H), 8.00-7.95 (m, 2H), 7.47-7.45 (m, 1H), 4.55-4.50 (m, 2H), 4.36-4.20 (m, 2H), 3.90-3.87 (m, 2H), 3.49-3.43 (m, 4H), 2.90-2.86 (m, 2H), 2.55-2.42 (m, 5H), 1.81-1.71 (m, 4H), 1.41-1.36 (m, 3H), 0.99-0.94 (m, 3H); MS: m/z=550.3 (M+1, ESI+); HRMS: 550.2081.

### Synthesis of Compound 63

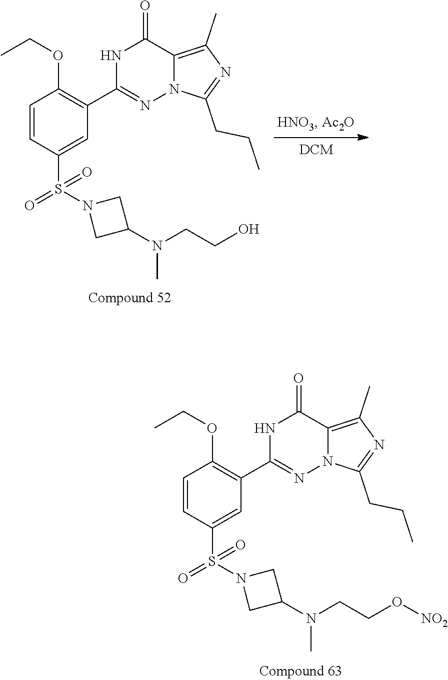

To a solution of compound 52, 2-(2-ethoxy-5-((3-((2-hydroxyethyl)(methyl)amino)azetidin-1-yl)sulfonyl)phenyl)-5-methyl-7-propylimidazo[5,1-f][1,2,4]triazin-4(3H)-one (300 mg, 596 umol) in DCM (6 mL) was added HNO₃ (188 mg, 2.98 mol) and Ac₂O (316 mg, 2.98 mol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 63, 2-((1-((4-ethoxy-3-(5-methyl-4-oxo-7-propyl-3,4-dihydroimidazo[5,1-f][1,2,4]triazin-2-yl)phenyl)sulfonyl)azetidin-3-yl)(methyl)amino)ethyl nitrate (40 mg, 12.12% yield) as a white solid. ¹H NMR (400 MHz, DMSO-d₆) δ 11.74 (bs, 1H), 7.97-7.94 (m, 2H), 7.42 (d, 1H), 4.49 (t, 2H), 4.24 (q, 2H), 3.78 (t, 2H), 3.52 (t, 2H), 3.34-3.29 (m, 1H), 2.83 (t, 2H), 2.54-2.51 (m, 2H), 2.49 (s, 3H), 2.00 (s, 3H), 1.76-1.71 (m, 2H), 1.35 (t, 3H), 0.92 (t, 3H); MS: m/z=550.2 (M+1, ESI+); HRMS: 550.2075.

### Synthesis of Compound 64

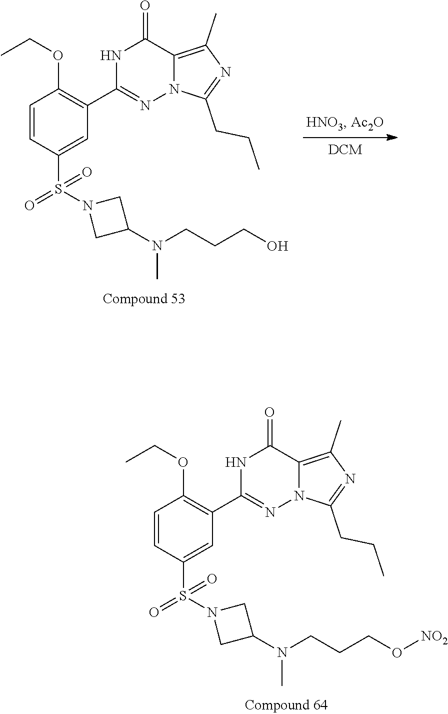 [SMILES: CCCc1nc(C)c2c(=O)[nH]c(-c3cc(S(=O)(=O)N4CC(N(C)CCCO)C4)ccc3OCC)nn12 ; CCCc1nc(C)c2c(=O)[nH]c(-c3cc(S(=O)(=O)N4CC(N(C)CCCO[N+](=O)[O-])C4)ccc3OCC)nn12]

To a solution of compound 53, 2-(2-ethoxy-5-((3-((3-hydroxypropyl)(methyl)amino)azetidin-1-yl)sulfonyl)phenyl)-5-methyl-7-propylimidazo[5,1-f][1,2,4]triazin-4(3H)-one (300 mg, 578 umol) in DCM (20 mL) was added HNO₃ (109 mg, 1.74 mol) and Ac₂O (177 mg, 1.74 mol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 64, 3-((1-((4-ethoxy-3-(5-methyl-4-oxo-7-propyl-3,4-dihydroimidazo[5,1-f][1,2,4]triazin-2-yl)phenyl)sulfonyl)azetidin-3-yl)(methyl) amino)propyl nitrate (200 mg, 61.34% yield) as a white solid. ¹H NMR (400 MHz, DMSO-d₆) δ 11.74 (bs, 1H), 7.93-7.91 (m, 2H), 7.41 (d, 1H), 4.41-4.38 (m, 2H), 4.25-4.22 (m, 2H), 3.81-3.78 (m, 2H), 3.49-3.46 (m, 2H), 3.17-3.14 (m, 1H), 2.85-2.81 (m, 2H), 2.48 (s, 3H), 2.16-2.13 (m, 2H), 1.91 (s, 3H), 1.76-1.671 (m, 4H), 1.35 (t, 3H), 0.93 (t, 3H); MS: m/z=564.0 (M+1, ESI+); HRMS: 564.2233.

### Example 4—Preparation of Substituted Amino-Azetidine-Linked imidazo[5,1-f][1,2,4]triazin-4(3H)-one Compounds

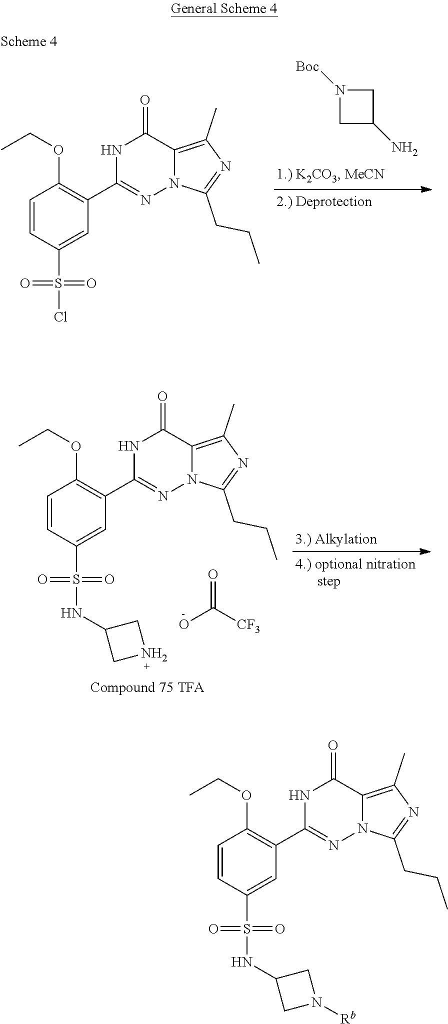

### Synthesis of Compound 46

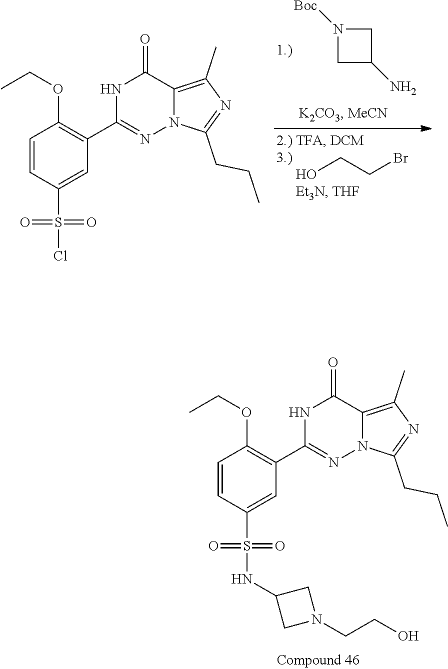

### Step 1:

To a solution of 4-ethoxy-3-(5-methyl-4-oxo-7-propyl-3,4-dihydroimidazo[5,1-f][1,2,4]triazin-2-yl) benzenesulfonyl chloride (2 g, 4.87 mmol) and tert-butyl 3-aminoazetidine-1-carboxylate (1.26 g, 7.30 mmol) in MeCN (20 mL) was added K₂CO₃ (2.02 g, 14.60 mmol), the reaction mixture was stirred at 100° C. for 4 h. The reaction mixture was poured into water (200 mL), extracted with EA (50 mL×3), washed by brine (50 mL×3), dried over Na₂SO₄ and concentrated, the residue was purified by column chromatography to afford tert-butyl 3-((4-ethoxy-3-(5-methyl-4-oxo-7-propyl-3,4-dihydroimidazo[5,1-f] [1,2,4]triazin-2-yl)phenyl)sulfonamido)azetidine-1-carboxylate (2.5 g, 93.95% yield) as a white solid. MS: m/z=547.4 (M+1, ESI+).

### Step 2:

A mixture of tert-butyl 3-((4-ethoxy-3-(5-methyl-4-oxo-7-propyl-3,4-dihydroimidazo[5,1-f][1,2,4] triazin-2-yl)phenyl)sulfonamido)azetidine-1-carboxylate (2.5 g, 4.57 mmol) in DCM (10 mL) was added TFA (5 mL) and stirred at 25° C. for 4 h. The reaction mixture was evaporated under reduced pressure to afford compound 75; 2,2,2-trifluoroacetate salt, N-(azetidin-3-yl)-4-ethoxy-3-(5-methyl-4-oxo-7-propyl-3,4-dihydroimidazo[5,1-f][1,2,4]triazin-2-yl) benzenesulfonamide; 2,2,2-trifluoroacetate salt (1.9 g, 93.04% yield) as a yellow oil. MS: m/z=447.1 (M+1, ESI+).

### Step 3:

To a solution of compound 75; 2,2,2-trifluoroacetate salt, N-(azetidin-3-yl)-4-ethoxy-3-(5-methyl-4-oxo-7-propyl-3,4-dihydroimidazo[5,1-f] [1,2,4] triazin-2-yl)benzenesulfonamide; 2,2,2-trifluoroacetate salt (500 mg, 1.12 mmol) and 2-bromoethan-1-ol (280 mg, 2.24 mmol) in THF (10 mL) was added TEA (340 mg, 3.36 mmol), the reaction mixture was stirred at 80° C. for 16 h. The reaction mixture was poured into water (50 mL), extracted with EA (20 mL×3), washed by brine (30 mL×3), dried over Na₂SO₄ and concentrated, the residue was purified by prep-HPLC to afford compound 46, 4-ethoxy-N-(1-(2-hydroxyethyl)azetidin-3-yl)-3-(5-methyl-4-oxo-7-propyl-3,4-dihydroimidazo[5,1-f] [1,2,4]triazin-2-yl)benzenesulfonamide (230 mg, 41.87% yield) as a white solid. ¹H NMR (400 MHz, DMSO-d₆) δ 11.70 (bs, 1H), 8.16 (bs, 1H), 7.91-7.89 (m, 2H), 7.34 (d, 1H), 4.34 (bs, 1H), 4.20 (q, 2H), 3.76-3.73 (m, 1H), 3.37-3.34 (m, 2H), 3.38-3.24 (m, 2H), 2.84 (t, 2H), 2.70-2.66 (m, 2H), 2.49 (s, 3H), 2.36-2.34 (m, 2H), 1.79-1.70 (m, 2H), 1.33 (t, 3H), 0.93 (t, 3H); MS: m/z=491.2 (M+1, ESI+); HRMS: 491.2073.

### Synthesis of Compound 47

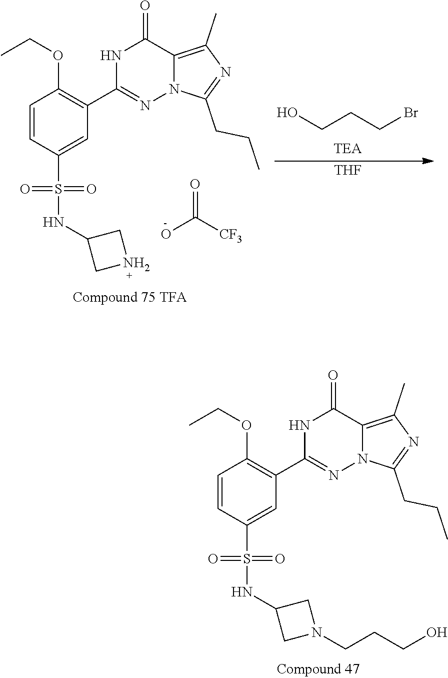 [SMILES: CCCc1nc(C)c2c(=O)[nH]c(-c3cc(S(=O)(=O)NC4CNC4)ccc3OCC)nn12 ; O=C(O)C(F)(F)F ; OCCCBr ; CCCc1nc(C)c2c(=O)[nH]c(-c3cc(S(=O)(=O)NC4CN(CCCO)C4)ccc3OCC)nn12 ; "THF"]

To a solution of compound 75; 2,2,2-trifluoroacetate salt, N-(azetidin-3-yl)-4-ethoxy-3-(5-methyl-4-oxo-7-propyl-3,4-dihydroimidazo[5,1-f] [1,2,4]triazin-2-yl)benzenesulfonamide; 2,2,2-trifluoroacetate salt (500 mg, 1.12 mmol) and 3-bromopropan-1-ol (311 mg, 2.24 mmol) in THF (10 mL) was added TEA (340 mg, 3.36 mmol), the reaction mixture was stirred at 80° C. for 16 h. The reaction mixture was poured into water (50 mL), extracted with EA (20 mL×3), washed by brine (30 mL×3), dried over Na₂SO₄ and concentrated, the residue was purified by prep-HPLC to afford compound 47, 4-ethoxy-N-(1-(3-hydroxypropyl)azetidin-3-yl)-3-(5-methyl-4-oxo-7-propyl-3,4-dihydro imidazo[5,1-f][1,2,4]triazin-2-yl)benzenesulfonamide (270 mg, 47.78% yield) as a white solid. ¹H NMR (400 MHz, DMSO-d₆) δ 11.71 (bs, 1H), 8.15 (bs, 1H), 7.91-7.89 (m, 2H), 7.34 (d, 1H), 4.34 (bs, 1H), 4.20 (q, 2H), 3.76-3.72 (m, 1H), 3.35-3.30 (m, 4H), 2.84 (t, 2H), 2.58 (t, 2H), 2.48 (s, 3H), 2.30 (t, 2H), 1.77-1.70 (m, 2H), 1.34-1.31 (m, 5H), 0.93 (t, 3H); MS: m/z=505.2 (M+1, ESI+); HRMS: 505.2230

### Synthesis of Compound 48

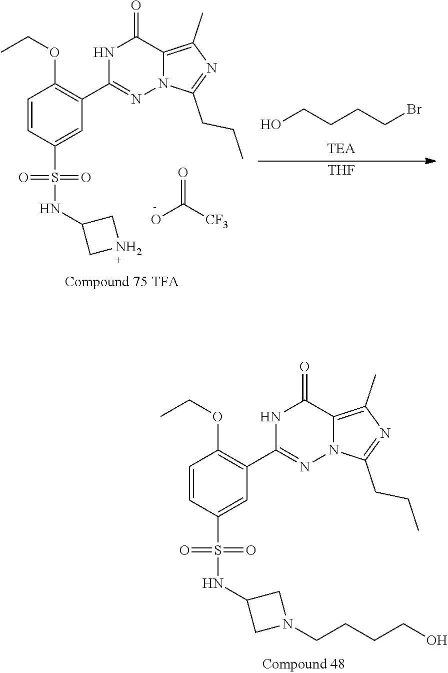

To a solution of compound 75; 2,2,2-trifluoroacetate salt, N-(azetidin-3-yl)-4-ethoxy-3-(5-methyl-4-oxo-7-propyl-3,4-dihydroimidazo[5,1-f] [1,2,4] triazin-2-yl)benzenesulfonamide; 2,2,2-trifluoroacetate salt (800 mg, 1.79 mmol) and 4-bromobutan-1-ol (549 mg, 3.59 mmol) in THF (10 mL) was added TEA (544 mg, 5.37 mmol), the reaction mixture was stirred at 80° C. for 16 h. The reaction mixture was poured into water (50 mL), extracted with EA (20 mL×3), washed by brine (30 mL×3), dried over Na₂SO₄ and concentrated, the residue was purified by prep-HPLC to afford compound 48, 4-ethoxy-N-(1-(4-hydroxybutyl)azetidin-3-yl)-3-(5-methyl-4-oxo-7-propyl-3,4-dihydroimidazo[5,1-f] [1,2,4]triazin-2-yl)benzenesulfonamide (420 mg, 45.20% yield) as a white solid. ¹H NMR (400 MHz, DMSO-d₆) δ 11.71 (bs, 1H), 8.15 (bs, 1H), 7.91-7.89 (m, 2H), 7.34 (d, 1H), 4.34 (bs, 1H), 4.22-4.17 (m, 2H), 3.76-3.72 (m, 1H), 3.35-3.30 (m, 4H), 2.85-2.82 (m, 2H), 2.60-2.56 (m, 2H), 2.48 (s, 3H), 2.32-2.29 (m, 2H), 1.77-1.70 (m, 2H), 1.34-1.18 (m, 7H), 0.95-0.91 (m, 3H); MS: m/z=519.2 (M+1, ESI+); HRMS: 519.2388.

### Synthesis of Compound 59

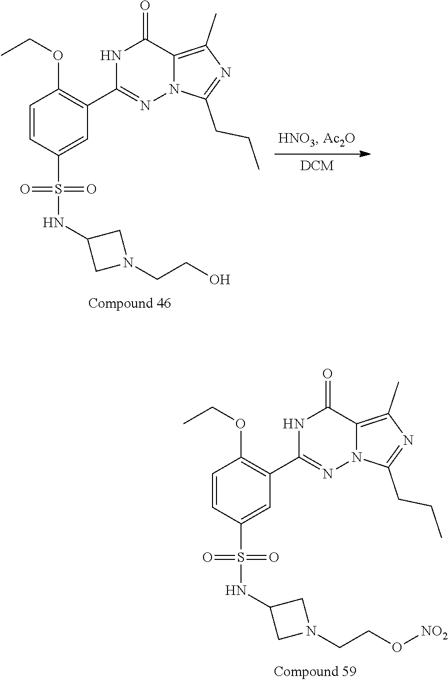 [SMILES: CCCc1nc(C)c2c(=O)[nH]c(-c3cc(S(=O)(=O)NC4CN(CCO)C4)ccc3OCC)nn12 ; CCCc1nc(C)c2c(=O)[nH]c(-c3cc(S(=O)(=O)NC4CN(CCO[N+](=O)[O-])C4)ccc3OCC)nn12]

To a solution of compound 46, 4-ethoxy-N-(1-(2-hydroxyethyl)azetidin-3-yl)-3-(5-methyl-4-oxo-7-propyl-3,4-dihydro imidazo[5,1-f][1,2,4]triazin-2-yl)benzenesulfonamide (250 mg, 510 umol) in DCM (10 mL) was added HNO₃ (96 mg, 1.53 mmol) and Ac₂O (162 mg, 1.53 mmol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 59, 2-(3-((4-ethoxy-3-(5-methyl-4-oxo-7-propyl-3,4-dihydro imidazo[5,1-f][1,2,4]triazin-2-yl)phenyl)sulfonamido)azetidin-1-yl)ethyl nitrate (84 mg, 30.78% yield) as a white solid. ¹H NMR (400 MHz, DMSO-d₆) δ 11.66 (bs, 1H), 8.18 (bs, 1H), 7.91-7.89 (m, 2H), 7.34 (d, 1H), 4.42-4.40 (m, 2H), 4.22-4.17 (m, 2H), 3.79-3.75 (m, 1H), 3.42-3.34 (m, 5H), 2.85-2.82 (m, 2H), 2.77-2.74 (m, 2H), 2.64-2.62 (m, 2H), 1.77-1.70 (m, 2H), 1.35-1.31 (m, 3H), 0.93 (t, 3H); MS: m/z=536.3 (M+1, ESI+); HRMS: 536.1923.

### Synthesis of Compound 60

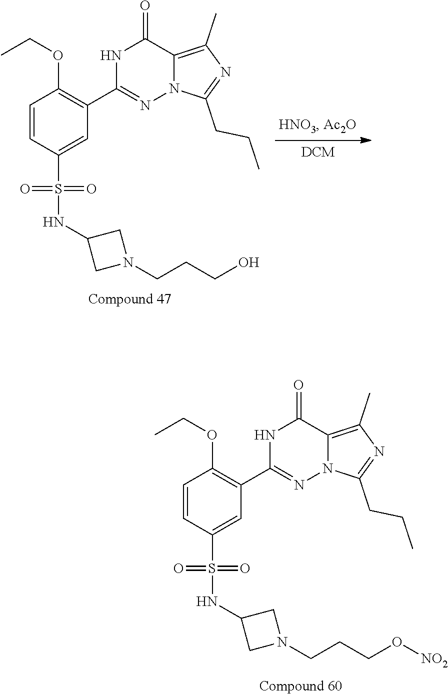

To a solution of compound 47, 4-ethoxy-N-(1-(3-hydroxypropyl)azetidin-3-yl)-3-(5-methyl-4-oxo-7-propyl-3,4-dihydro imidazo[5,1-f][1,2,4]triazin-2-yl)benzenesulfonamide (140 mg, 277 umol) in DCM (10 mL) was added HNO₃ (52 mg, 832 umol) and Ac₂O (88 mg, 832 umol), the reaction mixture was stirred at 25° C. for 16 h. The resulting solution was poured into water (50 mL) and extracted with DCM (20 mL×3). The combined organic layers were dried over Na₂SO₄ and concentrated under reduced pressure, the residue was purified by Prep-HPLC to afford compound 60, 3-(3-((4-ethoxy-3-(5-methyl-4-oxo-7-propyl-3,4-dihydroimidazo[5,1-f][1,2,4]triazin-2-yl)phenyl)sulfonamido)azetidin-1-yl) propyl nitrate (62 mg, 40.66% yield) as a white solid. ¹H NMR (400 MHz, DMSO-d₆) δ 7.92-7.90 (m, 2H), 7.34 (d, 1H), 4.46 (t, 2H), 4.20 (q, 2H), 3.77-3.74 (m, 1H), 3.38-3.34 (m, 2H), 2.84 (t, 2H), 2.63 (t, 2H), 2.49 (s, 3H), 2.35 (t, 2H), 1.77-1.71 (m, 2H), 1.62-1.56 (m, 2H), 1.33 (t, 3H), 0.93 (t, 3H); MS: m/z=550.2 (M+1, ESI+); HRMS: 550.2081.

### Example 5—Human PDE-5A1 and/or -6C Inhibition Assay

This example illustrates the in vitro inhibition of human PDE-5A1 and/or -6C by the compounds as described herein.

### Materials

Sildenafil citrate (Catalog no. LKT-S3313, Axxora, San Diego, Calif.), Vardenafil hydrochloride trihydrate (Catalog no. SML2103, Sigma-Aldrich, St. Louis, Mo.), PDE Assay Buffer (Catalog no. 60393, BPS bioscience, San Diego, Calif.), PDE Binding Agent (Catalog no. 60390, BPS bioscience, San Diego, Calif.) and PDE Binding Agent Diluent (cGMP, Catalog no. 60392, BPS bioscience, San Diego, Calif.) were used for assays. Test compounds were supplied by Ildong Pharmaceuticals Co., Ltd.

### Experimental Protocols

The enzymes and substrates used in this experiment are summarized in Table 2.

TABLE 2
Enzymes and Substrates

| | | Enzyme | Enzyme Used | |
|---|---|---|---|---|
| Assay | Catalog # | Lot# | (ng/reaction) | Substrate |
| PDE5A1 | 60050 | 170404-G | 0.2 | 100 nM FAM-cGMP |
| | | and | | |
| | | 181008-G | | |
| PDE6C | 60060 | 160928-AC | 0.5 | 100 nM FAM-cGMP |
| | | and | | |
| | | 190912-A | | |

The serial dilution of the compounds was first performed in 100% DMSO with the highest concentration at 1 mM and 0.1 mM. Each intermediate compound dilution (in 100% DMSO) will then get directly diluted 10× fold into assay buffer for 10% DMSO and 5 μL of the dilution was added to a 50 μL reaction so that the final concentration of DMSO is 1% in all reactions.

The enzymatic reactions were conducted at room temperature for 60 minutes in a 50 μL mixture containing PDE assay buffer, 100 nM FAM-cGMP, a PDE enzyme (Table 2) and the test compounds.

After enzymatic reaction, 100 μL of a binding solution (1:100 dilution of the binding agent with the binding agent diluent) was added to each reaction and the reaction was performed at room temperature for 60 minutes.

Fluorescence intensity was measured at excitation of 485 nm and an emission of 528 nm using a Tecan Infinite M1000 microplate reader.

### Data Analysis

PDE activity assays were performed in duplicate at each concentration. Fluorescence intensity is converted to fluorescence polarization using the Tecan Magellan6 software. The fluorescence polarization (FPₜ) in absence of the compound in each data set was defined as 100% activity. In the absence of PDE and the compound, the value fluorescent polarization (FP_{b}) in each data set was defined as 0% activity. The percent activity in the presence of compound was calculated according to Equation 1:

$$\% activity = ( \frac{FP - {FP}_{b}}{{FP}_{t} - {FP}_{b}} ) \times 100 \qquad ( eqn . 1 )$$

where FP=the fluorescence polarization in the presence of the compound.

The values of % activity versus a series of compound concentrations were then plotted using non-linear regression analysis of Sigmoidal dose-response curve generated with Equation 2:

$$Y = B + ( \frac{T - B}{1 + {10}^{( LogEC 50 - X ) \times Hill Slope}} ) \times 100 \qquad ( eqn . 2 )$$

where Y=percent activity, B=minimum percent activity, T=maximum percent activity, X=logarithm of compound, and Hill Slope=slope factor or Hill coefficient. The IC₅₀ value was determined by the concentration causing a half-maximal percent activity.

### Results

The results are tabulated in Table 3 with IC₅₀ values shown as ranges.

TABLE 3
In Vitro Inhibition of PDE-5A1 and/or -6C Activities

| | IC₅₀ (μM) | | | IC₅₀ (μM) | |
|---|---|---|---|---|---|
| | A: IC₅₀ ≤ 0.010 μM | | | A: IC₅₀ ≤ 0.010 μM | |
| | B: 0.010 μM < IC₅₀ ≤ | | | B: 0.010 μM < IC₅₀ ≤ | |
| | 0.1 μM | | | 0.1 μM | |
| Cmpd | C: 0.1 μM < IC₅₀ | | Cmpd | C: 0.1 μM < IC₅₀ | |
| No. | PDE5A1 | PDE6C | No. | PDE5A1 | PDE6C |
| 1 | B | B | 2 | B | B |
| 3 | A | A | 4 | A | A |
| 5 | C | B | 6 | B | B |
| 7 | B | B | 8 | B | B |
| 9 | B | B | 10 | A | A |
| 11 | B | B | 12 | A | A |
| 13 | A | B | 14 | A | A |
| 15 | B | B | 16 | A | B |
| 17 | A | A | IS | A | A |
| 19 | A | A | 20 | B | A |
| 21 | A | A | 22 | A | A |
| 23 | A | A | 24 | B | A |
| 25 | B | B | 26 | B | B |
| 27 | B | A | 28 | B | B |
| 29 | B | B | 30 | A | A |
| 31 | B | A | 32 | A | B |
| 33 | A | A | 34 | B | A |
| 35 | B | A | 36 | B | B |
| 37 | B | B | 38 | B | B |
| 39 | A | A | 40 | B | A |
| 41 | A | — | 42 | A | — |
| 43 | A | — | 44 | A | — |
| 45 | A | — | 46 | A | — |
| 47 | A | — | 48 | — | — |
| 49 | A | — | 50 | A | — |
| 51 | — | — | 52 | A | — |
| 53 | A | — | 54 | — | — |
| 55 | A | — | 56 | A | — |
| 57 | A | — | 58 | A | — |
| 59 | A | — | 60 | A | — |
| 61 | A | — | 62 | A | — |
| 63 | A | — | 64 | A | — |
| 65 | A | A | 66 | A | A |
| 67 | A | A | 68 | B | A |
| 69 | A | A | 70 | A | A |
| 71 | A | A | 72 | A | A |
| 73 | A | A | Sildenafil | A | B |
| Vardenafil | A | — | | | |

Sildenafil and Vardenafil were used as reference compounds in the human PDE-5A1 and/or -6C assays.

The chemical structure of Sildenafil is:

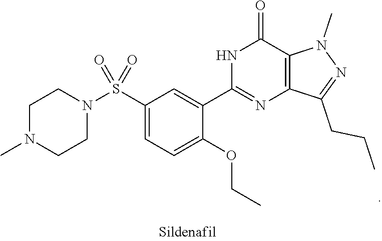 [SMILES: CCCc1nn(C)c2c(=O)[nH]c(-c3cc(S(=O)(=O)N4CCN(C)CC4)ccc3OCC)nc12]

The chemical structure of Vardenafil is:

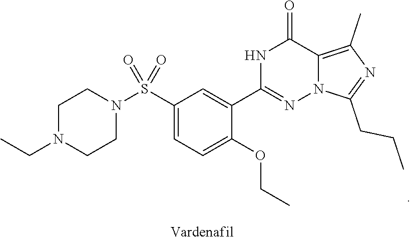 [SMILES: CCCc1nc(C)c2c(=O)[nH]c(-c3cc(S(=O)(=O)N4CCN(CC)CC4)ccc3OCC)nn12]

### Conclusion

The PDE-5A1 and/or -6C inhibitory activities of the tested compounds were comparable, and in some cases superior, to those of Sildenafil and Vardenafil.

### Example 6—Metabolic Stability Assays in Human Liver Microsomes

This example illustrates the metabolic stability of selected compounds in human liver microsomes samples.

### Materials

Both test and control compound solutions were prepared by diluting 5 μL of the respective stock solutions (10 mM in DMSO) containing either the test or control compound with 495 μL of acetonitrile (ACN) to give intermediate solutions with concentrations of 100 μM (99% ACN).

β-Nicotinamide adenine dinucleotide phosphate tetrasodium salt (NADPH.4Na) was purchased from BONTAC (cat. No. BT04). The NADPH working solution (10 unit/mL) was prepared by combining the appropriate amount of NADPH powder and a MgCl₂ solution (10 mM) to give final concentration in the reaction system of 1 unit/mL.

The appropriate concentrations of the microsome working solutions were prepared in 100 mM potassium phosphate buffer.

Cold (4° C.) acetonitrile solution containing 200 ng/mL tolbutamide and 200 ng/mL labetalol (internal standard) was used as the stop solution.

### Experimental Protocols

Liver microsomes solution was diluted to 0.56 mg/mL in 100 mM phosphate buffer, and 445 μL of this solution was transferred into pre-warmed (10 minutes) “incubation” plates T60 and NCF60; the “incubation” plates T60 and NCF60 were pre-warmed for 10 minutes at 37° C. with constant shaking.

54 μL of the liver microsome solutions was transferred to the blank plate, followed by addition of 6 μL of the NAPDH cofactor solution and 180 μL of the quenching solution to the same blank plate.

5 μL of the compound working solution (100 μM concentration) was next added to into the “incubation” plates (T60 and NCF60) containing microsomes and mixed 3 times thoroughly. For the NCF60 plate, 50 μL of the buffer solution was added, mixed 3 times thoroughly, and incubated at 37° C. for 60 minutes under constant shaking.

In the “Quenching” plate at T0 (T=0 min), 180 μL of quenching solution and 6 μL of the NAPDH cofactor solution were added, and the resulting plate was chilled to prevent evaporation.

In the T60 plate, after it was thoroughly mixed, 54 μL of the mixture was immediately transferred to the “quenching” plate for the 0-minute time point, followed by addition of 44 μL of NAPDH cofactor solution to the incubation plate (T60). The resulting mixture was then incubated at 37° C. for 60 minutes under constant shaking. At time points of 5, 10, 20, 30 and 60 minutes, 180 μL of the quenching solution was added to the “quenching” plates, followed by serial transfer of 60 μL of the mixture (per time point) from the T60 plate to the “Quenching” plates.

For the NCF60 plates, 60 μL of the sample solution was transferred from the NCF60 incubation plate to the “Quenching” plate containing quenching solution at T=60 min time point.

All sampling plates were shaken for 10 minutes and then centrifuged at 4,000 rpm for 20 minutes at 4° C., followed by the transfer of 60 μL of the supernatant into 180 μL of High-Pressure Liquid Chromatography (HPLC) water and mixed for 10 minutes by a plate shaker. Each bioanalysis plate was then sealed and shaken for 10 minutes prior to liquid chromatography-mass spectrometry (LC-MS)/mass spectrometry (MS) analysis.

### Results

The metabolic stability assay data for compounds 4, 10, 18 and 22 in human liver microsomes are shown in Table 4.

TABLE 4
Metabolic Stability in Human
Liver Microsomes

| Compound No. | Clᵢₙₜ₍ₗᵢᵥₑᵣ₎ (mL/min/kg) |
|---|---|
| 4 | 357.0 |
| 10 | 55.0 |
| 18 | 934.3 |
| 22 | 357.8 |

### Conclusion

The high clearance observed in human liver microsomes of the tested compounds demonstrated reduction in off-target effect of the tested compounds and reduction in the compounds' effects for other targets other than PDE-5 and/or -6.

### Example 7—Plasma Binding Assay

This example shows the procedures and results of the plasma protein binding assays of selected compounds.

### Equipment

The dialysis device used in this example is a 96-well equilibrium dialysis plate (Cat #1006, HT Dialysis LLC, Gales Gerry, CT), and HTD 96 a/b dialysis membrane strips (Cat #1101, MWCO 12-14 kDa, HT Dialysis LLC). The dialysis device was assembled following the manufacturer's instructions.

### Materials

The dialysis membrane strips were soaked in ultra-pure water at room temperature for approximately 1 hour. Each membrane strip containing 2 membranes was separated and soaked in 20:80 ethanol/water (v/v) for approximately 20 minutes, after which they were ready for used or were stored in the solution at 2-8° C. for up to a month. Prior to experiment, the membrane was rinsed and soaked for 20 minutes in ultra-pure water.

On the day of the experiment, the plasma was thawed by running under col tap water and centrifuged at 3220 rpm for 5 minutes to remove any clos. The pH value of the resulting plasma was checked. Only plasma with pH value with 7.0-8.0 could be used.

Both test and control compounds were dissolved in DMSO to achieve 10 mM stock solutions. Working solutions (400 μM) if test and control compounds were prepared by diluting 10μ of stock solutions with 240 μL of DMSO. Loading matrix solutions (2 μM) of both test and control compounds were prepared by diluting 5 μL of working solutions with 995 μL of blank matrix.

### Dialysis Protocols

To prepare the loading matrix containing the test compound or control compounds, aliquots of either test compound working solutions or control compound working solution were spiked into blank matrix to achieve final test concentrations. The concentration of organic solvent in the final solutions were no more than 1% (normally 0.5%). The samples were mixture thoroughly before being used.

To prepare the time zero (T0) samples to be used for recovery determinations. 50 μL aliquots of loading matrix solution were transferred in triplicate to the sample collection plate. The samples were immediately matched with opposite blank buffer to obtain a final volume of 100 μL of 1:1 matrix/dialysis buffer (v/v) in each well. 500 μL of stop solution were added to these T0 samples. They were then stored at 2-8° C. pending further processes along with other post-dialysis samples.

To load the dialysis device, an aliquot of 150 μL of the loading matrix was transferred to the donor side of each dialysis well in triplicate, and 150 μL of the dialysis buffer was loaded to the received side of the well. The dialysis was placed in humidified incubator at 37° C. with 5% CO₂ on a shaking platform that rated slowly (about 100 rpm) for 4 hours.

At the end of the dialysis, aliquots of 50 μL of samples were taken from both the buffer side and the matrix side of the dialysis deice. These samples were transferred into new 96-well plates (the sample collection plates). Each sample was mixed with an equal volume of opposite blank matrix (buffer or matrix) to reach a final volume of 100 μL of 1:1 matrix/dialysis buffer (v/v) in each well. All samples were further processed by adding 500 μL of stop solution containing internal standards. The mixture was vortexed and centrifuged at 4000 rpm for about 20 minutes. An aliquot of 100 μL of supernatant of all the samples were then removed for LC-MS/MS analysis.

The single blank samples were prepared by transferring 50 μL of blank matrix to a 96 well plate and adding 50 μL of blank PBS buffer to each well. The blank plasma must match the species of plasma used in the plasma side of the well. Then the matrix-matched samples were further processed by adding 500 μL of stop solution containing internal standards, following the same sample processing method as the dialysis samples.

### Results

The results of the human plasma protein binding assay of selected compounds are shown in Table 5.

TABLE 5
Plasma Protein Binding

| Cmpd No. | % Unbound | % Bound |
|---|---|---|
| 4 | 3.99 | 96.01 |
| 10 | 13.81 | 86.19 |
| 18 | 0.48 | 99.52 |
| 22 | 2.47 | 97.53 |

### Conclusion

The tested compounds exhibited moderate to high binding to human plasma proteins and the results demonstrated that the tested compounds were acting in a localized fashion and are amenable to localized applications and administrations.

### Example 8—In Vivo Intraocular Pressure (IOP) Lowering Effect in Rabbit Subjects

This example illustrates the procedures and results of the intraocular pressure (IOP) lowering effect of compound 18 as compared to Latanoprostene bunod and latanoprost at different concentrations in ocular normotensive rabbits.

### Materials

Forty (40) male New Zealand white rabbits were divided into 4 groups with 10 animals per group. Animals were then randomly assigned to groups based on body weight.

### Experimental Procedures

Latanoprostene bunod ophthalmic solution (LBN, 0.024%) and latanoprost eye drops (0.005%) were used as positive controls and dosed with the same volumes into the right eyes of the tested animals in groups 1 and 2 once.

Compound 18 was instilled into the right eyes of the tested animals in groups 3 (10 mg/mL) and 4 (20 mg/mL) at 50 μL per eye once.

All left eyes of the tested animals in each group were dosed with vehicle solution at 50 μL per eye.

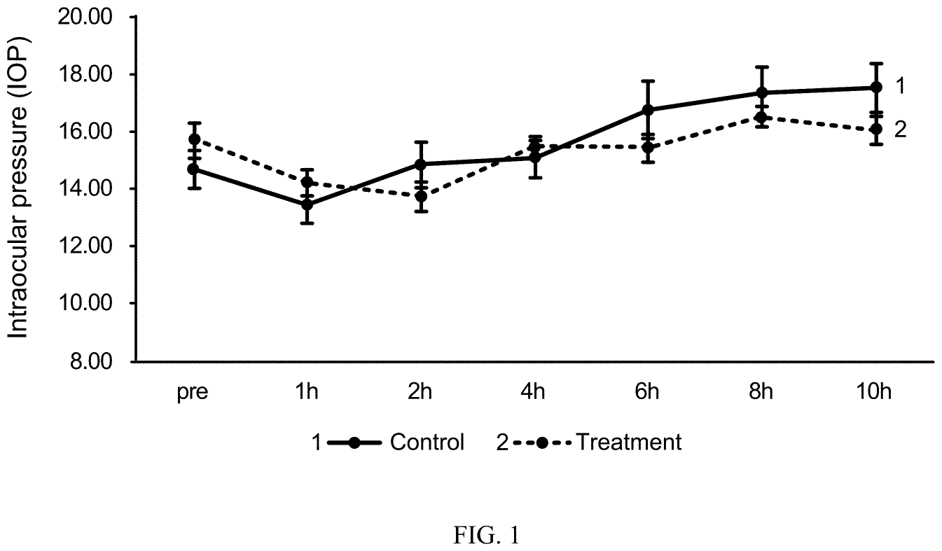

The intraocular pressures (IOPs) were measured once at pre-dose and then once at 1, 2, 4, 6, 8, and 10 hours post dose for each group of animals. FIGS. 1 to 4 show the results from the IOP lowering studies for all four tested groups.

FIG. 1 shows the results from control group 1 of the intraocular pressure (IOP) lowering effect study (mean IOP +/−SEM) with Latanoprostene bunod (0.024%) in ocular normotensive rabbits at various time points after instillation of ophthalmic solutions (control solutions into left eyes and treatment solution into right eyes).

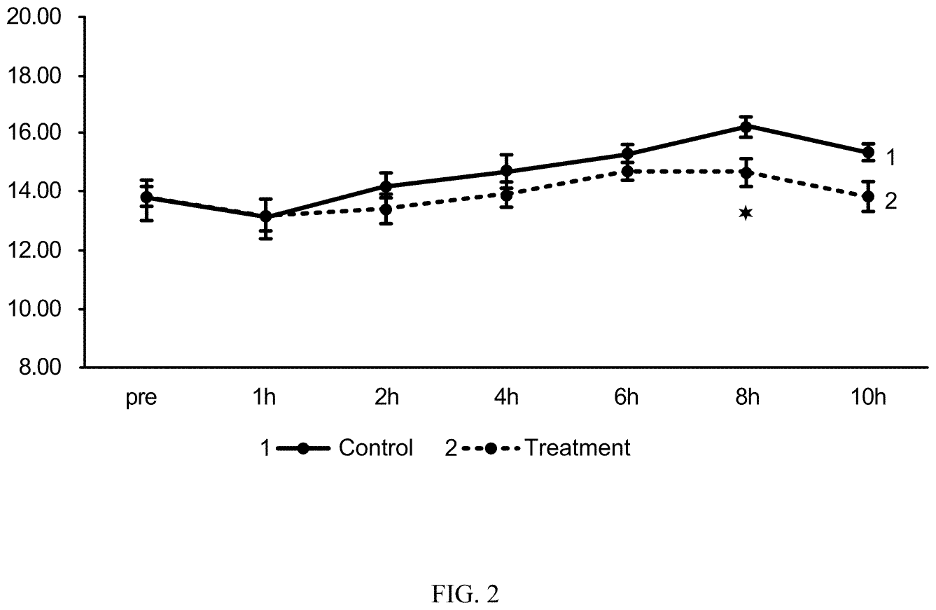

FIG. 2 shows the results from control group 2 of the IOP lowering effect (mean IOP +/−SEM) study with latanoprost (0.005%) in rabbits at various time points after instillation of ophthalmic solutions (control solutions into left eyes and treatment solution into right eyes).

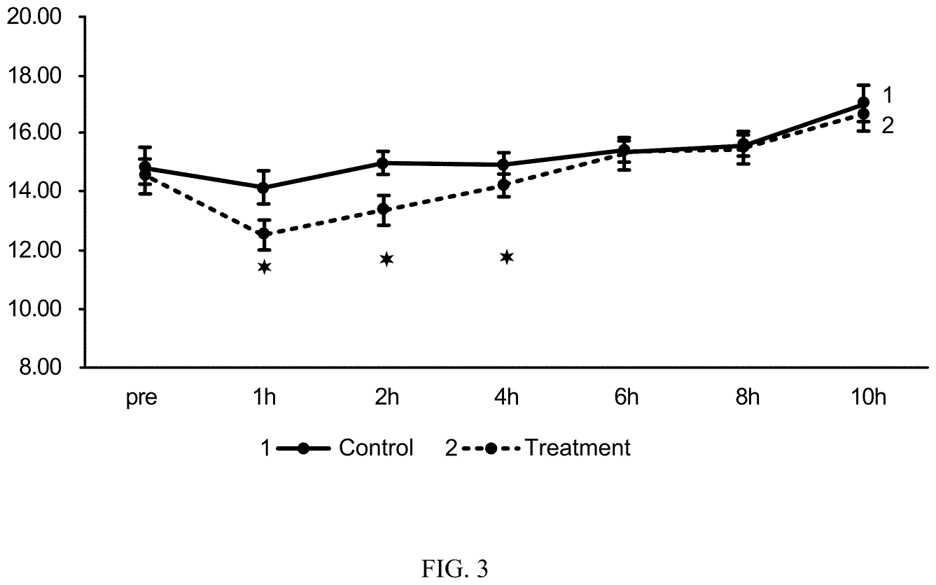

FIG. 3 shows the results from test group 3 of the IOP lowering effect study (mean IOP +/−SEM) with compound 18 (10 mg/mL) in rabbits at various time points after instillation of ophthalmic solutions (control solutions into left eyes and treatment solution into right eyes).

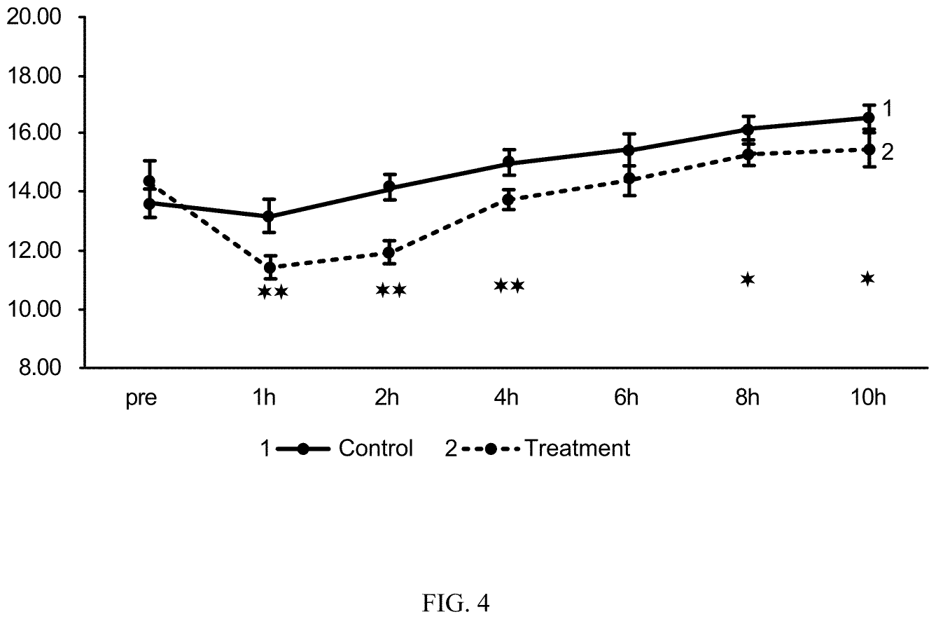

FIG. 4 shows the results from test group 4 of the IOP lowering effect study (mean IOP +/−SEM) with compound 18 (20 mg/mL) in rabbits at various time points after instillation of ophthalmic solutions (control solutions into left eyes and treatment solution into right eyes).

### Conclusion

Compound 18 was demonstrated to significantly lower the IOP after its administration at both 10 mg/mL and 20 mg/mL doses.

### Example 9. Intraocular Pressure (IOP)-Lowering Effect of Compound 18 in Rabbit Model

### Summary

The purposes of this study were to explore the intraocular pressure (IOP) lowering effect of Compound 18 when administered by topical instillation to ocular normotensive and ocular hypertensive rabbits, respectively.

Forty (40) male New Zealand white rabbits were divided into 4 groups, which included 10 animals/group. Animals were randomly assigned to groups by Provantis based on body weight.

Phase 1: To confirm the IOP lowering effect of test article at different concentrations in ocular normotensive rabbits. Compound 18 was instilled into right eyes of animals in groups 3 and 4, 50 μL/eye, once. 0.024% latanoprostene bunod ophthalmic solution (LBN) and 0.005% latanoprost eye drops were served as positive control and be dosed with same volume into right eyes of groups 1 and 2 animals once. All left eyes of animals in each group were dosed with vehicle once, 50 μL/eye. IOPs was measured once at pre-dose and once at 1, 2, 4, 6, 8, 10 hours post dose.

Phase 2: To confirm the IOP lowering effect of test article at different concentrations in ocular hypertensive rabbits. Modeling was induced by intracameral injection of elastic substance in both eyes, once. Then, positive control articles/test article was instilled into right eyes of animals, 50 μL/eye, once at 5-15 minutes, 3 and 6 hours post modeling, respectively. All left eyes of animals in each group were dosed with vehicle, 50 μL/eye, once at 5-15 minutes, 3 and 6 hours post modeling, respectively. IOPs was measured at pre-modeling, 1, 2, 4, 6, 8, and 10 hours post first dose.

During phase 1 measurement, compared with the vehicle treated eyes, no significant decreased IOPs were noted in the eyes dosed with 0.024% Latanoprostene Bunod at each time points (1, 2, 4, 6, 8, 10 h) post dosing.

Compared with the vehicle treated eyes, significant decreased IOPs were noted in the eyes dosed with 0.005% Latanoprost at the time points (2, 8 h) post dosing (2, 8 h, p≤0.05).

Compared with the vehicle treated eyes, significant decreased IOPs were noted in the eyes dosed with 10 mg/mL Compound 18 at the time points (1, 2, 4 h) post dosing (1, 2, 4 h, p≤0.05)

Compared with the vehicle treated eyes, significant decreased IOPs were noted in the eyes dosed with 20 mg/mL Compound 18 at the time points (1, 2, 4, 8, 10 h) post dosing (1, 2, 4 h, p≤0.05) (8, 10 h, p≤0.05; 1, 4 h, p≤0.01; 2 h, p≤0.001).

During phase 2 measurement, compared with the vehicle treated eyes, no significant decreased IOPs were noted in the eyes dosed with 0.024% Latanoprostene Bunod and 0.005% Latanoprost at each time points (1, 2, 4, 6, 8, 10 h) post dosing.

Compared with the vehicle treated eyes, significant decreased IOPs were noted in the eyes dosed with 10 mg/mL Compound 18 at the time point 2 h post dosing (p≤0.05).

Compared with the vehicle treated eyes, significant decreased IOPs were noted in the eye dosed with 20 mg/mL Compound 18 at the time point 1 h post dosing (p≤0.05).

For further analysis of the IOP reduction effect, the IOP reduction ratio was calculated using the difference between the TA/PA treated and vehicle treated eyes at each time points post first dosing. The results were tabulated below:

TABLE 6

| | Reduction ratio of IOP | | | | | |
|---|---|---|---|---|---|---|
| Group | 1 h | 2 h | 4 h | 6 h | 8 h | 10 h |
| 5—Latanoprostene bunod (0.024%) | 24.15% | 5.69% | 5.38% | 5.83% | 26.62% | 28.69% |
| 6—Latanoprost (0.005%) | −10.40% | −2.49% | −10.23% | −8.01% | 26.53% | 30.28% |
| 7—Compound 18 (10 mg/mL) | 23.36% | 35.65% | 21.68% | 28.06% | 23.97% | 21.67% |
| 8—Compound 18 (20 mg/mL) | 45.31% | 34.59% | 26.95% | 18.04% | 10.85% | −5.73% |

More than twenty percent reduction of IOPs were noted in the eyes dosed with 10 mg/mL Compound 18 at each time points (1, 2, 4, 6, 8, 10 h) post dosing. While a relative higher reduction ratio of IOPs were noted in the eyes dosed with 20 mg/mL Compound 18 at first three time points. Relative lower IOP reduction ratios were noted in the 20 mg/ml TA treated eyes at the last time points. It may be attributed to the fast IOP reduction at the very beginning after modeling in high dose group.

In conclusion, administration of Compound 18 to ocular normotensive rabbits once by topical instillation at concentrations of 10 and 20 mg/mL could result in IOP reduction. The IOP lowering effect is dose and time dependent. While administration of Compound 18 to ocular hypertensive rabbits thrice by topical instillation after modeling at concentrations of 10 and 20 mg/mL could result in IOP reduction without significant difference from unilateral eyes.

A stable IOP lowering effect (↓>20%) was noted when dose at the concentration of 10 mg/mL Compound 18.

### Study Design

The study design is outlines in Table 7 and 8 below:

TABLE 7
Phase I

| | Animals^{b} | Treatment (50 uL/eye/time) | | Volume, | Numbering of |
|---|---|---|---|---|---|
| Group | (Males) | Right Eye | Left Eye | Frequency | Animals^{a} |
| 1 | 10 | 0.024% Latanoprostene bunod | Gel-Blank | 50 μL/eye | 1001-1010 |
| | | ophthalmic solution | | once | |
| 2 | 10 | 0.005% latanoprost Eye Drops | Gel-Blank | | 2001-2010 |
| 3 | 10 | 1% Compound 18 | Gel-Blank | | 3001-3010 |
| 4 | 10 | 2% Compound 18 | Gel-Blank | | 4001-4010 |

TABLE 8
Phase 2

| | Animals | Treatment (50 uL/eye/time) | | Volume, | Numbering of |
|---|---|---|---|---|---|
| Group | (Males) | Right Eye | Left Eye | Frequency | Animals^{a} |
| 5 | 10 | 0.024% Latanoprostene bunod | Gel-Blank | 50 μL/eye, t.i.d. | 5001-5010 |
| | | ophthalmic solution | | 5-15 mins, 3 | |
| 6 | 10 | 0.005% latanoprost Eye Drops | Gel-Blank | and 6 hours | 6001-6010 |
| 7 | 10 | 1% Compound 18 | Gel-Blank | post modeling | 7001-7010 |
| 8 | 10 | 2% Compound 18 | Gel-Blank | | 8001-8010 |

### Route of Administration and Justification

The test article was administered topically. Topical ocular administration is the intended route of administration to humans.

### Dose Level Selection

Latanoprost eye drops and Latanoprostene bunod ophthalmic solution are used to lower the intraocular pressure, which are commercially available drugs. The concentrations of Compound 18 were selected by the sponsor base on the previous formulation stability study and to explore the target effective concentration of the test article in the present study.

### Modeling

The ocular hypertensive model was induced as following steps:

Animals were sedated by Ketamine (30˜50 mg/kg) and anesthetized by Xylazine (5˜10 mg/kg), intramuscularly. Disinfected the conjunctival sac by washing with 5% povidone iodine, and then washed the conjunctival sac with saline. Fixed the eyeball with forceps, then inserted needle from the temporal limbus horizontally, avoided damaging the corneal endothelial and iris. Held the syringe still and injected the Medical Sodium Hyaluronate Gel slowly into the anterior chamber, 100 μl/eye, waited for at least 10 seconds, then pulled out the needle slowly. Pressed the injection spot immediately with swab for at least 3 minutes, blocked the injection spot with one drop of tissue adhesive.

### Drug Administration and Duration of the Study

For Phase 1, 50 μL of 1% and 2% Compound 18, 0.024% latanoprostene bunod ophthalmic solution and 0.005% latanoprost eye drops were instilled into the right eyes and the same volume of Vehicle (Gel-blank) into the left eyes in Group 1 to Group 4 of Ocular Hypertensive Rabbits, 50 μL/eye, once.

For Phase 2, 50 μL of 1% and 2% Compound 18, 0.024% latanoprostene bunod ophthalmic solution and 0.005% latanoprost eye drops were instilled into the right eyes and the same volume of Vehicle (Gel-blank) into the left eyes in Group 5 to Group 8 of Ocular Hypertensive Rabbits, 50 μL/eye, three times. The first day of dosing in phase 1 was designated as Day 1.

Delivery Method: Drawed 50 μL of the articles with a pipette, pulled out the lower eyelid of the animal, dropped the article into the conjunctiva sac, and gently closed the upper and lower eyelids for a few times.

### Animals

Species/Strain: New Zealand White Rabbits (Oryctolagus cuniculus). The New Zealand White Rabbit was used in this study. This is commonly used specie for non-clinical studies of ophthalmic formulations, for which there is a large historical database, and the relative large cornea size is suitable for topical operations.

Number and Sex: Forty male New Zealand White Rabbits were divided into 4 groups, 10 animals/group. An additional 8 male rabbits were ordered for possible replacement during the whole in-life experiment. The disposition of all animals were documented in the study records.

Age and Body Weight at Dosing Initiation: Approximately 3 to 4 months, Males: 2.4 to 2.9 kg. Animals in poor health or with notable physical abnormalities were not assigned to the study. All animals placed on study had body weights that fell within ±20% of the mean body weight at randomization. Each animal was weighed at least once during pre-test, and once weekly throughout the in-life experiment.

### IOP Measurement

Intraocular pressure was measured as following steps:

Restrained the animal, made the testing eye perpendicular to the probe of the TonoVet tonometer, measured the IOP for at least three times until the values are stable. The mean of the last three stable values were calculated as the final IOP value.

Animal Acclimation for the IOP Measurement Handling: Intraocular pressure (IOP) was measured in both eyes of all animals on 3 separate days during acclimation prior to dosing initiation.

IOP Measurement in Ocular Normotensive Rabbits: IOP was measured at pre-dose and once at 1, 2, 4, 6, 8, 10 hours post dose in study phase 1.

IOP Measurement in Ocular Hypertensive Rabbits: IOP was measured at pre-modeling, and once at 1, 2, 4, 6, 8, and 10 hours post first daily dose in study phase 2.

### Results

Clinical Observations: No abnormalities were observed throughout the in-life duration of the study. Further, there was no unscheduled deaths during the in-life period.

Body weights: There were no abnormal body weight changes observed throughout the in-life duration.

### Intraocular Pressure (IOP) Measurements:

Animal acclimation for the IOP measurement handling: at the early stage of the experiment (Day 1 to Day 3), intraocular pressure adaption was measured for all animals and no abnormal intraocular pressure was observed.

### Phase 1: IOP Measurements in Ocular Normotensive Rabbits:

During phase 1 measurement, no significant decreases in IOP were observed in the eyes dosed with 0.024% latanoprostene bunod at each time point post dosing (e.g., 1, 2, 4, 6, 8 and 10 h post dosing), as compared with the vehicle treated eyes.

Compared with the vehicle treated eyes, significant decreased IOPs were observed in the eyes dosed with 0.005% latanoprost at the time points 2 and 8 hours post dosing (2, 8 h, p≤0.05).

Compared with the vehicle treated eyes, significant decreased IOPs were observed in the eyes dosed with 10 mg/mL compound 18 at the time points 1, 2 and 4 hours post dosing (1, 2, 4 h, p≤0.05)

Compared with the vehicle treated eyes, significant decreased IOPs were observed in the eyes dosed with 20 mg/mL compound 18 at the time points 1, 2, 4, 8 and 10 hours post dosing (1, 2, 4 h, p≤0.05) (8, 10 h, p≤0.05; 1, 4 h, p≤0.01; 2 h, p≤0.001).

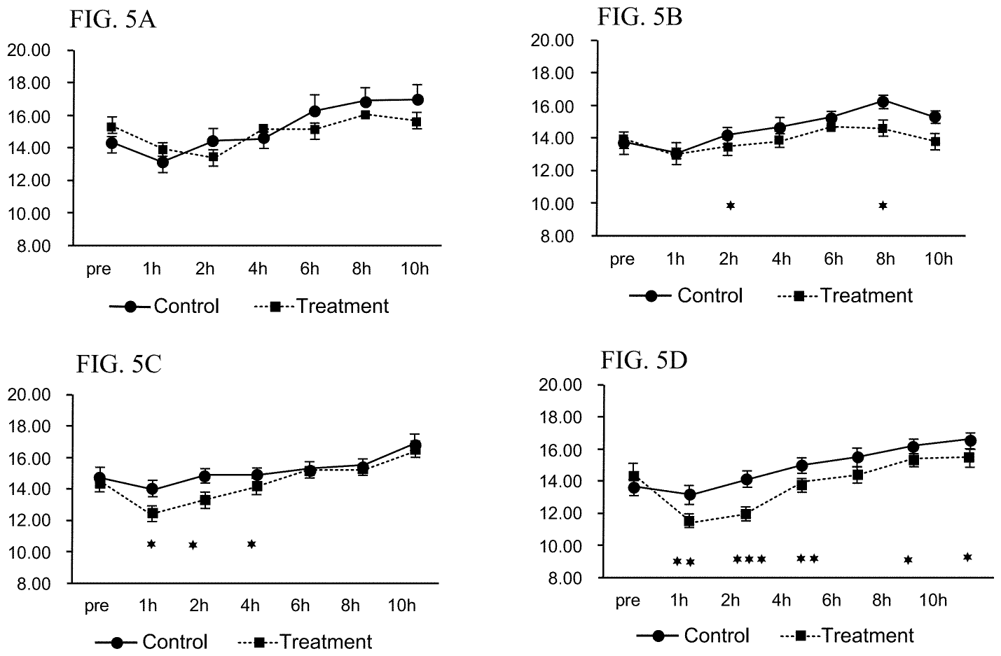

The change in IOP before and after treatment in each of groups 1-4 on day 1 of study (Mean±SEM) is shown in FIGS. 5A-5D, where * represents T test for pairs in each group. Comparison between right eye and left eye is P≤0.05; ** represents T test for pairs in each group, and P≤0.01 for comparison between right eye and left eye; and *** represents T test for pairs in each group, and P≤0.001 for comparison between right and left eyes. FIG. 5A shows the change in IOP before and after treatment with 0.024% latanoprostene bunod on day 1 (group 1). FIG. 5B shows the change in IOP before and after treatment with 0.005% latanoprostene bunod on day 1 (group 2). FIG. 5C shows the change in IOP before and after treatment with 10 mg/mL of compound 18 on day 1 (group 3). FIG. 5D shows the change in IOP before and after treatment with 20 mg/mL of compound 18 on day 1 (group 3).

Table 9 summarizes the results of the phase 1 intraocular pressure measurements for each of groups 1-4 in the right eye (OD IOP) and left eye (OS IOP).

TABLE 9
Summary of IOP measurements—Phase 1
Sex: Male Day: 1 Relative to Start Date

| | | PTFD | 1 h | 2 h | 4 h | 6 h | 8 h | 10 h |
|---|---|---|---|---|---|---|---|---|
| | | OD IOP | | | | | | |
| Group 1 | Mean | 15.73 | 14.23 | 13.73 | 15.47 | 15.43 | 16.50 | 16.07 |
| 50 | SD | 1.96 | 1.47 | 1.67 | 1.00 | 1.48 | 1.06 | 1.57 |
| μL/eye | N | 10 | 10 | 10 | 10 | 10 | 10 | 10 |
| Group 2 | Mean | 13.87 | 13.57 | 12.97 | 13.47 | 14.13 | 15.20 | 14.80 |
| 50 | SD | 1.53 | 1.93 | 1.38 | 1.55 | 1.52 | 1.37 | 1.68 |
| μL/eye | N | 10 | 10 | 10 | 10 | 10 | 10 | 10 |
| Group 3 | Mean | 14.50 | 12.50 | 13.30 | 14.17 | 15.27 | 15.57 | 16.53 |
| 50 | SD | 1.78 | 1.52 | 1.67 | 1.30 | 1.73 | 1.28 | 1.52 |
| μL/eye | N | 10 | 10 | 10 | 10 | 10 | 10 | 10 |
| Group 4 | Mean | 14.27 | 11.47 | 11.93 | 13.70 | 14.33 | 15.20 | 15.40 |
| 50 | SD | 2.45 | 1.21 | 1.07 | 0.97 | 1.60 | 1.23 | 1.73 |
| μL/eye | N | 10 | 10 | 10 | 10 | 10 | 10 | 10 |
| | | OS TOP | | | | | | |
| Group 1 | Mean | 14.70 | 13.50 | 14.83 | 15.10 | 16.77 | 17.37 | 17.53 |
| 50 | SD | 2.00 | 2.24 | 2.51 | 2.31 | 3.22 | 2.86 | 2.77 |
| μL/eye | N | 10 | 10 | 10 | 10 | 10 | 10 | 10 |
| Group 2 | Mean | 13.87 | 13.10 | 14.20* | 14.70 | 15.27 | 16.23* | 15.33 |
| 50 | SD | 1.11 | 2.07 | 1.36 | 1.87 | 1.11 | 1.12 | 0.89 |
| μL/eye | N | 10 | 10 | 10 | 10 | 10 | 10 | 10 |
| Group 3 | Mean | 14.80 | 14.07* | 14.93* | 14.90* | 15.37 | 15.43 | 16.93 |
| 50 | SD | 2.01 | 1.72 | 1.25 | 1.18 | 1.19 | 1.44 | 1.97 |
| μL/eye | N | 10 | 10 | 10 | 10 | 10 | 10 | 10 |
| Group 4 | Mean | 13.60 | 13.14** | 14.10*** | 14.93** | 15.37 | 16.10* | 16.43* |
| 50 | SD | 1.55 | 1.79 | 1.42 | 1.39 | 1.79 | 1.26 | 1.30 |
| μL/eye | N | 10 | 10 | 10 | 10 | 10 | 10 | 10 |

Paired T test: * = p < 0.05, OSIOP vs ODIOP

Phase 2: IOP Measurements in Ocular Hypertensive Rabbits:

During phase 2 measurement, no significant decreases in IOP were observed in the eyes dosed with 0.024% latanoprostene bunod and 0.005% latanoprost at each time point post dosing (e.g., 1, 2, 4, 6, 8 and 10 h post dosing), as compared with the vehicle treated eyes.

Compared with the vehicle treated eyes, significant decreased IOPs were observed in the eyes dosed with 10 mg/mL compound 18 at the time point 2 hours post dosing (p≤0.05)

Compared with the vehicle treated eyes, significant decreased IOPs were observed in the eyes dosed with 20 mg/mL compound 18 at the time point 1 hour post dosing (p≤0.05).

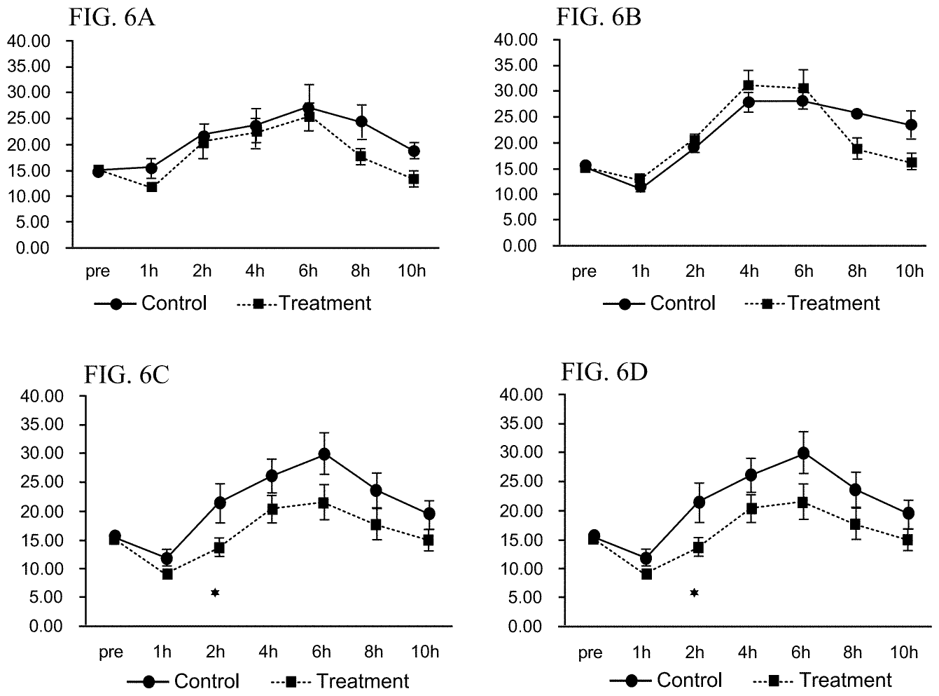

The change in IOP before and after treatment in each of groups 5-8 on day 10 and day 11 of study (Mean±SEM) is shown in FIGS. 6A-6D, where * represents T test for pairs in each group. Comparison between right eye and left eye is P≤0.05. FIG. 6A shows the change in IOP before and after treatment with 0.024% latanoprostene bunod (group 5). FIG. 6B shows the change in IOP before and after treatment with 0.005% latanoprost (group 6). FIG. 6C shows the change in IOP before and after treatment with 10 mg/mL of compound 18 (group 7). FIG. 6D shows the change in IOP before and after treatment with 20 mg/mL of compound 18 (group 8).

Table 10 summarizes the results of the phase 2 intraocular pressure measurements for each of groups 5-8 in the right eye (OD IOP) and left eye (OS IOP).

TABLE 10
Table of IOP measurements—Phase 2
Sex: Male Day: 10/11 Relative to Start Date

| | | PTFD | 1 h | 2 h | 4 h | 6 h | 8 h | 10 h |
|---|---|---|---|---|---|---|---|---|
| | | OD IOP | | | | | | |
| Group 5 | Mean | 14.87 | 11.63 | 20.43 | 23.73 | 25.37 | 17.67 | 13.37 |
| 50 | SD | 1.66 | 1.63 | 9.92 | 9.98 | 7.97 | 4.54 | 4.65 |
| μL/eye | N | 10 | 9 | 10 | 10 | 10 | 10 | 10 |
| Group 6 | Mean | 15.03 | 12.96 | 20.20 | 31.00 | 30.47 | 18.93 | 16.40 |
| 50 | SD | 1.84 | 2.78 | 4.86 | 9.07 | 11.08 | 6.55 | 4.64 |
| μL/eye | N | 10 | 9 | 10 | 10 | 10 | 9 | 10 |
| Group 7 | Mean | 15.17 | 9.19 | 13.80 | 20.47 | 21.53 | 17.82 | 15.22 |
| 50 | SD | 1.71 | 1.69 | 4.76 | 7.17 | 9.60 | 8.06 | 6.02 |
| μL/eye | N | 10 | 9 | 10 | 10 | 10 | 9 | 9 |
| Group 8 | Mean | 15.50 | 11.74 | 19.67 | 25.57 | 28.93 | 27.40 | 22.63 |
| 50 | SD | 2.20 | 2.32 | 7.49 | 6.20 | 6.73 | 6.59 | 6.08 |
| μL/eye | N | 10 | 9 | 10 | 10 | 10 | 10 | 10 |
| | | OS IOP | | | | | | |
| Group 5 | Mean | 15.03 | 15.33 | 21.67 | 23.41 | 26.93 | 24.07 | 18.74 |
| 50 | SD | 1.26 | 6.10 | 6.73 | 10.37 | 14.18 | 10.57 | 4.77 |
| μL/eye | N | 10 | 9 | 9 | 9 | 10 | 9 | |
| Group 6 | Mean | 15.67 | 11.74 | 19.71 | 28.13 | 26.52 | 25.76 | 23.52 |
| 50 | SD | 1.48 | 3.53 | 4.99 | 6.51 | 6.64 | 7.13 | 8.44 |
| μL/eye | N | 9 | 0 | 8 | 8 | 8 | 8 | 7 |
| Group 7 | Mean | 15.47 | 12.03 | 21.44* | 26.13 | 29.93 | 23.43 | 19.43 |
| 50 | SD | 1.40 | 4.38 | 10.86 | 9.03 | 11.26 | 9.60 | 7.70 |
| μL/eye | N | 10 | 10 | 9 | 10 | 10 | 10 | 10 |
| Group 8 | Mean | 16.80* | 21.47* | 30.07 | 35.00 | 35.30 | 30.73 | 21.41 |
| 50 | SD | 1.76 | 11.65 | 15.37 | 14.66 | 13.51 | 13.57 | 11.88 |
| μL/eye | N | 10 | 10 | 10 | 10 | 10 | 10 | 9 |

Paired T test: * = p < 0.05, OSIOP vs OD1OP

### Summary

In summary, administration of Compound 18 to ocular normotensive rabbits once by topical instillation at concentrations of 10 and 20 mg/mL can result in IOP reduction. The IOP lowering effect is dose and time dependent. Administration of Compound 18 to ocular hypertensive rabbits (e.g., thrice by topical instillation after modeling at concentrations of 10 and 20 mg/mL) can result in IOP reduction. A stable IOP lowering effect (e.g., a decrease in IOP of greater than 20%) was observed for dosage of Compound 18 at a concentration of 10 mg/mL.

These results show that an exemplary compound lowered IOP in vivo in a rabbit eye model, and indicates that the compounds of this disclosure would be effective in treating eye diseases characterized by an increase in IOP, such as glaucoma.

### Example 10—Pharmacokinetics and Tissue Distribution of Compound 18 Following Once Daily Treatment in Rabbits for 7 Consecutive Days

This example illustrates the procedures and results for the levels of Compound 18 and its metabolite Compound 4 in plasma and ocular tissues of normotensive rabbits following ocular instillation of Compound 18 once daily for 7 consecutive days.

### Materials and Study Design

Twenty five (25) male New Zealand white rabbits were divided into 5 groups of varying amounts of animals per group.

The Test article, Compound 18 was formulated as a 2% nanosuspension in DI water containing 0.2% PS80 (polyoxyethylenesorbitan, monooleate (Tween 80)) and 0.4% HPMC (hydroxypropylmethylcellulose) at pH 6.77. Referred to herein as “Compound 18 nanosuspension.”

The Study design is summarized in Table 11 below:

TABLE 11
Study Design

| | | | Number | | |
|---|---|---|---|---|---|
| | | | of | | Dose |
| | Study | Group | Animals | | Level |
| Route | Phase^{a} | Number | (Male) | Test Article/Vehicle | (Drop)^{b} |
| Ocular | Pilot^{a} | 1 | 3 | Compound 18 | 1^{b} |
| instillation | | | | nanosuspension | |
| | PK^{a} | 2 | 4 | Compound 18 | 1^{b} |
| | | | | nanosuspension | |
| | TD^{a} | 3 | 12 | Compound 18 | 1^{b} |
| | | | | nanosuspension | |
| None | Control^{c} | 4 | 3 | No Treatment | NA |
| | Control^{d} | 5 | 3 | No Treatment | NA |

^{a}Group 1 animals were allocated to the pilot study. Group 2 animals were allocated to the Pharmacokinetics (PK) phase and Group 3 animals were allocated to the Tissue Distribution (TD) Phase. Blood samples were collected from all three groups and tissue samples were collected from Groups 1 and 3 after animal termination.
^{b}Instill one drop to each eye once daily in the morning for 7 consecutive days (7 drops total per eye).
^{c}Group 4 animals were allocated as control animals for providing control ocular tissues to prepare matrix matched standard and QC samples for Pilot Study sample analysis. These animals were not dosed.
^{d}Group 5 animals were allocated as control animals for providing control ocular tissues to prepare matrix matched standard and QC samples for TD Phase study sample analysis. These animals were not dosed.

### Experimental Procedures

The study consisted of three groups (Groups 1-3) of 19 male rabbits (NZW) that underwent once daily morning ocular installation (1 eyedrop) for 7 consecutive days (Day 0-6). Groups 4-5 each consisted of 3 male rabbits (NZW) that were not dosed.

### Blood Sample Collection

Blood samples (1 mL) were collected from the jugular vein at the intervals set out below for each group. The samples were then inverted several times following collection and held on wet ice until centrifuged. Samples were centrifuged within 60 minutes of collection under refrigeration (set at 5° C. for 10 minutes at 2000 g.

Group 1 (Pilot Study, terminal blood collection): blood was collected from 3 animals at 1 hour post day 7 dose of Compound 18 nanosuspension.

Group 2 (PK Phase): blood was collected from each animal at 0.5, 1, 2, 4, 8 and 25 hours post day 1 dose and post day 7 dose of Compound 18 nanosuspension.

Group 3 (TD Phase, terminal blood collection): blood was collected from 3 animals at 0.5 hour, 3 animals at 2 hours, 3 animals at 4 hours, and 3 animals at 8 hours post day 7 dose of Compound 18 nanosuspension.

Group 4 (Control, terminal blood collection): blood was collected from 3 control animals when the pilot study was conducted.

Group 5 (Control, terminal blood collection): blood was collected from 3 control animals when the PK and TD phase was conducted.

### Ocular Tissue Sample Collection

Ocular tissue samples were collected from terminated animals at the intervals set out below for each group.

Group 1 (Pilot Study): 3 animals were terminated at 1 hour post Day 7 dose of Compound 18 nanosuspension. Following blood collection and termination, ocular tissues (aqueous humor, vitreous humor, cornea, iris/ciliary body, lens, optic nerve, retina, sclera/choroid, and trabecular meshwork) were collected from each animal and the tissue weights were recorded. Note: sclera and choroid were collected together. Iris and ciliary body were collected together.

Group 3 (TD Phase): 3 animals were terminated at 0.5 hour, 3 animals at 2 hours, 3 animals at 4 hours, and 3 animals at 8 hours post Day 7 dose of Compound 18 nanosuspension. Following blood collection and termination, ocular tissues (aqueous humor, vitreous humor, cornea, iris/ciliary body, lens, optic nerve, retina, sclera/choroid, and trabecular meshwork) were collected from each animal and the tissue weights were recorded. Note: sclera and choroid were collected together. Iris and ciliary body were collected together.

Group 4 (Control): 3 animals were terminated along with Group 1 Pilot Study animals. Following blood collection and termination, ocular tissues (aqueous humor, vitreous humor, cornea, iris/ciliary body, lens, optic nerve, retina, sclera/choroid, and trabecular meshwork) were collected from each animal and the tissue weights were recorded. Note: sclera and choroid were collected together. Iris and ciliary body were collected together.

Group 5 (Control): 3 animals were terminated along with Group 2 Phase PK and Group 3 TD Phase animals. Following blood collection and termination, ocular tissues (aqueous humor, vitreous humor, cornea, iris/ciliary body, lens, optic nerve, retina, sclera/choroid, and trabecular meshwork) were collected from each animal and the tissue weights were recorded. Note: sclera and choroid were collected together. Iris and ciliary body were collected together.

### Bioanalysis and Pharmacokinetic Analysis

Plasma and tissue samples from treated animals (Groups 1-3) were analyzed for Compound 18 and its metabolite Compound 4 concentrations by LC-MS/MS.

Pharmacokinetic analysis was conducted using WinNonlin Version 6.2.1 (Pharsight, Mountain View, Calif.), operating as a validated software system. Noncompartmental analysis was conducted using the extravascular administration model for ocular instillation dosing.

The peak plasma concentration, time to achieve peak plasma concentration, half-life, and area under the plasma concentration-time curve (Cₘₐₓ, Tₘₐₓ, T_{1/2}, and AUC) were calculated from the PK phase individual animal plasma concentrations for each sampling time/dose group for Compound 18 and its metabolite Compound 4.

The peak plasma and ocular tissue concentration, time to achieve peak plasma and ocular tissue concentration, half-life, and area under the plasma and ocular tissue concentration-time curve (Cₘₐₓ, Tₘₐₓ, T_{1/2}, and AUC) were calculated from the TD phase combined with the pilot study mean animal plasma and ocular tissue concentrations for each sampling time/dose group for Compound 18 and its metabolite Compound 4.

### Bioanalytical Method for Plasma, Aqueous Humor and Vitreous Humor

Compound 18 (A) Stock Solution Preparation: Compound 18 primary stock solutions (SA01/CA01) were prepared in 50% Acetonitrile in Water:DMSO (1:1, v/v) at a nominal concentration of 1.5 mg/mL. Compound 18 primary stock solutions were diluted in diluent solution (50% Acetonitrile in Water) to prepare Compound 18 secondary stock solutions (SA02/CA02) at 160,000 ng/mL.

Compound 4 (B) Stock Solution Preparation: Compound 4 primary stock solutions (SB01/CB01) were prepared in 50% Acetonitrile in Water:DMSO (1:1, v/v) at a nominal concentration of 1.5 mg/mL. Compound 4 primary stock solutions were diluted in diluent solution to prepare Compound 4 secondary stock solutions (SB02/CB02) at 160,000 ng/mL.

Alprazolam Internal Standard (IS) Stock Solution Preparation: Alprazolam primary stock solution (I01, 1.0 mg/mL in methanol) was purchased from Sigma. The internal standard stock solution I01 was diluted in diluent solution to 20,000 ng/mL (I02). I02 solution was diluted in acetonitrile to prepare the working internal standard solution (WIS) at 10.0 ng/mL.

BA Assay: Quantitation Range 0.05 to 100 ng/mL for Compound 18, 0.15 to 100 ng/mL for Compound 4.

Compound 18 Plasma Standard (PLSTD-A) and Plasma QC (PLQC-A) Samples Preparation: Calibration curve spiking solutions (SS-A-1 to SS-A-10) ranging from 1.00 to 2000 ng/mL were prepared by dilution of SA02 and subsequent solutions with diluent solution. QC-A spiking solutions (SS-A-QC-Low, Mid-1, Mid-2, High, Dil) ranging from 3.00 to 16,000 ng/mL were prepared by dilution of CA02 and subsequent solutions with diluent solution.

Plasma calibration standards (PLSTD-A) with Compound 18 concentrations ranging from 0.0500 to 100 ng/mL were prepared by spiking control rabbit plasma with appropriate calibration curve spiking solutions (SS-A). PLQC-A samples with concentrations of 0.150, 5.00, 50.0, and 80.0 ng/mL were prepared by spiking control rabbit plasma with appropriate SS-A-QC spiking solutions.

Compound 18 Aqueous Humor QC (AH QC-A) Sample Preparation: AH QC-A samples with concentrations of 0.150, 5.00, 50.0, and 80.0 ng/mL were prepared by spiking control rabbit aqueous humor with appropriate QC-A spiking solutions.

Compound 18 Vitreous Humor QC (VH QC-A) Samples Preparation: VH QC-A samples with concentrations of 0.150, 5.00, 50.0, and 80.0 ng/mL were prepared by spiking control rabbit vitreous humor with appropriate QC-A spiking solutions.

Compound 4 Plasma Standards (PLSTD-B) and Plasma QC (PLQC-B) Samples Preparation: Calibration curve spiking solutions (SS-B-1 to SS-B-10) ranging from 3.00 to 2000 ng/mL were prepared by dilution of SB02 and subsequent solutions with diluent solution. QC-B spiking solutions (SS-B-QC-Low, Mid-1, Mid-2, High, Dil) ranging from 9.00 to 16,000 ng/mL were prepared by dilution of CB02 and subsequent solutions with diluent solution.

Plasma calibration standards (PLSTD-B) with Compound 4 concentrations ranging from 0.150 to 100 ng/mL were prepared by spiking control rabbit plasma with appropriate calibration curve spiking solutions (SS-B). PLQC-B samples with concentrations of 0.450, 5.00, 50.0, and 80.0 ng/mL were prepared by spiking control rabbit plasma with appropriate SS-B-QC spiking solutions. PLQC-B-Dil samples at 800 ng/mL were prepared by spiking control rabbit plasma with the SS-B-QC-Dil spiking solution at 16,000 ng/mL, and then diluted 20× with control rabbit plasma prior to extraction.

Compound 4 Aqueous Humor QC (AH QC-B) Samples Preparation: AH QC-B samples with concentrations of 0.450, 5.00, 50.0, and 80.0 ng/mL were prepared by spiking control rabbit aqueous humor with appropriate QC-B spiking solutions. AH QC-B-Dil samples at 800 ng/mL were prepared by spiking control rabbit aqueous humor with the QC-B-Dil spiking solution at 16,000 ng/mL, and then diluted 20× with control rabbit aqueous humor prior to extraction.

Compound 4 Vitreous Humor QC (VH QC-B) Samples Preparation: VH QC-B samples with concentrations of 0.450, 5.00, 50.0, and 80.0 ng/mL were prepared by spiking control rabbit vitreous humor with appropriate QC-B spiking solutions.

### Extraction Procedure

1. Thaw plasma standards, plasma QC samples, ocular tissue QC samples, incurred plasma samples and ocular tissue samples (AH/VH) (if applicable) on wet ice. Control plasma can be thawed in tepid water. Vortex before taking aliquots.

2. Pipet 50 μL of water for Reagent Blank, 50 μL of control plasma for Double Blank and Blank, 50 μL of pre-spiked plasma for Standards and QC samples, 50 μL of incurred plasma samples (if applicable) into a 96 well plate.

3. Pipet 50 μL of each type of pre-spiked ocular tissue samples (AH/VH) for QC samples, 50 μL of incurred aqueous humor, and vitreous humor sample (if applicable) into a 96 well plate.

4. Add 200 μL of acetonitrile (ACN) to the Reagent Blank and Double Blank samples.

5. Add 200 μL of working internal standard (WIS) solution to all remaining samples.

6. Seal the plate and vortex for approximately 5 minutes. Centrifuge the plate at 3500 rpm for 10 minutes at ˜4° C.

7. Transfer ˜200 μL of each sample supernatant into the appropriate wells of a 96 well plate. Submit for LC MS/MS analysis.

### Analytical Runs:

The incurred plasma, aqueous humor, vitreous humor samples from the study were analyzed in an analytical run, where the run consisted of calibration standards, QC samples (low, mid-1, mid-2 and high concentrations), blanks, and incurred samples. When a run required incurred sample dilution, QC-Dil 20-fold dilutions were also included.

### Bioanalytical Method for Rabbit Ocular Tissues Sclera/Choroid, Iris/Ciliary Body, Trabecular Meshwork Lens, Cornea, Retina, Optic Nerve.

Ocular Tissues Homogenate Preparation: Incurred rabbit ocular tissues were weighed in Genogrinder tubes (plastic tubes with small beads) at the time of removal from the animal and stored frozen (<−70° C.) until analysis. The rabbit incurred ocular tissues were thawed on wet ice. Phosphate-buffered saline (PBS, pH 7.2, Gibco) was added to each type of ocular tissue sample with each specified dilution factor (see the Table 12 below). The tubes were homogenized on Genogrinder GG1 set for 2.5 minutes at 1750 rpm, chilled in wet ice and homogenized for another 2.5 minutes. Tissues that appeared to not be homogenized well were further homogenization (1 min/time) until good homogenate observed.

TABLE 12
Ocular tissue sample preparation

| Ocular Tissue | PBS (mL) per g of Tissue | Dilution Factor |
|---|---|---|
| Sclera/Choroid | 7 | 8X |
| Cornea | 14 | 15X |
| Iris/Ciliary Body | 19 | 20X |
| Trabecular Meshwork | 19 | 20X |
| Retina | 19 | 20X |
| Optic Nerve | 19 | 20X |
| Lens | 4 | 5X |

BA Assay: Quantitation Range 0.05 to 100 ng/mL for Compound 18, 0.15 to 100 ng/mL for Compound 4.

Compound 18 Sclera/Choroid Homogenate (S/CH) Standards and QC Samples Preparation: Calibration curve spiking solutions (SS-A-1 to SS-A-10) ranging from 1.00 to 2000 ng/mL were prepared by dilution of SA02 and subsequent solutions with diluent solution. QC-A spiking solutions (SS-A-QC-Low, Mid-1, Mid-2, High, Dil) ranging from 3.00 to 16,000 ng/mL were prepared by dilution of CA02 and subsequent solutions with diluent solution.

Sclera/Choroid Homogenate calibration standards (S/CHSTD-A) with Compound 18 concentrations ranging from 0.0500 to 100 ng/mL were prepared by spiking control rabbit S/CH with appropriate calibration curve spiking solutions (SS-A). S/CHQC-A samples with concentrations of 0.150, 5.00, 50.0, and 80.0 ng/mL were prepared by spiking control rabbit S/CH with appropriate SS-A-QC spiking solutions. S/CHQC-A-Dil samples at 800 ng/mL were prepared by spiking control rabbit S/CH with the SS-A-QC-Dil spiking solution at 16,000 ng/mL, and then diluted 20× with control rabbit S/CH prior to extraction.

Compound 18 Ocular Tissue Homogenate (OT2 to OT6) QC Samples Preparation: Ocular tissues (OT2 to OT6) QC-A samples with concentrations of 0.150, 5.00, 50.0, and 80.0 ng/mL were prepared by spiking each type of control rabbit ocular tissue homogenate (OT2 to OT6) with appropriate QC-A spiking solutions.

Compound 18 Lens Homogenate (LH) Standards and QC Samples Preparation: Calibration curve spiking solutions (SS-A-1 to SS-A-10) ranging from 1.00 to 2000 ng/mL were prepared by dilution of SA02 and subsequent solutions with diluent solution. QC-A spiking solutions (SS-A-QC-Low, Mid-1, Mid-2, High, Dil) ranging from 3.00 to 16,000 ng/mL were prepared by dilution of CA02 and subsequent solutions with diluent solution.

LH Homogenate calibration standards (LHSTD-A) with Compound 18 concentrations ranging from 0.0500 to 100 ng/mL were prepared by spiking control rabbit LH with appropriate calibration curve spiking solutions (SS-A). LHQC-A samples with concentrations of 0.150, 5.00, 50.0, and 80.0 ng/mL were prepared by spiking control rabbit LH with appropriate SS-A-QC spiking solutions.

Compound 4 Sclera/Choroid Homogenate (S/CH) Standards and QC Samples Preparation: Calibration curve spiking solutions (SS-B-1 to SS-B-10) ranging from 3.00 to 2000 ng/mL were prepared by dilution of SB02 and subsequent solutions with diluent solution. QC-A spiking solutions (SS-A-QC-Low, Mid-1, Mid-2, High, Dil) ranging from 9.00 to 16,000 ng/mL were prepared by dilution of CB02 and subsequent solutions with diluent solution.

Sclera/Choroid Homogenate calibration standards (S/CHSTD-B) with Compound 4 concentrations ranging from 0.150 to 100 ng/mL were prepared by spiking control rabbit S/CH with appropriate calibration curve spiking solutions (SS-B). S/CHQC-B samples with concentrations of 0.150, 5.00, 50.0, and 80.0 ng/mL were prepared by spiking control rabbit S/CH with appropriate SS-B-QC spiking solutions. S/CHQC-B-Dil samples at 800 ng/mL were prepared by spiking control rabbit S/CH with the SS-B-QC-Dil spiking solution at 16,000 ng/mL, and then diluted 20× with control rabbit S/CH prior to extraction.

Compound 4 Ocular Tissue Homogenate (OT2 to OT7) QC Samples Preparation: Ocular tissues (OT2 to OT7) QC-B samples with concentrations of 0.450, 5.00, 50.0, and 80.0 ng/mL were prepared by spiking each type of control rabbit ocular tissue homogenate (OT2 to OT7) with appropriate QC-A spiking solutions. QC-B-Dil samples at 800 ng/mL were prepared by spiking control rabbit ocular tissue homogenate (OT2) with the QC-B-Dil spiking solution at 16,000 ng/mL, and then diluted 20× with control rabbit ocular tissue homogenate (OT2) prior to extraction.

### Extraction Procedure

1. Thaw ocular tissue homogenate standards, QC samples, and incurred ocular tissue homogenate samples (if applicable) on wet ice. Control ocular tissue homogenate can be thawed in tepid water. Vortex before taking aliquots.

2. Pipet 50 μL of water for Reagent Blank, 50 μL of S/CH control ocular tissue homogenate for Double Blank and Blank, 50 μL of pre-spiked solvent standards, S/CH Standards, S/CH QC samples. LH Standards, and LH QC samples, 50 μL of each type of pre-spiked other ocular tissue samples for other ocular tissue QC samples, 50 μL of incurred ocular tissue homogenate samples (if applicable) into a 96 well plate.

3. Add 200 μL of ACN to the Reagent Blank and Double Blank samples.

4. Add 200 μL of working internal standard (WIS) solution to all remaining samples.

5. Seal the plate and vortex for approximately 5 minutes. Centrifuge the plate at 3500 rpm for 10 minutes at ˜4° C.

6. Transfer ˜200 μL of each sample supernatant into the appropriate wells of a 96 well plate. Submit for LC MS/MS analysis.

### Results

### Dose Administration

Data for the animal dosing are summarized in Table 13 below.

TABLE 13
Dosing data

| | | | | | Compound | |
|---|---|---|---|---|---|---|
| | | | | Body | 18 | Dose |
| | Time | Group | Animal | Weight | Dosed^{a} | Level |
| Phase | Point (h) | Number | Number | (kg) | (mg/day) | (mg/kg) |
| PK^{b} | 0.5, 1, 2, 4, | 2 | 207664 | 2.56 | 2 | 0.782 |
| | 8, 24 | | | | | |
| | 0.5, 1, 2, 4, | 2 | 207665 | 2.27 | 2 | 0.880 |
| | 8, 24 | | | | | |
| | 0.5, 1, 2, 4, | 2 | 207666 | 2.37 | 2 | 0.842 |
| | 8, 24 | | | | | |
| | 0.5, 1, 2, 4, | 2 | 207667 | 2.56 | 2 | 0.781 |
| | 8, 24 | | | | | |
| | | | | | Mean | 0.821 |
| | | | | | SD | 0.0487 |
| TD^{c} | 0.5 | 3 | 207668 | 10.3 | 113.3 | 0.723 |
| | 0.5 | 3 | 207669 | 8.65 | 95.3 | 0.724 |
| | 0.5 | 3 | 207670 | 10.3 | 113.8 | 0.815 |
| | 1 | 1 | 207658 | 2.77 | 2 | 0.831 |
| | 1 | 1 | 207659 | 2.76 | 2 | 0.892 |
| | 1 | 1 | 207660 | 2.45 | 2 | 0.935 |
| | 2 | 3 | 207671 | 2.41 | 1 | 0.816 |
| | 2 | 3 | 207672 | 2.24 | 1 | 0.737 |
| | 2 | 3 | 207673 | 2.14 | 1 | 0.810 |
| | 4 | 2 | 207674 | 2.45 | 1 | 0.862 |
| | 4 | 3 | 207675 | 2.71 | 1 | 0.712 |
| | 4 | 3 | 207676 | 2.47 | 1 | 0.816 |
| | 8 | 3 | 207677 | 2.32 | 1 | 0.811 |
| | 8 | 3 | 207678 | 2.81 | 1 | 0.699 |
| | 8 | 3 | 207679 | 2.45 | 1 | 0.788 |
| | | | | | Mean | 0.798 |
| | | | | | SD | 0.0688 |
| Control | NA^{d} | 4 | 207661 | 2.29 | NA | NA |
| | NA | 4 | 207662 | 2.19 | NA | NA |
| | NA | 4 | 207663 | 2.35 | NA | NA |
| | NA | 5 | 207680 | 2.47 | NA | NA |
| | NA | 5 | 207681 | 2.89 | NA | NA |
| | NA | 5 | 207682 | 2.77 | NA | NA |

^{a}Test Article: Compound 18 nanosuspension, 1 drop/eye/day for 7 consecutive days (0.05 mL/eye/day or 0.1 mL/day for both eyes).
Compound 18 dosed per day = 2% (i.e., 2 g/100 mL)* 0.1 mL = 0.002 g = 2 mg/day
^{b}PK = Pharmacokinetics
^{c}TD = Tissue Distribution
^{d}NA = Not Applicable

A total of 25 rabbits were used for this study, including 19 treated rabbits and 6 control rabbits. The 19 treated rabbits received an eye drop dose formulation, Compound 18 nanosuspension, 1 drop/eye/day for 7 consecutive days (0.05 mL/eye/day or 0.1 mL/day for both eyes). The Compound 18 dosed per day is equivalent to 2% (i.e. 2 g/100 mL)*0.1 mL=0.002 g=2 mg/day.

The 19 treated rabbits include 3 rabbits for Group 1 pilot study (blood and ocular tissues collected at Day 7, 1 h), 4 rabbits for Group 2 PK Phase (blood only collected on Day 1 and Day 7, 0.5, 1, 2, 4, 8, and 24 h), and 12 rabbits for Group 3 TD phase (blood and ocular tissues collected at Day 7, 0.5, 2, 4, and 8 h, n=3 per time point). As set out below, Groups 1 and 3 data were combined as TD phase Day 7, 0.5, 1, 2, 4, and 8 h, n=3 per time point.

The 6 untreated control rabbits were divided into Group 4 (n=3) and Group 5 (n=3). Group 4 rabbits were sacrificed along with Group 1; and Group 5 rabbits were sacrificed along with Groups 2 and 3 to provide control ocular tissues for preparation of standard and QC samples.

The daily doses (mean±SD) were 0.821±0.0487 mg/kg/day for Group 2 (PK Phase, n=4) and 0.798±0.0688 mg/kg/day for Groups 1 and 3 (TD Phase, n=15). These doses were based on the nominal concentration of Compound 18 in the eye drop dose formulation. The mean doses at 0.8 mg/kg/day were used for all pharmacokinetic calculations.

### In-Life Observations

Groups 1-3 animals were normal following dosing, except animal #207664 (Group 2, PK Phase) had discolored skin and rapid respiratory rate on Day 7. Groups 4 and 5 control animals were also normal.

### Bioanalytical Standard Curve and Quality Control Data for Compound 18 in Rabbit Plasma, Aqueous Humor, and Vitreous Humor

The test method for Compound 18 analysis is detailed herein above.

The calibration standards prepared in rabbit plasma were used to quantify plasma, aqueous humor, and vitreous humor QC and incurred samples. The plasma calibration curve ranged from 0.0500 to 100 ng/mL. In order for a calibration curve to be acceptable, at least 75% of the standards must be within ±20% of their nominal value (±25% for LLOQ) with an acceptable standard at both the LLOQ and upper limit of quantitation (ULOQ) and a coefficient of determination (r2) greater than 0.9700. The mean coefficient of determination (r2) was 0.9975.

Plasma standard curve regression acceptance criteria were achieved for each accepted run. The back-calculated concentration of 30 of 30 (100%) standards were within the acceptance criteria for standard curve samples. The % bias of the mean concentrations for the standards ranged from −3.8 to 4.8.

The plasma, aqueous humor, and vitreous humor quality control samples (QCs) were analyzed in two replicates at the Low, Mid-1, Mid-2, and High concentration levels. For run acceptance, at least two thirds of the total numbers of these QC samples were required to have calculated concentrations within ±20% (plasma) or ±30% (aqueous humor and vitreous humor) of nominal values with at least 50% acceptable at each concentration level. QC samples met the acceptance criteria.

Acceptance criteria were satisfied in 24 of the 24 (100%) plasma QC samples evaluated. The % bias of the respective mean plasma QC sample concentrations ranged from −2.0 to 2.3 with CV ≤8.2%.

Acceptance criteria were satisfied in 13 of the 16 (81.3%) aqueous humor QC samples evaluated. The % bias of the respective mean aqueous humor QC sample concentrations ranged from −22.0 to −16.8 with CV ≤17.9%. For pilot study (TD phase, Day 7, 1 h) sample analysis in WR1, the run was accepted despite both aqueous humor QC-Mid-1 at 5.00 ng/mL did not meet the acceptance criteria. As all other aqueous humor QC levels met the acceptance criteria and the pilot study sample concentrations were BQL (two samples) or around the QC-Low concentration at 0.15 ng/mL (one sample), the data were accepted.

Acceptance criteria were satisfied in 8 of the 8 (100%) vitreous humor QC samples evaluated. The % bias of the respective mean vitreous humor QC sample concentrations ranged from 7.8 to 15.2 with CV ≤5.9%.

### Bioanalytical Standard Curve and Quality Control Data for Compound 18 in Rabbit Sclera/Choroid, Cornea, Iris/Ciliary Body, Optic Nerve, Retina, and Trabecular Meshwork Homogenate

The test method for Compound 18 analysis is detailed herein above.

The calibration standards prepared in rabbit sclera/choroid homogenate were used to quantify sclera/choroid, cornea, iris/ciliary body, optic nerve, retina, and trabecular meshwork homogenate QC and incurred samples. The sclera/choroid calibration curve ranged from 0.0500 to 100 ng/mL. In order for a calibration curve to be acceptable, at least 75% of the standards must be within ±20% of their nominal value (±25% for LLOQ) with an acceptable standard at both the LLOQ and upper limit of quantitation (ULOQ) and a coefficient of determination (r2) greater than 0.9700. The mean coefficient of determination (r2) was 0.9950.

Sclera/choroid standard curve regression acceptance criteria were achieved for each accepted run. The back-calculated concentration of 30 of 30 (100%) standards were within the acceptance criteria for standard curve samples. The % bias of the mean concentrations for the standards ranged from −5.1 to 6.0.

The sclera/choroid, cornea, iris/ciliary body, optic nerve, retina, and trabecular meshwork quality control samples (QCs) were analyzed in two replicates at the Low, Mid-1, Mid-2, and High concentration levels. For run acceptance, at least two thirds of the total numbers of these QC samples were required to have calculated concentrations within ±30% of nominal values with at least 50% acceptable at each concentration level. QC samples met the acceptance criteria.

Acceptance criteria were satisfied in 27 of the 27 (100%) sclera/choroid QC samples evaluated. The % bias of the respective mean sclera/choroid QC sample concentrations ranged from −26.6 to 5.0 with CV ≤10.6%.

Acceptance criteria were satisfied in 12 of the 19 (63.2%) cornea QC samples evaluated. The % bias of the respective mean cornea QC sample concentrations ranged from −22.1 to 21.5 with CV ≤18.0.

Acceptance criteria were satisfied in 16 of the 16 (100%) iris/ciliary body QC samples evaluated. The % bias of the respective mean iris/ciliary body QC sample concentrations ranged from −6.6 to 6.4 with CV ≤21.1%.

Acceptance criteria were satisfied in 16 of the 16 (100%) optic nerve QC samples evaluated. The % bias of the respective mean optic nerve QC sample concentrations ranged from −16.0 to −8.0 with CV ≤9.7%.

Acceptance criteria were satisfied in 13 of the 16 (81.3%) retina QC samples evaluated. The % bias of the respective mean retina QC sample concentrations ranged from 2.2 to 17.3 with CV ≤35.0%.

Acceptance criteria were satisfied in 13 of the 16 (81.3%) trabecular meshwork QC samples evaluated. The % bias of the respective mean trabecular meshwork QC sample concentrations ranged from −24.2 to 6.4 with CV ≤29.8%.

### Bioanalytical Standard Curve and Quality Control Data for Compound 18 in Rabbit Lens Homogenate

The test method for Compound 18 analysis is detailed herein above.

The calibration standards prepared in rabbit lens homogenate were used to quantify lens homogenate QC and incurred samples. The lens calibration curve ranged from 0.0500 to 100 ng/mL. In order for a calibration curve to be acceptable, at least 75% of the standards must be within ±20% of their nominal value (±25% for LLOQ) with an acceptable standard at both the LLOQ and upper limit of quantitation (ULOQ) and a coefficient of determination (r2) greater than 0.9700. The coefficient of determination (r2) was 0.9982.

Lens standard curve regression acceptance criteria were achieved for each accepted run. The back-calculated concentration of 10 of 10 (100%) standards were within the acceptance criteria for standard curve samples. The % bias of the mean concentrations for the standards ranged from −8.0 to 3.8.

The lens quality control samples (QCs) were analyzed in two replicates at the Low, Mid-1, Mid-2, and High concentration levels. For run acceptance, at least two thirds of the total numbers of these QC samples were required to have calculated concentrations within ±30% of nominal values with at least 50% acceptable at each concentration level. QC samples met the acceptance criteria.

Acceptance criteria were satisfied in 8 of the 8 (100%) lens QC samples evaluated. The % bias of the respective mean lens QC sample concentrations ranged from 4.5 to 25.3.

### Bioanalytical Standard Curve and Quality Control Data for Compound 4 in Rabbit Plasma, Aqueous Humor, and Vitreous Humor

The test method for Compound 4 analysis is detailed herein above.

The calibration standards prepared in rabbit plasma were used to quantify plasma, aqueous humor, and vitreous humor QC and incurred samples. The plasma calibration curve ranged from 0.150 to 100 ng/mL. In order for a calibration curve to be acceptable, at least 75% of the standards must be within ±20% of their nominal value (±25% for LLOQ) with an acceptable standard at both the LLOQ and upper limit of quantitation (ULOQ) and a coefficient of determination (r2) greater than 0.9700. The mean coefficient of determination (r2) was 0.9985.

Plasma standard curve regression acceptance criteria were achieved for each accepted run. The back-calculated concentration of 40 of 40 (100%) standards were within the acceptance criteria for standard curve samples. The % bias of the mean concentrations for the standards ranged from −4.2 to 3.8.

The plasma, aqueous humor, and vitreous humor quality control samples (QCs) were analyzed in two replicates at the Low, Mid-1, Mid-2, and High concentration levels. For run acceptance, at least two thirds of the total numbers of these QC samples were required to have calculated concentrations within ±30% of nominal values with at least 50% acceptable at each concentration level. QC samples met the acceptance criteria.

Acceptance criteria were satisfied in 38 of the 38 (100%) plasma QC samples evaluated. The % bias of the respective mean plasma QC sample concentrations ranged from −4.3 to 3.3 with CV ≤7.6%.

Acceptance criteria were satisfied in 30 of the 30 (100%) aqueous humor QC samples evaluated. The % bias of the respective mean aqueous humor QC sample concentrations ranged from −10.8 to 5.1 with CV ≤9.4%.

Acceptance criteria were satisfied in 8 of the 8 (100%) vitreous humor QC samples evaluated. The % bias of the respective mean vitreous humor QC sample concentrations ranged from 3.8 to 8.4 with CV ≤2.3%.

### Bioanalytical Standard Curve and Quality Control Data for Compound 4 in Rabbit Sclera/Choroid, Cornea, Iris/Ciliary Body, Lens, Optic Nerve, Retina, and Trabecular Meshwork Homogenate

The test method for Compound 4 analysis is detailed herein above. The calibration standards prepared in rabbit sclera/choroid homogenate were used to quantify sclera/choroid, cornea, iris/ciliary body, lens, optic nerve, retina, and trabecular meshwork homogenate QC and incurred samples. The sclera/choroid calibration curve ranged from 0.1500 to 100 ng/mL. In order for a calibration curve to be acceptable, at least 75% of the standards must be within ±20% of their nominal value (±25% for LLOQ) with an acceptable standard at both the LLOQ and upper limit of quantitation (ULOQ) and a coefficient of determination (r2) greater than 0.9700. The mean coefficient of determination (r2) was 0.9972.

Sclera/choroid standard curve regression acceptance criteria were achieved for each accepted run. The back-calculated concentration of 39 of 40 (97.5%) standards were within the acceptance criteria for standard curve samples. The % bias of the mean concentrations for the standards ranged from −4.8 to 5.3.

The sclera/choroid, cornea, iris/ciliary body, lens, optic nerve, retina, and trabecular meshwork quality control samples (QCs) were analyzed in two replicates at the Low, Mid-1, Mid-2, and High concentration levels. For run acceptance, at least two thirds of the total numbers of these QC samples were required to have calculated concentrations within ±30% of nominal values with at least 50% acceptable at each concentration level. QC samples met the acceptance criteria.

Acceptance criteria were satisfied in 37 of the 38 (97.4%) sclera/choroid QC samples evaluated. The % bias of the respective mean sclera/choroid QC sample concentrations ranged from −19.4 to 13.3 with CV ≤11.4%.

Acceptance criteria were satisfied in 22 of the 22 (100%) cornea QC samples evaluated. The % bias of the respective mean cornea QC sample concentrations ranged from −13.1 to 14.2 with CV ≤6.9%.

Acceptance criteria were satisfied in 16 of the 16 (100%) iris/ciliary body QC samples evaluated. The % bias of the respective mean iris/ciliary body QC sample concentrations ranged 10.0 to 17.3 with CV ≤5.2%.

Acceptance criteria were satisfied in 16 of the 16 (100%) lens QC samples evaluated. The % bias of the respective mean lens QC sample concentrations ranged from 12.0 to 15.3 with CV ≤6.8%.

Acceptance criteria were satisfied in 16 of the 16 (100%) optic nerve QC samples evaluated. The % bias of the respective mean optic nerve QC sample concentrations ranged from −2.6 to 1.1 with CV ≤9.1%.

Acceptance criteria were satisfied in 16 of the 16 (100%) retina QC samples evaluated. The % bias of the respective mean retina QC sample concentrations ranged from 3.0 to 13.0 with CV ≤26.1%.

Acceptance criteria were satisfied in 16 of the 16 (100%) trabecular meshwork QC samples evaluated. The % bias of the respective mean trabecular meshwork QC sample concentrations ranged from 5.1 to 18.2 with CV ≤6.6%.

### PK Phase (Group 2): Plasma Concentrations and Pharmacokinetic Parameters Compound 18 and Compound 4

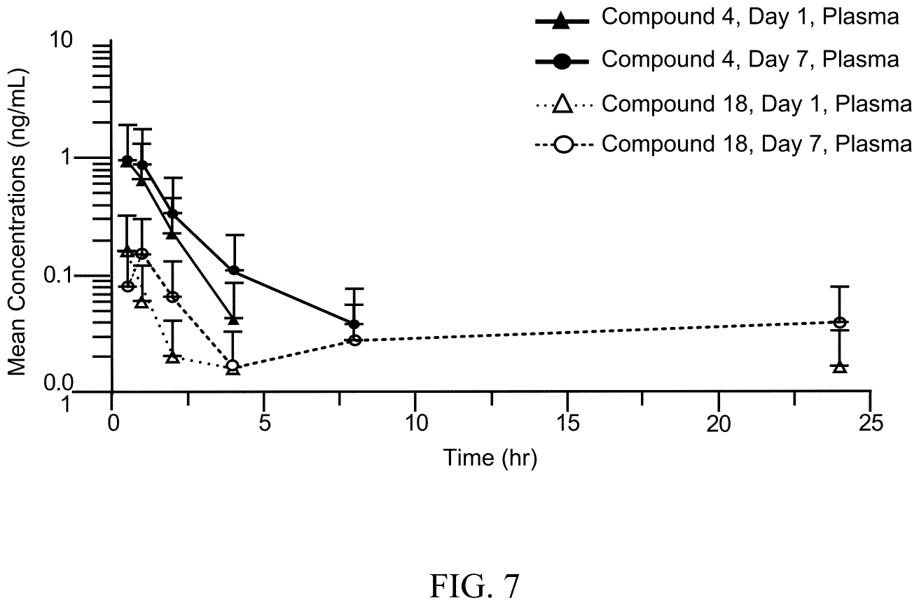

Individual animal and mean plasma concentration data are shown in Table 14. A graph of mean plasma concentrations (Compound 18 and Compound 4) versus time for days 1 and 7 is shown in FIG. 7.

TABLE 14
PK Phase (Group 2) Plasma Compound 18 and Compound 14
Concentration Data

| | Time | Animal # | | | | | Watson |
|---|---|---|---|---|---|---|---|
| Group | (h) | 207664 | 207665 | 207666 | 207667 | Mean | Run |

Day 1 Compound 18 Plasma Concentration (ng/mL)

| | | | | | | | |
|---|---|---|---|---|---|---|---|
| 2 | 0.5 | 0.0863 | 0.0721 | 0.313 | 0.171 | 0.161 | 13 |
| | 1 | BQL^{a} | BQL | 0.183 | 0.0575 | 0.0601 | 13 |
| | 2 | BQL | BQL | BQL | 0.0830 | 0.0208 | 13 |
| | 4 | BQL | BQL | 0.0655 | BQL | 0.0164 | 13 |
| | 8 | BQL | BQL | BQL | BQL | 0 | 13 |
| | 24 | BQL | BQL | 0.0682 | BQL | 0.0171 | 13 |

Day 7 Compound 18 Plasma Concentration (ng/mL)

| | | | | | | | |
|---|---|---|---|---|---|---|---|
| 2 | 0.5 | 0.0978 | BQL | 0.163 | 0.0634 | 0.0811 | 13 |
| | 1 | 0.306 | BQL | 0.131 | 0.161 | 0.150 | 13 |
| | 2 | 0.188 | BQL | 0.0747 | BQL | 0.0657 | 13 |
| | 4 | BQL | BQL | 0.0678 | BQL | 0.0170 | 13 |
| | 8 | BQL | 0.0505 | 0.0611 | BQL | 0.0279 | 13 |
| | 24 | BQL | BQL | 0.0989 | 0.0614 | 0.0401 | 13 |

Day 1 Compound 4 Plasma Concentration (ng./mL)

| | | | | | | | |
|---|---|---|---|---|---|---|---|
| 2 | 0.5 | 0.497 | 0.527 | 1.70 | 1.05 | 0.944 | 13 |
| | 1 | 0.310 | 0.228 | 1.52 | 0.535 | 0.648 | 13 |
| | 2 | 0.188 | BQL | 0.202 | 0.517 | 0.227 | 13 |
| | 4 | BQL | BQL | 0.171 | BQL | 0.0428 | 13 |
| | 8 | BQL | BQL | BQL | BQL | 0 | 13 |
| | 24 | BQL | BQL | BQL | BQL | 0 | 13 |

Day 7 Compound 4 Plasma Concentration (ng/mL)

| | | | | | | | |
|---|---|---|---|---|---|---|---|
| 2 | 0.5 | 0.851 | 0.938 | 0.794 | 1.18 | 0.941 | 13 |
| | 1 | 0.689 | 0.714 | 0.851 | 1.21 | 0.866 | 13 |
| | 2 | 0.373 | 0.273 | 0.423 | 0.266 | 0.334 | 13 |
| | 4 | BQL | BQL | 0.254 | 0.180 | 0.109 | 13 |
| | 8 | BQL | 0.153 | BQL | BQL | 0.0383 | 13 |
| | 24 | BQL | BQL | BQL | BQL | 0 | 13 |

^{a}BQL = Below lower limit of quantitation (0.0500 ng/mL for Compound 18 and 0.150 ng/mL for Compound 4). BQL values were set to 0 for calculation of mean and pharmacokinetics.

Pharmacokinetic parameters for Compound 18 and Compound 4 in plasma are shown in Table 15. The plasma PK parameters were calculated from the individual animal plasma concentrations on Day 1 and Day 7.

TABLE 15
PK Phase (Group 2) Plasma Compound 18 and Compound 4 Pharmacokinetic
Parameters

| | | | Dose | | | | | | |
|---|---|---|---|---|---|---|---|---|---|
| | | | Level | Dose | Animal | Tₘₐₓ | T_{1/2} | Cₘₐₓ | AUC₀₋₂₄ |
| Group | Analyte | Day | (mg/kg) | Route | Number | (h) | (h) | (ng/mL) | (h*ng/mL) |
| 2 | Compound | 1 | 0.8 | Ocular | 207664 | 0.5 | NC | 0.0863 | 0.0216 |
| | 18 | | | Instillation | 207665 | 0.5 | NC | 0.0721 | 0.0180 |
| | | | | | 207666 | 0.5 | NC | 0.313 | 1.04 |
| | | | | | 207667 | 0.5 | NC | 0.171 | 0.170 |
| | | | | | Mean | 0.5 | | 0.161 | 0.311 |
| | | | | | SD | 0.0 | | 0.111 | 0.488 |
| | | 7 | 0.8 | Ocular | 207664 | 1 | NC | 0.306 | 0.372 |
| | | | | Instillation | 207665 | 8 | NC | 0.0505 | 0.101 |
| | | | | | 207666 | 0.5 | NC | 0.163 | 1.897 |
| | | | | | 207667 | 1 | NC | 0.161 | 0.644 |
| | | | | | Mean | 2.6 | | 0.170 | 0.754 |
| | | | | | SD | 3.6 | | 0.105 | 0.794 |
| | Compound | 1 | 0.8 | Ocular | 207664 | 0.5 | NR | 0.497 | 0.575 |
| | 4 | | | Instillation | 207665 | 0.5 | 0.414 | 0.527 | 0.321 |
| | | | | | 207666 | 0.5 | NR | 1.70 | 2.46 |
| | | | | | 207667 | 0.5 | NR | 1.05 | 1.18 |
| | | | | | Mean | 0.5 | | 0.944 | 1.14 |
| | | | | | SD | 0.0 | | 0.565 | 0.957 |
| | | 7 | 0.8 | Ocular | 207664 | 0.5 | NR | 0.851 | 1.13 |
| | | | | Instillation | 207665 | 0.5 | 0.823 | 0.938 | 1.72 |
| | | | | | 207666 | 1 | 1.82 | 0.851 | 1.92 |
| | | | | | 207667 | 1 | NR | 1.21 | 2.08 |
| | | | | | Mean | 0.75 | 132 | 0.963 | 1.71 |
| | | | | | SD | 0.29 | 0.702 | 0.170 | 0.416 |

For Compound 18 in plasma, the Tₘₐₓ values (mean±SD, n=4) were 0.5±0.0 hour on Day 1 and 2.6±3.6 hours on Day 7, indicating rapid absorption of Compound 18 after ocular instillation. The T_{1/2} values were not calculated due to insufficient data points in the elimination phase. The Cₘₐₓ values (mean±SD, n=4) were 0.161±0.111 ng/mL on Day 1 and 0.170±0.105 ng/mL on Day 7. The AUC0-24 values (mean±SD, n=4) were 0.311±0.488 h*ng/mL on Day 1 and 0.754±0.794 h*ng/mL on Day 7.

For Compound 4 in plasma, the Tₘₐₓ values (mean±SD, n=4) were 0.5±0.0 hour for Day 1 and 0.75±0.29 hour for Day 7, indicating rapid metabolism to Compound 4 after ocular instillation of Compound 18 followed by rapid absorption. The T_{1/2} values were reportable for one rabbit (T_{1/2}=0.414 h) on Day 1 and for two rabbits (T_{1/2}=1.32±0.702 h, n=2) on Day 7, due to extensive extrapolation (>30%) for the elimination phase for the others. The Cₘₐₓ values (mean±SD, n=4) were 0.944±0.565 ng/mL on Day 1 and 0.963±0.170 ng/mL on Day 7. The AUC₀₋₂₄ values (mean±SD, n=4) were 1.14±0.957 h*ng/mL on Day 1 and 1.71±0.416 h*ng/mL on Day 7.

### Tissue Distribution Phase (Groups 1 and 3): Plasma and Ocular Tissue Concentrations and Pharmacokinetic Parameters (Compound 18 and Compound 4)

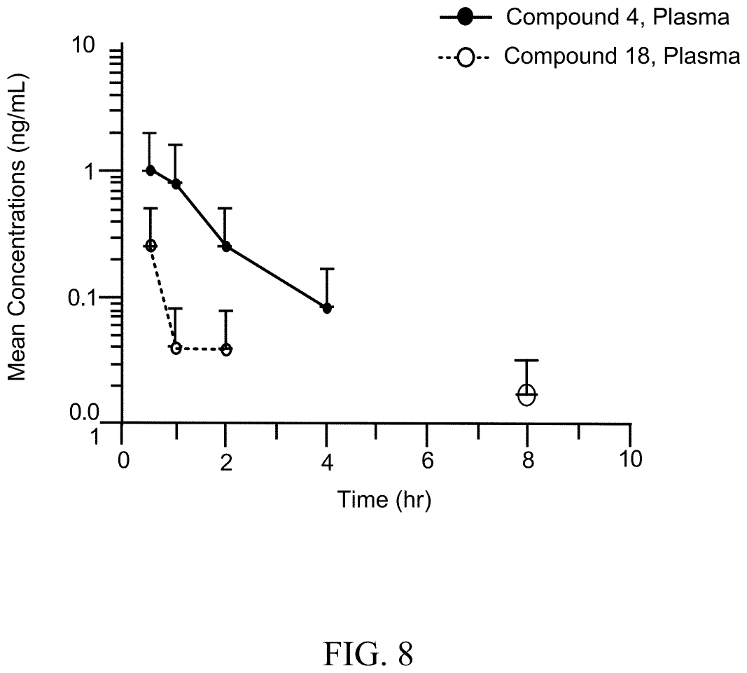

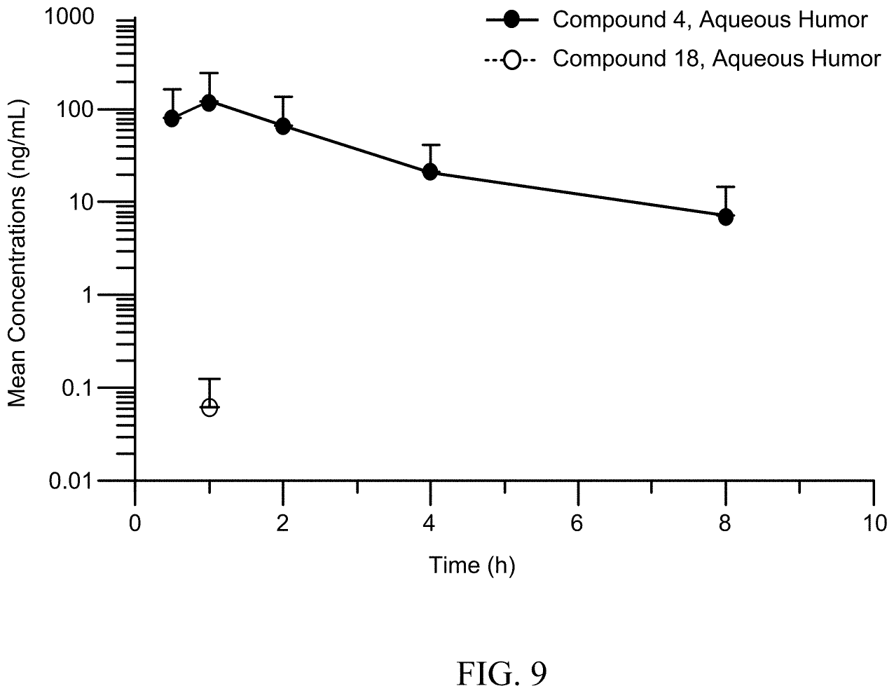

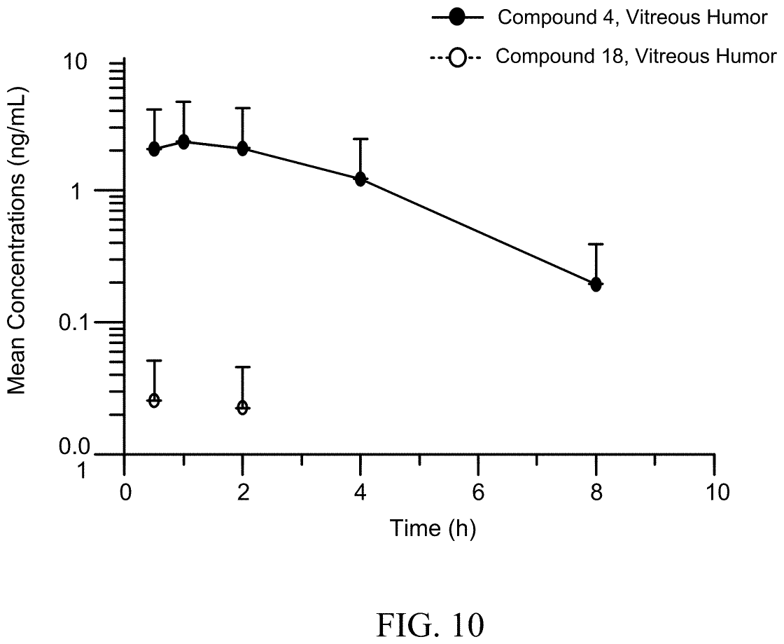

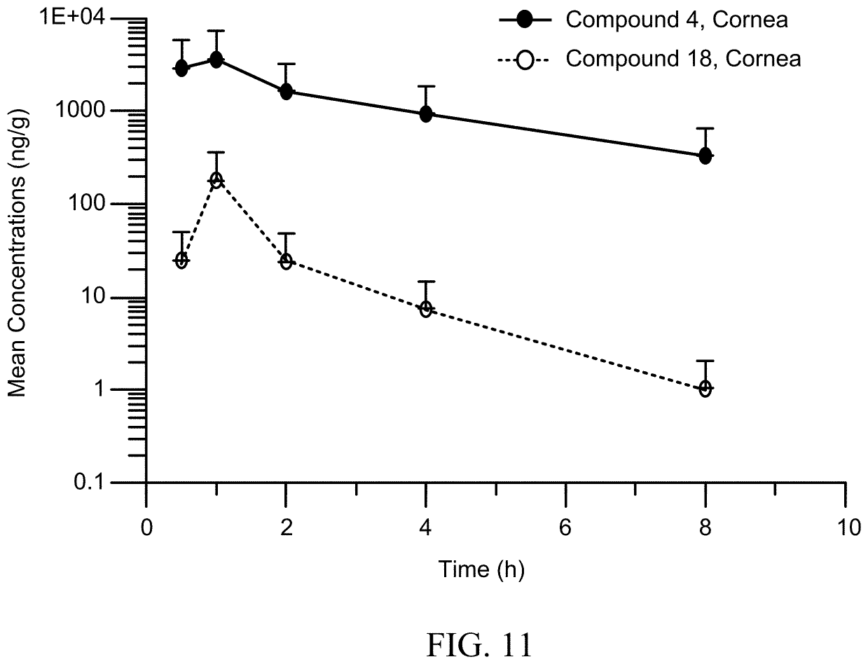

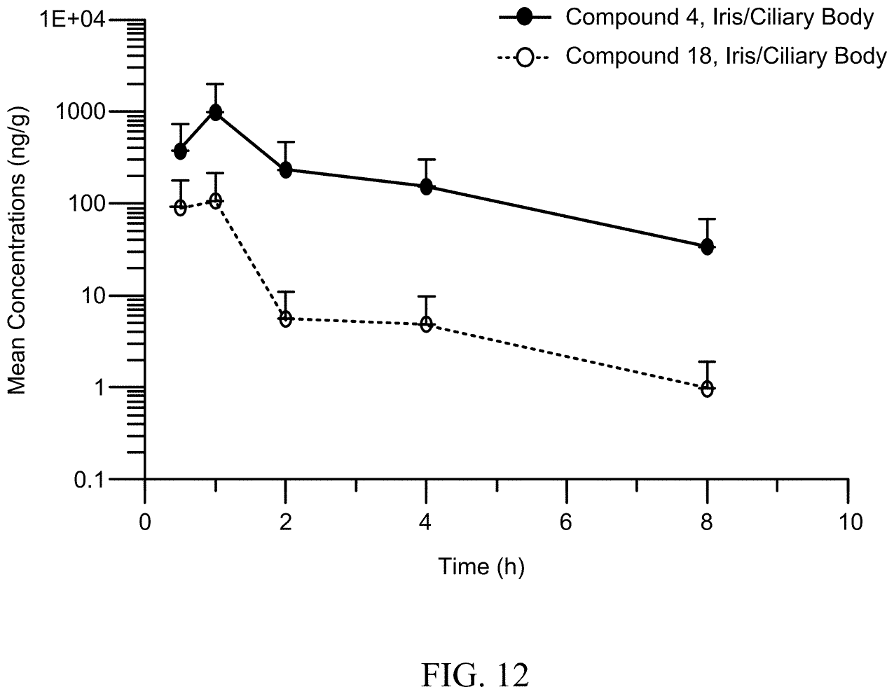

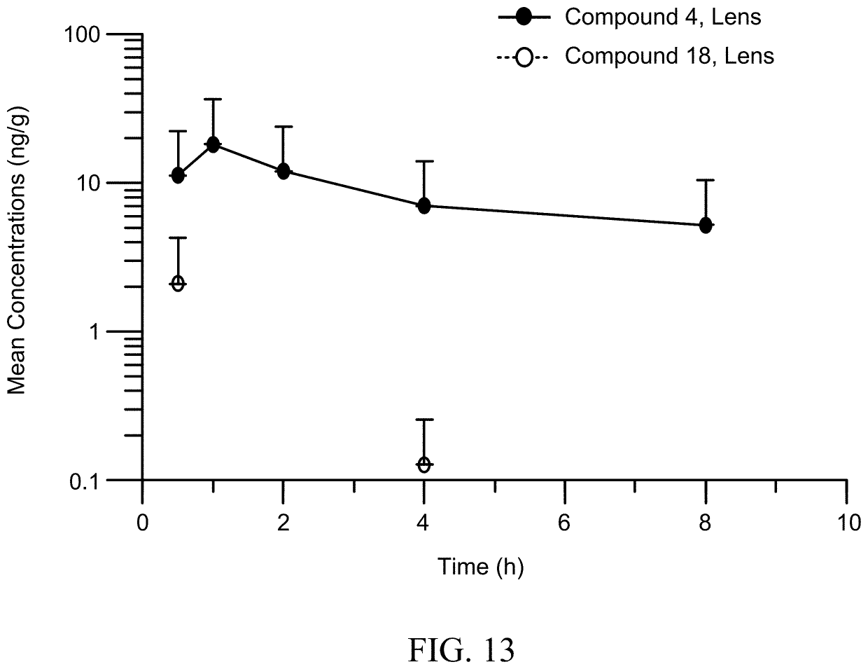

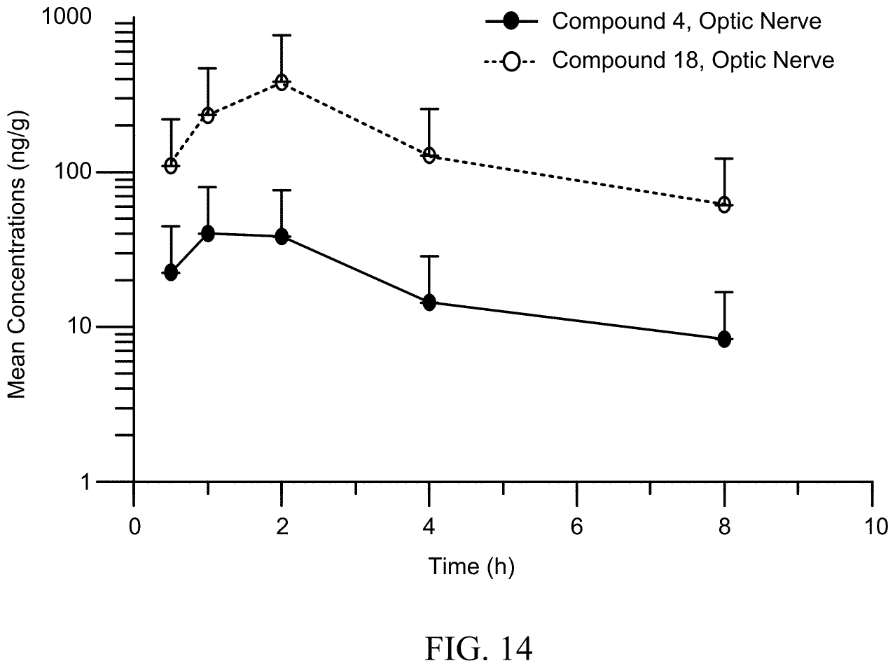

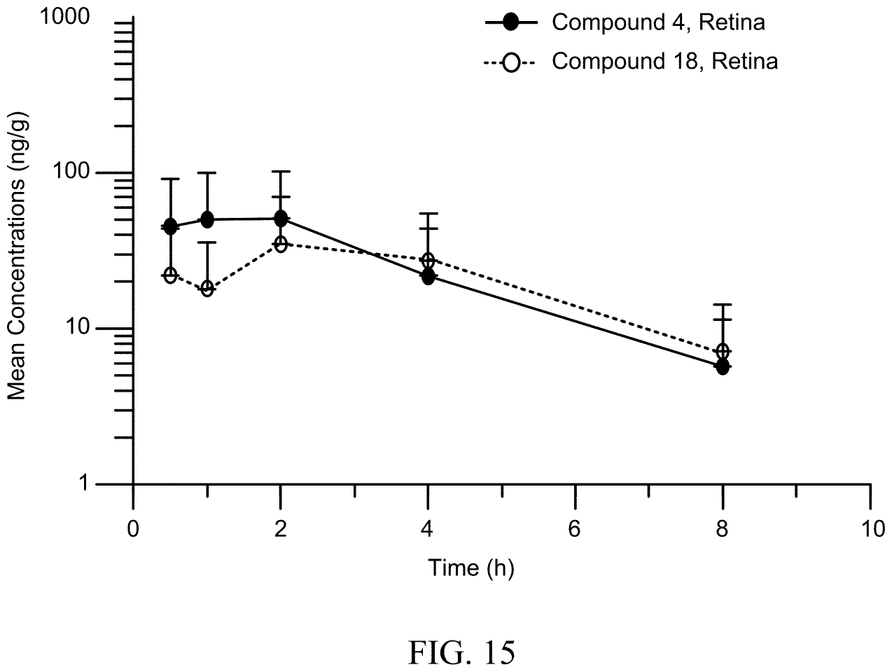

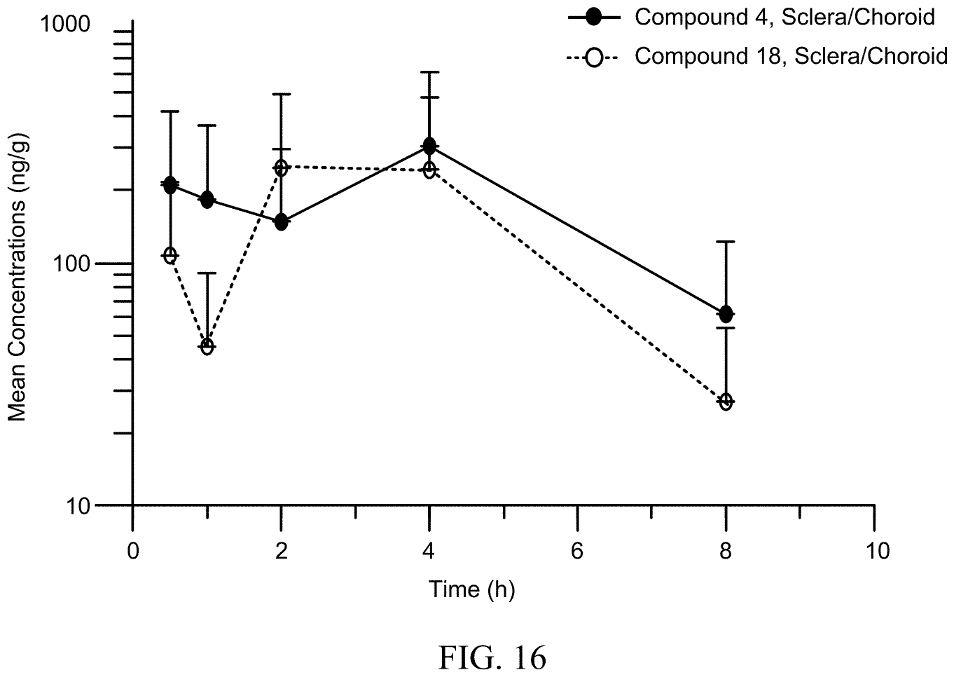

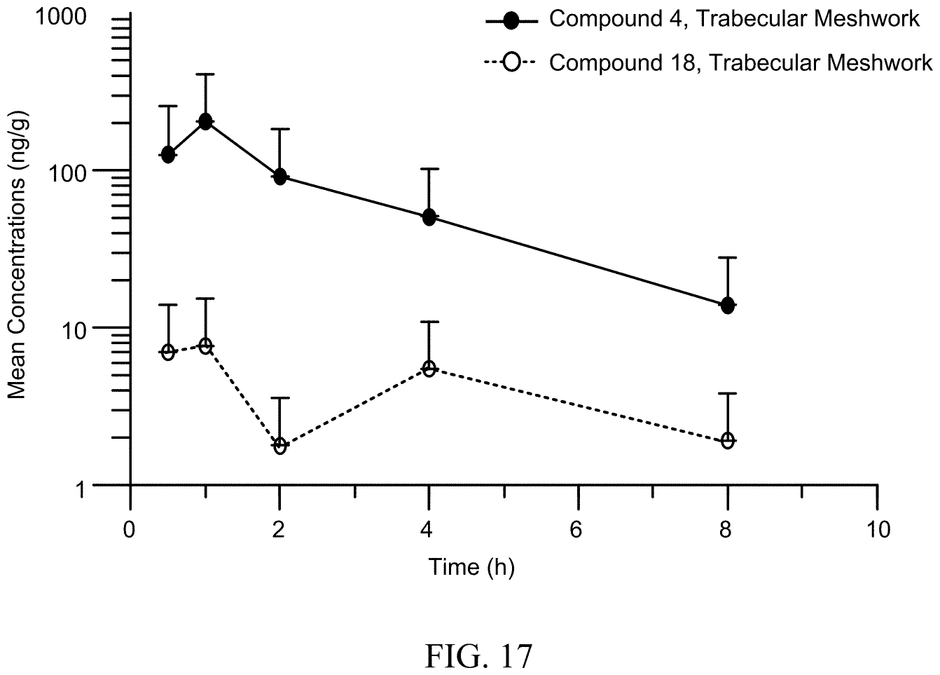

Individual animal and mean plasma and ocular tissue Compound 18 and Compound 4 concentration data are shown below in Table 16 (Plasma), Table 17 (Aqueous Humor), Table 18 (vitreous humor). Table 19 (Cornea), Table 20 (Iris/Ciliary Body), Table 21 (Lens), Table 22 (Optic Nerve), Table 23 (Retina), Table 24 (Sclera/Choroid), and Table 25 (Trabecular Meshwork). Graphs of mean plasma and ocular tissue Compound 18 and Compound 4 concentrations versus time are shown in the following figures: FIG. 8 (Plasma), FIG. 9 (Aqueous Humor), FIG. 10 (vitreous humor), FIG. 11 (Cornea), FIG. 12 (Iris/Ciliary Body), FIG. 13 (Lens), FIG. 14 (Optic Nerve), FIG. 15 (Retina), FIG. 16 (Sclera/Choroid), and FIG. 17 (Trabecular Meshwork).

TABLE 16
TD Phase (Groups 1 and 3) Plasma Compound 18 and Compound 4
Concentration data

| | | | Compound | | |
|---|---|---|---|---|---|
| | | | 18 | Compound 4 | |
| | Time Point | Animal | Plasma Conc. | Plasma Conc. | |
| Group | (h) | Number | (ng/mL) | (ng/mL) | Watson Run |
| 3 | 0.5 | 207668 | 0.0599 | 0.812 | 12 |
| | | 207669 | 0.627 | 1.22 | 12 |
| | | 207670 | 0.0972 | 1.03 | 12 |
| | | Mean | 0.261 | 1.02 | |
| 1 | 1 | 207658 | BQL^{a} | 0.412 | 1 |
| | | 207659 | 0.0641 | 1.14 | 1 |
| | | 207660 | 0.0581 | 0.839 | 1 |
| | | Mean | 0.0407 | 0.797 | |
| 3 | 2 | 207671 | BQL | 0.309 | 12 |
| | | 207672 | BQL | BQL | 12 |
| | | 207673 | 0.119 | 0.457 | 12 |
| | | Mean | 0.0397 | 0.255 | |
| 3 | 4 | 207674 | BQL | 0.251 | 12 |
| | | 207675 | BQL | BQL | 12 |
| | | 207676 | BQL | BQL | 12 |
| | | Mean | BQL | 0.0837 | |
| 3 | 8 | 207677 | BQL | BQL | 12 |
| | | 207678 | BQL | BQL | 12 |
| | | 207679 | 0.0517 | BQL | 12 |
| | | Mean | 0.0172 | BQL | |

^{a}BQL = Below lower limit of quantitation (0.0500 ng/mL for Compound 18 and 0.150 ng/mL, for Compound 4). BQL values were set to 0 for calculation of mean and pharmacokinetics.

TABLE 17
TD Phase (Groups 1 and 3) Aqueous Humor Compound 18 and
Compound 4 Concentration data

| | | | Compound | | |
|---|---|---|---|---|---|
| | | | 18 | Compound 4 | |
| | | | Aqueous | Aqueous | |
| | Time Point | Animal | Humor Conc. | Humor Conc. | |
| Group | (h) | Number | (ng/mL) | (ng/mL) | Watson Run^{b} |
| 3 | 0.5 | 207668 | BQL^{a} | 80.7 | 12 |
| | | 207669 | BQL | 81.2 | 12 |
| | | 207670 | BQL | 83.0 | 12 |
| | | Mean | BQL | 81.6 | |
| 1 | 1 | 207658 | BQL | 137 | 1, 2^{b} |
| | | 207659 | BQL | 106 | 1, 2 |
| | | 207660 | 0.189 | 129 | 1, 2 |
| | | Mean | 0.0630 | 124 | |
| 3 | 2 | 207671 | BQL | 97.3 | 12 |
| | | 207672 | BQL | 43.7 | 12 |
| | | 207673 | BQL | 62.2 | 12 |
| | | Mean | BQL | 67.7 | |
| 3 | 4 | 207674 | BQL | 27.9 | 12 |
| | | 207675 | BQL | 22.8 | 12 |
| | | 207676 | BQL | 12.9 | 12 |
| | | Mean | BQL | 21.2 | |
| 3 | 8 | 207677 | BQL | 3.61 | 12 |
| | | 207678 | BQL | 4.69 | 12 |
| | | 207679 | BQL | 13.6 | 12 |
| | | Mean | BQL | 7.30 | |

^{a}BQL = Below lower limit of quantitation (0.0500 ng/mL for Compound 18 and 0.150 ng/mL for Compound 4). BQL values were set to 0 for calculation of mean and pharmacokinetics.
^{b}Compound 18 concentrations were determined in WR1 and WR12. Compound 4 concentrations were determined in WR2 and WR12.

TABLE 18
TD Phase (Groups 1 and 3) Vitreous Humor Compound 18 and
Compound 4 Concentration Data

| | | | Compound | Compound | |
|---|---|---|---|---|---|
| | | | 18 | 4 | |
| | | | Vitreous | Vitreous | |
| | Time Point | Animal | Humor Conc. | Humor Conc. | |
| Group | (h) | Number | (ng/mL) | (ng/mL) | Watson Run |
| 3 | 0.5 | 207668 | BQL^{a} | 0.743 | 12 |
| | | 207669 | 0.0771 | 3.24 | 12 |
| | | 207670 | BQL | 2.17 | 12 |
| | | Mean | 0.0257 | 2.05 | |
| 1 | 1 | 207658 | BQL | 2.05 | 12 |
| | | 207659 | BQL | 2.22 | 12 |
| | | 207660 | BQL | 2.69 | 12 |
| | | Mean | BQL | 2.32 | |
| 3 | 2 | 207671 | BQL | 3.42 | 12 |
| | | 207672 | 0.0683 | 2.06 | 12 |
| | | 207673 | BQL | 0.717 | 12 |
| | | Mean | 0.0228 | 2.07 | |
| 3 | 4 | 207674 | BQL | 1.04 | 12 |
| | | 207675 | BQL | 2.36 | 12 |
| | | 207676 | BQL | 0.245 | 12 |
| | | Mean | BQL | 1.22 | |
| 3 | 8 | 207677 | BQL | 0.317 | 12 |
| | | 207678 | BQL | BQL | 12 |
| | | 207679 | BQL | 0.267 | 12 |
| | | Mean | BQL | 0.195 | |

^{a}BQL = Below lower limit of quantitation (0.0500 ng/mL for Compound 18 and 0.150 ng/mL for Compound 4). BQL values were set to 0 for calculation of mean and pharmacokinetics.

TABLE 19
TD Phase (Groups 1 and 3) Cornea Compound 18 and Compound 4 Concentration
Data

| | | | Compound 8 | Compound 4 | | | | |
|---|---|---|---|---|---|---|---|---|
| | | | Cornea | Cornea | | | Final | Final |
| | Time | | Homogenate | Homogenate | | Homogenate | Compound 8 | Compound 4 |
| | Point | Animal | Conc. | Conc. | Watson | Dilution | Cornea | Cornea |
| Group | (hr) | # | (ng/mL) | (ng/mL) | Run | Factor | Conc. (ng/g) | Conc. (ng/g) |
| 3 | 0.5 | 207668 | 0.187 | 165 | 11 | 15 | 2.81 | 2475 |
| | | 207669 | BQL^{a} | 166 | 11 | 15 | 0 | 2490 |
| | | 207670 | 4.80 | 253 | 11 | 15 | 72.0 | 3795 |
| | | Mean | | | | | 24.9 | 2920 |
| 1 | 1 | 207658 | 0.495 | 201 | 5 | 15 | 7.43 | 3015 |
| | | 207659 | 0.592 | 155 | 5 | 15 | 8.88 | 2325 |
| | | 207660 | 35.1 | 365 | 5 | 15 | 527 | 5475 |
| | | Mean | | | | | 181 | 3605 |
| 3 | 2 | 207671 | 2.80 | 131 | 11 | 15 | 42.0 | 1965 |
| | | 207672 | 2.05 | 97.8 | 11 | 15 | 30.8 | 1467 |
| | | 207673 | BQL | 97.5 | 11 | 15 | 0 | 1463 |
| | | Mean | | | | | 24.3 | 1632 |
| 3 | 4 | 207674 | 0.528 | 96.4 | 11 | 15 | 7.92 | 1446 |
| | | 207675 | BQL | 42.7 | 11 | 15 | 0 | 641 |
| | | 207676 | 0.961 | 48.7 | 11 | 15 | 14.4 | 731 |
| | | Mean | | | | | 7.44 | 939 |
| 3 | 8 | 207677 | 0.156 | 15.1 | 11 | 15 | 2.34 | 227 |
| | | 207678 | 0.0516 | 18.5 | 11 | 15 | 0.774 | 278 |
| | | 207679 | BQL | 33.0 | 11 | 15 | 0 | 495 |
| | | Mean | | | | | 1.04 | 333 |

^{a}BQL = Below lower limit of quantitation (0.0500 ng/mL for Compound 18 and 0.150 ng/mL for Compound 4 in homogenates, corresponding to 0.750 ng/g for Compound 18 and 2.25 ng/g for Compound 4 in cornea tissues). BQL values were set to 0 for calculation of concentrations (ng/g) in tissues.

TABLE 20
TD Phase (Groups 1 and 3) Iris/Ciliary Body Compound 18 and Compound 4
Concentration Data

| | | | Compound | Compound | | | | |
|---|---|---|---|---|---|---|---|---|
| | | | 18 | 4 | | | Final | |
| | | | Iris/Ciliary | Iris/Ciliary | | | Compound | Final |
| | | | Body | Body | | | 18 | Compound 4 |
| | Time | | Homogenate | Homogenate | | Homogenate | Iris/Ciliary | Iris/Ciliary |
| | Point | Animal | Conc. | Conc. | Watson | Dilution | Body Conc. | Body Conc. |
| Group | (hr) | Number | (ng/mL) | (ng/mL) | Run | Factor | (ng/g) | (ng/g) |
| 3 | 0.5 | 207668 | 0.112 | 16.7 | 10 | 20 | 2.24 | 334 |
| | | 207669 | BQL^{a} | 13.5 | 10 | 20 | 0 | 270 |
| | | 207670 | 13.4 | 25.5 | 10 | 20 | 268 | 510 |
| | | Mean | | | | | 90.1 | 371 |
| 1 | 1 | 207658 | BQL | 48.8 | 5 | 20 | 0 | 976 |
| | | 207659 | 0.934 | 29.4 | 5 | 20 | 18.7 | 588 |
| | | 207660 | 15.1 | 68.8 | 5 | 20 | 302 | 1376 |
| | | Mean | | | | | 107 | 980 |
| 3 | 2 | 207671 | 0.0637 | 13.4 | 10 | 20 | 1.27 | 268 |
| | | 207672 | 0.772 | 9.56 | 10 | 20 | 15.4 | 191 |
| | | 207673 | BQL | 11.9 | 10 | 20 | 0 | 238 |
| | | Mean | | | | | 5.56 | 232 |
| 3 | 4 | 207674 | 0.289 | 12.4 | 10 | 20 | 5.78 | 248 |
| | | 207675 | BQL | 4.86 | 10 | 20 | 0 | 97.2 |
| | | 207676 | 0.442 | 5.64 | 10 | 20 | 8.84 | 113 |
| | | Mean | | | | | 4.87 | 153 |
| 3 | 8 | 207677 | 0.145 | 0.992 | 10 | 20 | 2.90 | 19.8 |
| | | 207678 | BQL | 1.95 | 10 | 20 | 0 | 39.0 |
| | | 207679 | BQL | 2.16 | 10 | 20 | 0 | 43.2 |
| | | Mean | | | | | 0.967 | 34.0 |

^{a}BQL = Below lower limit of quantitation (0.0500 ng/mL for Compound 18 and 0.150 ng/mL for Compound 4 in homogenates, corresponding to 1.00 ng/g for Compound 18 and 3.00 ng/g for Compound 4 in iris/ciliary body tissues). BQL values were set to 0 for calculation of concentrations (ng/g) in tissues.

TABLE 21
TD Phase Groups 1 and 3) Lens Compound 18 and
Compound 4 Concentration Data

| | | | Compound | Compound | | | | |
|---|---|---|---|---|---|---|---|---|
| | | | 18 | 4 | | | Final | Final |
| | | | Lens | Lens | | | Compound | Compound |
| | Time | | Homogenate | Homogenate | | Homogenate | 18 | 4 |
| | Point | Animal | Conc. | Conc. | Watson | Dilution | Lens Conc. | Lens Conc. |
| Group | (hr) | Number | (ng/mL | (ng/mL) | Run^{b} | Factor | (ng/g) | (ng/g) |
| 3 | 0.5 | 207668 | BQL¹ | 0.893 | 8, 9 | 5 | 0 | 4.47 |
| | | 207669 | BQL | 1.97 | 8, 9 | 5 | 0 | 9.85 |
| | | 207670 | 1.27 | 3.87 | 8, 9 | 5 | 6.35 | 19.4 |
| | | Mean | | | | | 2.12 | 11.2 |
| 1 | 1 | 207658 | BQL | 2.78 | 5, 9 | 5 | 0 | 13.9 |
| | | 207659 | BQL | 4.79 | 5, 9 | 5 | 0 | 24.0 |
| | | 207660 | BQL | 3.26 | 5, 9 | 5 | 0 | 16.3 |
| | | Mean | | | | | 0 | 18.1 |
| 3 | 2 | 207671 | BQL | 2.94 | 8, 9 | 5 | 0 | 14.7 |
| | | 207672 | BQL | 2.04 | 8, 9 | 5 | 0 | 10.2 |
| | | 207673 | BQL | 2.22 | 8, 9 | 5 | 0 | 11.1 |
| | | Mean | | | | | 0 | 12.0 |
| 3 | 4 | 207674 | BQL | 1.52 | 8, 9 | 5 | 0 | 7.60 |
| | | 207675 | 0.0767 | 1.64 | 8, 9 | 5 | 0.384 | 8.20 |
| | | 207676 | BQL | 1.06 | 8, 9 | 5 | 0 | 5.30 |
| | | Mean | | | | | 0.128 | 7.03 |
| 3 | 8 | 207677 | BQL | 0.94 | 8, 9 | 5 | 0 | 4.70 |
| | | 207678 | BQL | 0.681 | 8, 9 | 5 | 0 | 3.41 |
| | | 207679 | BQL | 1.50 | 8, 9 | 5 | 0 | 7.50 |
| | | Mean | | | | | 0 | 5.20 |

¹BQL = Below lower limit of quantitation (0.0500 ng/mL for Compound 18 and 0.150 ng/mL for Compound 4 in homogenates, corresponding to 0.250 ng/g for Compound 18 and 0.75 ng/mL for Compound 4 in lens tissues). BQL values were set to 0 for calculation of concentrations (ng/g) in tissues.
^{b}Compound 18 concentrations were determined in WR9. Compound 4 concentrations were determined in WR5 and WR8.

TABLE 22
TD Phase (Groups 1 and 3) Optic Nerve Compound 18 and Compound 4
Concentration Data

| | | | Compound | | | | | |
|---|---|---|---|---|---|---|---|---|
| | | | 18 | Compound 4 | | | Final | Final |
| | | | Optic Nerve | Optic Nerve | | | Compound | Compound |
| | Time | | Homogenate | Homogenate | | Homogenate | 18 | 4 Optic |
| | Point | Animal | Conc. | Conc. | Watson | Dilution | Optic Nerve | Nerve Conc. |
| Group | (hr) | Number | (ng/mL)a | (ng/mL)^{a} | Run | Factor | Conc. (ng/g) | (ng/g) |
| 3 | 0.5 | 207668 | 5.79 | 1.53 | 10 | 20 | 116 | 30.6 |
| | | 207669 | 1.52 | 0.622 | 10 | 20 | 30.4 | 12.4 |
| | | 707670 | 9.17 | 1.24 | 10 | 20 | 183 | 74.8 |
| | | Mean | | | | | 110 | 22.6 |
| 1 | 1 | 207658 | 11.8 | 1.43 | 5 | 20 | 236 | 28.6 |
| | | 207659 | 9.59 | 2.35 | 5 | 20 | 192 | 47.0 |
| | | 207660 | 13.3 | 2.2.5 | 5 | 20 | 266 | 45.0 |
| | | Mean | | | | | 231 | 40.2 |
| 3 | 2 | 707671 | 1.22 | 0.486 | 10 | 20 | 24.4 | 9.72 |
| | | 207672 | 51.5 | 4.28 | 10 | 20 | 1030 | 85.6 |
| | | 207673 | 3.95 | 0.997 | 10 | 20 | 79.0 | 19.9 |
| | | Mean | | | | | 378 | 38.4 |
| 3 | 4 | 207674 | 12.4 | 1.09 | 10 | 20 | 248 | 21.8 |
| | | 207675 | 1.57 | 0.438 | 10 | 20 | 31.4 | 8.76 |
| | | 207676 | 5.20 | 0.639 | 10 | 20 | 104 | 12.8 |
| | | Mean | | | | | 128 | 14.5 |
| 3 | 8 | 707677 | 1.31 | 0.286 | 10 | 20 | 26.2 | 5.72 |
| | | 207678 | 5.41 | 0.555 | 10 | 20 | 108 | 11.1 |
| | | 707679 | 2.53 | 0.418 | 10 | 20 | 50.6 | 8.36 |
| | | Mean | | | | | 61.6 | 8.39 |

^{a}Limit of quantitation is 0.0500 ng/mL for Compound 18 and 0.150 ng/mL for Compound 4 in homogenates, corresponding to 1.00 ng/g for Compound 18 and 3.00 ng/g for Compound 4 in optic nerve tissues.

TABLE 23
TD Phase (Groups 1 and 3) Retina Compound 18 and Compound 4 Concentration
Data

| | | | Compound | Compound | | | | |
|---|---|---|---|---|---|---|---|---|
| | | | 18 | 4 | | | Final | Final |
| | | | Retina | Retina | | | Compound | Compound |
| | Time | | Homogenate | Homogenate | | Homogenate | 18 | 4 |
| | Point | Animal | Conc. | Conc. | Watson | Dilution | Retina | Retina |
| Group | (hr) | Number | (ng/mL) | (ng/mL) | Run | Factor | Conc. (ng/g) | Conc.(ng/g) |
| 3 | 0.5 | 207668 | 0.798 | 1.29 | 11 | 20 | 16.0 | 25.8 |
| | | 207669 | 1.01 | 3.41 | 11 | 20 | 20.2 | 68.2 |
| | | 207670 | 1.49 | 2.09 | 11 | 20 | 29.8 | 41.8 |
| | | Mean | | | | | 22.0 | 45.3 |
| 1 | 1 | 207658 | 0.312 | 1.28 | 5 | 20 | 6.24 | 25.6 |
| | | 707659 | 1.01 | 4.51 | 5 | 20 | 20.2 | 90.7 |
| | | 207660 | 1.39 | 1.74 | 5 | 20 | 27.8 | 34.8 |
| | | Mean | | | | | 18.1 | 50.2 |
| 3 | 2 | 207671 | 0.192 | 0.719 | 11 | 164 | 31.5 | 118 |
| | | 207672 | 3.47 | 1.17 | 11 | 20 | 69.4 | 23.4 |
| | | 207673 | 0.17 | 0.568 | 11 | 20 | 3.40 | 11.4 |
| | | Mean | | | | | 34.8 | 50.9 |
| 3 | 4 | 207674 | 1.98 | 0.835 | 11 | 20 | 39.6 | 16.7 |
| | | 207675 | 0.301 | 1.73 | 11 | 20 | 6.02 | 34.6 |
| | | 207676 | 1.81 | 0.702 | 11 | 20 | 36.2 | 14.0 |
| | | Mean | | | | | 27.3 | 21.8 |
| 3 | 8 | 207677 | 0.435 | 0.445 | 11 | 20 | 8.70 | 8.90 |
| | | 207678 | 0.182 | BQL^{a} | 11 | 20 | 3.64 | 0 |
| | | 207679 | 0.458 | 0.417 | 11 | 20 | 9.16 | 8.34 |
| | | Mean | | | | | 7.17 | 5.75 |

^{a}BQL = Below lower limit of quantitation (0.0500 ng/mL for Compound 18 and 0.150 ng/mL for Compound 4 in homogenates, corresponding to 1.00 ng/g for Compound 18 and 3.00 ng/g for Compound 4 in retina tissues). BQL values were set to 0 for calculation of concentrations (ng/g) in tissues.

TABLE 24
TD Phase (Groups 1 and 3) Sclera/Choroid Compound 18 and Compound 4
Concentration Data

| | | | Compound | | | | | |
|---|---|---|---|---|---|---|---|---|
| | | | 18 | Compound | | | Final | Final |
| | | | Sclera/ | 4 Sclera/ | | | Compound | Compound |
| | | | Choroid | Choroid | | | 18 | 4 Sclera/ |
| | Time | | Homogenate | Homogenate | | Homogenate | Sclera/ | Choroid |
| | Point | Animal | Conc. | Conc. | Watson | Dilution | Choroid | Conc. |
| Group | (hr) | Number | (ng/mL)^{a} | (ng/mL)^{a} | Run | Factor | Conc. (ng/g) | (ng/g) |
| 3 | 0.5 | 207668 | 11.1 | 19.3 | 10 | 8 | 88.8 | 154 |
| | | 207669 | 5.90 | 17.7 | 10 | 8 | 47.2 | 142 |
| | | 707670 | 23.3 | 41.4 | 10 | 8 | 186 | 331 |
| | | Mean | | | | | 107 | 209 |
| 1 | 1 | 207658 | 3.89 | 17.0 | 5 | 8 | 31.1 | 136 |
| | | 207659 | 5.57 | 21.4 | 5 | 8 | 44.6 | 171 |
| | | 207660 | 7.51 | 30.0 | 5 | 8 | 60.1 | 240 |
| | | Mean | | | | | 45.3 | 182 |
| 3 | 2 | 207671 | 3.93 | 15.1 | 10 | 8 | 31.4 | 121 |
| | | 207672 | 84.2 | 29.2 | 10 | 8 | 674 | 234 |
| | | 207673 | 3.99 | 11.0 | 10 | 8 | 31.9 | 88.0 |
| | | Mean | | | | | 246 | 148 |
| 3 | 4 | 707674 | 7.93 | 14.5 | 10 | 8 | 63.4 | 116 |
| | | 207675 | 2.57 | 19.8 | 10 | 8 | 20.6 | 158 |
| | | 207676 | 80.0 | 79.2 | 11 | 8 | 640 | 634 |
| | | Mean | | | | | 241 | 303 |
| 3 | 8 | 207677 | 2.02 | 3.80 | 10 | 8 | 16.2 | 30.4 |
| | | 207678 | 3.97 | 9.53 | 10 | 8 | 31.8 | 76.2 |
| | | 207679 | 4.06 | 9.76 | 10 | 8 | 32.5 | 78.1 |
| | | Mean | | | | | 26.8 | 61.6 |

^{a}Limit of quantitation is 0.0500 ng/mL for Compound 18 and 0.150 ng/mL for Compound 4 in homogenates, corresponding to 1.00 ng/g for Compound 18 and 3.00 ng/g for Compound 4 in optic nerve tissues.

TABLE 25
TD Phase (Groups 1 and 3) Trabecular Meshwork Compound 18 and Compound 4
Concentration Data

| | | | Compound | Compound | | | | Final |
|---|---|---|---|---|---|---|---|---|
| | | | 18 | 4 | | | Final | Compound |
| | | | Trahecular | Trabecular | | | Compound | 4 |
| | | | Meshwork | Meshwork | | | 18 | Trabecular |
| | Time | | Homogenate | Homogenate | | Homogenate | Trabecular | Meshwork |
| | Point | Animal | Conc. | Conc. | Watson | Dilution | Meshwork | Conc. |
| Group | (hr) | Number | (ng/mL) | (ng/mL) | Run | Factor | Conc. (ng/g) | (ng/g) |
| 3 | 0.5 | 207668 | 0.146 | 5.61 | 10 | 20 | 2.92 | 112 |
| | | 207669 | BQL^{a} | 2.19 | 10 | 20 | 0 | 49.8 |
| | | 707670 | 0.907 | 10.8 | 10 | 20 | 18.1 | 216 |
| | | Mean | | | | | 7.01 | 126 |
| 1 | 1 | 207658 | BQL | 7.25 | 5 | 20 | 0 | 145 |
| | | 207659 | 0.785 | 10.7 | 5 | 20 | 15.7 | 214 |
| | | 207660 | 0.366 | 12.7 | 5 | 20 | 7.32 | 254 |
| | | Mean | | | | | 7.67 | 204 |
| 3 | 2 | 207671 | 0.0890 | 6.50 | 10 | 20 | 1.78 | 130 |
| | | 207672 | 0.179 | 3.52 | 10 | 20 | 3.58 | 70.1 |
| | | 207673 | BQL | 3.66 | 10 | 20 | 0 | 73.2 |
| | | Mean | | | | | 11.79 | 91.2 |
| 3 | 4 | 207674 | 0.231 | 2.65 | 10 | 20 | 4.62 | 53.0 |
| | | 207675 | 0.162 | 2.28 | 10 | 20 | 3.24 | 45.6 |
| | | 207676 | 0.435 | 2.66 | 10 | 20 | 8.70 | 53.2 |
| | | Mean | | | | | 5.52 | 50.6 |
| 3 | 8 | 207677 | BQL | 0.571 | 10 | 20 | 0 | 11.4 |
| | | 207678 | 0.237 | 0.684 | 10 | 20 | 4.74 | 13.7 |
| | | 207679 | 0.0532 | 0.837 | 10 | 20 | 1.06 | 16.7 |
| | | Mean | | | | | 1.93 | 13.9 |

^{a}BQL = Below lower limit of quantitation (0.0500 ng/mL for Compound 18 and 0.150 ng/mL for Compound 4 in homogenates, corresponding to 1.00 ng/g for Compound 18 and 3.00 ng/g for Compound 4 in trabecular meshwork tissues). BQL values were set to 0 for calculation of concentrations (ng/g) in tissues.

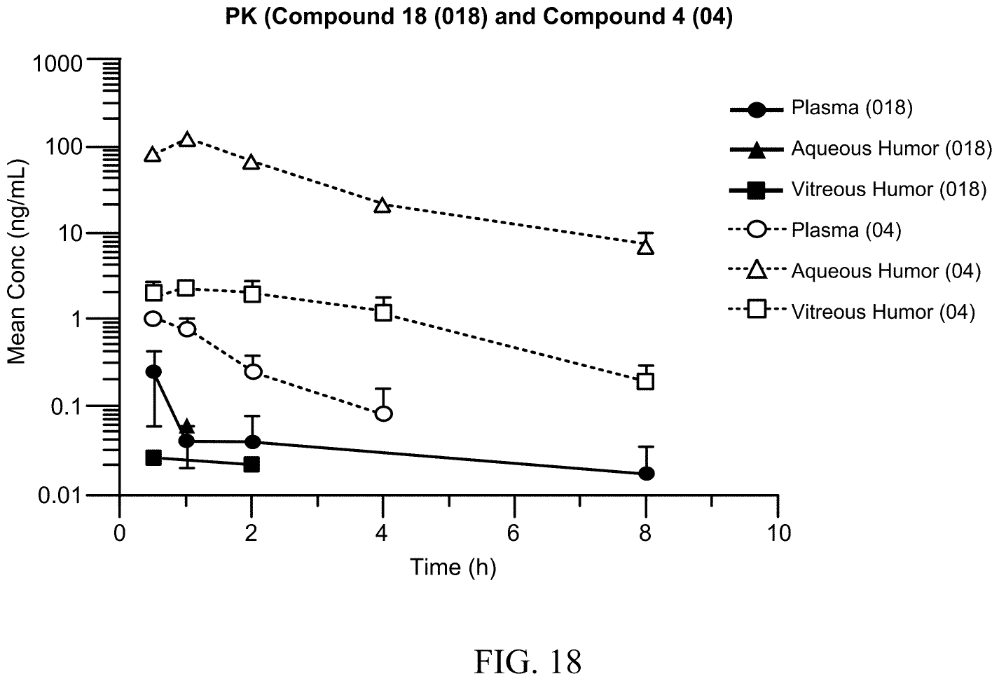

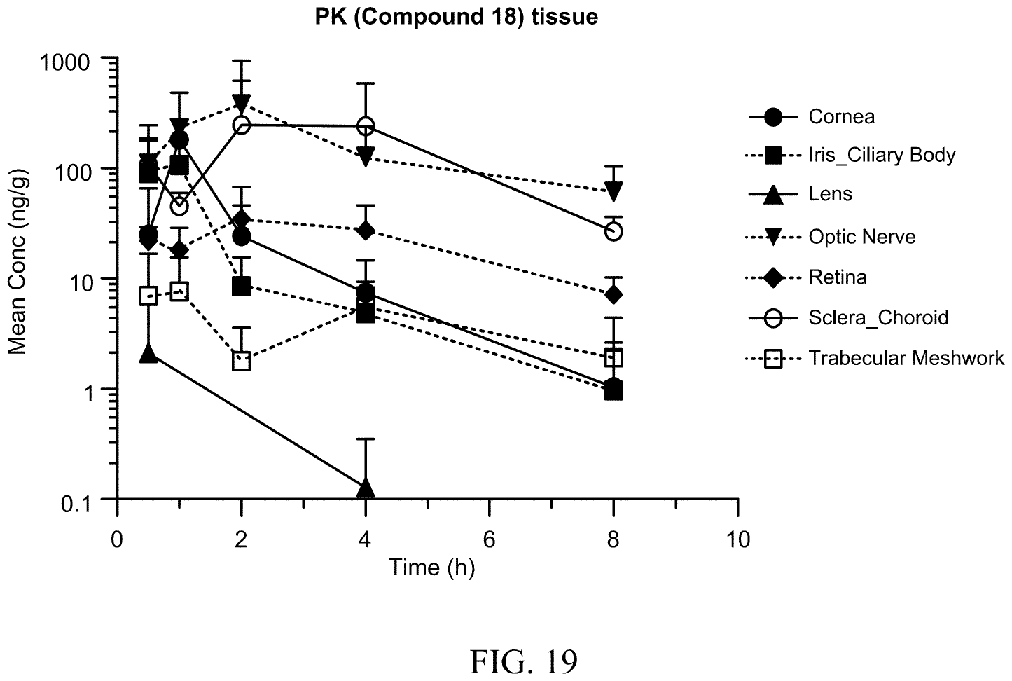

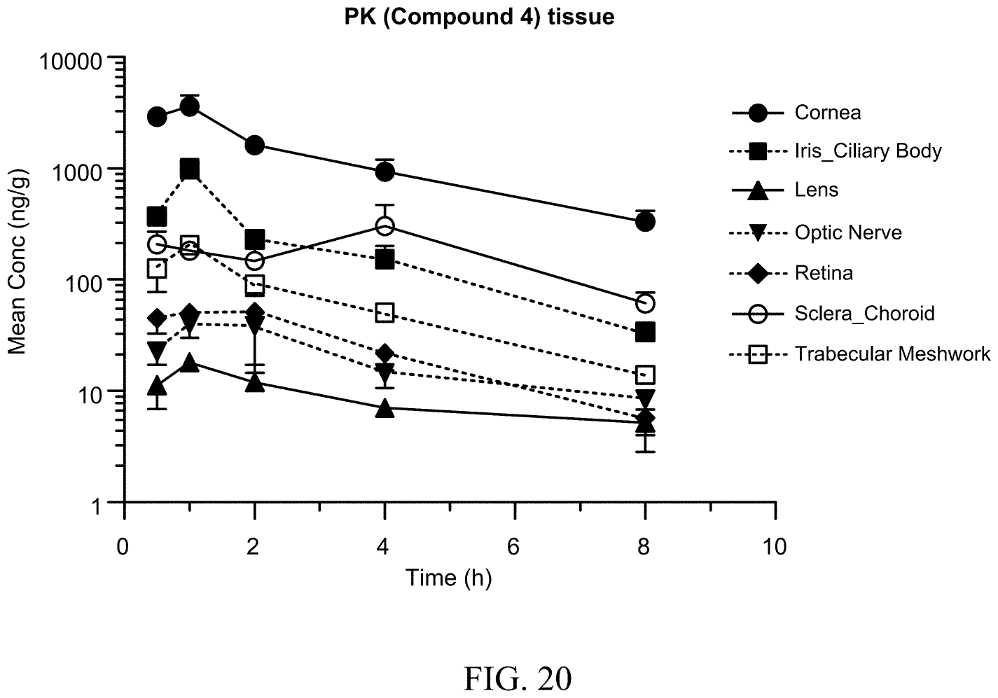

Distribution of Compound 18 and Compound 4 in the TD phase plasma and ocular tissues are graphed in FIG. 18 (Compound 18 and Compound 4, plasma, aqueous humor, and vitreous humor), FIG. 19 (Compound 18, sclera/choroid, cornea, iris/ciliary body, lens, optic nerve, retina, and trabecular meshwork), and FIG. 20 (Compound 4, sclera/choroid, cornea, iris/ciliary body, lens, optic nerve, retina, and trabecular meshwork). Compound 18 (parent) concentration was highest in optic nerve and sclera/choroid. Compound 4 (metabolite) concentration was highest in cornea.

Pharmacokinetic parameters for Compound 18 and Compound 4 in plasma are shown in Table 26.

TABLE 26
TD Phase (Groups 1 and 3) Plasma and Ocular Tissues Compound 18 and Compound
4 Pharmacokinetic Parameters

| | | | | | | | | Cₘₐₓ | AUC₀₋₈ | |
|---|---|---|---|---|---|---|---|---|---|---|
| | | | Dose | | | | | (ng/mL | (h*ng/mL | Metabolite/ |
| | | | Level | Dose | | Tₘₐₓ | T_{1/2} | or | or | Parent |
| Group | Analyte | Day | (mg/kg) | Route | Matrix | (h) | (h) | ng/g) | h*ng/g | AUC Ratio |
| 1 and 3 | 8 | 7 | 0.8 | Ocular | Plasma | 0.5 | NR | 0.261 | 0.255 | |
| | | | | Instillation | Aqueous | 1 | NC | 0.0630 | 0.0158 | |
| | | | | | Humor | | | | | |
| | | | | | Vitreous | 0.5 | NC | 0.0257 | 0.0242 | |
| | | | | | Humor | | | | | |
| | | | | | Cornea | 1 | 1.33 | 181 | 209 | |
| | | | | | Iris/Ciliary | 1 | 2.25 | 107 | 150 | |
| | | | | | Body | | | | | |
| | | | | | Lens | 0.5 | 0.865 | 2.12 | 1.19 | |
| | | | | | Optic Nerve | 2 | 2.43 | 378 | 1302 | |
| | | | | | Retina | 2 | 2.54 | 34.8 | 173 | |
| | | | | | Sclera/Choroid | 2 | 1.76 | 246 | 1234 | |
| | | | | | Trabecular | 1 | 2.64 | 7.67 | 32.4 | |
| | | | | | Meshwork | | | | | |
| | 4 | 7 | 0.8 | Ocular | Plasma | 0.5 | 0.958 | 1.02 | 1.57 | 6.72 |
| | | | | Instillation | Aqueous | 1 | 1.95 | 124 | 314 | 21700 |
| | | | | | Humor | | | | | |
| | | | | | Vitreous | 1 | 1.72 | 2.32 | 9.90 | 447 |
| | | | | | Humor | | | | | |
| | | | | | Cornea | 1 | 2.63 | 3605 | 10096 | 52.7 |
| | | | | | Iris/Ciliary | 1 | 2.11 | 980 | 1795 | 13.1 |
| | | | | | Body | | | | | |
| | | | | | Lens | 1 | 2.25 | 18.1 | 68.7 | 63.0 |
| | | | | | Optic Nerve | 1 | 2.93 | 40.2 | 159 | 0.133 |
| | | | | | Retina | 2 | 1.93 | 50.9 | 213 | 1.34 |
| | | | | | Sclera/Choroid | 4 | 1.74 | 303 | 1494 | 1.32 |
| | | | | | Trabecular | 1 | 2.20 | 204 | 533 | 18.0 |
| | | | | | Meshwork | | | | | |

NR = Not reported due to extensive extrapolation (>30%) for the elimination phase
NC = Not calculated (insufficient data points in the elimination phase)
AUC Ratio = [(AUC metabolite)/(MW metabolite)/[(AUC parent)/(MW parent)]
Where MW of metabolite Compound 4 = 489.59, MW of parent Compound 18 = 534.59

The plasma PK parameters were calculated from the mean plasma concentrations on Day 7. The Tₘₐₓ was 0.5 hour for both Compound 18 and Compound 4. The T_{1/2} was not reportable for Compound 18, due to extensive extrapolation (>30%) for the elimination phase. The T_{1/2} was 0.958 hour for Compound 4. The Cₘₐₓ values were 0.261 ng/mL for Compound 18 and 1.02 ng/mL for Compound 4. The AUC₀₋₈ values were 0.255 h*ng/mL for Compound 18 and 1.57 h*ng/mL for Compound 4.

The aqueous humor PK parameters were calculated from the mean aqueous humor concentrations on Day 7. The Tₘₐₓ was 1 hour for both Compound 18 and Compound 4. The T_{1/2} was not calculated for Compound 18, due to insufficient data points in the elimination phase. The T_{1/2} was 1.95 hours for Compound 4. The Cₘₐₓ values were 0.0630 ng/mL for Compound 18 and 124 ng/mL for Compound 4. The AUC₀₋₈ values were 0.0158 h*ng/mL for Compound 18 and 314 h*ng/mL for Compound 4.

The vitreous humor PK parameters were calculated from the mean vitreous humor concentrations on Day 7. The Tₘₐₓ was 0.5 hour for Compound 18 and 1 hour for Compound 4. The T_{1/2} was not calculated for Compound 18, due to insufficient data points in the elimination phase. The T_{1/2} was 1.72 hours for Compound 4. The Cₘₐₓ values were 0.0257 ng/mL for Compound 18 and 2.32 ng/mL for Compound 4. The AUC₀₋₈ values were 0.0242 h*ng/mL for Compound 18 and 9.90 h*ng/mL for Compound 4.

The cornea PK parameters were calculated from the mean cornea concentrations on Day 7. The Tₘₐₓ was 1 hour for both Compound 18 and Compound 4. The T_{1/2} was 1.33 hours for Compound 18 and 2.63 hours for Compound 4. The Cₘₐₓ values were 181 ng/g for Compound 18 and 3605 ng/g for Compound 4. The AUC₀₋₈ values were 209 h*ng/g for Compound 18 and 10096 h*ng/g for Compound 4.

The iris/ciliary body PK parameters were calculated from the mean iris/ciliary body concentrations on Day 7. The Tₘₐₓ was 1 hour for both Compound 18 and Compound 4. The T_{1/2} was 2.25 hours for Compound 18 and 2.11 hours for Compound 4. The Cₘₐₓ values were 107 ng/g for Compound 18 and 980 ng/g for Compound 4. The AUC₀₋₈ values were 150 h*ng/g for Compound 18 and 1795 h*ng/g for Compound 4.

The lens PK parameters were calculated from the mean lens concentrations on Day 7. The Tₘₐₓ was 0.5 hour for Compound 18 and 1 hour for Compound 4. The T_{1/2} was 0.865 hour for Compound 18 and 2.25 hours for Compound 4. The Cₘₐₓ values were 2.12 ng/g for Compound 18 and 18.1 ng/g for Compound 4. The AUC₀₋₈ values were 1.19 h*ng/g for Compound 18 and 68.7 h*ng/g for Compound 4.

The optic nerve PK parameters were calculated from the mean optic nerve concentrations on Day 7. The Tₘₐₓ was 2 hours for Compound 18 and 1 hour for Compound 4. The T_{1/2} was 2.43 hours for Compound 18 and 2.93 hours for Compound 4. The Cₘₐₓ values were 378 ng/g for Compound 18 and 40.2 ng/g for Compound 4. The AUC₀₋₈ values were 1302 h*ng/g for Compound 18 and 159 h*ng/g for Compound 4.

The retina PK parameters were calculated from the mean retina concentrations on Day 7. The Tₘₐₓ was 2 hours for both Compound 18 and Compound 4. The T_{1/2} was 2.54 hours for Compound 18 and 1.93 hours for Compound 4. The Cₘₐₓ values were 34.8 ng/g for Compound 18 and 50.9 ng/g for Compound 4. The AUC₀₋₈ values were 173 h*ng/g for Compound 18 and 213 h*ng/g for Compound 4.

The sclera/choroid PK parameters were calculated from the mean sclera/choroid concentrations on Day 7. The Tₘₐₓ was 2 hours for Compound 18 and 4 hours for Compound 4. The T_{1/2} was 1.76 hours for Compound 18 and 1.74 hours for Compound 4. The Cₘₐₓ values were 246 ng/g for Compound 18 and 303 ng/g for Compound 4. The AUC₀₋₈ values were 1234 h*ng/g for Compound 18 and 1494 h*ng/g for Compound 4.

The trabecular meshwork PK parameters were calculated from the mean trabecular meshwork concentrations on Day 7. The Tₘₐₓ was 1 hour for both Compound 18 and Compound 4. The T_{1/2} was 2.64 hours for Compound 18 and 2.20 hours for Compound 4. The Cₘₐₓ values were 7.67 ng/g for Compound 18 and 204 ng/g for Compound 4. The AUC₀₋₈ a values were 32.4 h*ng/g for Compound 18 and 533 h*ng/g for Compound 4.

### Conclusion

Following 7 consecutive daily ocular instillation of Compound 18 in rabbits, exposure to Compound 18 (parent) was highest in optic nerve (AUC₀₋₈=1302 h*ng/g) and sclera/choroid (AUC₀₋₈=1234 h*ng/g), followed by similar exposure in cornea (AUC₀₋₈=209 h*ng/g), retina (AUC₀₋₈=173 h*ng/g), and iris/ciliary body (AUC₀₋₈=150 h*ng/g), and then trabecular meshwork (AUC₀₋₈=32.4 h*ng/g) (see Table 26). Exposure to Compound 18 (parent) was relatively low in aqueous humor, vitreous humor, plasma, and lens (AUC₀₋₈=0.02-1.2 h*ng/mL or h*ng/g).

Compound 4 (metabolite) vs Compound 18 (parent) AUC ratios in plasma and ocular tissues from the TD phase are shown in Table 29 above. Metabolite/parent AUC ratios were highest in aqueous humor (21700), followed by vitreous humor (447), lens (63.0), cornea (52.7), trabecular meshwork (18.0), iris/ciliary body (13.1), plasma (6.72), retina (1.34), sclera/choroid (1.32), and optic nerve (0.133). Extensive metabolism of Compound 18 to Compound 4 has resulted in low Compound 18 exposure in some ocular tissues. The total exposure to a combination of Compound 18 and Compound 4 was highest in cornea and lowest in vitreous humor, plasma, and lens.

These results show that ocular instillation of Compound 18 is highest in the optic nerve and sclera/choroid, and that the total exposure to a combination of Compound 18 and Compound 4 is highest in the cornea.

Compound 18 is rarely metabolized in the optic nerve tissue where it is distributed in large amounts. This indicates that compound 18 can be applied to treat various ophthalmic diseases in that it can protect optic nerve cells by activating PKG signaling pathway in appropriate tissues.

Compound 18 is rapidly metabolized to compound 4 in most other ocular tissues. Since compound 4 itself is an inhibitor of PDE-5 and -6, this metabolite can itself also have a beneficial effect in treating various ophthalmic diseases.

Compound 18 was highly distributed in the trabecular meshwork (TM), a key target tissue of glaucoma, and moderately distributed in the retina, a core tissue of AMD. These results indicate that compound 18 would be an effective agent for treating such eye diseases.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification, are herein incorporated by reference in their entirety for all purposes.

1. A method of treating an eye disease, the method comprising administering to an eye of a subject a therapeutically effective amount of a compound an ophthalmic composition comprising the compound, wherein the compound is of formula (I): 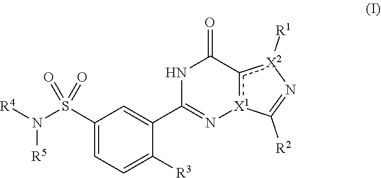
or a pharmaceutically acceptable salt, a solvate, a hydrate, a prodrug, or a stereoisomer thereof, wherein:
X¹ and X² are independently selected from N and C and at least one of X¹ and X² is N;
R¹ is —H, or optionally substituted (C₁-C₅)alkyl;
R² is optionally substituted (C₁-C₅)alkyl;
R³ is optionally substituted (C₁-C₅)alkoxy;
R⁴ is —H or optionally substituted (C₁-C₅)alkyl, and R⁵ is a 4-membered carbocycle or heterocycle ring that is substituted with one or more R⁶,
or R⁴ and R⁵ together with the nitrogen atom to which they are attached are cyclically linked to form a 4-membered heterocycle that is substituted with one or more R⁶; and
and each R⁶ is independently selected from —OH, —O—NO₂, optionally substituted (C₁-C₅)alkyl, optionally substituted (C₁-C₁₀)alkylene, optionally substituted (C₂-C₁₀) alkenyl, optionally substituted (C₂-C₁₀)alkynyl, optionally substituted (C₁-C₅)alkoxy, optionally substituted (C₃-C₅)heterocycle, optionally substituted (C₁-C₅)alkyl-(C₃-C₅)heterocycle-, optionally substituted (C₃-C₅)heterocycle-(C₁-C₅)alkyl-, optionally substituted (C₁-C₅)alkyl-Z¹—(C₁-C₅)alkyl-, optionally substituted (C₁-C₅)alkyl-Z¹—(C₁-C₅)alkoxy-, optionally substituted (C₁-C₁₀)alkyl-NR¹—, optionally substituted (C₁-C₁₀)alkyl-Z¹—(C₁-C₅)alkyl-NR¹—, optionally substituted (C₁-C₁₀)alkoxy-Z¹—(C₁-C₅)alkyl-NR¹—, substituted (C₁-C₅)alkyl-(C₃-C₅)heterocycle-(C₁-C₅)alkyl-, substituted linear linker, and substituted branched linker, wherein Z¹ is —CO₂—, —O—, —OCO—, —CONH—, —NHCO—, or —NH—, and the substituents of each R⁶ are independently selected from —O—NO₂, —ONO, —OH, —NH₂, —COOH, halogen, (C₁-C₃)alkoxy and (C₁-C₃)alkyl;
wherein at least one R⁶ is substituted with —O—NO₂, —ONO, —OH or —NH₂.

2. The method of claim 1, wherein the subject has an eye disease.

3. The method of claim 1, wherein the eye disease is selected from glaucoma, age-related macular degeneration (AMD), diabetic retinopathy (DR), xerophthalmia, dry eye syndrome (DES), cataracts, uveitis, ischemic retinopathy, optic neuropathy, diabetic macular edema (DME), senile cataracts, conjunctivitis, Stevens-Johnson Syndrome, Sjogren's Syndrome, trauma, and trauma of the eye due to eye surgery.

4. The method of claim 3, wherein the eye disease is glaucoma.

5. The method of claim 3, wherein the eye disease is AMD.

6. The method of claim 3, wherein the eye disease is dry AMD.

7. The method of claim 4 or 5, further comprising identifying the subject as suffering from glaucoma or AMD.

8. The method of claim 1, wherein the ophthalmic composition comprises a physiologically compatible ophthalmic vehicle.

9. The method of claim 8, wherein the ophthalmic composition is an eye drop composition.

10. The method of claim 1, wherein the compound or composition is topically administered to the eye daily or as needed.

11. The method of claim 10, wherein the compound or composition is topically administered to the eye once a day.

12. The method of claim 10, wherein the compound or composition is topically administered to the eye two times or more daily.

13. A method of treating a PDE-5 and/or -6-related indication, the method comprising administering to a subject in need thereof a therapeutically effective amount of a compound of formula (I): 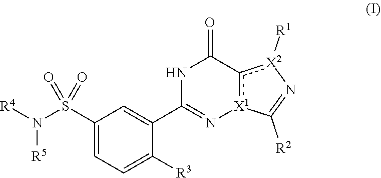
or a pharmaceutically acceptable salt, a solvate, a hydrate, a prodrug, or a stereoisomer thereof, wherein:
X¹ and X² are independently selected from N and C and at least one of X¹ and X² is N;
R¹ is —H, or optionally substituted (C₁-C₅)alkyl;
R² is optionally substituted (C₁-C₅)alkyl;
R³ is optionally substituted (C₁-C₅)alkoxy;
R⁴ is —H or optionally substituted (C₁-C₅)alkyl, and R⁵ is a 4-membered carbocycle or heterocycle ring that is substituted with one or more R⁶,
or R⁴ and R⁵ together with the nitrogen atom to which they are attached are cyclically linked to form a 4-membered heterocycle that is substituted with one or more R⁶; and
and each R⁶ is independently selected from —OH, —O—NO₂, optionally substituted (C₁-C₅)alkyl, optionally substituted (C₁-C₁₀)alkylene, optionally substituted (C₂-C₁₀) alkenyl, optionally substituted (C₂-C₁₀)alkynyl, optionally substituted (C₁-C₅)alkoxy, optionally substituted (C₃-C₅)heterocycle, optionally substituted (C₁-C₅)alkyl-(C₃-C₅)heterocycle-, optionally substituted (C₃-C₅)heterocycle-(C₁-C₅)alkyl-, optionally substituted (C₁-C₅)alkyl-Z¹—(C₁-C₅)alkyl-, optionally substituted (C₁-C₅)alkyl-Z¹—(C₁-C₅)alkoxy-, optionally substituted (C₁-C₁₀)alkyl-NR¹—, optionally substituted (C₁-C₁₀)alkyl-Z¹—(C₁-C₅)alkyl-NR¹—, optionally substituted (C₁-C₁₀)alkoxy-Z¹—(C₁-C₅)alkyl-NR¹—, substituted (C₁-C₅)alkyl-(C₃-C₅)heterocycle-(C₁-C₅)alkyl-, substituted linear linker, and substituted branched linker, wherein Z¹ is —CO₂—, —O—, —OCO—, —CONH—, —NHCO—, or —NH—, and the substituents of each R⁶ are independently selected from —O—NO₂, —ONO, —OH, —NH₂, —COOH, halogen, (C₁-C₃)alkoxy and (C₁-C₃)alkyl;
wherein at least one R⁶ is substituted with —O—NO₂, —ONO, —OH or —NH₂.

14. (canceled)

15. (canceled)

16. The method of claim 1, wherein in formula (I) at least one R⁶ is substituted with —O—NO₂.

17. The method of claim 1, wherein R¹ is (C₁-C₅)alkyl.

18. The method of claim 17, wherein R¹ is methyl.

19. The method of claim 1, wherein R² is n-propyl.

20. The method of claim 19, wherein R³ is ethoxy.

21. The method of claim 20, wherein the compound is of formula (Ia): 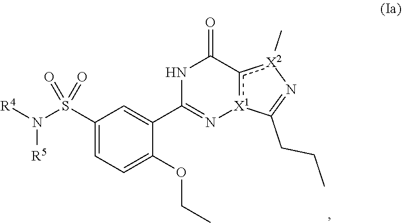
or a pharmaceutically acceptable salt, a solvate, a hydrate, a prodrug, or a stereoisomer thereof.

22. The method of claim 1, wherein R⁴ is —H and R⁵ is substituted azetidine.

23. The method of claim 1, wherein R⁴ and R⁵ together with the nitrogen atom to which they are attached are cyclically linked to form substituted azetidine.

24. The method of claim 1, wherein X¹ is N and X² is C.

25. The method of claim 1, wherein X¹ is C and X² is N.

26. The method of claim 22, wherein the compound is of formula (II): 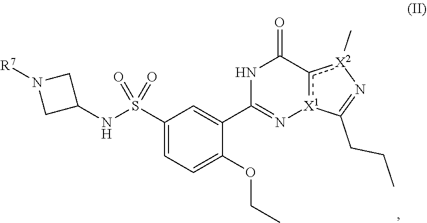
wherein:
R⁷ is selected from —H, R⁷⁰, and R⁷¹—Z²—R⁷²;
R⁷⁰, R⁷¹ and R⁷² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, and optionally substituted (C₁-C₅)alkoxy, wherein the optional substituent is selected from —OH, —NH₂, and —O—NO₂; and
Z² is —CO₂—, —O—, —OCO—, —CONH—, —NHCO—, or —NH—.

27. The method of claim 26, wherein the compound is of formula (IIa): 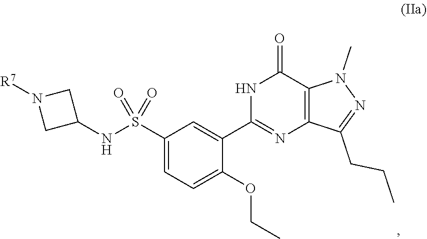
or a pharmaceutically acceptable salt, a solvate, a hydrate, a prodrug, or a stereoisomer thereof.

28. The method of claim 27, wherein:
R⁷ is 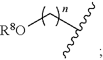
R⁸ is —H or —NO₂; and
n is 1, 2, 3, 4, or 5.

29. The method of claim 28, wherein the compound is selected from: 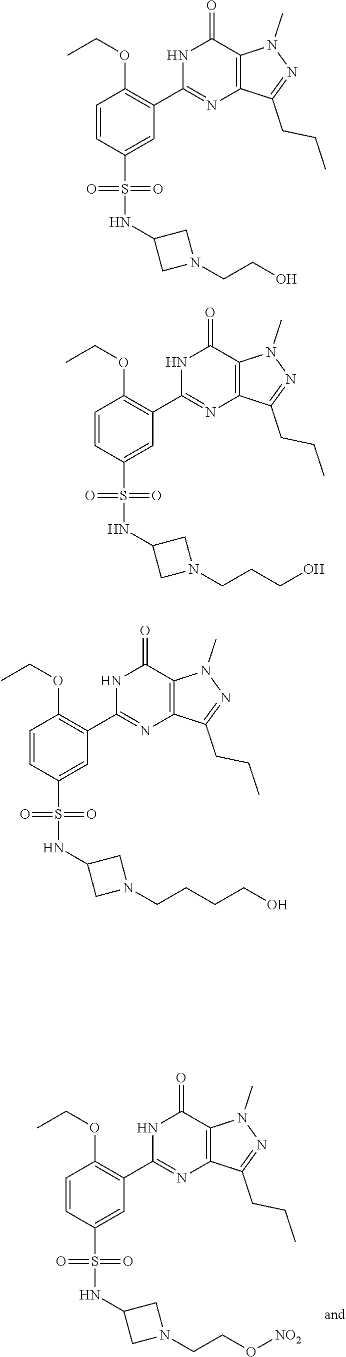 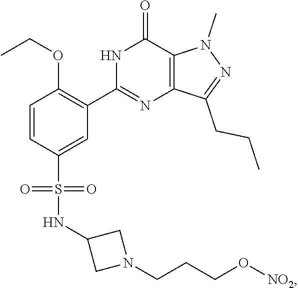 [SMILES: CCCc1nn(C)c2c(=O)[nH]c(-c3cc(S(=O)(=O)NC4CN(CCCO[N+](=O)[O-])C4)ccc3OCC)nc12]
or a pharmaceutically acceptable salt, a solvate, a hydrate, a prodrug, or a stereoisomer thereof.

30. The method of claim 26, wherein the compound is of formula (IIb): 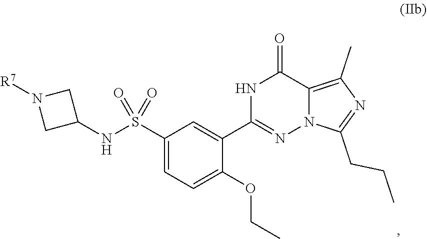
or a pharmaceutically acceptable salt, a solvate, a hydrate, a prodrug, or a stereoisomer thereof, wherein:
R⁷ is selected from —H, R⁷⁰, and R⁷¹—Z²—R⁷²;
R⁷⁰, R⁷¹ and R⁷² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, and optionally substituted (C₁-C₅)alkoxy, wherein the optional substituent is selected from —OH, —NH₂, and —O—NO₂; and
Z² is —CO₂—, —O—, —OCO—, —CONH—, —NHCO—, or —NH—.

31. The method of claim 30, wherein:
R⁷ is 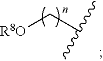
R⁸ is —H or —NO₂; and
n is 1, 2, 3, 4, or 5.

32. The method of claim 31, wherein the compound is selected from: 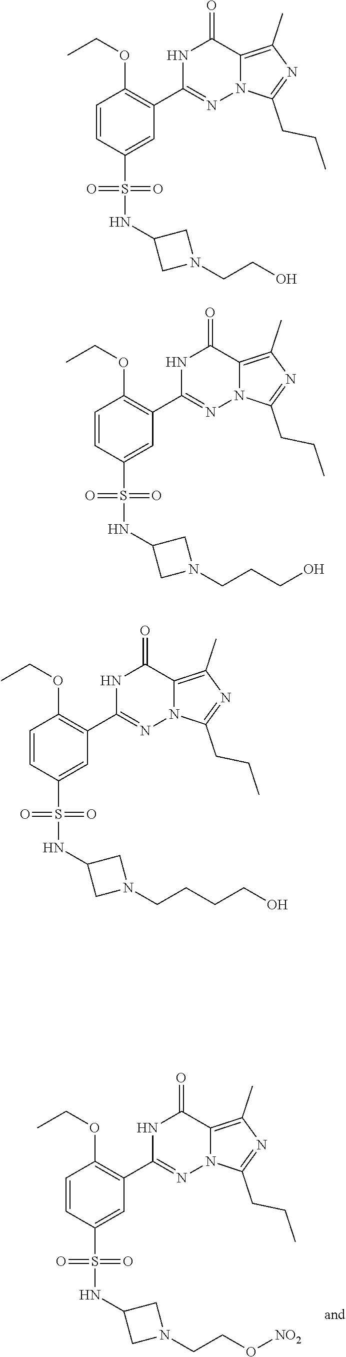 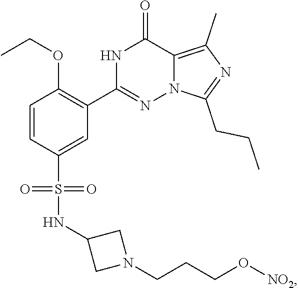 [SMILES: CCCc1nc(C)c2c(=O)[nH]c(-c3cc(S(=O)(=O)NC4CN(CCCO[N+](=O)[O-])C4)ccc3OCC)nn12]
or a pharmaceutically acceptable salt thereof, a solvate, a hydrate, a prodrug, or a stereoisomer.

33. The method of claim 23, wherein the compound is of formula (III): 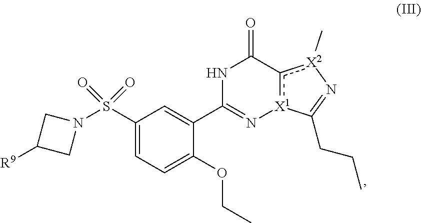
or a pharmaceutically acceptable salt, a solvate, a hydrate, a prodrug, or a stereoisomer thereof, wherein:
R⁹ is selected from —O—NO₂, —NR¹⁰R¹¹, —OR¹², R⁹⁰, and R⁹¹—Z³—R⁹²;
R⁹⁰, R⁹¹ and R⁹² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, optionally substituted (C₁-C₅)alkoxy, optionally substituted (C₃-C₅)heterocycle-(C₁-C₅)alkyl-, and optionally substituted (C₁-C₅)alkyl-(C₃-C₅)heterocycle-(C₁-C₅)alkyl-, wherein the optional substituent is selected from —OH, —NH₂, and —O—NO₂;
Z³ is —CO₂—, —O—, —OCO—, —CONH—, —NHCO—, or —NH—; and
R¹⁰, R¹¹, and R¹² are independently H, optionally substituted (C₁-C₅)alkyl, or optionally substituted (C₁-C₅)alkyl-Z¹—(C₁-C₅)alkyl, wherein the optional substituent is selected from —OH, —NH₂, and —O—NO₂;
or R¹⁰ and R¹¹ together with the nitrogen atom to which they are attached are cyclically linked to form an optionally substituted heterocycle, wherein the optional substituent is selected from —OH, —O—NO₂, —CH₂OH, —CH₂CH₂OH, and —CH₂ONO₂.

34. The method of claim 33, wherein the compound is of formula (IIIa): 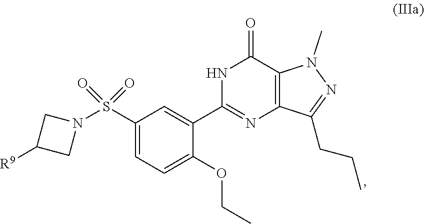
or a pharmaceutically acceptable salt, a solvate, a hydrate, a prodrug, or a stereoisomer thereof.

35. The method of claim 34, wherein R⁹ is 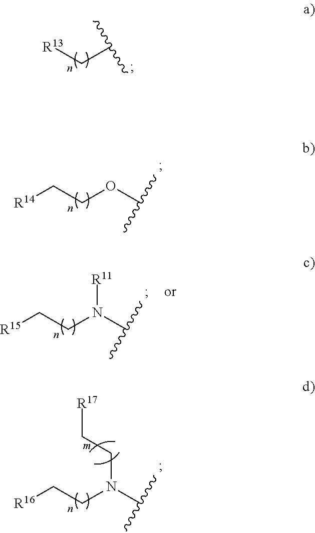
and wherein:
R¹¹ is H or methyl;
R¹³, R¹⁴, R¹⁵, R¹⁶, and R¹⁷ are independently selected from —OH, —NH₂, and —O—NO₂; and
n and m are independently selected from 0, 1, 2, 3, 4, or 5.

36. The method of claim 35, wherein R⁹ is 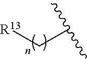
selected from:

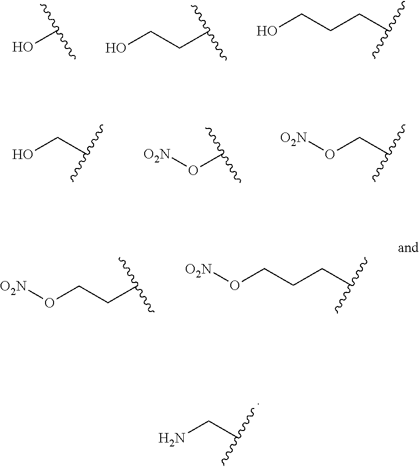

37. The method of claim 36, wherein the compound is selected from: 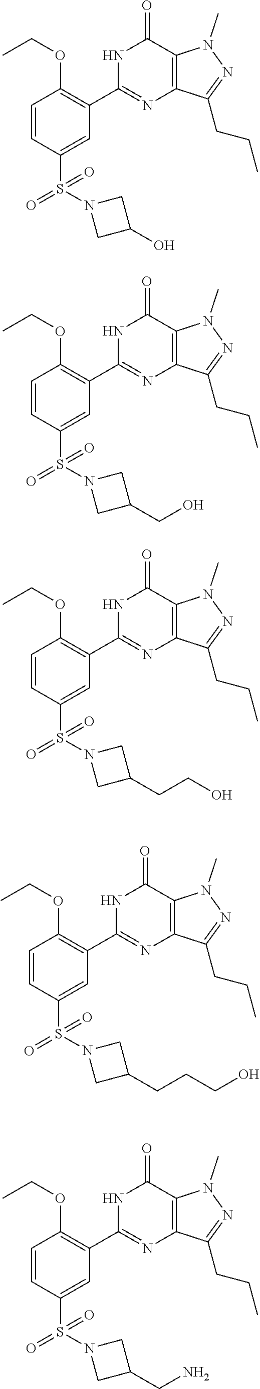 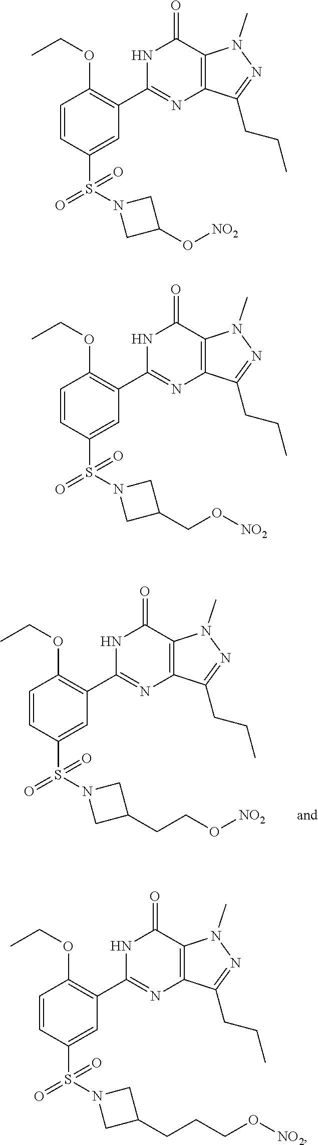
or a pharmaceutically acceptable salt, a solvate, a hydrate, a prodrug, or a stereoisomer thereof.

38. The method of claim 35, wherein R⁹ is 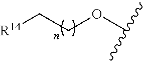
selected from:

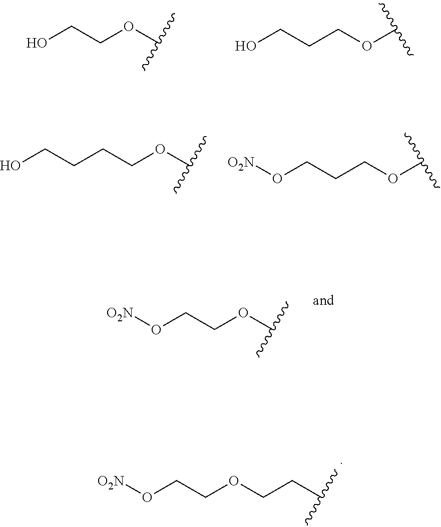 [SMILES: *OCCO ; *OCCCO ; *OCCCCO ; *OCCCO[N+](=O)[O-] ; *OCCO[N+](=O)[O-] ; *CCOCCO[N+](=O)[O-]]

39. The method of claim 38, wherein the compound is selected from: 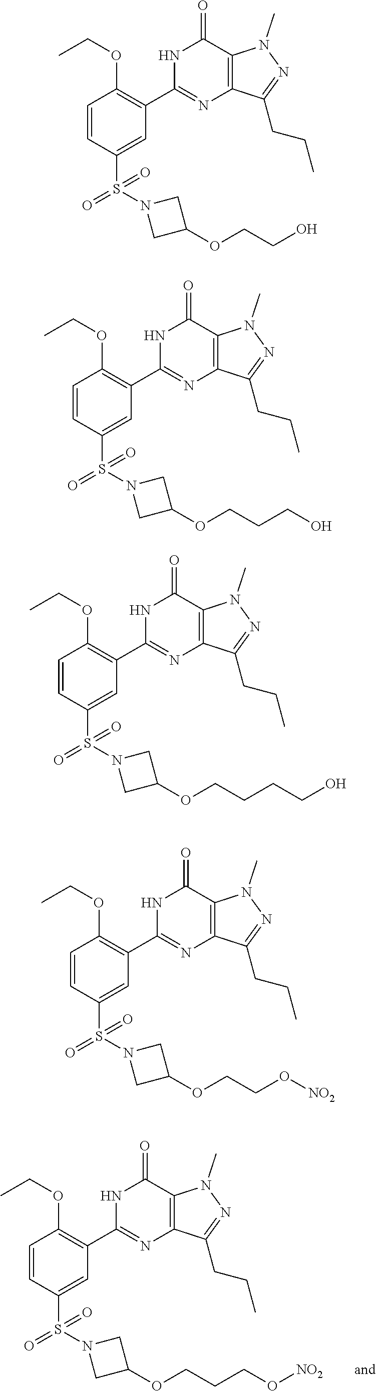 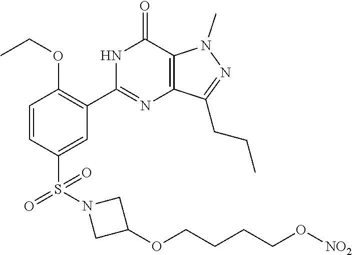 [SMILES: CCCc1nn(C)c2c(=O)[nH]c(-c3cc(S(=O)(=O)N4CC(OCCCCO[N+](=O)[O-])C4)ccc3OCC)nc12]
a pharmaceutically acceptable salt, a solvate, a hydrate, a prodrug, or a stereoisomer thereof.

40. The method of claim 35, wherein R⁹ is 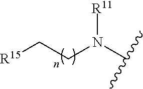
selected from:

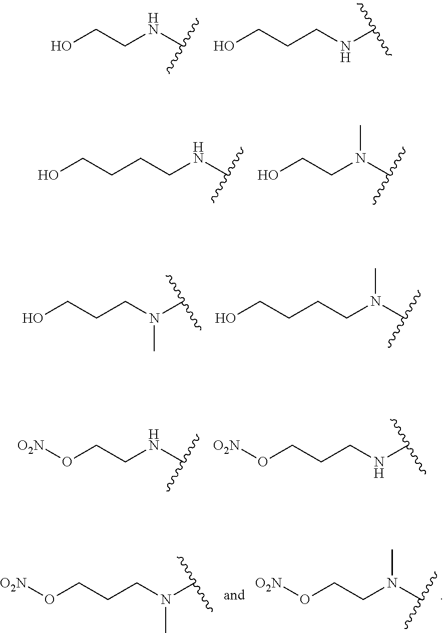

41. The method of claim 40, wherein the compound is selected from: 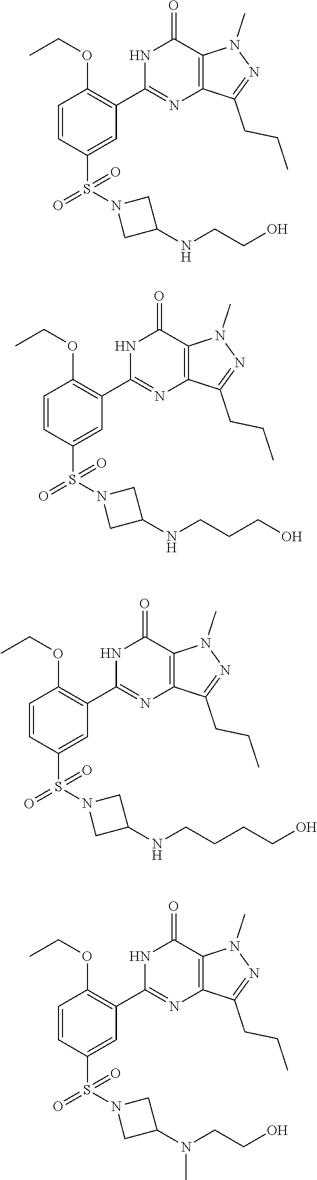 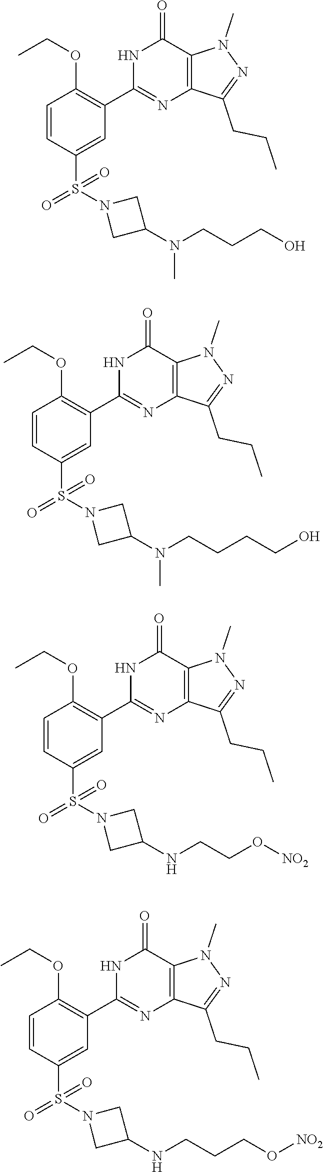 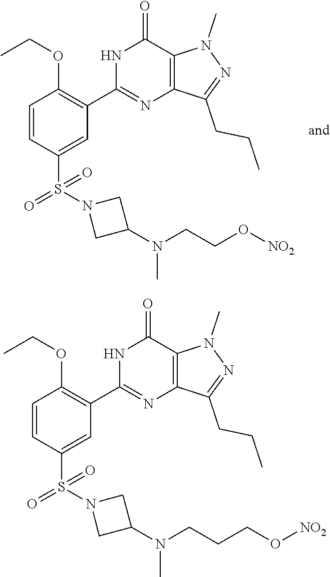
or a pharmaceutically acceptable salt, a solvate, a hydrate, a prodrug, or a stereoisomer thereof.

42. The method of claim 35, wherein R⁹ is 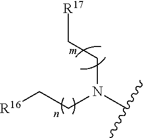
selected from:

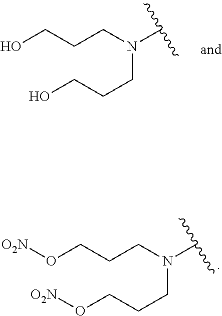 [SMILES: *N(CCCO)CCCO ; *N(CCCO[N+](=O)[O-])CCCO[N+](=O)[O-]]

43. The method of claim 42, wherein the compound is selected from: 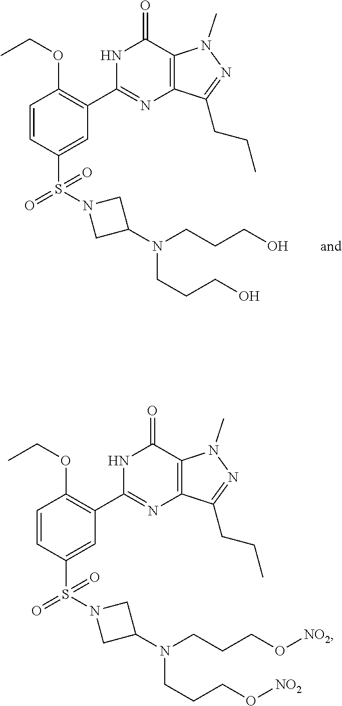 [SMILES: CCCc1nn(C)c2c(=O)[nH]c(-c3cc(S(=O)(=O)N4CC(N(CCCO)CCCO)C4)ccc3OCC)nc12 ; CCCc1nn(C)c2c(=O)[nH]c(-c3cc(S(=O)(=O)N4CC(N(CCCO[N+](=O)[O-])CCCO[N+](=O)[O-])C4)ccc3OCC)nc12]
or a pharmaceutically acceptable salt, a solvate, a hydrate, a prodrug, or a stereoisomer thereof.

44. The method of claim 34, wherein R⁹ is 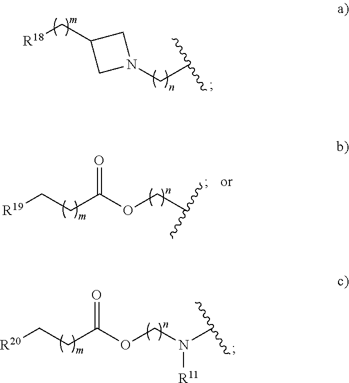
wherein:
R¹¹ is —H or -methyl;
R¹⁸ is selected from —OH, —NH₂, and —O—NO₂;
R¹⁹ and R²⁰ are independently selected from —OH, —NH₂, —O—NO₂, and 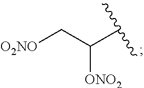
and
n and m are independently selected from 0, 1, 2, 3, 4, 5 or 6.

45. The method of claim 44, wherein R⁹ is 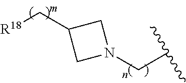
selected from:

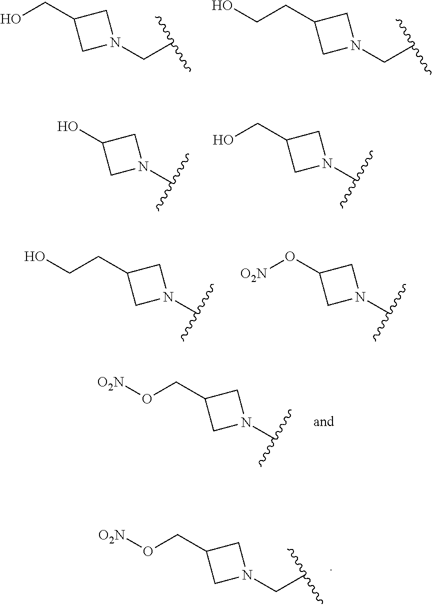

46. The method of claim 45, wherein the compound is selected from: 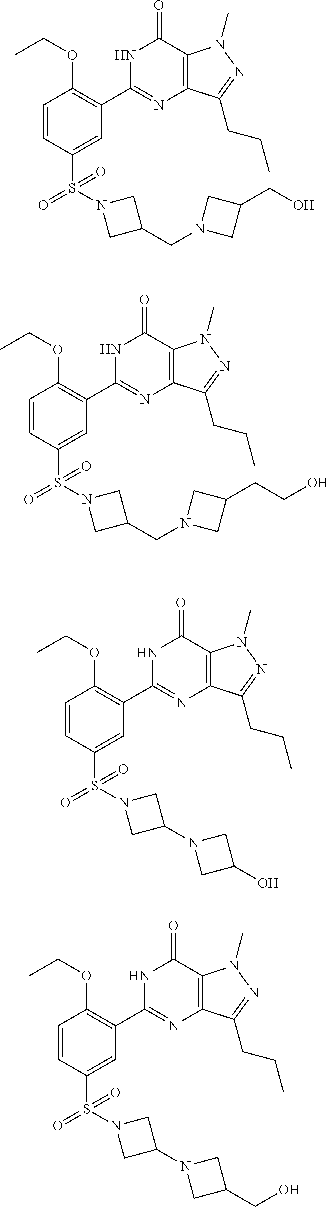 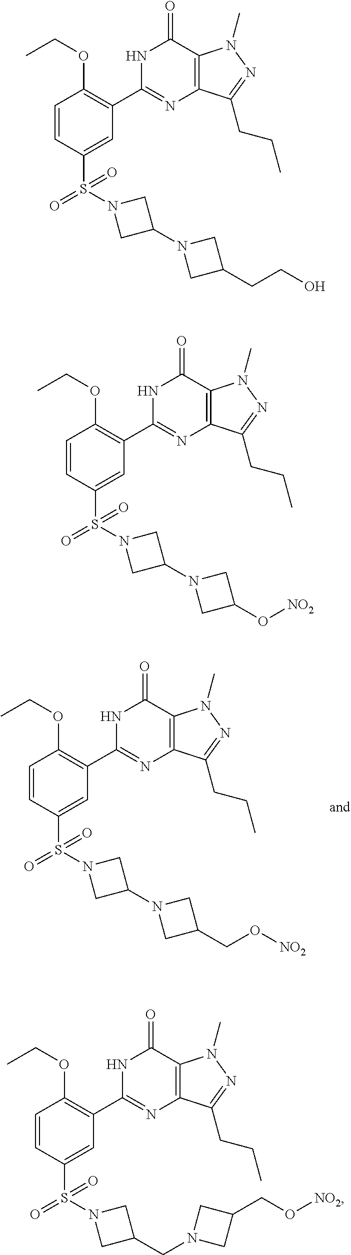 [SMILES: CCCc1nn(C)c2c(=O)[nH]c(-c3cc(S(=O)(=O)N4CC(N5CC(CCO)C5)C4)ccc3OCC)nc12 ; CCCc1nn(C)c2c(=O)[nH]c(-c3cc(S(=O)(=O)N4CC(N5CC(O[N+](=O)[O-])C5)C4)ccc3OCC)nc12 ; CCCc1nn(C)c2c(=O)[nH]c(-c3cc(S(=O)(=O)N4CC(N5CC(CO[N+](=O)[O-])C5)C4)ccc3OCC)nc12 ; CCCc1nn(C)c2c(=O)[nH]c(-c3cc(S(=O)(=O)N4CC(CN5CC(CO[N+](=O)[O-])C5)C4)ccc3OCC)nc12]
or a pharmaceutically acceptable salt, a solvate, a hydrate, a prodrug, or a stereoisomer thereof.

47. The method of claim 44, wherein R⁹ is 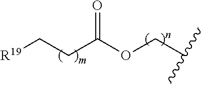
selected from:

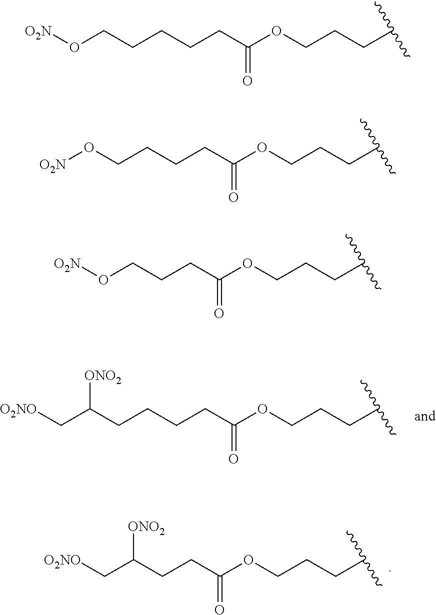

48. The method of claim 47, wherein the compound is selected from: 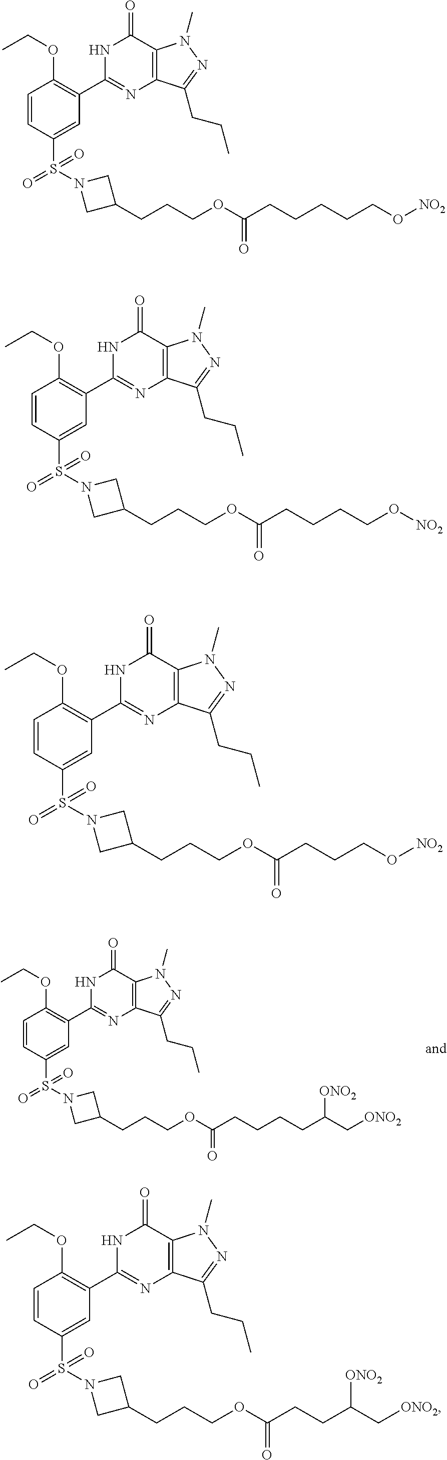
or a pharmaceutically acceptable salt, a solvate, a hydrate, a prodrug, or a stereoisomer thereof.

49. The method of claim 44, wherein R⁹ is 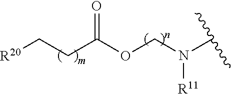
selected from:

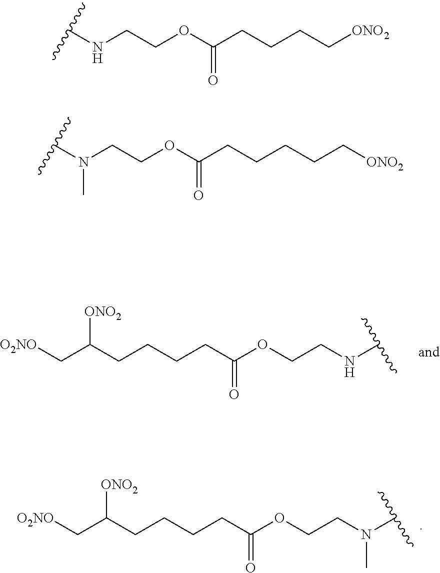

50. The method of claim 49, wherein the compound is selected from: 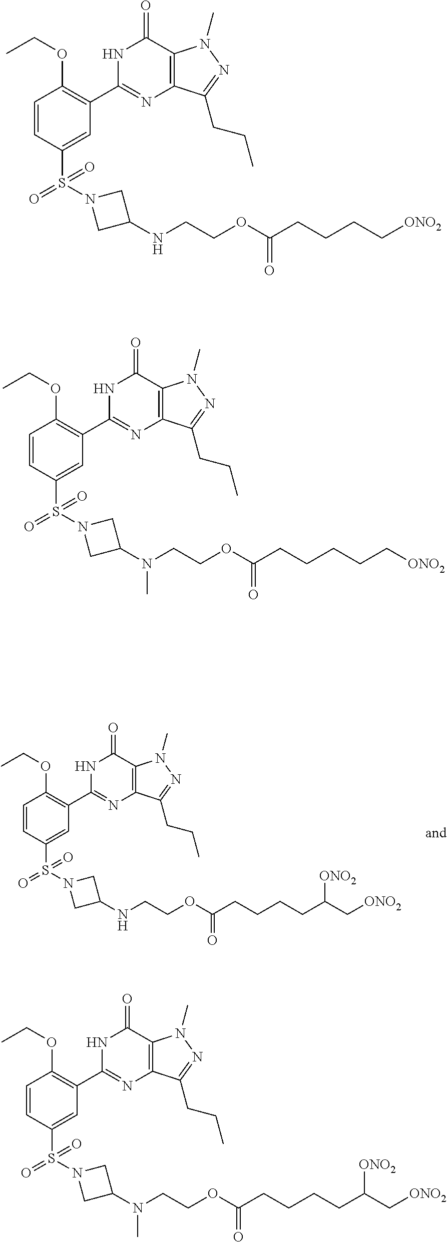
or a pharmaceutically acceptable salt, a solvate, a hydrate, a prodrug, or a stereoisomer thereof.

51. The method of claim 33, wherein the compound is of formula (IIIb): 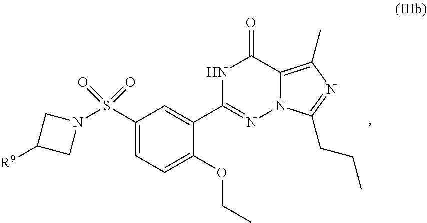
wherein:
R⁹ is selected from —O—NO₂, —NR¹⁰R¹¹, —OR¹², R⁹⁰, and R⁹¹—Z³—R⁹²;
R⁹⁰, R⁹¹ and R⁹² are independently selected from optionally substituted (C₁-C₅)alkyl, optionally substituted (C₁-C₁₀)alkylene, optionally substituted (C₂-C₁₀)alkenyl, optionally substituted (C₂-C₁₀)alkynyl, optionally substituted (C₁-C₅)alkoxy, and optionally substituted (C₃-C₅)heterocycle-(C₁-C₅)alkyl, wherein the optional substituent is selected from —OH, —NH₂, and —O—NO₂;
Z³ is —CO₂—, —O—, —OCO—, —CONH—, —NHCO—, or —NH—; and
R¹⁰, R¹¹, and R¹² are independently H, optionally substituted (C₁-C₅)alkyl, or optionally substituted (C₁-C₅)alkyl-Z¹—(C₁-C₅)alkyl, wherein the optional substituent is selected from —OH, —NH₂, and —O—NO₂;
or R¹⁰ and R¹¹ together with the nitrogen atom to which they are attached are cyclically linked to form an optionally substituted heterocycle, wherein the optional substituent is selected from —OH, —O—NO₂, —CH₂OH, —CH₂CH₂OH, and —CH₂O—NO₂.

52. The method of claim 51, wherein R⁹ is 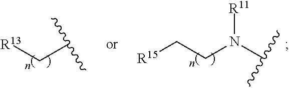
and wherein:
R¹¹ is H or methyl;
R¹³ and R¹⁵ are independently selected from —OH, —NH₂, and —O—NO₂; and
n is 0, 1, 2, 3, 4, or 5.

53. The method of claim 52, wherein R⁹ is 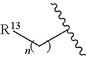
selected from:

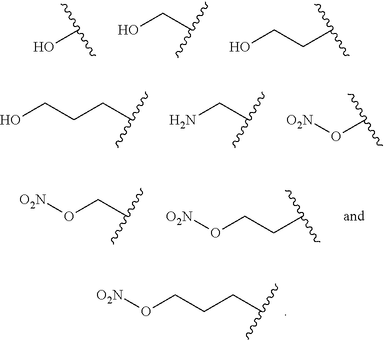

54. The method of claim 53, wherein the compound is selected from: 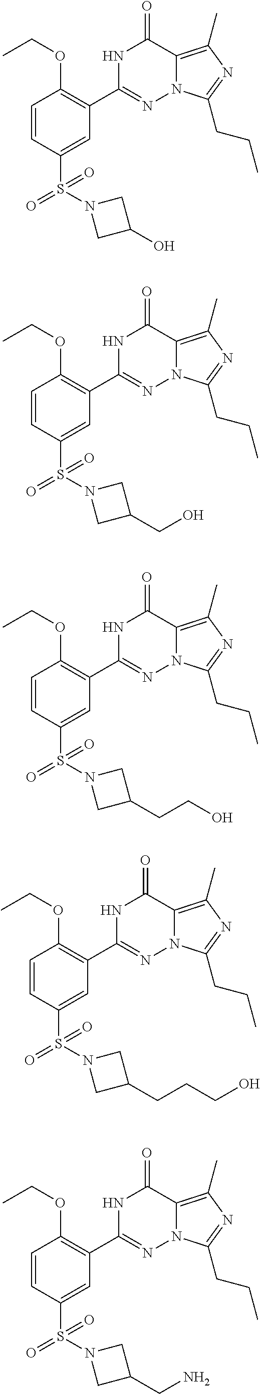 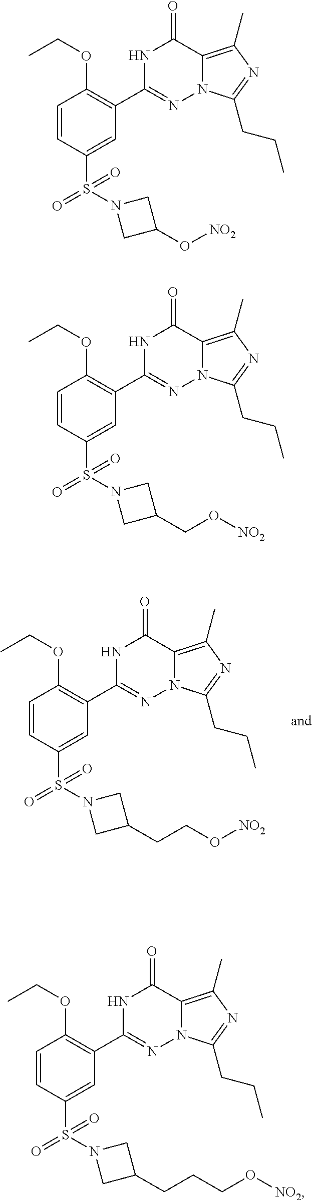
or a pharmaceutically acceptable salt, a solvate, a hydrate, a prodrug, or a stereoisomer thereof.

55. The method of claim 51, wherein R⁹ is 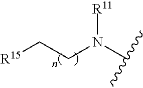
selected from:

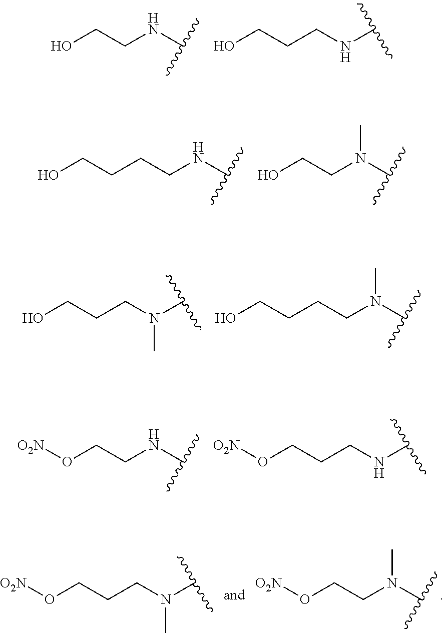

56. The method of claim 55, wherein the compound is selected from: 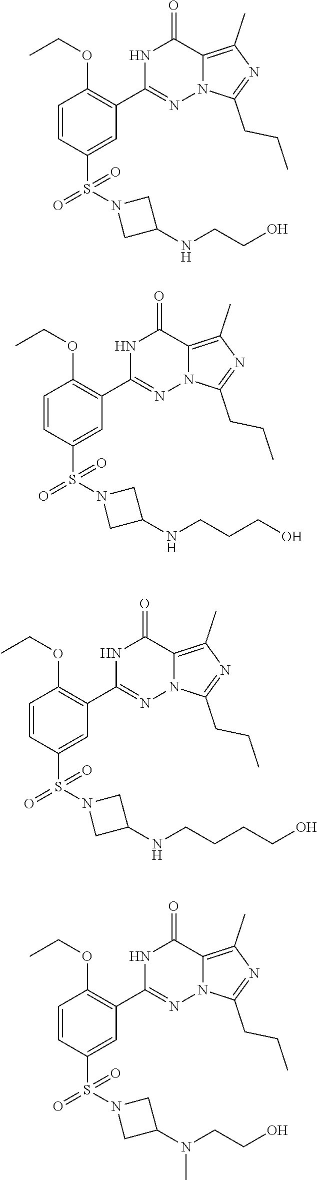 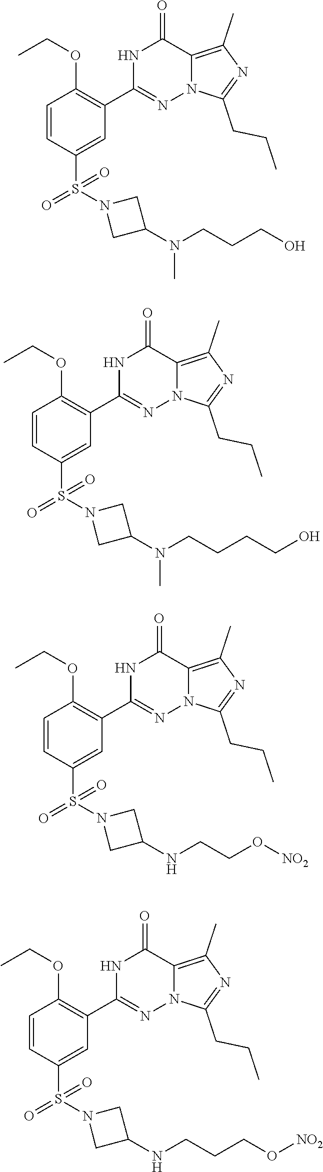 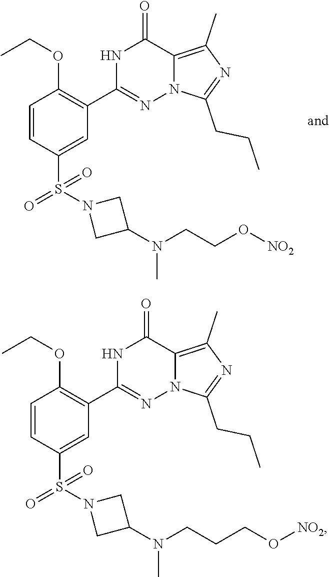
or a pharmaceutically acceptable salt, a solvate, a hydrate, a prodrug, or a stereoisomer thereof.

57. The method of claim 1, wherein the compound is any one of the compounds of Table 1, or a pharmaceutically acceptable salt, a solvate, a hydrate, a prodrug, or a stereoisomer thereof.

58. A compound, wherein the compound is selected from compounds 26 to 73 of Table 1, or a pharmaceutically acceptable salt, a solvate, a hydrate, a prodrug, or a stereoisomer thereof.

59. A pharmaceutical composition, comprising:
a therapeutically effective amount of a compound of claim 58, or a pharmaceutically acceptable salt, a solvate, a hydrate, a prodrug, or a stereoisomer thereof; and
a pharmaceutically acceptable excipient.

60-65. (canceled)
